(19) United States
(12) Patent Application Publication
(10) Pub. No.: US 2025/0063943 A1
(43) Pub. Date: Feb. 20, 2025

(54) ORGANIC LIGHT-EMITTING DEVICE HAVING HIGH EFFICIENCY AND LONG LIFESPAN

(71) Applicant: SFC CO., LTD., Cheongju-si (KR)

(72) Inventors: Seok-Bae PARK, Cheongju-si (KR); Se-Jin LEE, Cheongju-si (KR); Si-In KIM, Cheongju-si (KR); Hee-Dae KIM, Cheongju-si (KR); Yeong-Tae CHOI, Cheongju-si (KR); Yu-Rim LEE, Cheongju-si (KR); Ji-Yung KIM, Cheongju-si (KR); Seung-Soo LEE, Cheongju-si (KR); Kyungtae KIM, Cheongju-si (KR); Myeong-Jun KIM, Cheongju-si (KR); Kyeong-Hyeon KIM, Cheongju-si (KR)

(21) Appl. No.: 18/706,727

(22) PCT Filed: Nov. 14, 2022
(86) PCT No.: PCT/KR2022/017877

§ 371 Date: May 1, 2024

(30) Foreign Application Priority Data
Nov. 17, 2021 (KR) 10-2021-0158367

(51) Int. Cl. H10K 85/60; C09K 11/02; C09K 11/06; H10K 50/12; H10K 102/00

(52) U.S. Cl. CPC H10K 85/626; C09K 11/02; C09K 11/06; H10K 85/658; C09K 2211/1007; C09K 2211/1014; C09K 2211/1022; H10K 50/12; H10K 2102/351

(57) ABSTRACT

The present invention relates to an organic light-emitting device comprising, in a light-emitting layer thereof: a compound represented by [chemical formula A]; and a compound represented by any one of [chemical formula D-6] and [chemical formula D-7], wherein [chemical formula A], [chemical formula D-6], and [chemical formula D-7] are as described in the detailed description of the invention.

### TECHNICAL FIELD

The present disclosure relates to an organic light emitting diode with high efficiency and long life span and, more specifically, to an organic light emitting diode that employs specific types of host and dopant materials in a light emitting layer, whereby diode properties including high luminous efficiency and long lifespan can be achieved.

### BACKGROUND ART

Organic light-emitting diodes (OLEDs), based on self-luminescence, are used to create digital displays with the advantage of having a wide viewing angle and being able to be made thinner and lighter than liquid crystal displays. In addition, an OLED display exhibits a very fast response time. Accordingly, OLEDs find applications in the full color display field or the illumination field.

In general, the term “organic light-emitting phenomenon” refers to a phenomenon in which electrical energy is converted to light energy by means of an organic material. An organic light-emitting diode using the organic light-emitting phenomenon has a structure usually including an anode, a cathode, and an organic material layer interposed therebetween. In this regard, the organic material layer may have, for the most part, a multilayer structure consisting of different materials, for example, a hole injection layer, a hole transport layer, a light-emitting layer, an electron transport layer, and an electron injection layer in order to enhance the efficiency and stability of the organic light-emitting diode. In the organic light-emitting diode having such a structure, application of a voltage between the two electrodes injects a hole from the anode and an electron from the cathode to the organic layer. In the luminescent zone, the hole and the electron recombine to produce an exciton. When the exciton returns to the ground state from the excited state, the molecule of the organic layer emits light. Such an organic light-emitting diode is known to have characteristics such as self-luminescence, high luminance, high efficiency, low driving voltage, a wide viewing angle, high contrast, and high-speed response.

Materials used as organic layers in OLEDs may be divided according to functions into luminescent materials and charge transport materials, for example, a hole injection material, a hole transport material, an electron transport material, and an electron injection material. As for the luminescent materials, there are two main families of OLED: those based on small molecules and those employing polymers. The light-emitting mechanism forms the basis of classification of luminescent materials as fluorescent and phosphorescent materials, which use excitons in singlet and triplet states, respectively.

When a single material is employed as the luminescent material, intermolecular actions cause the maximum luminescence wavelength to shift toward a longer wavelength, resulting in a reduction in color purity and luminous efficiency due to light attenuation. In this regard, a host-dopant system may be used as a luminescent material so as to increase the color purity and the luminous efficiency through energy transfer.

This is based on the principle whereby, when a dopant which is smaller in energy band gap than a host forming a light-emitting layer is added in a small amount to the light-emitting layer, excitons are generated from the light-emitting layer and transported to the dopant, emitting light at high efficiency. Here, light with desired wavelengths can be obtained depending on the kind of the dopant because the wavelength of the host moves to the wavelength range of the dopant.

Meanwhile, studies have been made to use boron compounds as dopant compounds. With regard to related art pertaining to the use of boron compounds, reference may be made to Korean Patent No. 10-2016-0119683 A (Oct. 14, 2016), which discloses an organic light-emitting diode employing a novel polycyclic aromatic compound in which multiple aromatic rings are connected via boron and oxygen atoms. In addition, International Patent No. WO 2017/188111 (Nov. 2, 2017) disclosed an organic light emitting diode in which a compound structured to connect multiple polycondensed aromatic rings via boron and nitrogen atoms is used as a dopant in a light emitting layer while an anthracene derivative is used as a host.

Despite a variety of kinds of compounds prepared for use in light emitting layers in organic light emitting diodes including the related arts, there is still a continuing need to develop a novel compound that allows an OLED to be stably driven at a lower voltage and exhibits high efficiency, and an OLED including same.

### DISCLOSURE

### Technical Problem

Therefore, a primary technical task to be achieved by the present disclosure is to provide an organic light emitting diode (OLED) wherein a boron compound in a specific structure and an anthracene compound in a specific structure are applied as a dopant material and a host material, respectively, in a light-emitting layer of the organic light emitting diode, whereby the OLED can exhibit improved properties such as high luminous yield and longevity.

### Technical Solution

In order to accomplish the purpose, the present disclosure provides an organic light emitting diode, including: a first electrode; a second electrode facing the first electrode; and a light-emitting layer disposed between the first electrode and the second electrode, wherein the light-emitting layer contains a host and a dopant, the host including at least one of anthracene compounds represented by the following Chemical Formula A, the dopant including at least one of the compounds represented by the following Chemical Formulas D-6 to D-7:

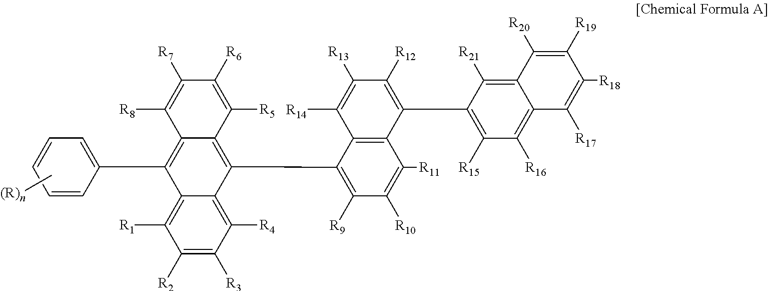

wherein,
R₁ to R₂₁, which are same or different, each are independently any one selected from the group consisting of a hydrogen atom, a deuterium atom, a substituted or unsubstituted alkyl of 1 to 30 carbon atoms, and a substituted or unsubstituted aryl of 6 to 50 carbon atoms;
R is a hydrogen atom or a deuterium atom; and
n is an integer of 1 to 5, wherein when n is 2 to 5, the corresponding R's are same or different and are independently a hydrogen atom or a deuterium atom, and

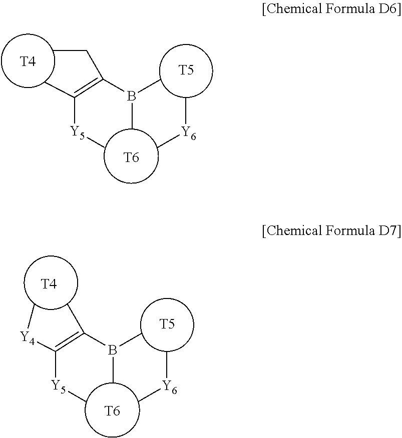

wherein,
T4 to T6 are same or different and are each independently a substituted or unsubstituted aromatic hydrocarbon ring of 6 to 50 carbon atoms, or a substituted or unsubstituted heteroaromatic ring of 2 to 40 carbon atoms;
Y₄ is any one selected from N—R₆₁, CR₆₂R₆₃, O, S, and SiR₆₄R₆₅;
Y₅ is any one selected from N—R₆₆, CR₆₇R₆₈, O, S, and SiR₆₉R₇₀;
Y₆ is any one selected from N—R₁, CR₇₂R₇₃, O, S, and SiR₇₄R₇₅;
R₆₁ to R₇₅ are same or different and are each independently any one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a cyano, and a halogen atom,
a linkage may be formed between R₆₂ and R₆₃, between R₆₄ and R₆₅, between R₆₇ and R₆₈, between R₆₉ and R₇₀, between R₇₂ and R₇₃, and/or between R₇₄ and R₇₅ to form respective aliphatic or aromatic mono- or polycyclic rings,
R₆₁ to R₇₅ may each be linked to any one ring moiety of T4 to T6 to additionally an aliphatic or aromatic mono- or polycyclic ring,
wherein the term “substituted” in the expression “substituted or unsubstituted” used for compounds of Chemical Formulas A, D6, and D7 means having at least one substituent selected from the group consisting of a deuterium atom, a cyano, a halogen atom, a hydroxy, a nitro, an alkyl of 1 to 24 carbon atoms, a halogenated alkyl of 1 to 24 carbon atoms, an alkenyl of 1 to 24 carbon atoms, an alkynyl of 1 to 24 carbon atoms, a cycloalkyl of 3 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 2 to 24 carbon atoms, an alkoxy of 1 to 24 carbon atoms, an alkylamino of 1 to 24 carbon atoms, a diarylamino of 12 to 24 carbon atoms, an diheteroarylamino of 2 to 24 carbon atoms, an aryl(heteroaryl)amino of 7 to 24 carbon atoms, an alkylsilyl of 1 to 24 carbon atoms, an arylsilyl of 6 to 24 carbon atoms, an aryloxy of 6 to 24 carbon atoms, and an arylthionyl of 6 to 24 carbon atoms.

### Advantageous Effects

The organic light emitting diode according to the present disclosure is characterized by a high emission yield and longevity, compared to conventional organic light emitting diodes.

### DESCRIPTION OF DRAWINGS

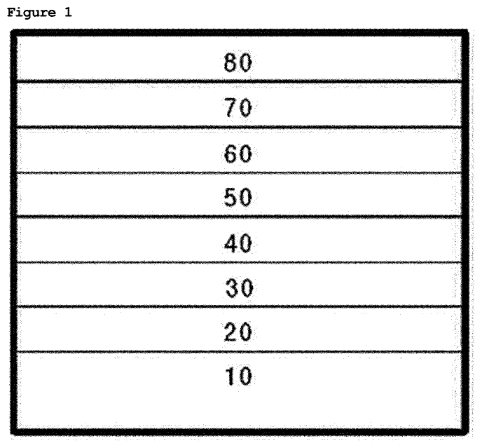

The FIG. 1 is a schematic diagram of an organic light-emitting diode according to an embodiment of the present disclosure.

### BEST MODE FOR INVENTION

Below, a detailed description will be given of the present disclosure. In each drawing of the present disclosure, sizes or scales of components may be enlarged or reduced from their actual sizes or scales for better illustration, and known components may not be depicted therein to clearly show features of the present disclosure. Therefore, the present disclosure is not limited to the drawings. When describing the principle of the embodiments of the present disclosure in detail, details of well-known functions and features may be omitted to avoid unnecessarily obscuring the presented embodiments.

In the drawing, for convenience of description, sizes of components may be exaggerated for clarity. For example, since sizes and thicknesses of components in drawings are arbitrarily shown for convenience of description, the sizes and thicknesses are not limited thereto. Furthermore, throughout the description, the terms “on” and “over” are used to refer to the relative positioning, and mean not only that one component or layer is directly disposed on another component or layer but also that one component or layer is indirectly disposed on another component or layer with a further component or layer being interposed therebetween. Also, spatially relative terms, such as “below”, “beneath”, “lower”, and “between” may be used herein for ease of description to refer to the relative positioning.

Throughout the specification, when a portion may “include” a certain constituent element, unless explicitly described to the contrary, it may not be construed to exclude another constituent element but may be construed to further include other constituent elements. Further, throughout the specification, the word “on” means positioning on or below the object portion, but does not essentially mean positioning on the lower side of the object portion based on a gravity direction.

The present disclosure provides an organic light emitting diode, including: a first electrode; a second electrode facing the first electrode; and a light-emitting layer disposed between the first electrode and the second electrode, wherein the light-emitting layer contains a host and a dopant, the host including at least one of anthracene compounds represented by the following Chemical Formula A, the dopant including at least one of the compounds represented by the following Chemical Formulas D-6 and D-7:

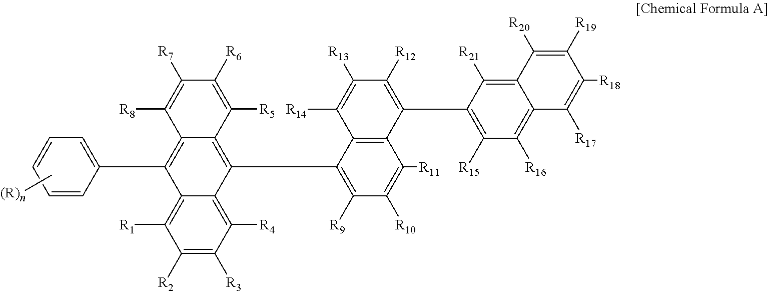

wherein,
R₁ to R₂₁, which are same or different, each are independently any one selected from the group consisting of a hydrogen atom, a deuterium atom, a substituted or unsubstituted alkyl of 1 to 30 carbon atoms, and a substituted or unsubstituted aryl of 6 to 50 carbon atoms;
R is a hydrogen atom or a deuterium atom; and
n is an integer of 1 to 5, wherein when n is 2 to 5, the corresponding R's are same or different and are independently a hydrogen atom or a deuterium atom, and

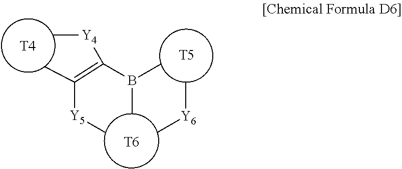

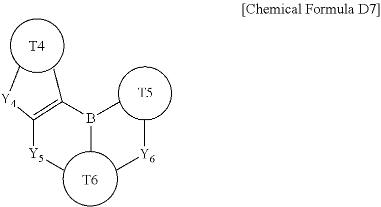

wherein,
T4 to T6 are same or different and are each independently a substituted or unsubstituted aromatic hydrocarbon ring of 6 to 50 carbon atoms, or a substituted or unsubstituted heteroaromatic ring of 2 to 40 carbon atoms;
Y₄ is any one selected from N—R₆₁, CR₆₂R₆₃, O, S, and SiR₆₄R₆₅;
Y₅ is any one selected from N—R₆₆, CR₆₇R₆₈, O, S, and SiR₆₉R₇₀;
Y₆ is any one selected from N—R₁, CR₇₂R₇₃, O, S, and SiR₇₄R₇₅;
R₆₁ to R₇₅ are same or different and are each independently any one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a cyano, and a halogen atom,
a linkage may be formed between R₆₂ and R₆₃, between R₆₄ and R₆₅, between R₆₇ and R₆₈, between R₆₉ and R₇₀, between R₇₂ and R₇₃, and/or between R₇₄ and R₇₅ to form respective aliphatic or aromatic mono- or polycyclic rings,
R₆₁ to R₇₅ may each be linked to any one ring moiety of T4 to T6 to additionally an aliphatic or aromatic mono- or polycyclic ring,
wherein the term “substituted” in the expression “substituted or unsubstituted” used for compounds of Chemical Formulas A, D6, and D7 means having at least one substituent selected from the group consisting of a deuterium atom, a cyano, a halogen atom, a hydroxy, a nitro, an alkyl of 1 to 24 carbon atoms, a halogenated alkyl of 1 to 24 carbon atoms, an alkenyl of 1 to 24 carbon atoms, an alkynyl of 1 to 24 carbon atoms, a cycloalkyl of 3 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 2 to 24 carbon atoms, an alkoxy of 1 to 24 carbon atoms, an alkylamino of 1 to 24 carbon atoms, a diarylamino of 12 to 24 carbon atoms, an diheteroarylamino of 2 to 24 carbon atoms, an aryl(heteroaryl)amino of 7 to 24 carbon atoms, an alkylsilyl of 1 to 24 carbon atoms, an arylsilyl of 6 to 24 carbon atoms, an aryloxy of 6 to 24 carbon atoms, and an arylthionyl of 6 to 24 carbon atoms.

The expression indicating the number of carbon atoms, such as “a substituted or unsubstituted alkyl of 1 to 30 carbon atoms”, “a substituted or unsubstituted aryl of 5 to 50 carbon atoms”, etc. means the total number of carbon atoms of, for example, the alkyl or aryl radical or moiety alone, exclusive of the number of carbon atoms of substituents attached thereto. For instance, a phenyl group with a butyl at the para position falls within the scope of an aryl of 6 carbon atoms, even though it is substituted with a butyl radical of 4 carbon atoms.

As used herein, the term “aryl” means an organic radical derived from an aromatic hydrocarbon by removing one hydrogen that is bonded to the aromatic hydrocarbon. The aromatic system may include a fused ring that is formed by adjacent substituents on the aryl radical.

Concrete examples of the aryl include phenyl, o-biphenyl, m-biphenyl, p-biphenyl, o-terphenyl, m-terphenyl, p-terphenyl, naphthyl, anthryl, phenanthryl, pyrenyl, indenyl, fluorenyl, tetrahydronaphthyl, perylenyl, chrysenyl, naphthacenyl, and fluoranthenyl. At least one hydrogen atom of the aryl may be substituted by a deuterium atom, a halogen atom, a hydroxy, a nitro, a cyano, a silyl, an amino (—NH₂, —NH(R), —N(R′)(R″) wherein R′ and R″ are each independently an alkyl of 1 to 10 carbon atoms, in this case, called “alkylamino”), an amidino, a hydrazine, a hydrazone, a carboxyl, a sulfonic acid, a phosphoric acid, an alkyl of 1 to 24 carbon atoms, a halogenated alkyl of 1 to 24 carbon atoms, an alkenyl of 1 to 24 carbon atoms, an alkynyl of 1 to 24 carbon atoms, a heteroalkyl of 1 to 24 carbon atoms, an aryl of 6 to 24 carbon atoms, an arylalkyl of 6 to 24 carbon atoms, a heteroaryl of 2 to 24 carbon atoms, or a heteroarylalkyl of 2 to 24 carbon atoms.

The substituent “heteroaryl” used in the compound of the present disclosure means a hetero aromatic radical of 2 to 24 carbon atoms, bearing one to three heteroatoms selected from among N, O, P, Si, S, Ge, Se, and Te. In the aromatic radical, two or more rings may be fused. One or more hydrogen atoms on the heteroaryl may be substituted by the same substituents as on the aryl.

In addition, the term “heteroaromatic ring”, as used herein, refers to an aromatic hydrocarbon ring bearing at least one heteroatom as aromatic ring member. In the heteroaromatic ring, one to three carbon atoms of the aromatic hydrocarbon may be substituted by at least one selected particularly from N, O, P, Si, S, Ge, Se, and Te.

As used herein, the term “alkyl” refers to an alkane missing one hydrogen atom and includes linear or branched structures. Examples of the alkyl substituent useful in the present disclosure include methyl, ethyl, propyl, isopropyl, isobutyl, sec-butyl, tert-butyl, pentyl, iso-amyl, hexyl, and the like. At least one hydrogen atom of the alkyl may be substituted by the same substituent as in the aryl.

The term “cyclo” as used in substituents of the compounds of the present disclosure, such as cycloalkyl, etc., refers to a structure responsible for a mono- or polycyclic ring of saturated hydrocarbons. Concrete examples of cycloalkyl radicals include cyclopropyl, cyclobutyl, cyclopentyl, cyclohexyl, methylcyclopentyl, methylcyclohexyl, ethylcyclopentyl, ethylcyclohexyl, adamantyl, dicyclopentadienyl, decahydronaphthyl, norbornyl, bornyl, isobornyl, and so on. One or more hydrogen atoms on the cycloalkyl may be substituted by the same substituents as on the aryl.

The term “alkoxy” as used in the compounds of the present disclosure refers to an alkyl or cycloalkyl singularly bonded to oxygen. Concrete examples of the alkoxy include methoxy, ethoxy, propoxy, isobutoxy, sec-butoxy, pentoxy, iso-amyloxy, hexyloxy, cyclobutyloxy, cyclopentyloxy, adamantyloxy, dicyclopentyloxy, bornyloxy, isobornyloxy, and the like. One or more hydrogen atoms on the alkoxy may be substituted by the same substituents as on the aryl.

Concrete examples of the arylalkyl used in the compounds of the present disclosure include phenylmethyl (benzyl), phenylethyl, phenylpropyl, naphthylmethyl, naphthylethyl, and the like. One or more hydrogen atoms on the arylalkyl may be substituted by the same substituents as on the aryl.

As used herein, the term “alkenyl” refers to an alkyl substituent containing a carbon-carbon double bond between two carbon atoms and the term “alkynyl” refers to an alkyl substituent containing a carbon-carbon triple bond between two carbon atoms.

As used herein, the term “alkylene” refers to an organic radical regarded as derived from an alkane by removal two hydrogen atoms from one carbon atom for methylene or different carbon atoms for ethylene or higher, such as propylene, isopropylene, isobutylene, sec-butylene, tert-butylene, pentylene, iso-amylene, hexylene, and the like. One or more hydrogen atoms on the alkylene may be substituted by the same substituents as on the aryl.

Concrete examples of the silyl radicals used in the compounds of the present disclosure include trimethylsilyl, triethylsilyl, triphenylsilyl, trimethoxysilyl, dimethoxyphenylsilyl, diphenylmethylsilyl, diphenylvinylsilyl, methylcyclobutylsilyl, and dimethylfurylsilyl. One or more hydrogen atoms on the silyl may be substituted by the same substituents as on the aryl.

The term “substituted” in the expression “substituted or unsubstituted” used for the compound of Chemical Formula A means having at least one substituent selected from the group consisting of a deuterium atom, a cyano, a halogen, a hydroxy, a nitro, an alkyl of 1 to 12 carbon atoms, a halogenated alkyl of 1 to 12 carbon atoms, an alkenyl of 2 to 12 carbon atoms, an alkynyl of 2 to 12 carbon atoms, a cycloalkyl of 3 to 12 carbon atoms, a heteroalkyl of 1 to 12 carbon atoms, an aryl of 6 to 18 carbon atoms, an arylalkyl of 7 to 20 carbon atoms, an alkylaryl of 7 to 20 carbon atoms, a heteroaryl of 2 to 18 carbon atoms, a heteroarylalkyl of 2 to 18 carbon atoms, an alkoxy of 1 to 12 carbon atoms, an alkylamino of 1 to 12 carbon atoms, a diarylamino of 12 to 20 carbon atoms, a diheteroarylamino of 5 to 20 carbon atoms, an aryl(heteroaryl)amino of 9 to 20 carbon atoms, an alkylsilyl of 1 to 12 carbon atoms, an arylsilyl of 6 to 20 carbon atoms, an aryloxy of 6 to 18 carbon atoms, and an arylthionyl of 6 to 18 carbon atoms.

In the compound represented by Chemical Formula A according to the present disclosure, the anthracene ring moiety has a deuterium-substituted or unsubstituted phenyl as a substituent on the carbon atom at position 9 thereof and a 1-naphthylenyl as a linker on the carbon atom at position 10 thereof, with a 2-naphthyl radical bonded to the carbon atom at position 5 of the 1-naphthylenyl linker, wherein the aromatic carbons within the anthracene ring moiety, 1-naphthylenyl linker, and 2-naphthyl radical may be bonded to any one substituent selected from a hydrogen atom, a deuterium atom, a substituted or unsubstituted alkyl of 1 to 30 carbon atoms, and a substituted or unsubstituted aryl of 6 to 50 carbon atoms.

According to an embodiment, in Chemical Formula A, R may be a deuterium atom and n may be 5.

According to an embodiment, R₁ to R₂₁ in Chemical Formula A may each be a hydrogen atom or a deuterium atom. In this case, a deuterium atom may be set for each of four or more of R₁ to R₂₁, particularly for each of five or more, more particularly for each of six or more, much more particularly for each of seven or more, even much more particularly for each of eight or more.

According to an embodiment, among R₁ to R₈ in Chemical Formula A, four or more, particularly five or more, more particularly six or more, much more particularly seven or more, and even much more particularly eight may each be a deuterium atom.

In an embodiment, the anthracene derivative represented by Chemical Formula A may have a degree of deuteration of 30% or higher, particularly 35% or higher, more particularly 40% or higher, much more particularly 45% or higher, even more particularly 50% or higher, 55% or higher, 60% or higher, or even much more particularly 65% or higher.

As for a degree of deuteration applied in the description, “a deuterated derivative” of compound X refers to the same structure of compound X with the exception that at least one deuterium atom (D), instead of a hydrogen atom (H), is bonded to a carbon atom, a nitrogen atom, or an oxygen atom within compound X.

As used herein, the term “yy % deuterated” or “a degree of deuteration of yy %” means that deuterium atoms bonded directly to carbon, nitrogen, and oxygen atoms within compound X exist at yy %, based on the total number of hydrogen and deuterium atoms bonded directly thereto.

For example, the benzene compound C₆H₄D₂, which has two deuterium atoms and four hydrogen atoms, is 33% deuterated because the degree of deuteration thereof is calculated as 2/(4+2)×100=33%.

When the anthracene derivative compound of the present disclosure is deuterated, the degree of deuteration is expressed as a percentage of the deuterium atoms bonded directly to the carbon atoms within the anthracene derivative relative to all hydrogen and deuterium atoms bonded directly to the carbon atoms within the anthracene derivative.

For the anthracene derivative represented by the following Chemical Formula 1, for example, there is a total of 10 deuterium atoms from 5 deuterium atoms on the phenyl radical bonded to the anthracene moiety and 5 deuterium atoms on the phenyl radical bonded to the dibenzofuran moiety while there are 8 hydrogen atoms on the anthracene moiety and 6 hydrogen atoms on the two 6-membered aromatic rings of the dibenzofuran moiety. Thus, the degree of deuteration is expressed as 100 10/(10+8+6)=41.7%.

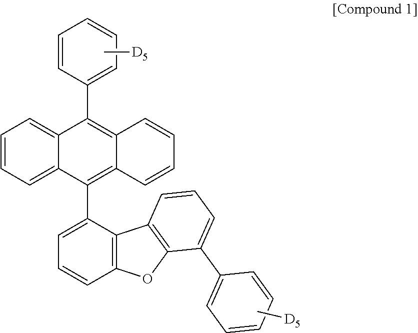

For a specific substituent, an average degree of deuteration may be given because degrees of deuteration may differ from one substituent to another.

An example is given by a partially deuterium-substituted anthracene radical. When a deuterium atom is intended to be substituted on all carbon atoms in an anthracene, the resulting anthracene derivative may be deuterated fully or partially according to reaction conditions. That is, there may be a mixture including fully deuterated anthracene molecules and partially deuterated anthracene molecules. It is very difficult to separate the fully deuterated anthracene molecules and the partially deuterate anthracene molecules from each other. In this case, the degree of deuteration can be calculated according to the entire structural formula with reference to an average degree of deuteration.

According to the present disclosure, the use of the anthracene derivative represented by Chemical Formula A as a material for a light-emitting layer in an organic light-emitting diode can further improve the lifespan of the organic light-emitting diode.

Concrete examples of the compound represented by Chemical Formula A according to the present disclosure include compounds represented by [A-1] to [A-153] below, but are not limited thereto:

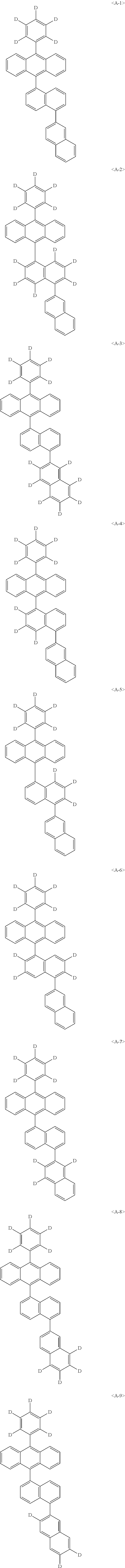

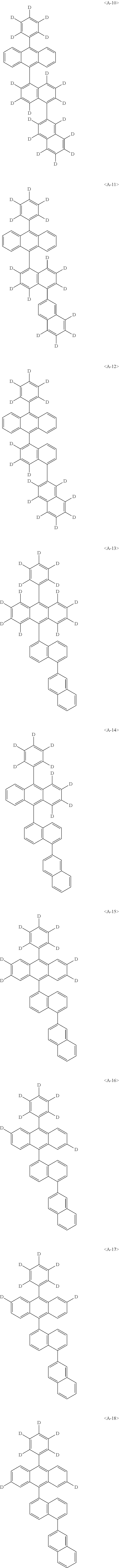

[chemical structure]

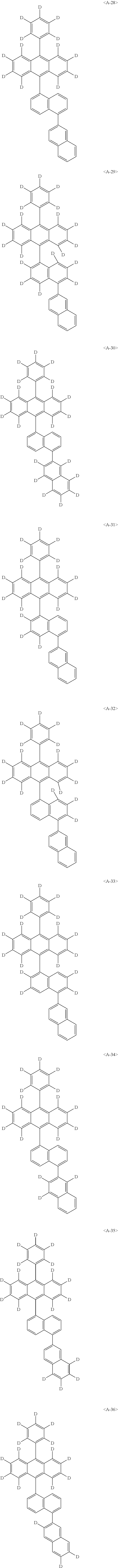

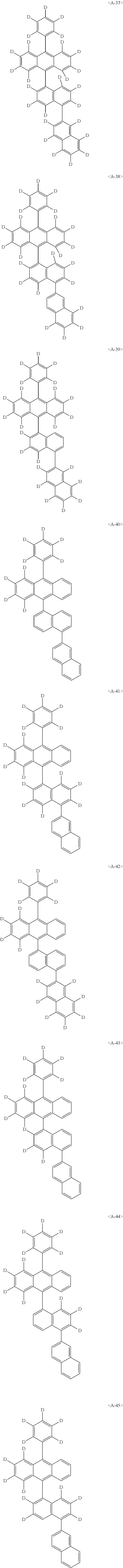

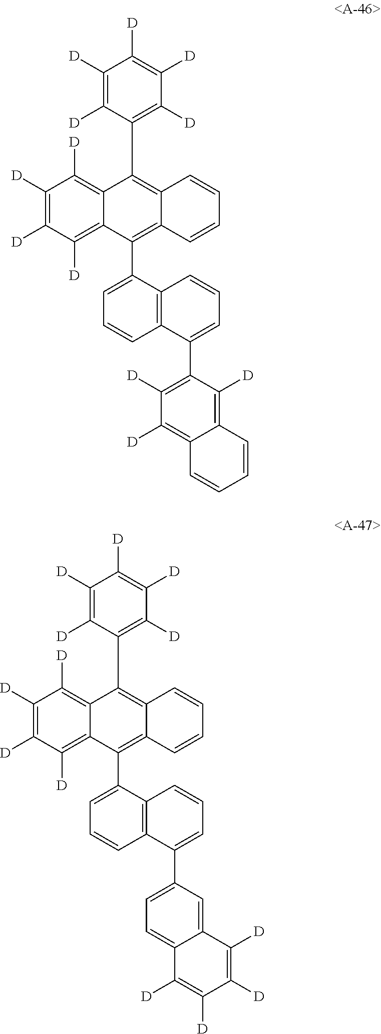 [SMILES: [2H]c1c([2H])c([2H])c(-c2c3ccccc3c(-c3cccc4c(-c5c([2H])c([2H])c6ccccc6c5[2H])cccc34)c3c([2H])c([2H])c([2H])c([2H])c23)c([2H])c1[2H] ; [2H]c1c([2H])c([2H])c(-c2c3ccccc3c(-c3cccc4c(-c5ccc6c([2H])c([2H])c([2H])c([2H])c6c5)cccc34)c3c([2H])c([2H])c([2H])c([2H])c23)c([2H])c1[2H]]

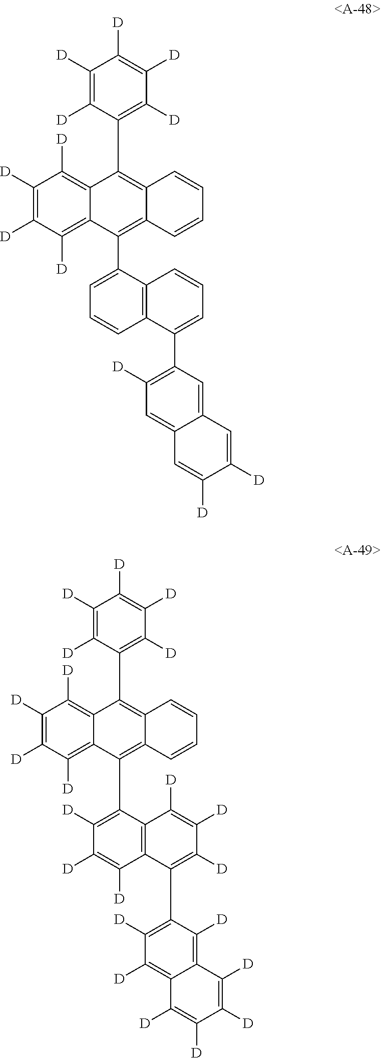 [SMILES: [2H]c1cc2cc([2H])c(-c3cccc4c(-c5c6ccccc6c(-c6c([2H])c([2H])c([2H])c([2H])c6[2H])c6c([2H])c([2H])c([2H])c([2H])c56)cccc34)cc2cc1[2H] ; [2H]c1c([2H])c([2H])c(-c2c3ccccc3c(-c3c([2H])c([2H])c([2H])c4c(-c5c([2H])c([2H])c6c([2H])c([2H])c([2H])c([2H])c6c5[2H])c([2H])c([2H])c([2H])c34)c3c([2H])c([2H])c([2H])c([2H])c23)c([2H])c1[2H]]

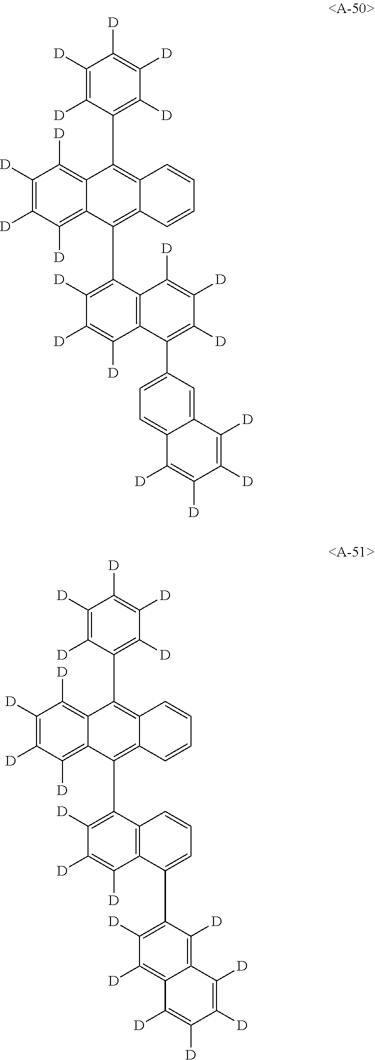 [SMILES: [2H]c1c([2H])c([2H])c(-c2c3ccccc3c(-c3c([2H])c([2H])c([2H])c4c(-c5ccc6c([2H])c([2H])c([2H])c([2H])c6c5)c([2H])c([2H])c([2H])c34)c3c([2H])c([2H])c([2H])c([2H])c23)c([2H])c1[2H] ; [2H]c1c([2H])c([2H])c(-c2c3ccccc3c(-c3c([2H])c([2H])c([2H])c4c(-c5c([2H])c([2H])c6c([2H])c([2H])c([2H])c([2H])c6c5[2H])cccc34)c3c([2H])c([2H])c([2H])c([2H])c23)c([2H])c1[2H]]

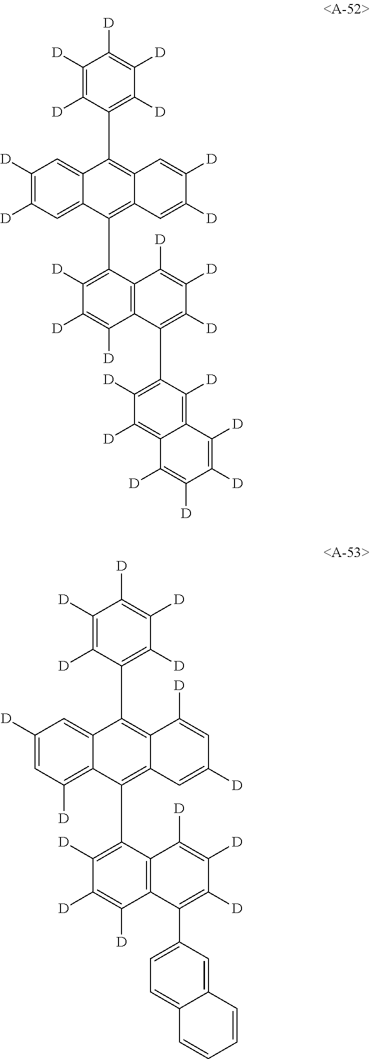 [SMILES: [2H]c1cc2c(-c3c([2H])c([2H])c([2H])c([2H])c3[2H])c3cc([2H])c([2H])cc3c(-c3c([2H])c([2H])c([2H])c4c(-c5c([2H])c([2H])c6c([2H])c([2H])c([2H])c([2H])c6c5[2H])c([2H])c([2H])c([2H])c34)c2cc1[2H] ; [2H]c1cc([2H])c2c(-c3c([2H])c([2H])c([2H])c([2H])c3[2H])c3cc([2H])cc([2H])c3c(-c3c([2H])c([2H])c([2H])c4c(-c5ccc6ccccc6c5)c([2H])c([2H])c([2H])c34)c2c1]

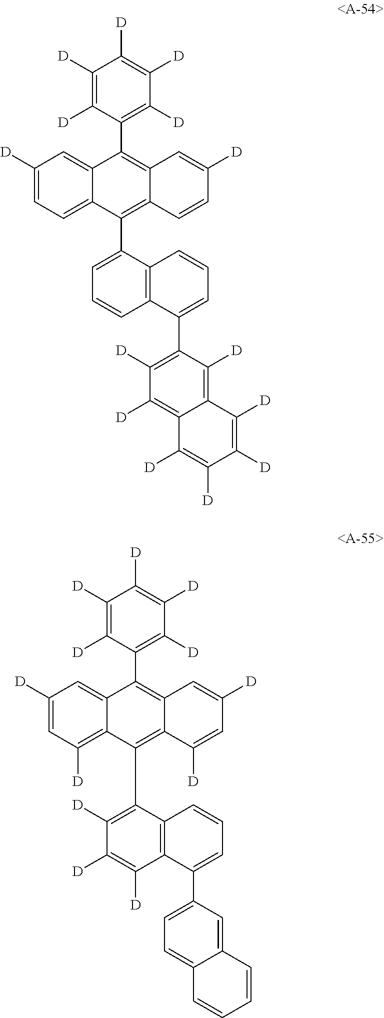 [SMILES: [2H]c1ccc2c(-c3cccc4c(-c5c([2H])c([2H])c6c([2H])c([2H])c([2H])c([2H])c6c5[2H])cccc34)c3ccc([2H])cc3c(-c3c([2H])c([2H])c([2H])c([2H])c3[2H])c2c1 ; [2H]c1cc([2H])c2c(-c3c([2H])c([2H])c([2H])c4c(-c5ccc6ccccc6c5)cccc34)c3c([2H])cc([2H])cc3c(-c3c([2H])c([2H])c([2H])c([2H])c3[2H])c2c1]

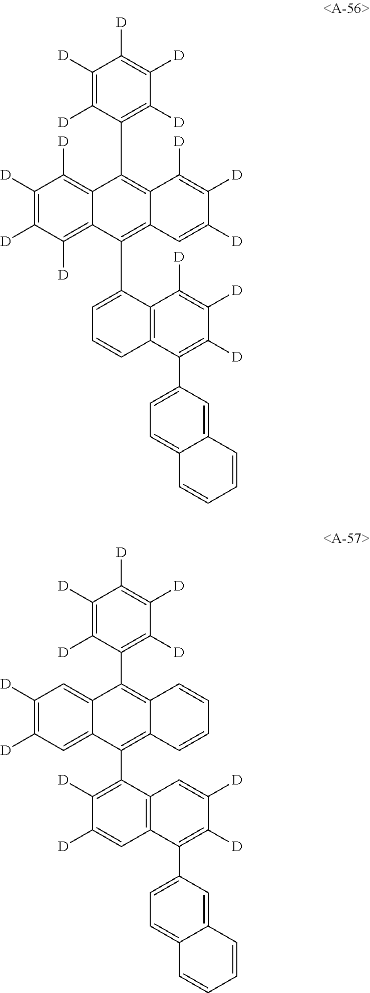 [SMILES: [2H]c1cc2c(-c3cccc4c(-c5ccc6ccccc6c5)c([2H])c([2H])c([2H])c34)c3c([2H])c([2H])c([2H])c([2H])c3c(-c3c([2H])c([2H])c([2H])c([2H])c3[2H])c2c([2H])c1[2H] ; [2H]c1cc2c(-c3c([2H])c([2H])c([2H])c([2H])c3[2H])c3ccccc3c(-c3c([2H])c([2H])cc4c(-c5ccc6ccccc6c5)c([2H])c([2H])cc34)c2cc1[2H]]

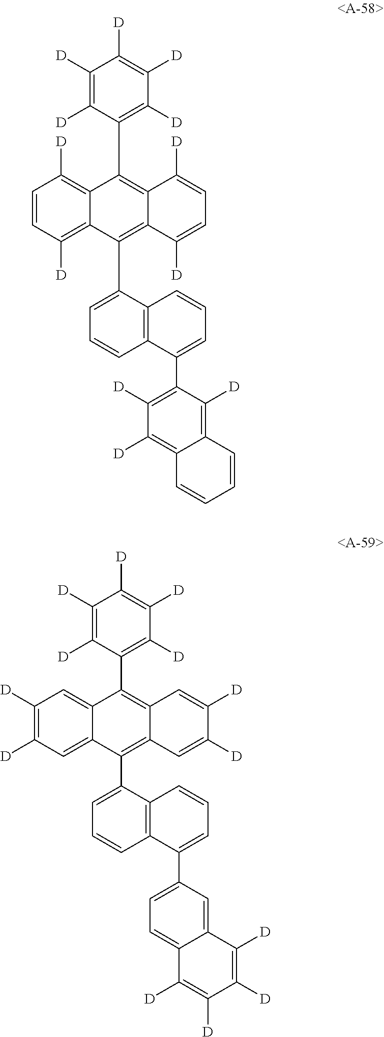 [SMILES: [2H]c1c([2H])c([2H])c(-c2c3c([2H])ccc([2H])c3c(-c3cccc4c(-c5c([2H])c([2H])c6ccccc6c5[2H])cccc34)c3c([2H])ccc([2H])c23)c([2H])c1[2H] ; [2H]c1cc2c(-c3c([2H])c([2H])c([2H])c([2H])c3[2H])c3cc([2H])c([2H])cc3c(-c3cccc4c(-c5ccc6c([2H])c([2H])c([2H])c([2H])c6c5)cccc34)c2cc1[2H]]

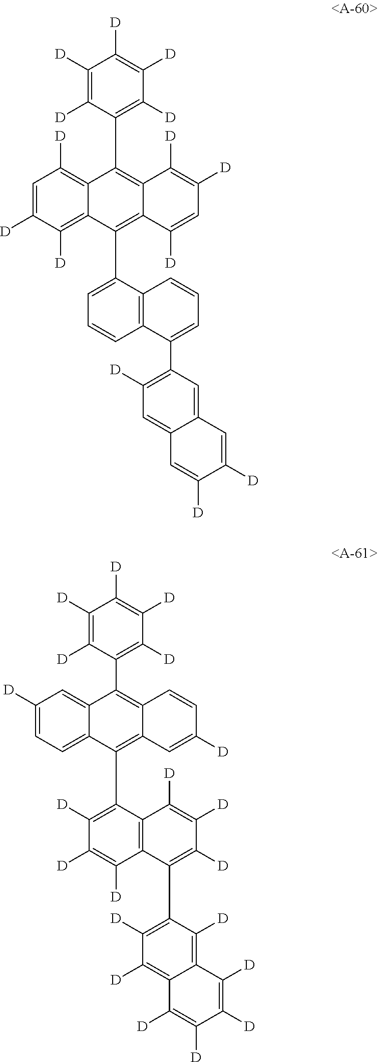 [SMILES: [2H]c1cc2cc([2H])c(-c3cccc4c(-c5c6c([2H])cc([2H])c([2H])c6c(-c6c([2H])c([2H])c([2H])c([2H])c6[2H])c6c([2H])cc([2H])c([2H])c56)cccc34)cc2cc1[2H] ; [2H]c1ccc2c(-c3c([2H])c([2H])c([2H])c4c(-c5c([2H])c([2H])c6c([2H])c([2H])c([2H])c([2H])c6c5[2H])c([2H])c([2H])c([2H])c34)c3cc([2H])ccc3c(-c3c([2H])c([2H])c([2H])c([2H])c3[2H])c2c1]

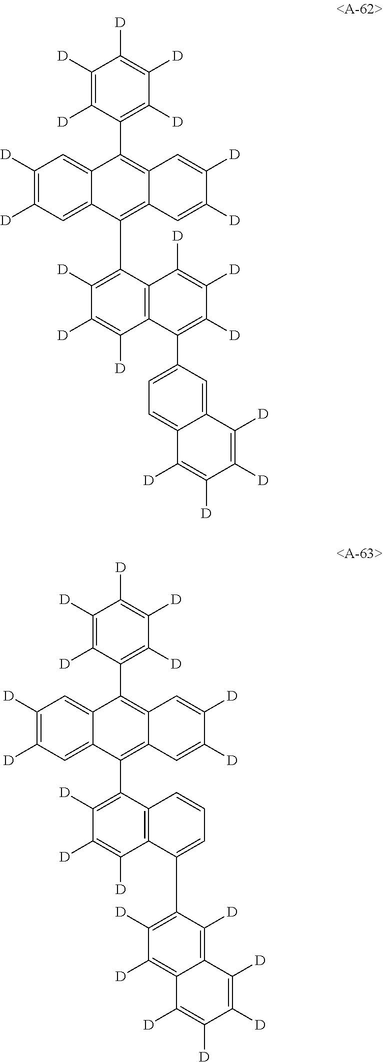 [SMILES: [2H]c1cc2c(-c3c([2H])c([2H])c([2H])c([2H])c3[2H])c3cc([2H])c([2H])cc3c(-c3c([2H])c([2H])c([2H])c4c(-c5ccc6c([2H])c([2H])c([2H])c([2H])c6c5)c([2H])c([2H])c([2H])c34)c2cc1[2H] ; [2H]c1cc2c(-c3c([2H])c([2H])c([2H])c([2H])c3[2H])c3cc([2H])c([2H])cc3c(-c3c([2H])c([2H])c([2H])c4c(-c5c([2H])c([2H])c6c([2H])c([2H])c([2H])c([2H])c6c5[2H])cccc34)c2cc1[2H]]

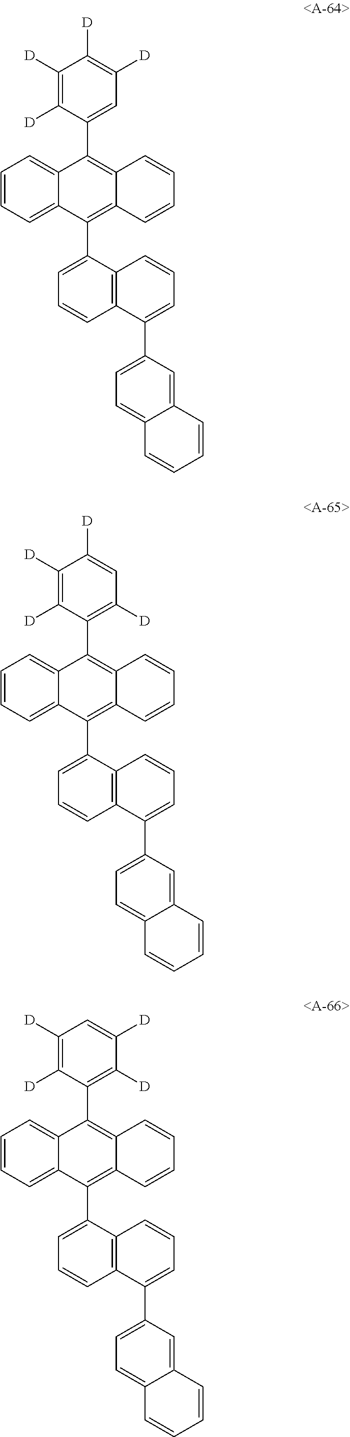 [SMILES: [2H]c1cc(-c2c3ccccc3c(-c3cccc4c(-c5ccc6ccccc6c5)cccc34)c3ccccc23)c([2H])c([2H])c1[2H] ; [2H]c1cc([2H])c(-c2c3ccccc3c(-c3cccc4c(-c5ccc6ccccc6c5)cccc34)c3ccccc23)c([2H])c1[2H] ; [2H]c1cc([2H])c([2H])c(-c2c3ccccc3c(-c3cccc4c(-c5ccc6ccccc6c5)cccc34)c3ccccc23)c1[2H]]

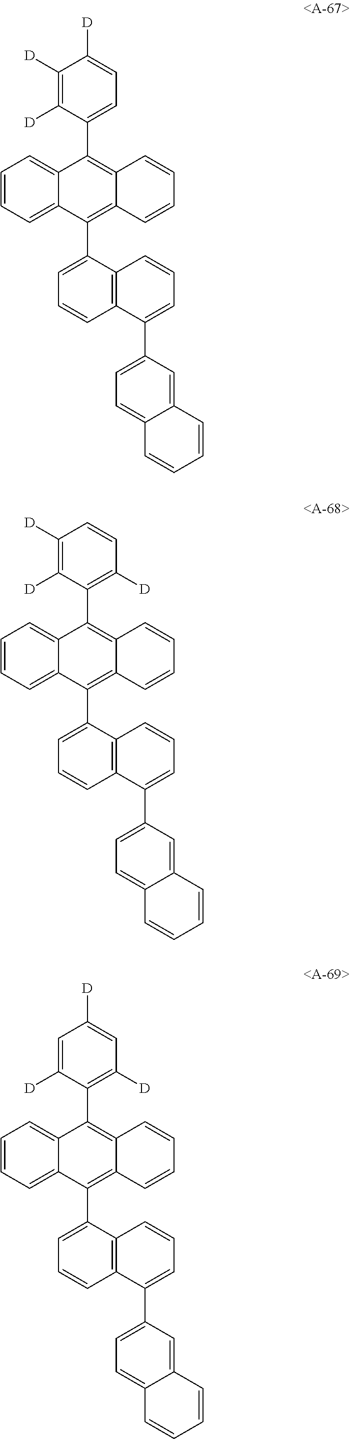 [SMILES: [2H]c1ccc(-c2c3ccccc3c(-c3cccc4c(-c5ccc6ccccc6c5)cccc34)c3ccccc23)c([2H])c1[2H] ; [2H]c1ccc([2H])c(-c2c3ccccc3c(-c3cccc4c(-c5ccc6ccccc6c5)cccc34)c3ccccc23)c1[2H] ; [2H]c1cc([2H])c(-c2c3ccccc3c(-c3cccc4c(-c5ccc6ccccc6c5)cccc34)c3ccccc23)c([2H])c1]

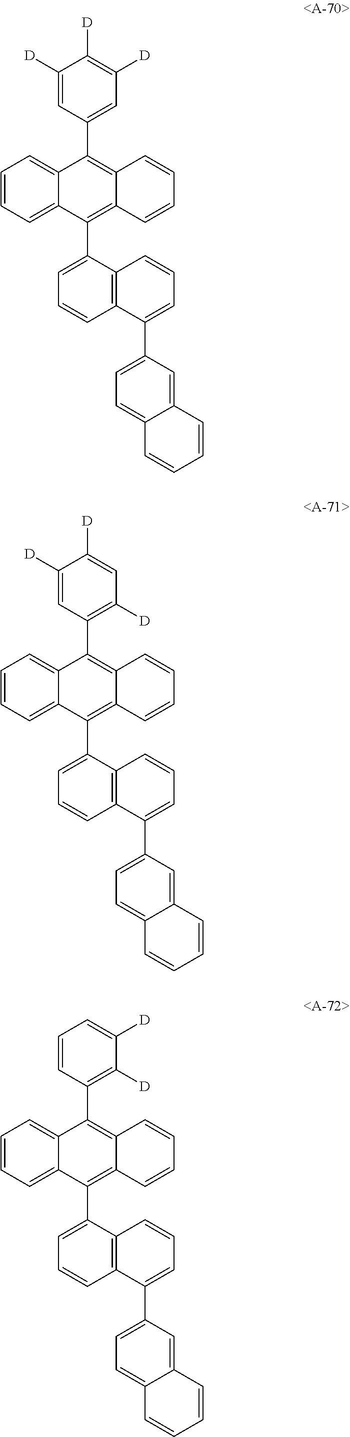 [SMILES: [2H]c1cc(-c2c3ccccc3c(-c3cccc4c(-c5ccc6ccccc6c5)cccc34)c3ccccc23)cc([2H])c1[2H] ; [2H]c1cc([2H])c(-c2c3ccccc3c(-c3cccc4c(-c5ccc6ccccc6c5)cccc34)c3ccccc23)cc1[2H] ; [2H]c1cccc(-c2c3ccccc3c(-c3cccc4c(-c5ccc6ccccc6c5)cccc34)c3ccccc23)c1[2H]]

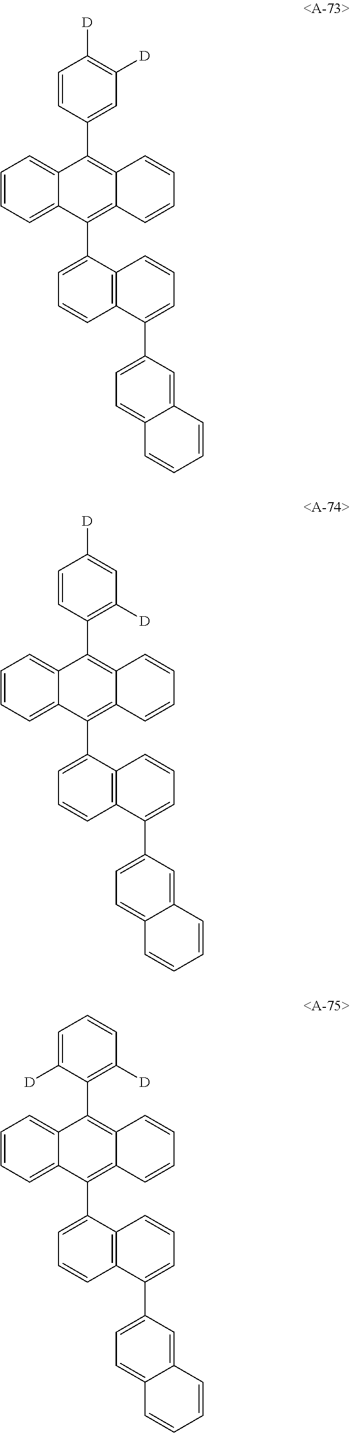 [SMILES: [2H]c1ccc(-c2c3ccccc3c(-c3cccc4c(-c5ccc6ccccc6c5)cccc34)c3ccccc23)cc1[2H] ; [2H]c1ccc(-c2c3ccccc3c(-c3cccc4c(-c5ccc6ccccc6c5)cccc34)c3ccccc23)c([2H])c1 ; [2H]c1cccc([2H])c1-c1c2ccccc2c(-c2cccc3c(-c4ccc5ccccc5c4)cccc23)c2ccccc12]

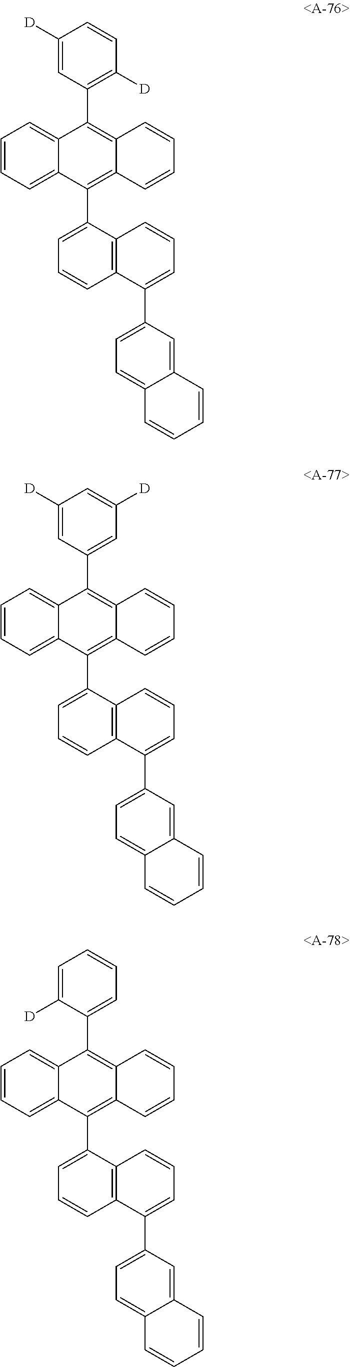 [SMILES: [2H]c1ccc([2H])c(-c2c3ccccc3c(-c3cccc4c(-c5ccc6ccccc6c5)cccc34)c3ccccc23)c1 ; [2H]c1cc([2H])cc(-c2c3ccccc3c(-c3cccc4c(-c5ccc6ccccc6c5)cccc34)c3ccccc23)c1 ; [2H]c1ccccc1-c1c2ccccc2c(-c2cccc3c(-c4ccc5ccccc5c4)cccc23)c2ccccc12]

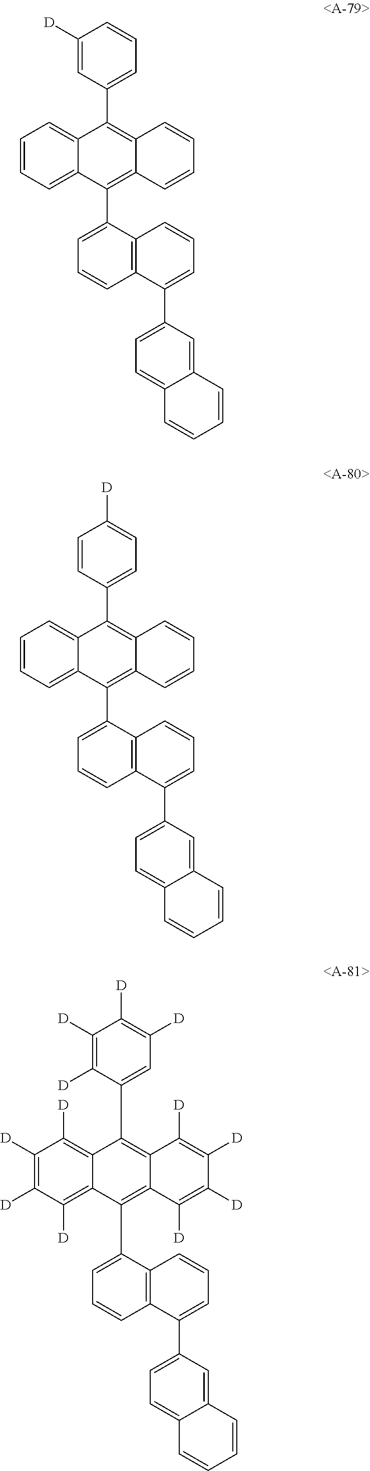 [SMILES: [2H]c1cccc(-c2c3ccccc3c(-c3cccc4c(-c5ccc6ccccc6c5)cccc34)c3ccccc23)c1 ; [2H]c1ccc(-c2c3ccccc3c(-c3cccc4c(-c5ccc6ccccc6c5)cccc34)c3ccccc23)cc1 ; [2H]c1cc(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c([2H])c1[2H]]

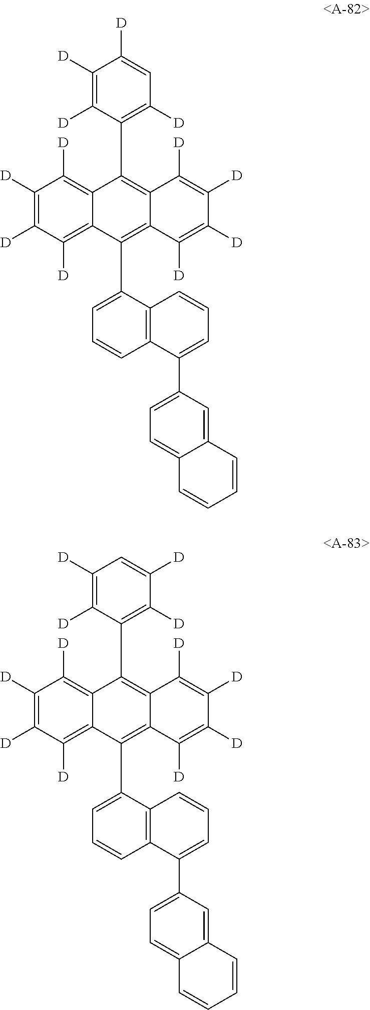 [SMILES: [2H]c1cc([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1[2H] ; [2H]c1cc([2H])c([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c1[2H]]

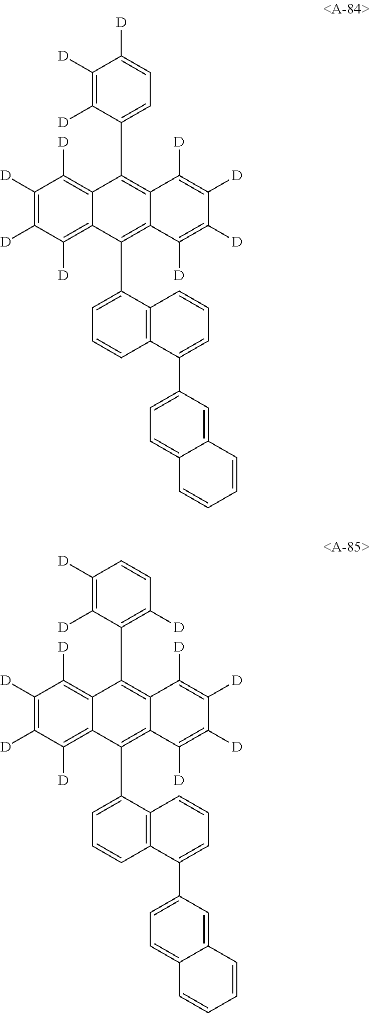 [SMILES: [2H]c1ccc(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1[2H] ; [2H]c1ccc([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c1[2H]]

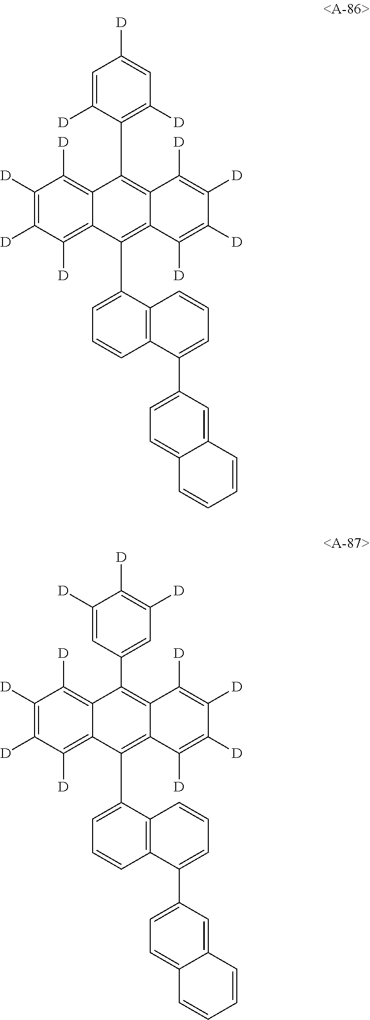 [SMILES: [2H]c1cc([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1 ; [2H]c1cc(-c2c3c([2H])c([2H])c([2H])c([2H])c3c(-c3cccc4c(-c5ccc6ccccc6c5)cccc34)c3c([2H])c([2H])c([2H])c([2H])c23)cc([2H])c1[2H]]

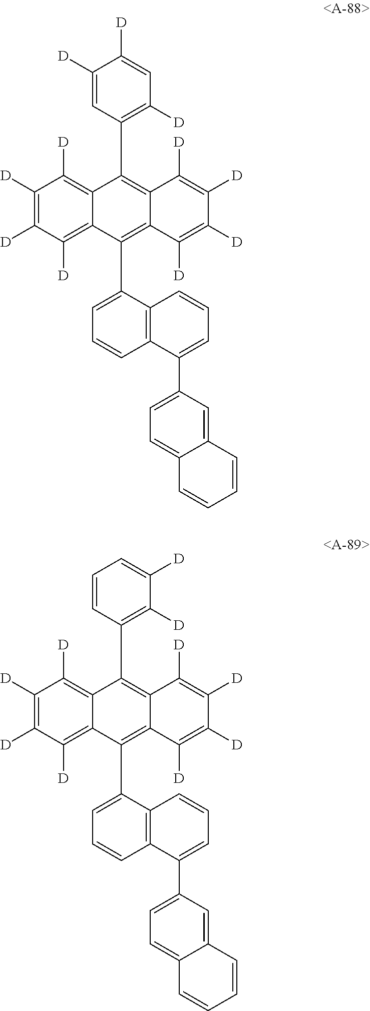 [SMILES: [2H]c1cc([2H])c(-c2c3c([2H])c([2H])c([2H])c([2H])c3c(-c3cccc4c(-c5ccc6ccccc6c5)cccc34)c3c([2H])c([2H])c([2H])c([2H])c23)cc1[2H] ; [2H]c1cccc(-c2c3c([2H])c([2H])c([2H])c([2H])c3c(-c3cccc4c(-c5ccc6ccccc6c5)cccc34)c3c([2H])c([2H])c([2H])c([2H])c23)c1[2H]]

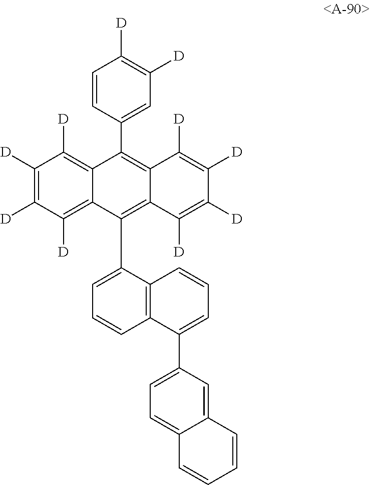 [SMILES: [2H]c1ccc(-c2c3c([2H])c([2H])c([2H])c([2H])c3c(-c3cccc4c(-c5ccc6ccccc6c5)cccc34)c3c([2H])c([2H])c([2H])c([2H])c23)cc1[2H]]

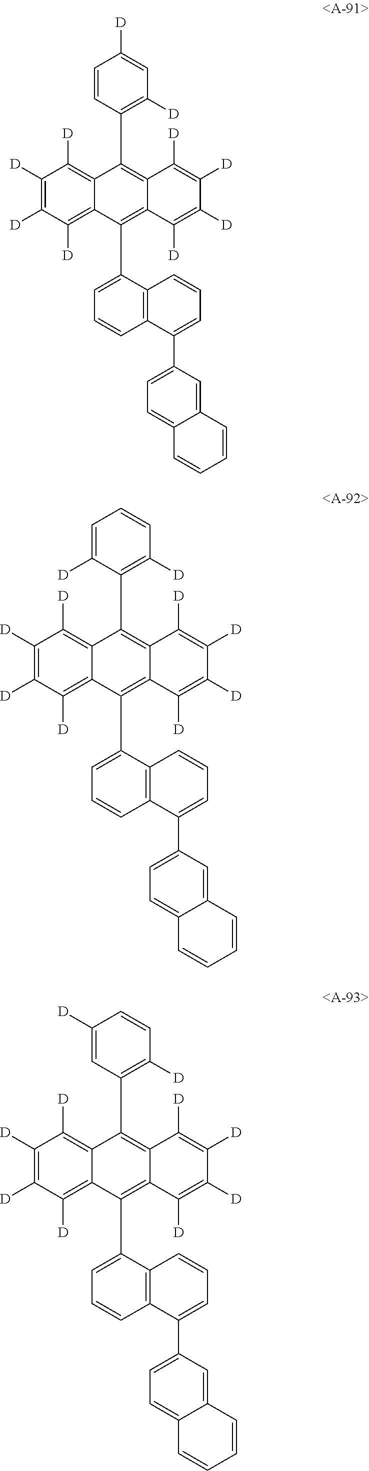 [SMILES: [2H]c1ccc(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1 ; [2H]c1cccc([2H])c1-c1c2c([2H])c([2H])c([2H])c([2H])c2c(-c2cccc3c(-c4ccc5ccccc5c4)cccc23)c2c([2H])c([2H])c([2H])c([2H])c12 ; [2H]c1ccc([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c1]

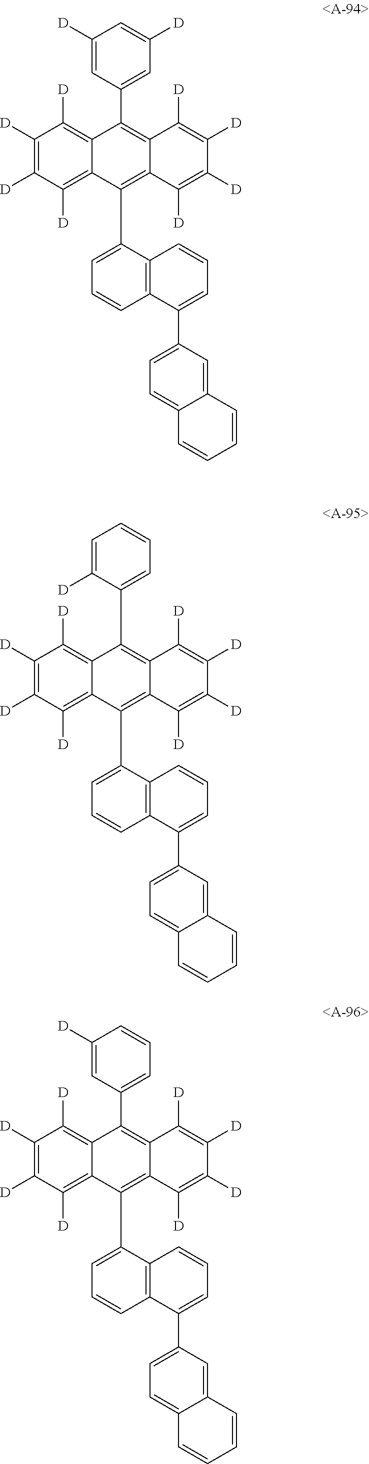 [SMILES: [2H]c1cc([2H])cc(-c2c3c([2H])c([2H])c([2H])c([2H])c3c(-c3cccc4c(-c5ccc6ccccc6c5)cccc34)c3c([2H])c([2H])c([2H])c([2H])c23)c1 ; [2H]c1ccccc1-c1c2c([2H])c([2H])c([2H])c([2H])c2c(-c2cccc3c(-c4ccc5ccccc5c4)cccc23)c2c([2H])c([2H])c([2H])c([2H])c12 ; [2H]c1cccc(-c2c3c([2H])c([2H])c([2H])c([2H])c3c(-c3cccc4c(-c5ccc6ccccc6c5)cccc34)c3c([2H])c([2H])c([2H])c([2H])c23)c1]

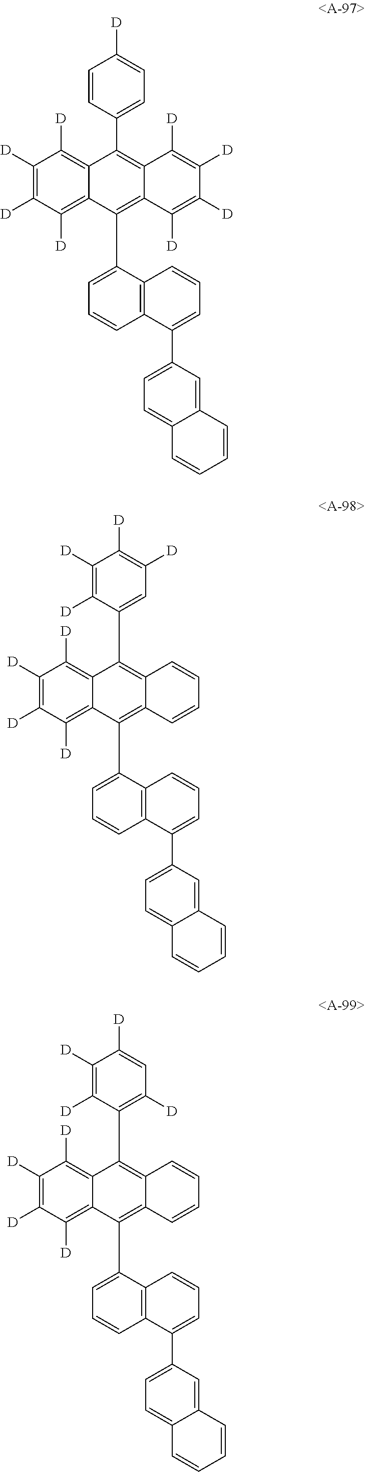 [SMILES: [2H]c1ccc(-c2c3c([2H])c([2H])c([2H])c([2H])c3c(-c3cccc4c(-c5ccc6ccccc6c5)cccc34)c3c([2H])c([2H])c([2H])c([2H])c23)cc1 ; [2H]c1cc(-c2c3ccccc3c(-c3cccc4c(-c5ccc6ccccc6c5)cccc34)c3c([2H])c([2H])c([2H])c([2H])c23)c([2H])c([2H])c1[2H] ; [2H]c1cc([2H])c(-c2c3ccccc3c(-c3cccc4c(-c5ccc6ccccc6c5)cccc34)c3c([2H])c([2H])c([2H])c([2H])c23)c([2H])c1[2H]]

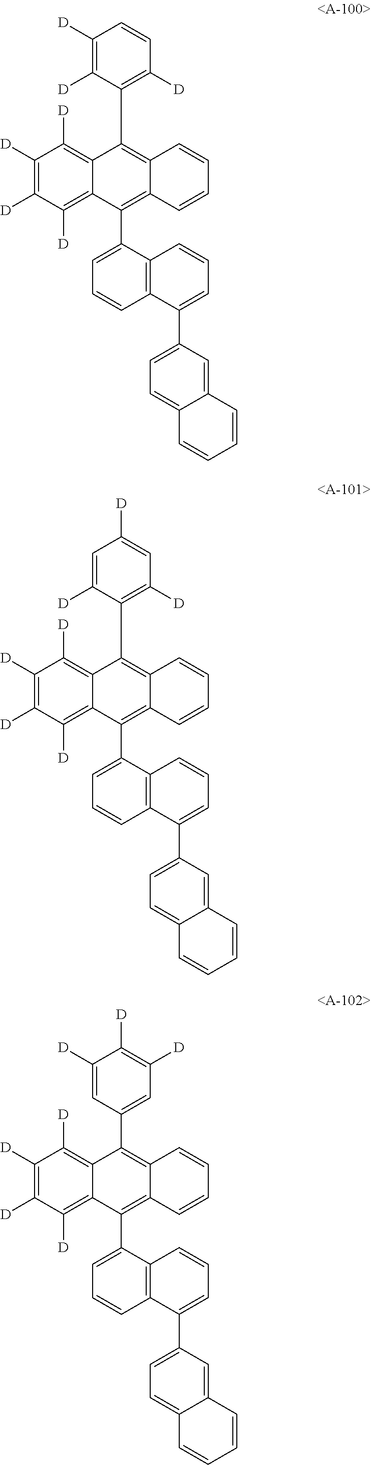 [SMILES: [2H]c1ccc([2H])c(-c2c3ccccc3c(-c3cccc4c(-c5ccc6ccccc6c5)cccc34)c3c([2H])c([2H])c([2H])c([2H])c23)c1[2H] ; [2H]c1cc([2H])c(-c2c3ccccc3c(-c3cccc4c(-c5ccc6ccccc6c5)cccc34)c3c([2H])c([2H])c([2H])c([2H])c23)c([2H])c1 ; [2H]c1cc(-c2c3ccccc3c(-c3cccc4c(-c5ccc6ccccc6c5)cccc34)c3c([2H])c([2H])c([2H])c([2H])c23)cc([2H])c1[2H]]

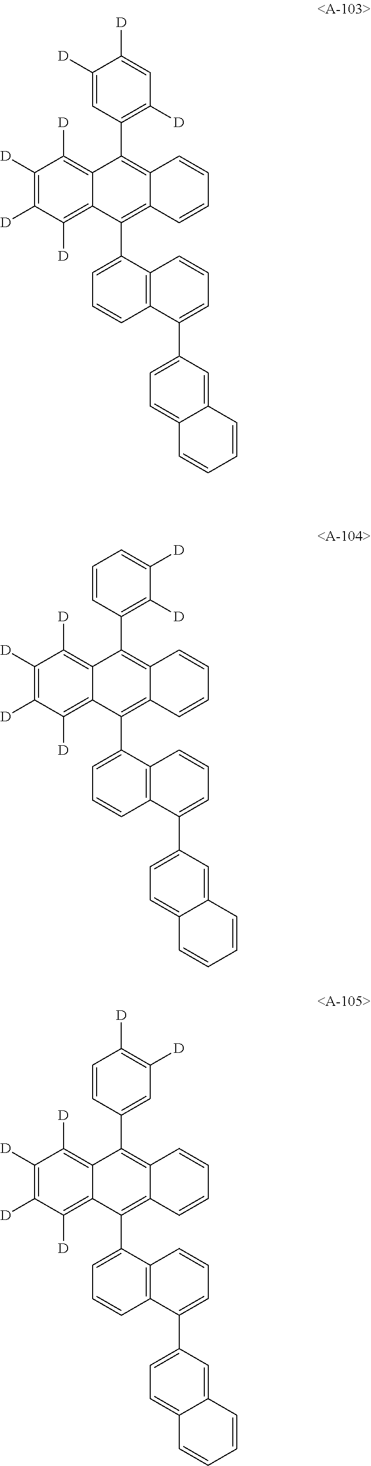 [SMILES: [2H]c1cc([2H])c(-c2c3ccccc3c(-c3cccc4c(-c5ccc6ccccc6c5)cccc34)c3c([2H])c([2H])c([2H])c([2H])c23)cc1[2H] ; [2H]c1cccc(-c2c3ccccc3c(-c3cccc4c(-c5ccc6ccccc6c5)cccc34)c3c([2H])c([2H])c([2H])c([2H])c23)c1[2H] ; [2H]c1ccc(-c2c3ccccc3c(-c3cccc4c(-c5ccc6ccccc6c5)cccc34)c3c([2H])c([2H])c([2H])c([2H])c23)cc1[2H]]

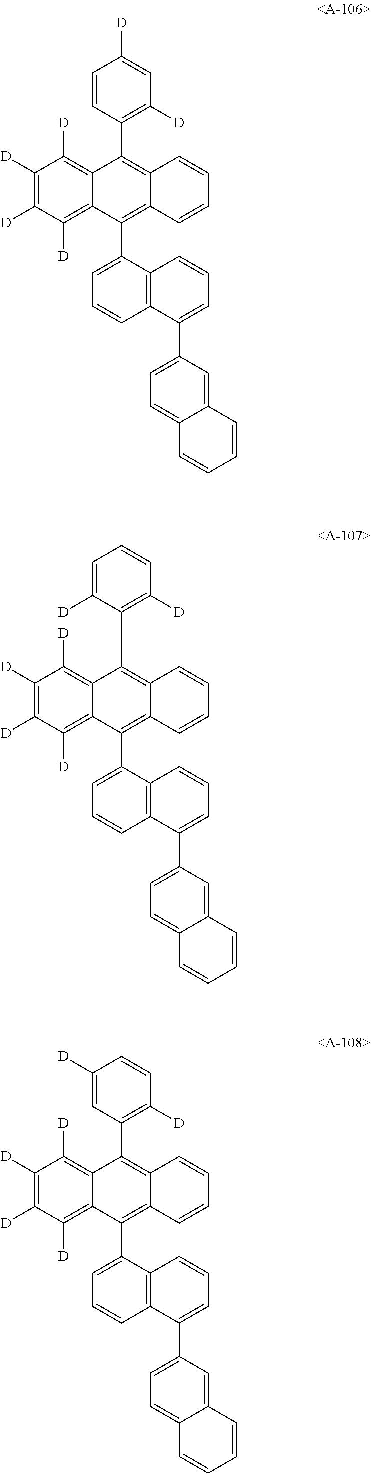 [SMILES: [2H]c1ccc(-c2c3ccccc3c(-c3cccc4c(-c5ccc6ccccc6c5)cccc34)c3c([2H])c([2H])c([2H])c([2H])c23)c([2H])c1 ; [2H]c1cccc([2H])c1-c1c2ccccc2c(-c2cccc3c(-c4ccc5ccccc5c4)cccc23)c2c([2H])c([2H])c([2H])c([2H])c12 ; [2H]c1ccc([2H])c(-c2c3ccccc3c(-c3cccc4c(-c5ccc6ccccc6c5)cccc34)c3c([2H])c([2H])c([2H])c([2H])c23)c1]

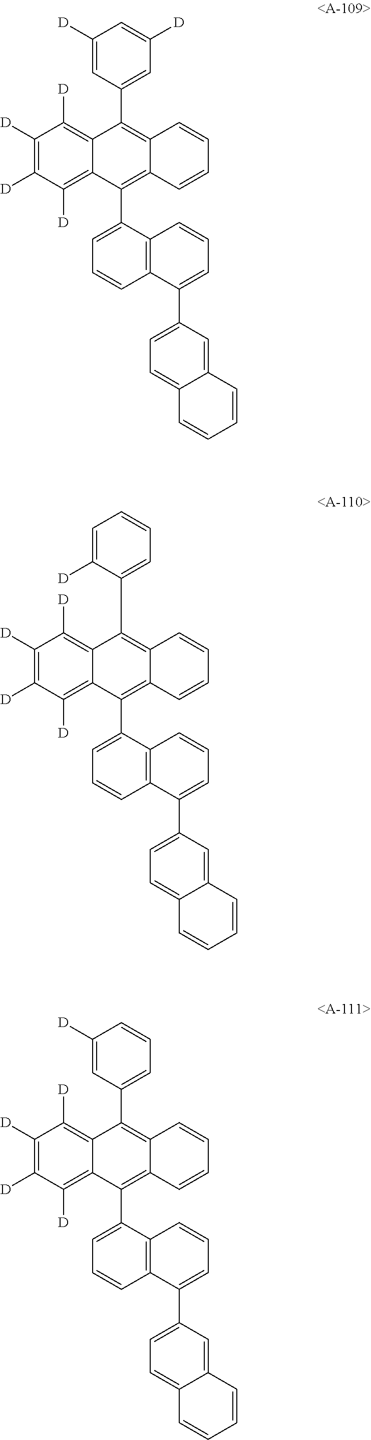 [SMILES: [2H]c1cc([2H])cc(-c2c3ccccc3c(-c3cccc4c(-c5ccc6ccccc6c5)cccc34)c3c([2H])c([2H])c([2H])c([2H])c23)c1 ; [2H]c1ccccc1-c1c2ccccc2c(-c2cccc3c(-c4ccc5ccccc5c4)cccc23)c2c([2H])c([2H])c([2H])c([2H])c12 ; [2H]c1cccc(-c2c3ccccc3c(-c3cccc4c(-c5ccc6ccccc6c5)cccc34)c3c([2H])c([2H])c([2H])c([2H])c23)c1]

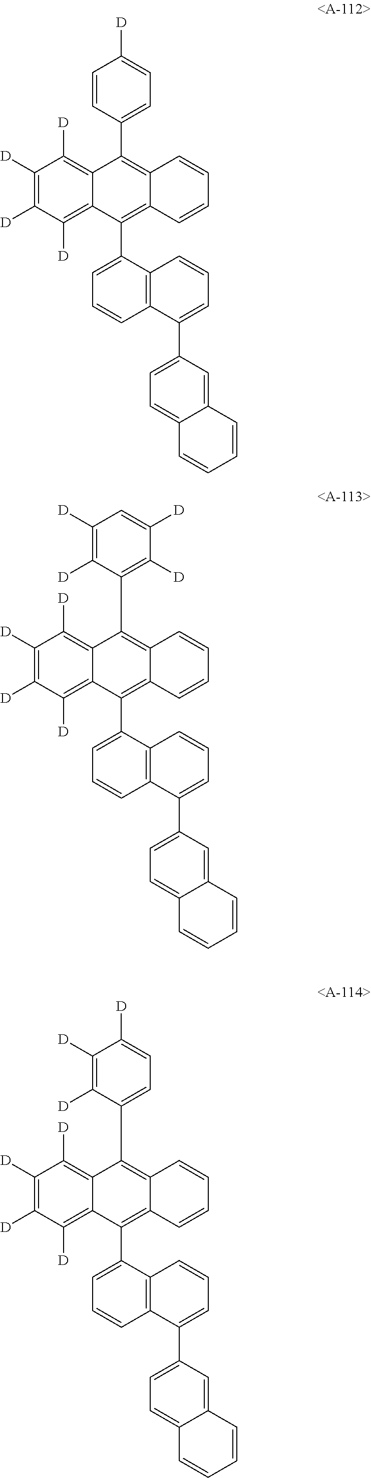 [SMILES: [2H]c1ccc(-c2c3ccccc3c(-c3cccc4c(-c5ccc6ccccc6c5)cccc34)c3c([2H])c([2H])c([2H])c([2H])c23)cc1 ; [2H]c1cc([2H])c([2H])c(-c2c3ccccc3c(-c3cccc4c(-c5ccc6ccccc6c5)cccc34)c3c([2H])c([2H])c([2H])c([2H])c23)c1[2H] ; [2H]c1ccc(-c2c3ccccc3c(-c3cccc4c(-c5ccc6ccccc6c5)cccc34)c3c([2H])c([2H])c([2H])c([2H])c23)c([2H])c1[2H]]

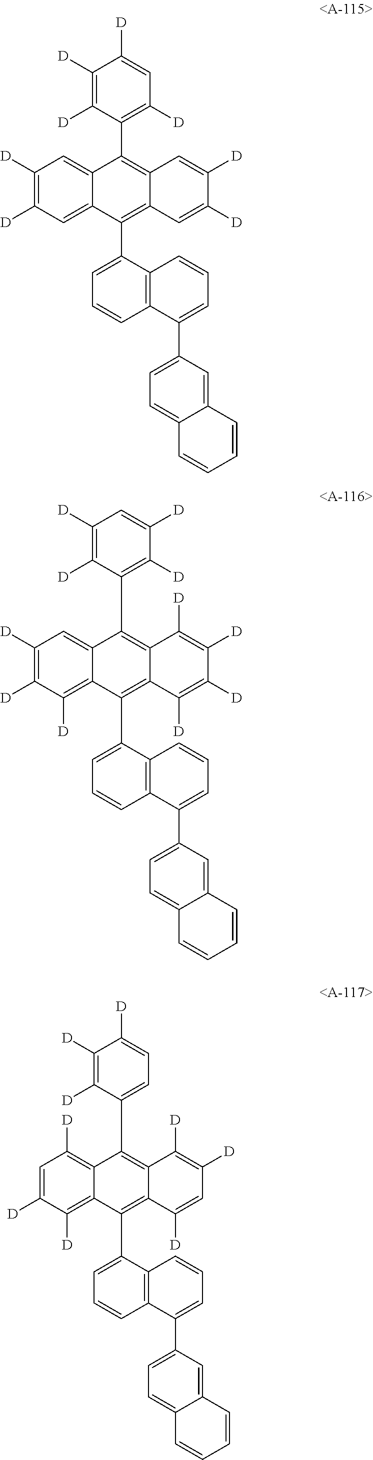 [SMILES: [2H]c1cc2c(-c3c([2H])cc([2H])c([2H])c3[2H])c3cc([2H])c([2H])cc3c(-c3cccc4c(-c5ccc6ccccc6c5)cccc34)c2cc1[2H] ; [2H]c1cc([2H])c([2H])c(-c2c3cc([2H])c([2H])c([2H])c3c(-c3cccc4c(-c5ccc6ccccc6c5)cccc34)c3c([2H])c([2H])c([2H])c([2H])c23)c1[2H] ; [2H]c1ccc(-c2c3c([2H])cc([2H])c([2H])c3c(-c3cccc4c(-c5ccc6ccccc6c5)cccc34)c3c([2H])cc([2H])c([2H])c23)c([2H])c1[2H]]

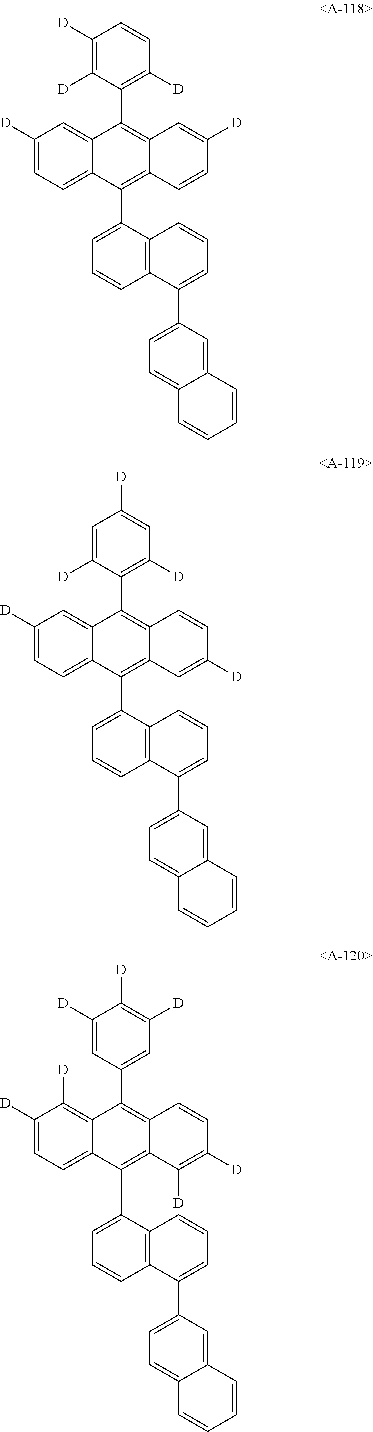 [SMILES: [2H]c1ccc2c(-c3cccc4c(-c5ccc6ccccc6c5)cccc34)c3ccc([2H])cc3c(-c3c([2H])ccc([2H])c3[2H])c2c1 ; [2H]c1cc([2H])c(-c2c3cc([2H])ccc3c(-c3cccc4c(-c5ccc6ccccc6c5)cccc34)c3cc([2H])ccc23)c([2H])c1 ; [2H]c1cc(-c2c3ccc([2H])c([2H])c3c(-c3cccc4c(-c5ccc6ccccc6c5)cccc34)c3ccc([2H])c([2H])c23)cc([2H])c1[2H]]

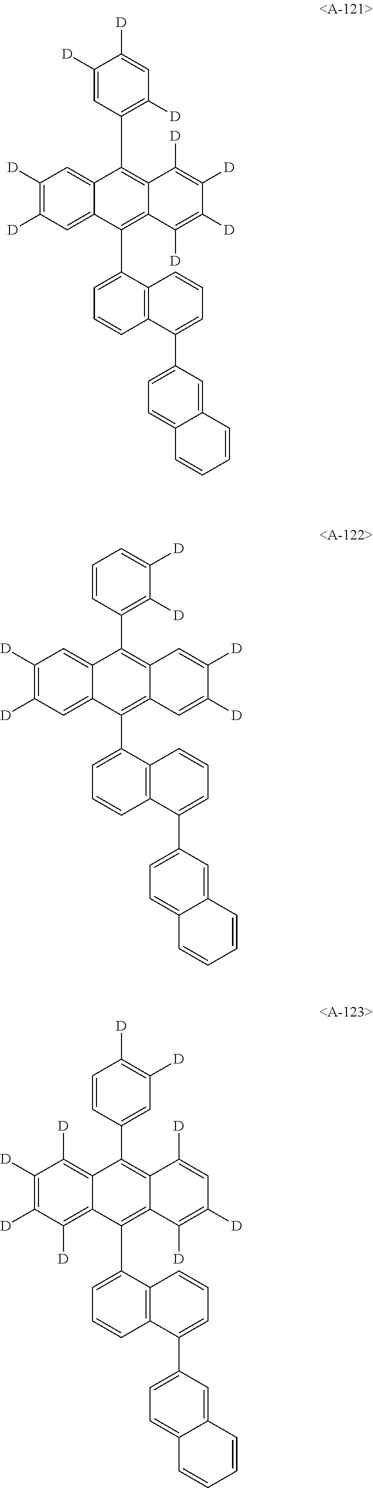 [SMILES: [2H]c1cc([2H])c(-c2c3cc([2H])c([2H])cc3c(-c3cccc4c(-c5ccc6ccccc6c5)cccc34)c3c([2H])c([2H])c([2H])c([2H])c23)cc1[2H] ; [2H]c1cc2c(-c3cccc([2H])c3[2H])c3cc([2H])c([2H])cc3c(-c3cccc4c(-c5ccc6ccccc6c5)cccc34)c2cc1[2H] ; [2H]c1ccc(-c2c3c([2H])cc([2H])c([2H])c3c(-c3cccc4c(-c5ccc6ccccc6c5)cccc34)c3c([2H])c([2H])c([2H])c([2H])c23)cc1[2H]]

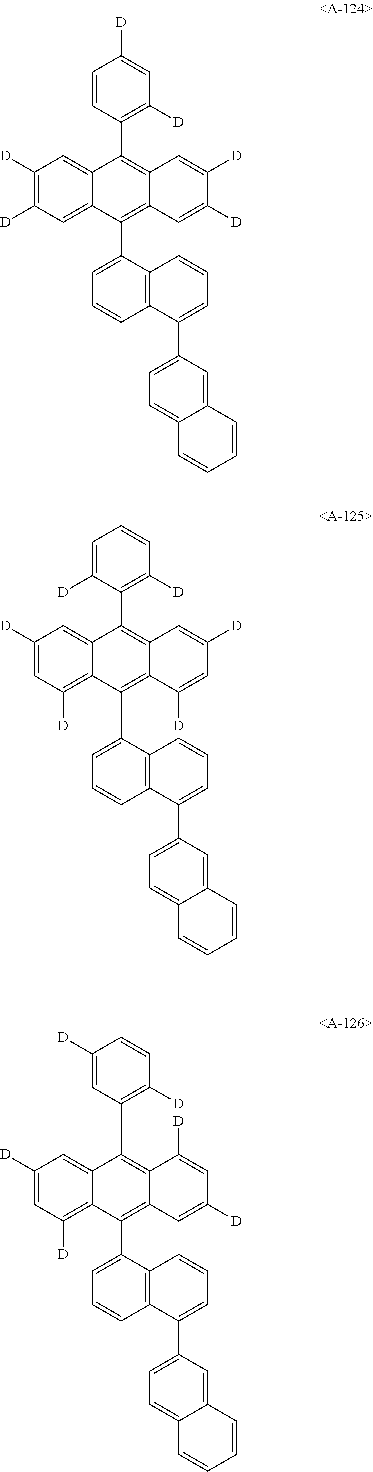 [SMILES: [2H]c1ccc(-c2c3cc([2H])c([2H])cc3c(-c3cccc4c(-c5ccc6ccccc6c5)cccc34)c3cc([2H])c([2H])cc23)c([2H])c1 ; [2H]c1cc([2H])c2c(-c3cccc4c(-c5ccc6ccccc6c5)cccc34)c3c([2H])cc([2H])cc3c(-c3c([2H])cccc3[2H])c2c1 ; [2H]c1ccc([2H])c(-c2c3cc([2H])cc([2H])c3c(-c3cccc4c(-c5ccc6ccccc6c5)cccc34)c3cc([2H])cc([2H])c23)c1]

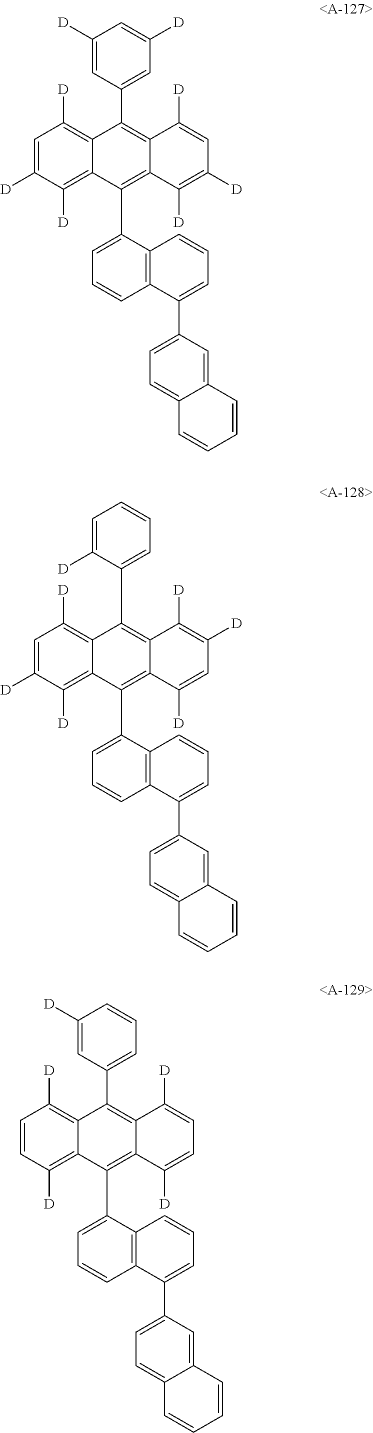 [SMILES: [2H]c1cc([2H])cc(-c2c3c([2H])cc([2H])c([2H])c3c(-c3cccc4c(-c5ccc6ccccc6c5)cccc34)c3c([2H])c([2H])cc([2H])c23)c1 ; [2H]c1ccccc1-c1c2c([2H])cc([2H])c([2H])c2c(-c2cccc3c(-c4ccc5ccccc5c4)cccc23)c2c([2H])cc([2H])c([2H])c12 ; [2H]c1cccc(-c2c3c([2H])ccc([2H])c3c(-c3cccc4c(-c5ccc6ccccc6c5)cccc34)c3c([2H])ccc([2H])c23)c1]

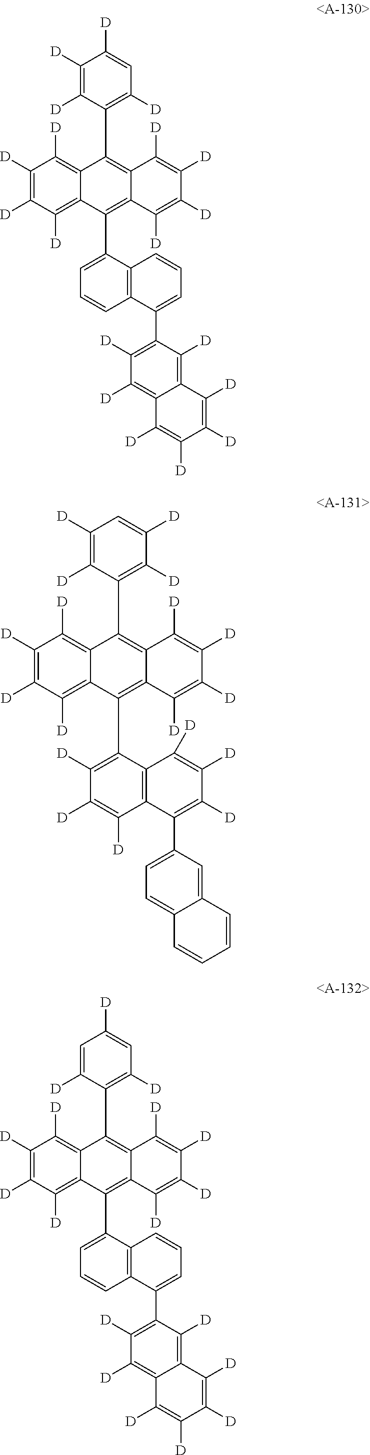

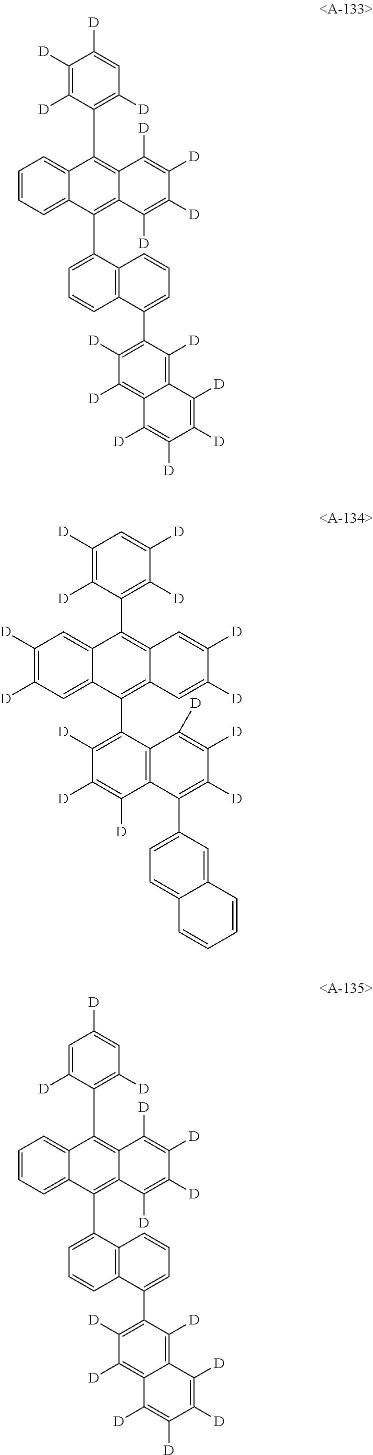 [SMILES: [2H]c1cc([2H])c(-c2c3ccccc3c(-c3cccc4c(-c5c([2H])c([2H])c6c([2H])c([2H])c([2H])c([2H])c6c5[2H])cccc34)c3c([2H])c([2H])c([2H])c([2H])c23)c([2H])c1[2H] ; [2H]c1cc2c(-c3c([2H])c([2H])cc([2H])c3[2H])c3cc([2H])c([2H])cc3c(-c3c([2H])c([2H])c([2H])c4c(-c5ccc6ccccc6c5)c([2H])c([2H])c([2H])c34)c2cc1[2H] ; [2H]c1cc([2H])c(-c2c3ccccc3c(-c3cccc4c(-c5c([2H])c([2H])c6c([2H])c([2H])c([2H])c([2H])c6c5[2H])cccc34)c3c([2H])c([2H])c([2H])c([2H])c23)c([2H])c1]

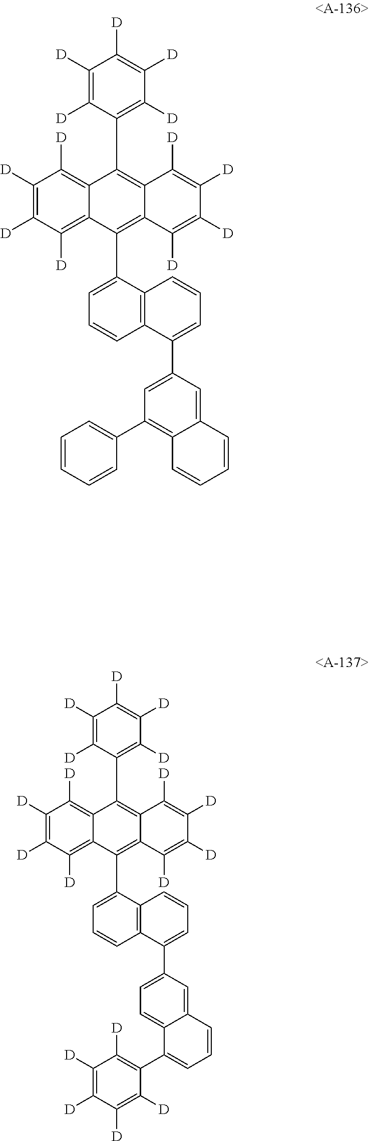 [SMILES: [2H]c1c([2H])c([2H])c(-c2c3c([2H])c([2H])c([2H])c([2H])c3c(-c3cccc4c(-c5cc(-c6ccccc6)c6ccccc6c5)cccc34)c3c([2H])c([2H])c([2H])c([2H])c23)c([2H])c1[2H] ; [2H]c1c([2H])c([2H])c(-c2cccc3cc(-c4cccc5c(-c6c7c([2H])c([2H])c([2H])c([2H])c7c(-c7c([2H])c([2H])c([2H])c([2H])c7[2H])c7c([2H])c([2H])c([2H])c([2H])c67)cccc45)ccc23)c([2H])c1[2H]]

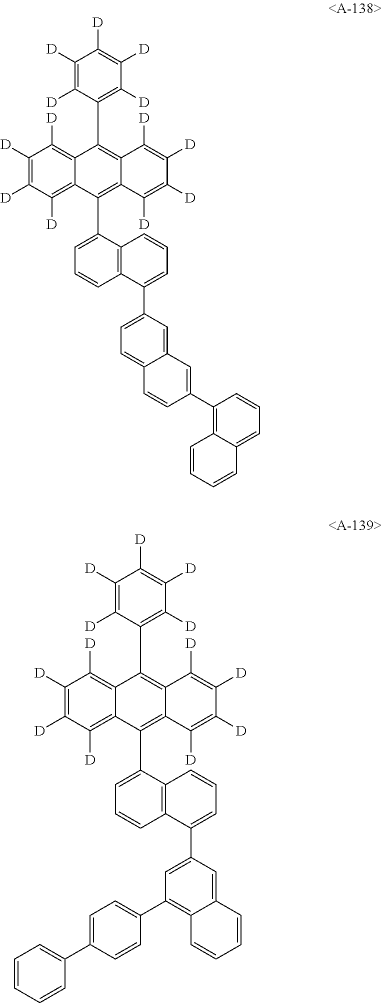 [SMILES: [2H]c1c([2H])c([2H])c(-c2c3c([2H])c([2H])c([2H])c([2H])c3c(-c3cccc4c(-c5ccc6ccc(-c7cccc8ccccc78)cc6c5)cccc34)c3c([2H])c([2H])c([2H])c([2H])c23)c([2H])c1[2H] ; [2H]c1c([2H])c([2H])c(-c2c3c([2H])c([2H])c([2H])c([2H])c3c(-c3cccc4c(-c5cc(-c6ccc(-c7ccccc7)cc6)c6ccccc6c5)cccc34)c3c([2H])c([2H])c([2H])c([2H])c23)c([2H])c1[2H]]

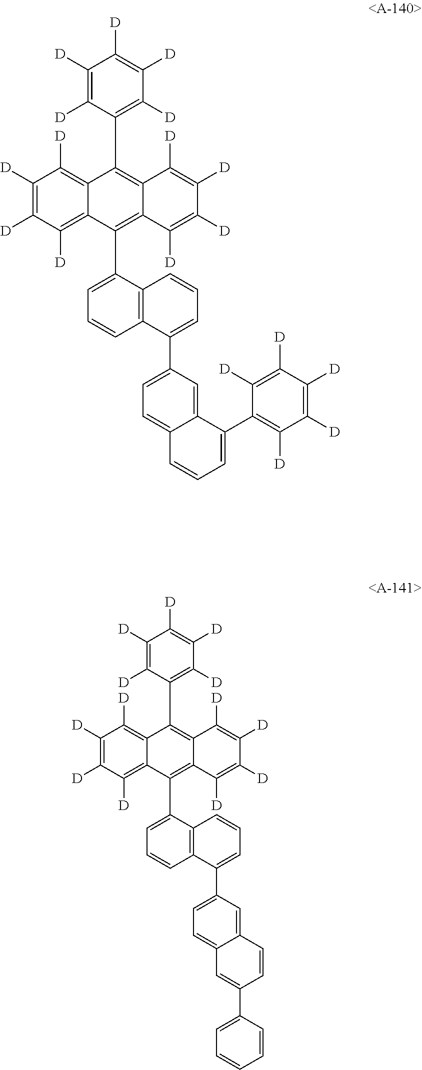 [SMILES: [2H]c1c([2H])c([2H])c(-c2cccc3ccc(-c4cccc5c(-c6c7c([2H])c([2H])c([2H])c([2H])c7c(-c7c([2H])c([2H])c([2H])c([2H])c7[2H])c7c([2H])c([2H])c([2H])c([2H])c67)cccc45)cc23)c([2H])c1[2H] ; [2H]c1c([2H])c([2H])c(-c2c3c([2H])c([2H])c([2H])c([2H])c3c(-c3cccc4c(-c5ccc6cc(-c7ccccc7)ccc6c5)cccc34)c3c([2H])c([2H])c([2H])c([2H])c23)c([2H])c1[2H]]

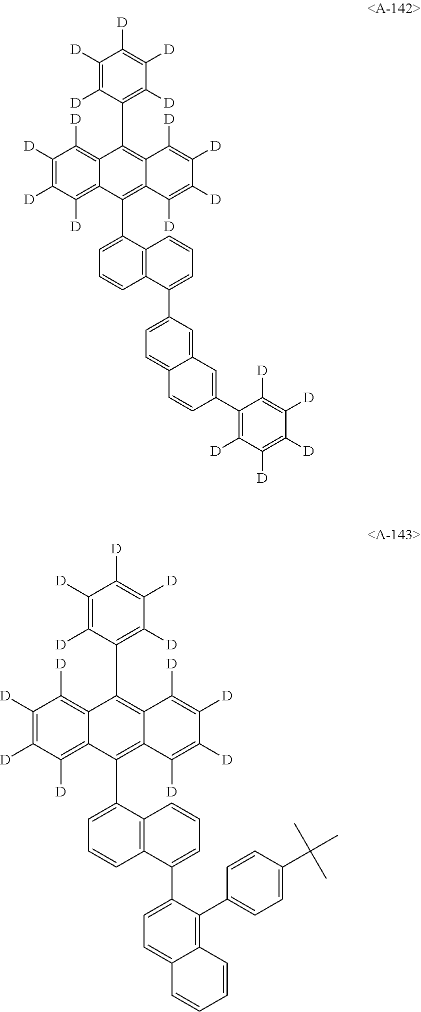 [SMILES: [2H]c1c([2H])c([2H])c(-c2ccc3ccc(-c4cccc5c(-c6c7c([2H])c([2H])c([2H])c([2H])c7c(-c7c([2H])c([2H])c([2H])c([2H])c7[2H])c7c([2H])c([2H])c([2H])c([2H])c67)cccc45)cc3c2)c([2H])c1[2H] ; [2H]c1c([2H])c([2H])c(-c2c3c([2H])c([2H])c([2H])c([2H])c3c(-c3cccc4c(-c5ccc6ccccc6c5-c5ccc(C(C)(C)C)cc5)cccc34)c3c([2H])c([2H])c([2H])c([2H])c23)c([2H])c1[2H]]

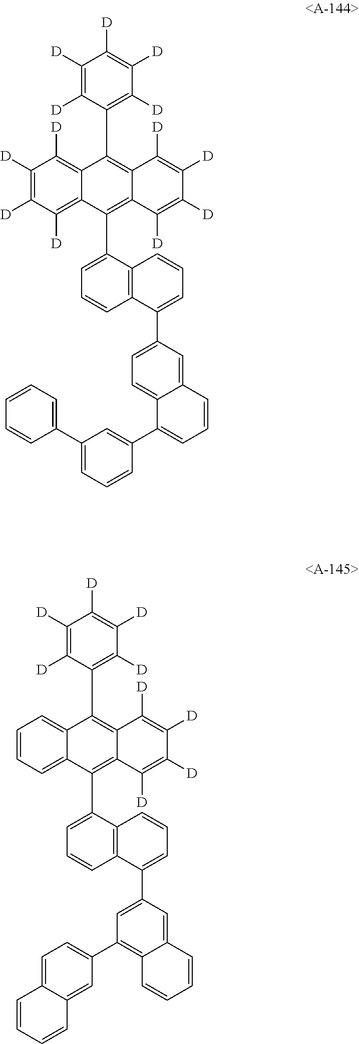 [SMILES: [2H]c1c([2H])c([2H])c(-c2c3c([2H])c([2H])c([2H])c([2H])c3c(-c3cccc4c(-c5ccc6c(-c7cccc(-c8ccccc8)c7)cccc6c5)cccc34)c3c([2H])c([2H])c([2H])c([2H])c23)c([2H])c1[2H] ; [2H]c1c([2H])c([2H])c(-c2c3ccccc3c(-c3cccc4c(-c5cc(-c6ccc7ccccc7c6)c6ccccc6c5)cccc34)c3c([2H])c([2H])c([2H])c([2H])c23)c([2H])c1[2H]]

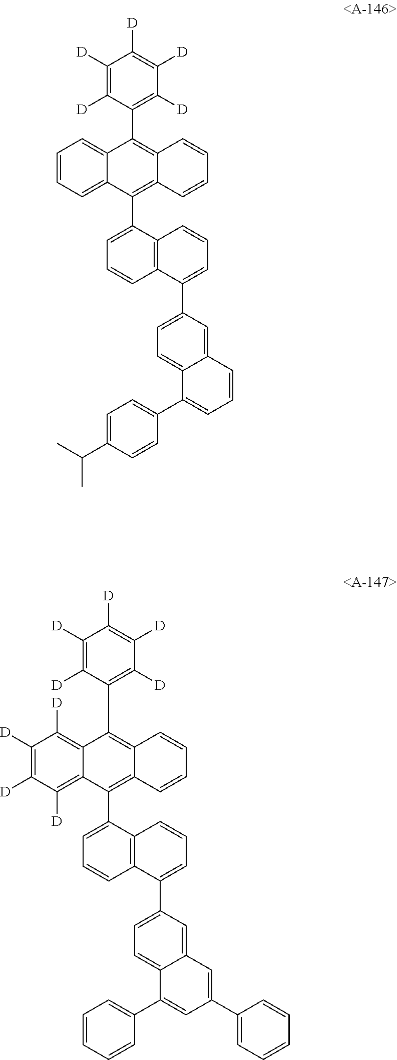 [SMILES: [2H]c1c([2H])c([2H])c(-c2c3ccccc3c(-c3cccc4c(-c5ccc6c(-c7ccc(C(C)C)cc7)cccc6c5)cccc34)c3ccccc23)c([2H])c1[2H] ; [2H]c1c([2H])c([2H])c(-c2c3ccccc3c(-c3cccc4c(-c5ccc6c(-c7ccccc7)cc(-c7ccccc7)cc6c5)cccc34)c3c([2H])c([2H])c([2H])c([2H])c23)c([2H])c1[2H]]

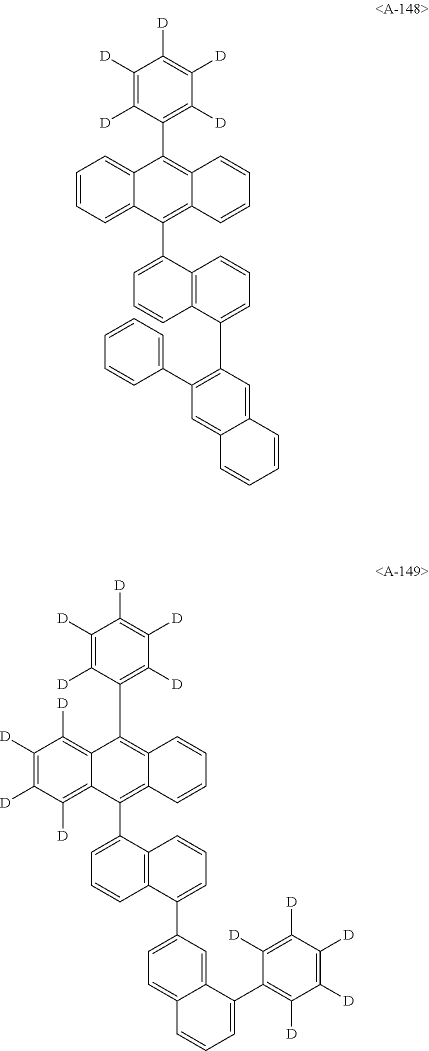 [SMILES: [2H]c1c([2H])c([2H])c(-c2c3ccccc3c(-c3cccc4c(-c5cc6ccccc6cc5-c5ccccc5)cccc34)c3ccccc23)c([2H])c1[2H] ; [2H]c1c([2H])c([2H])c(-c2cccc3ccc(-c4cccc5c(-c6c7ccccc7c(-c7c([2H])c([2H])c([2H])c([2H])c7[2H])c7c([2H])c([2H])c([2H])c([2H])c67)cccc45)cc23)c([2H])c1[2H]]

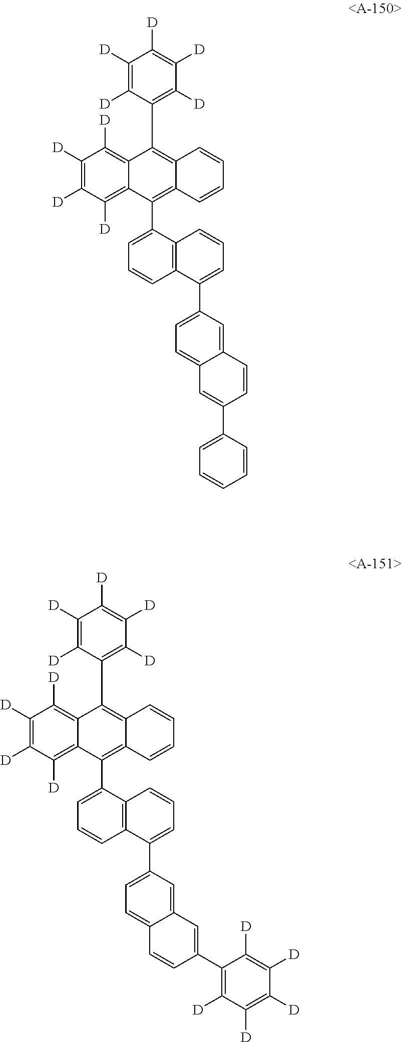 [SMILES: [2H]c1c([2H])c([2H])c(-c2c3ccccc3c(-c3cccc4c(-c5ccc6cc(-c7ccccc7)ccc6c5)cccc34)c3c([2H])c([2H])c([2H])c([2H])c23)c([2H])c1[2H] ; [2H]c1c([2H])c([2H])c(-c2ccc3ccc(-c4cccc5c(-c6c7ccccc7c(-c7c([2H])c([2H])c([2H])c([2H])c7[2H])c7c([2H])c([2H])c([2H])c([2H])c67)cccc45)cc3c2)c([2H])c1[2H]]

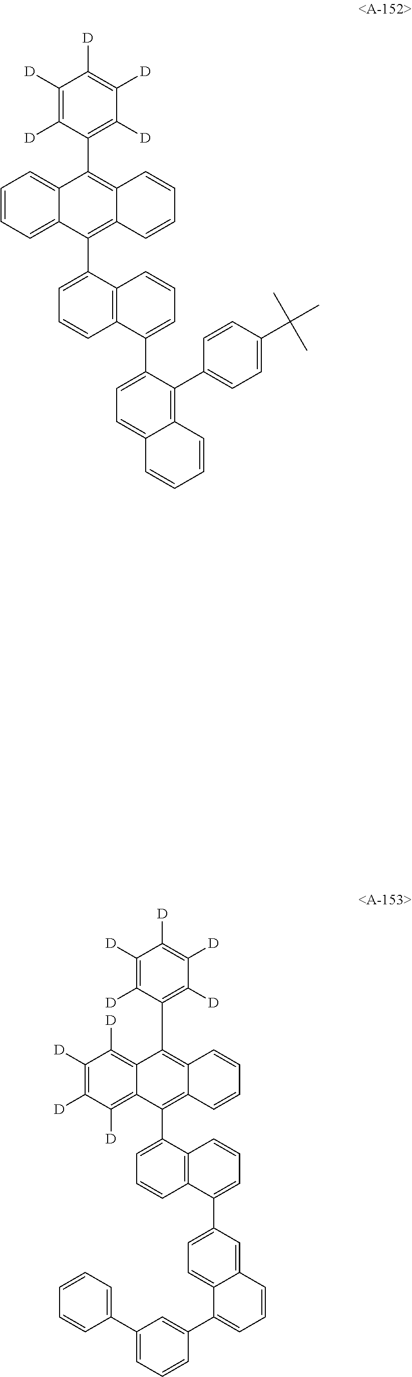 [SMILES: [2H]c1c([2H])c([2H])c(-c2c3ccccc3c(-c3cccc4c(-c5ccc6ccccc6c5-c5ccc(C(C)(C)C)cc5)cccc34)c3ccccc23)c([2H])c1[2H] ; [2H]c1c([2H])c([2H])c(-c2c3ccccc3c(-c3cccc4c(-c5ccc6c(-c7cccc(-c8ccccc8)c7)cccc6c5)cccc34)c3c([2H])c([2H])c([2H])c([2H])c23)c([2H])c1[2H]]

In the present disclosure, the compound represented by Chemical Formula D6 or Chemical Formula D7 is characterized by the structure in which the T5 and T6 ring moieties, which are each a substituted or unsubstituted aromatic hydrocarbon ring of 6 to 50 carbon atoms or a substituted or unsubstituted heteroaromatic ring of 2 to 50 carbon atoms, are each bonded to the central boron atom (B) and linked to each other via the linker Y₆; and the T4 ring moiety is bonded to the linker Y₄, wherein, of the two carbon atoms in double bond between the 5-membered ring bearing Y₄ and the 6-membered ring bearing Y₅, one is bonded to both T4 and Y₅ or to both Y₄ and Y₅ and, the other is bonded to both Y₄ and B or to both T4 and B whereby the 5-membered ring bearing Y₄ and the 6-membered ring bearing B and Y₅ forms a fused ring.

The ring moieties T4 and T5 in Chemical Formulas D6 and D7 may be same or different and are each independently a substituted or unsubstituted aromatic hydrocarbon ring of 6 to 30 carbon atoms and particularly a substituted or unsubstituted aromatic hydrocarbon ring of 6 to 18 carbon atoms.

According to an embodiment, at least one of Y₅ and Y₆ in Chemical Formulas D6 and D7 may be NR₆₆ or NR₇₁, wherein R₆₆ and R₇₁ are same or different and may each be independently a substituted or unsubstituted aryl of 6 to 30 carbon atom. In this regard, NR₆₆ and NR₇₁ are as defined above.

According to an embodiment, in Chemical Formulas D6 and D7, Y₅ may be NR₆₆ and Y₆ may be NR₇₁, wherein R₆₆ and R₇₁ are same or different and may each be independently a substituted or unsubstituted aryl of 6 to 30 carbon atoms. In this regard, NR₆₆ and NR₇ are as defined above.

In addition, the linkers Y₅ and Y₆ in Chemical Formulas D6 and D7 are same or different and may each be independently represented by the following Structural Formula A:

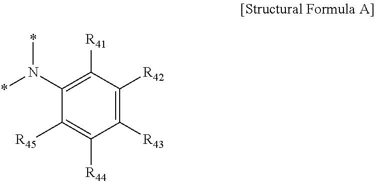

wherein “-*” denotes a bonding site at which the N atom is bonded to the doubly bonded carbon atom connected to Y₄, the doubly bonded carbon atom connected to Y₅ in the 5-membered ring bearing Y₄, an aromatic carbon atom in the T5 ring moiety, or an aromatic carbon atom in the T6 ring moiety;
R₄₁ to R₄₅, which may be same or different, are each independently any one selected from a hydrogen atom, a deuterium atom, a substituted or unsubstituted alkyl of 1 to 30 carbon atoms, an alkenyl of 2 to 24 carbon atoms, an alkynyl of 2 to 24 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 1 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a nitro, a cyano, and a halogen atom, and R₄₁ and R₄₅ may each independently be bonded to the T4, T5, or T6 ring moiety to form an additional aliphatic or aromatic mono- or polycyclic ring.

In the context that the linkers Y₅ and Y₆ in Chemical Formulas D6 and D7 may be same or different and at least one of them may be the linker represented by the following Structural Formula A, at least one of R₄₁ and R₄₅ in Structural Formula A may be bonded to the T6 ring moiety to form an additional aliphatic or aromatic mono- or polycyclic ring.

Furthermore, Y₅ and Y₆ in Chemical Formulas D6 and D7 are same or different from each other and may each be independently a linker represented by Structural Formula A, above.

In the compound represented by Chemical Formula D6 or D7 of the present disclosure, T4 to T6 ring moieties, which may be same or different, are each independently a substituted or unsubstituted aromatic hydrocarbon ring of 6 to 24 carbon atoms. In detail, the aromatic hydrocarbon ring may be any one selected from a benzene ring, a naphthalene ring, a biphenyl ring, a terphenyl ring, an anthracene ring, a phenanthrene ring, a indene ring, a fluorene ring, a pyrene ring, a perylene ring, a chrysene ring, a naphthacene ring, a fluoranthene ring, and a pentacene ring.

When the aromatic hydrocarbon rings of T4 to T6, which may be same or different, are each independently a substituted or unsubstituted aromatic hydrocarbon ring of 6 to 24 carbon atoms, the aromatic hydrocarbon rings of T4 and T5 in Chemical Formulas D6 and D7 may each independently any one selected from [Structural Formula 10] to [Structural Formula 21], below:

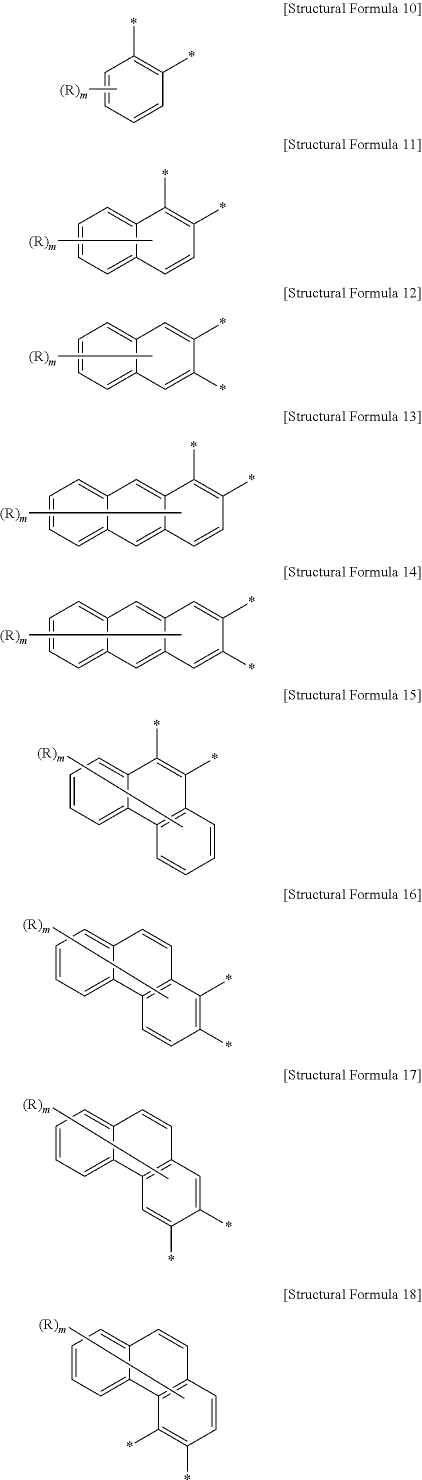

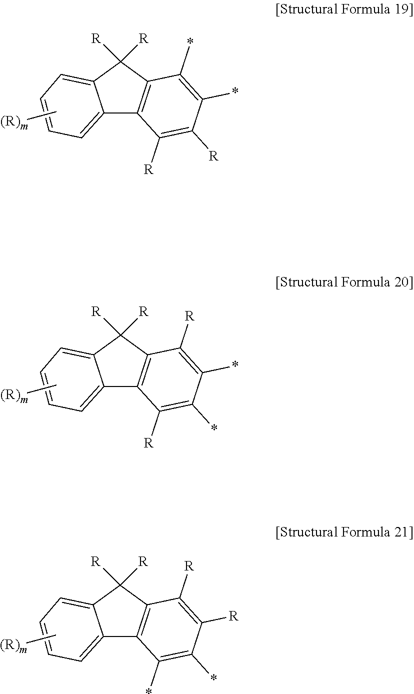

wherein,
“-*” denotes a bonding site at which the carbon ring member of T4 is bonded to Y₄ or a carbon member of the 5-membered ring bearing Y₄ or at which the carbon ring member of T5 is bonded to B or Y₆;
R's, which may be same or different, are each independently any one selected from a hydrogen atom, a deuterium atom, a substituted or unsubstituted alkyl of 1 to 30 carbon atoms, an alkenyl of 2 to 24 carbon atoms, an alkynyl of 2 to 24 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 1 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a nitro, a cyano, and a halogen atom; and
m is an integer of 1 to 8 wherein when m is 2 or greater or when two or more R's exist, the individual R's may be same or different.

In addition, when the T4 to T6 ring moieties, which may be same or different, are each independently a substituted or unsubstituted aromatic hydrocarbon ring of 6 to 50 carbon atoms, the aromatic hydrocarbon ring of T6 in Chemical Formulas D6 and D7 may be a ring represented by the following [Structural Formula B]:

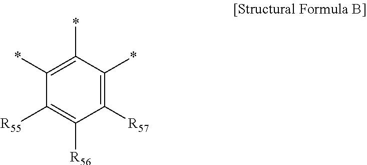

wherein,
“-*” denotes a bonding site at which the corresponding aromatic carbon ring members of T6 are bonded to Y₆, B, and Y₅, respectively; and
R₅₅ to R₅₇, which may be same or different, are each independently any one selected from a hydrogen atom, a deuterium atom, a substituted or unsubstituted alkyl of 1 to 30 carbon atoms, an alkenyl of 2 to 24 carbon atoms, an alkynyl of 2 to 24 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 1 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a nitro, a cyano, and a halogen atom, and R₅₅ to R₅₇ may each be linked to an adjacent substituent to form an additional aliphatic or aromatic mono- or polycyclic ring.

In Chemical Formulas D6 and D7 of the present disclosure, the aromatic hydrocarbon ring of 6 to 50 carbon atoms or the heteroaromatic ring of 2 to 40 carbon atoms of at least one of the T4 to T6 ring moieties may be bonded to an aryl amino radical represented by the following Structural Formula F:

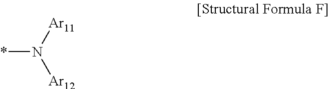

wherein,
“-*” denotes a bonding site participating in forming a bond to a carbon aromatic ring member of any one of T4 to T6, and
Ar₁₁ and Ar₁₂, which may be same or different, are each independently a substituted or unsubstituted aryl of 6 to 18 carbon atoms, and particularly a substituted or unsubstituted aryl of 6 to 12 carbon atoms, and may be linked to each other to form a ring.

According to an embodiment, Y₄ in Chemical Formulas D6 and D7 may be S.

In addition, the compounds represented by Chemical Formula D6 or D7 may each be any one selected from <D201> to <D350>, but are not limited thereto:

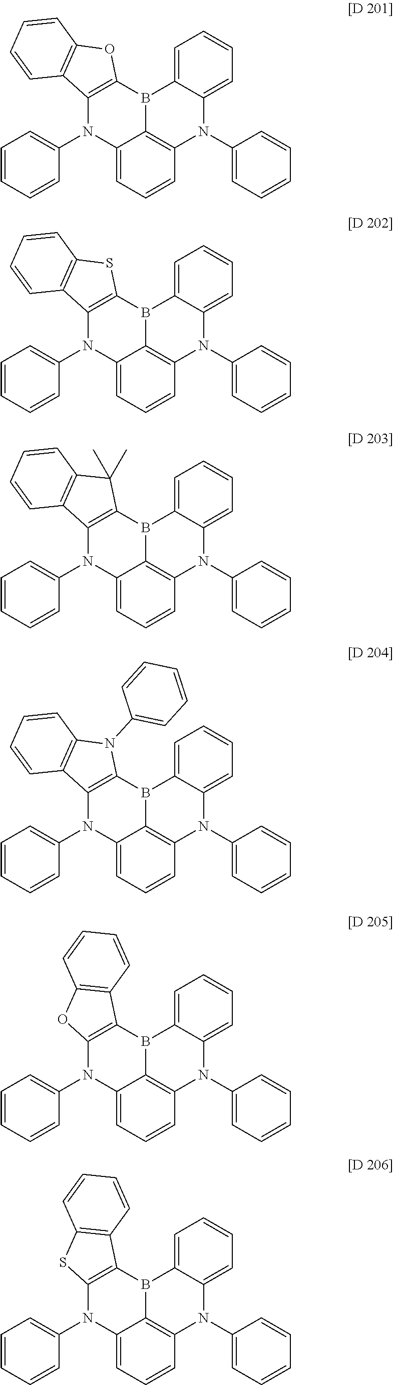

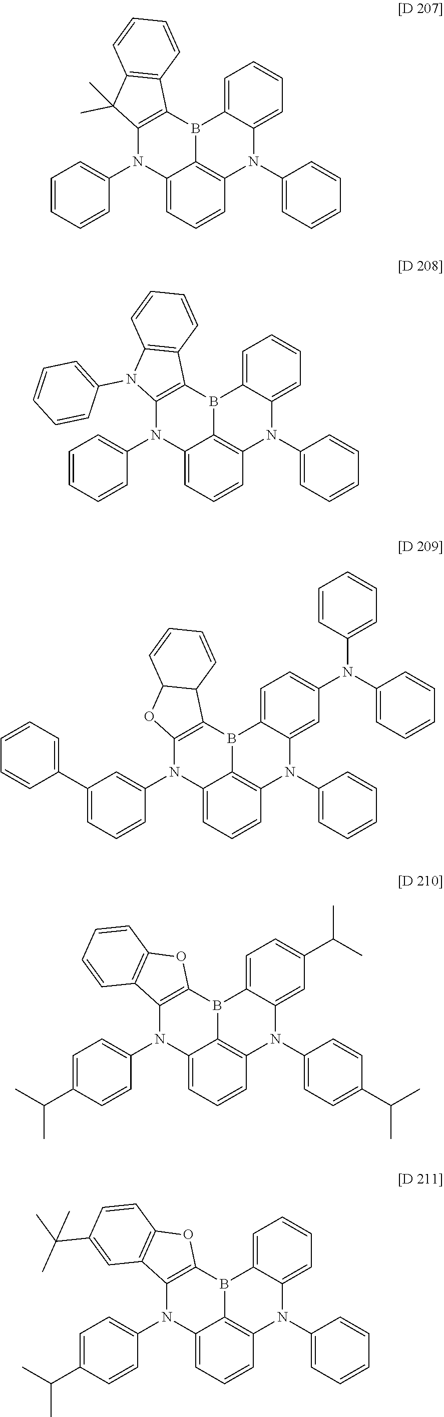

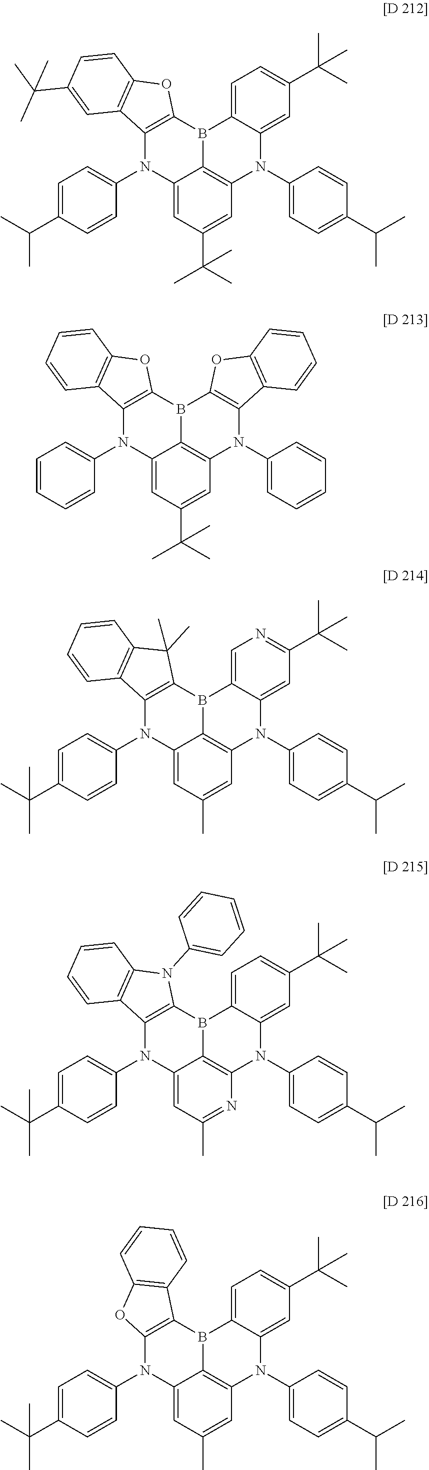

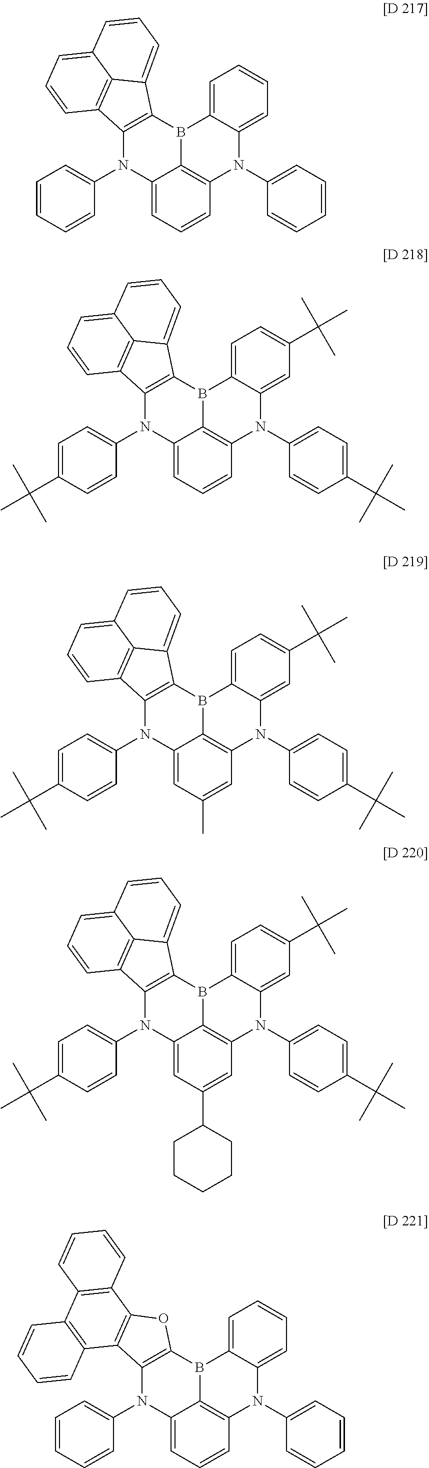

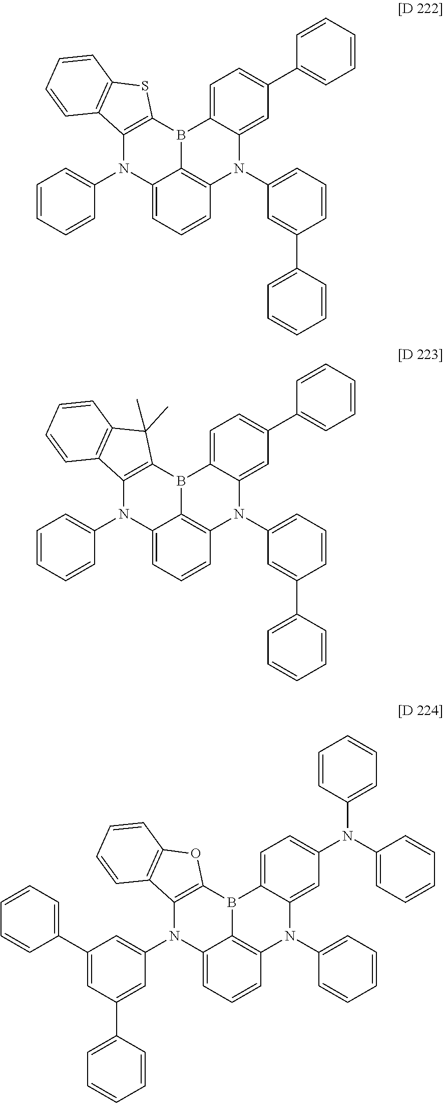

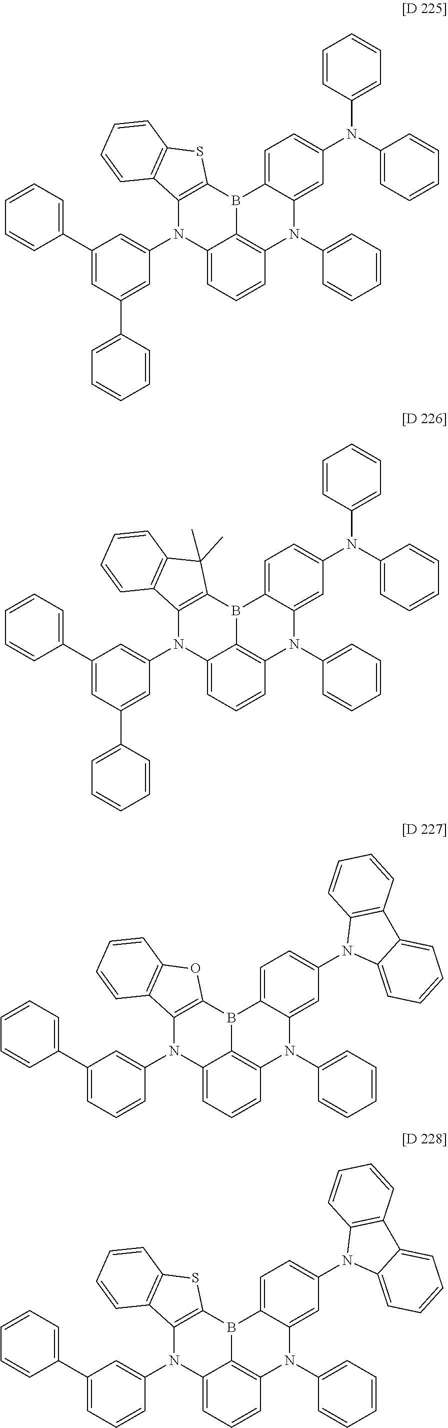 [SMILES: c1ccc(-c2cc(-c3ccccc3)cc(N3c4cccc5c4B(c4ccc(N(c6ccccc6)c6ccccc6)cc4N5c4ccccc4)c4sc5ccccc5c43)c2)cc1 ; CC1(C)C2=C(c3ccccc31)N(c1cc(-c3ccccc3)cc(-c3ccccc3)c1)c1cccc3c1B2c1ccc(N(c2ccccc2)c2ccccc2)cc1N3c1ccccc1 ; c1ccc(-c2cccc(N3c4cccc5c4B(c4ccc(-n6c7ccccc7c7ccccc76)cc4N5c4ccccc4)c4oc5ccccc5c43)c2)cc1 ; c1ccc(-c2cccc(N3c4cccc5c4B(c4ccc(-n6c7ccccc7c7ccccc76)cc4N5c4ccccc4)c4sc5ccccc5c43)c2)cc1]

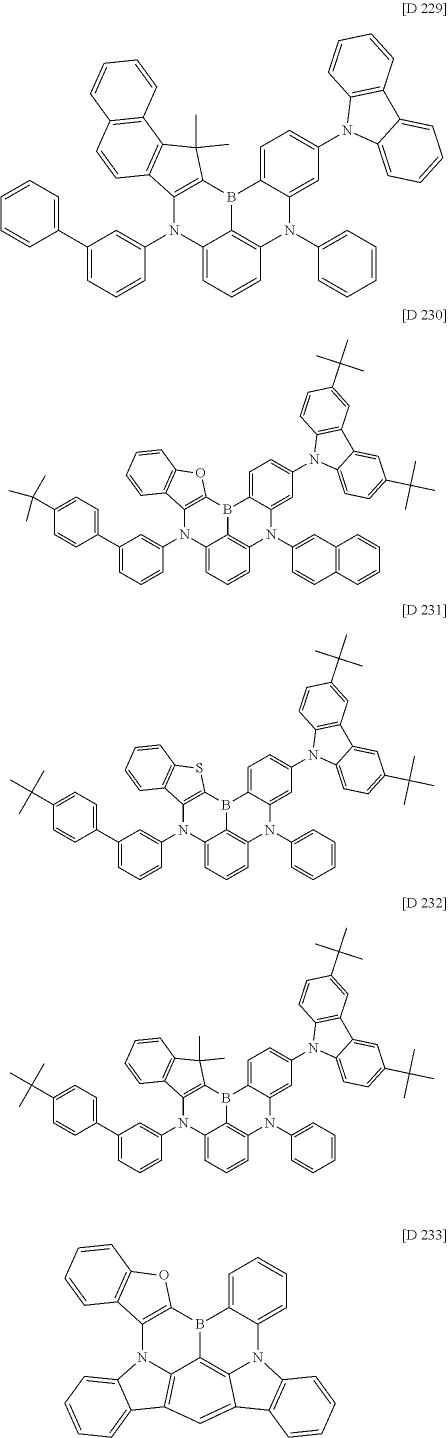

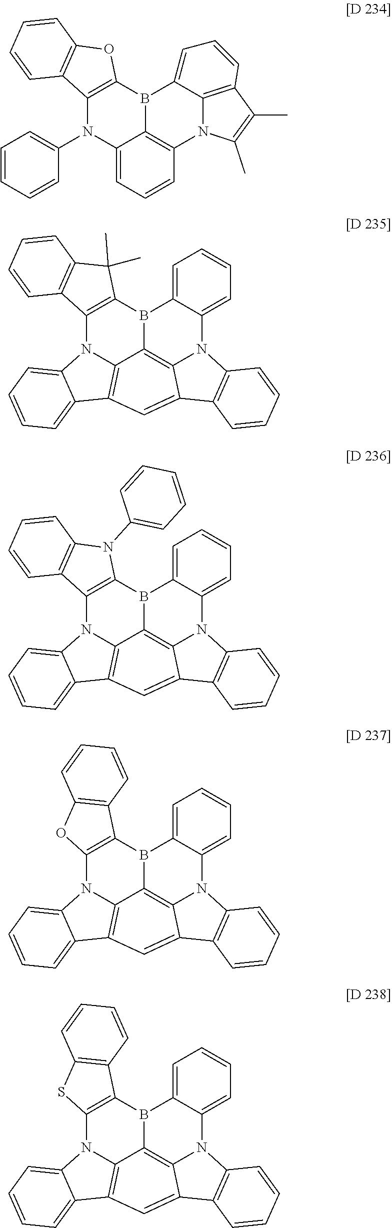

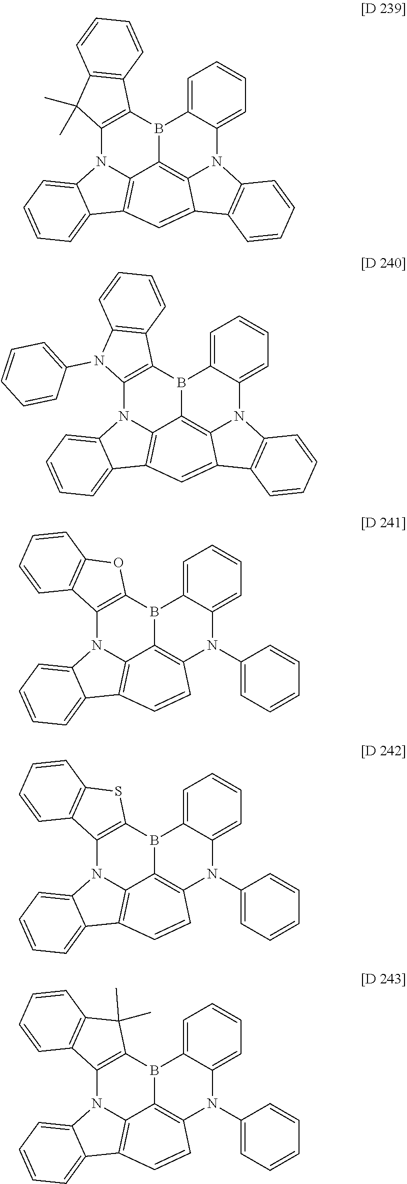

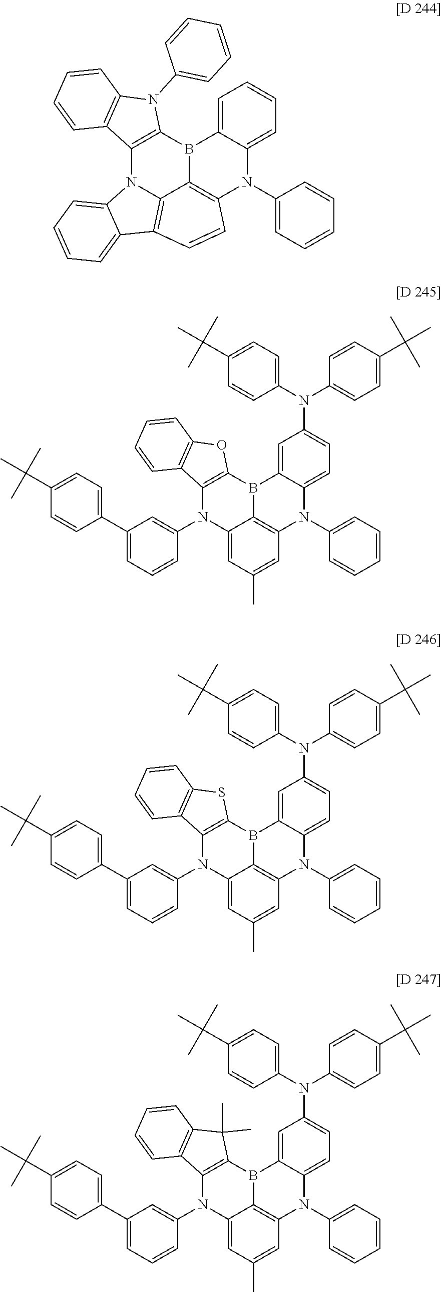

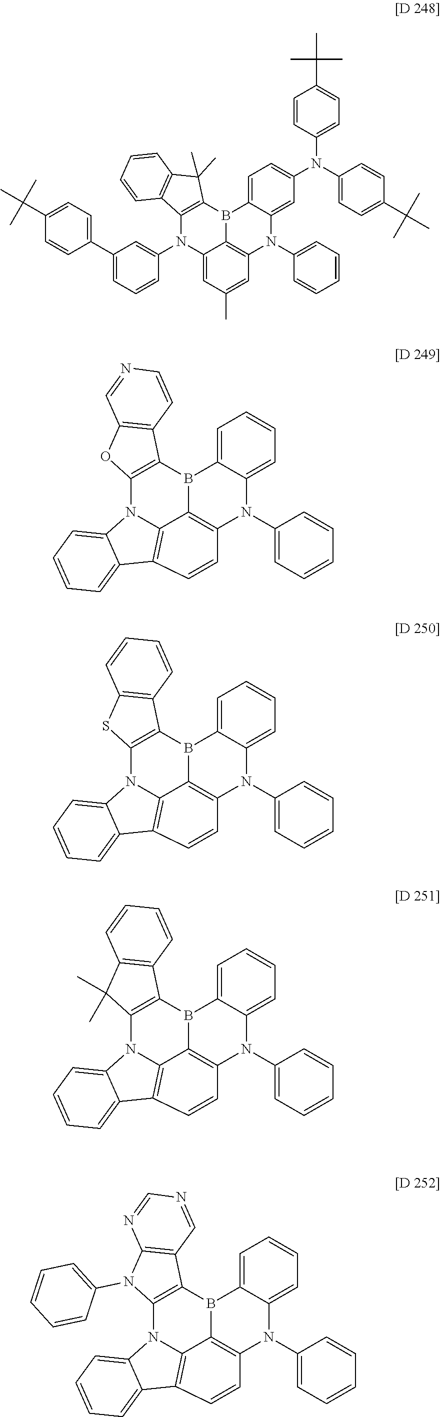 [SMILES: Cc1cc2c3c(c1)N(c1ccccc1)c1cc(N(c4ccc(C(C)(C)C)cc4)c4ccc(C(C)(C)C)cc4)ccc1B3C1=C(c3ccccc3C1(C)C)N2c1cccc(-c2ccc(C(C)(C)C)cc2)c1 ; c1ccc(N2c3ccccc3B3c4c(oc5cnccc45)-n4c5ccccc5c5ccc2c3c54)cc1 ; c1ccc(N2c3ccccc3B3c4c(sc5ccccc45)-n4c5ccccc5c5ccc2c3c54)cc1 ; CC1(C)C2=C(B3c4ccccc4N(c4ccccc4)c4ccc5c6ccccc6n2c5c43)c2ccccc21 ; c1ccc(N2c3ccccc3B3c4c(n(-c5ccccc5)c5ncncc45)-n4c5ccccc5c5ccc2c3c54)cc1]

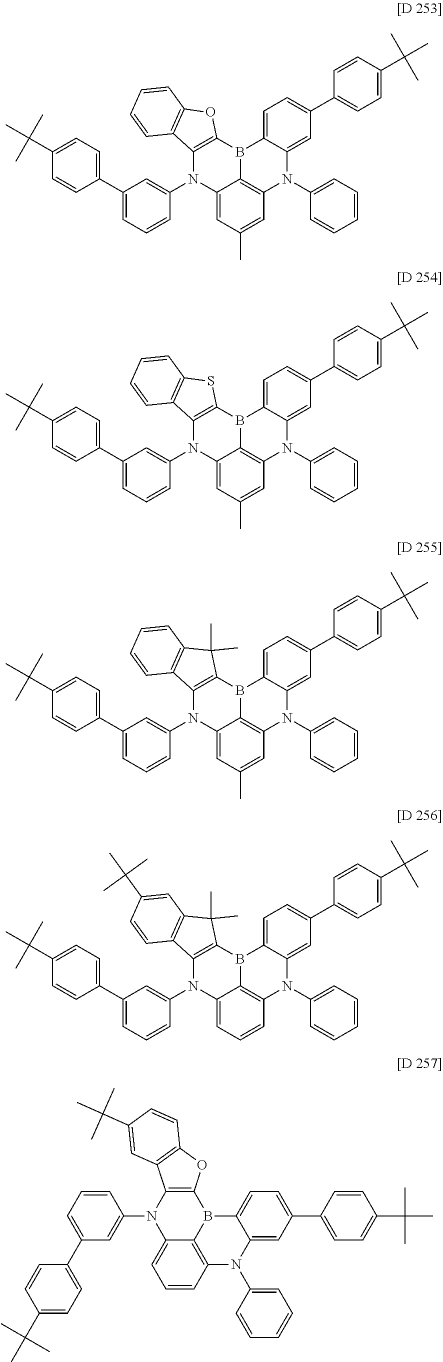

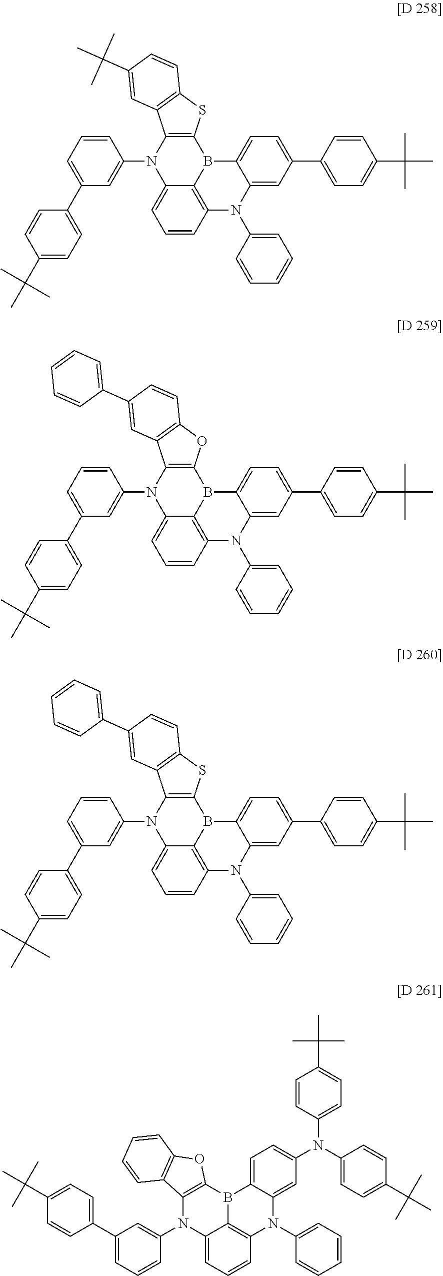

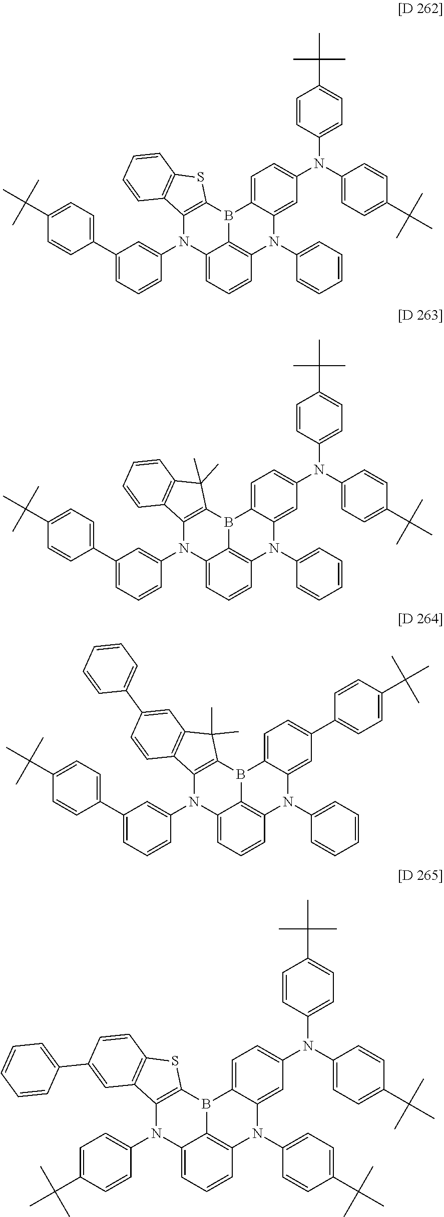

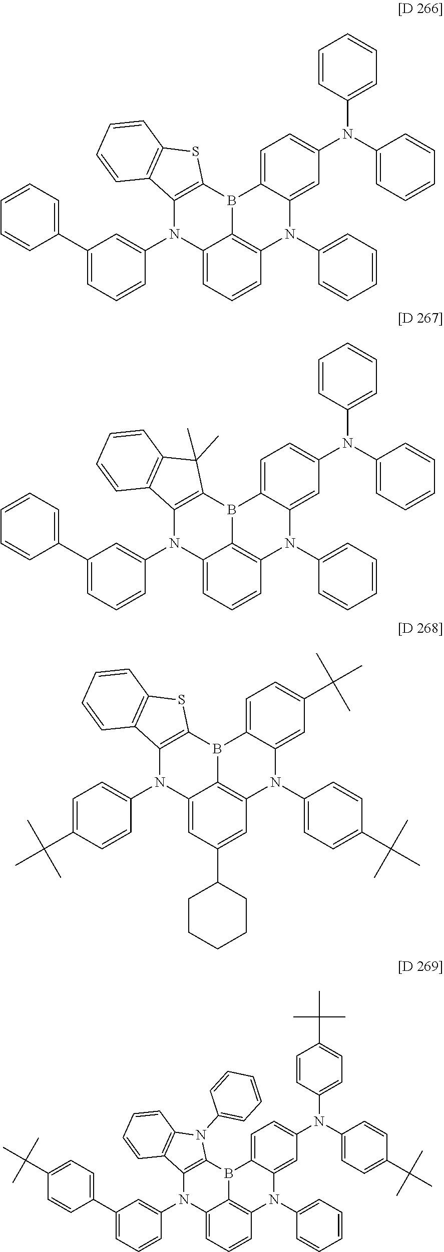

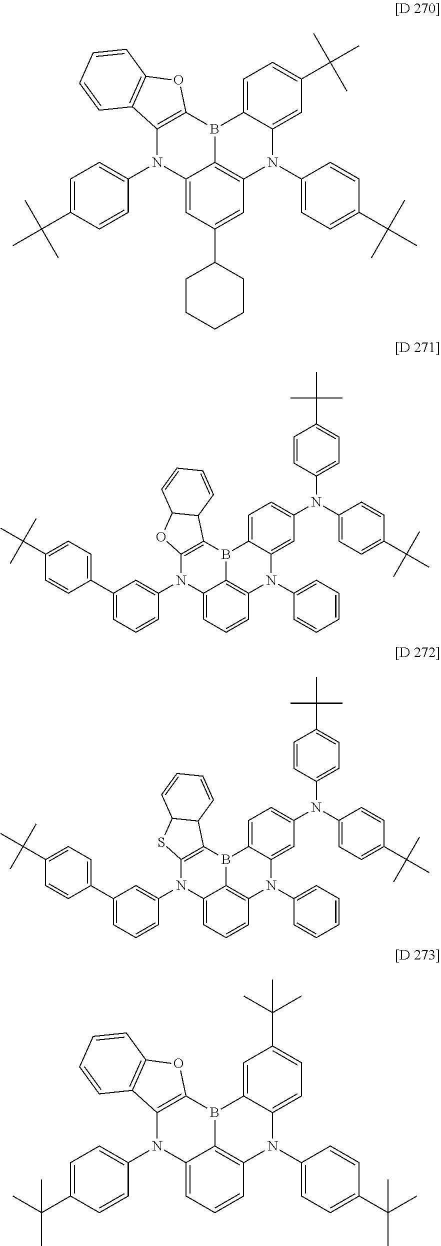

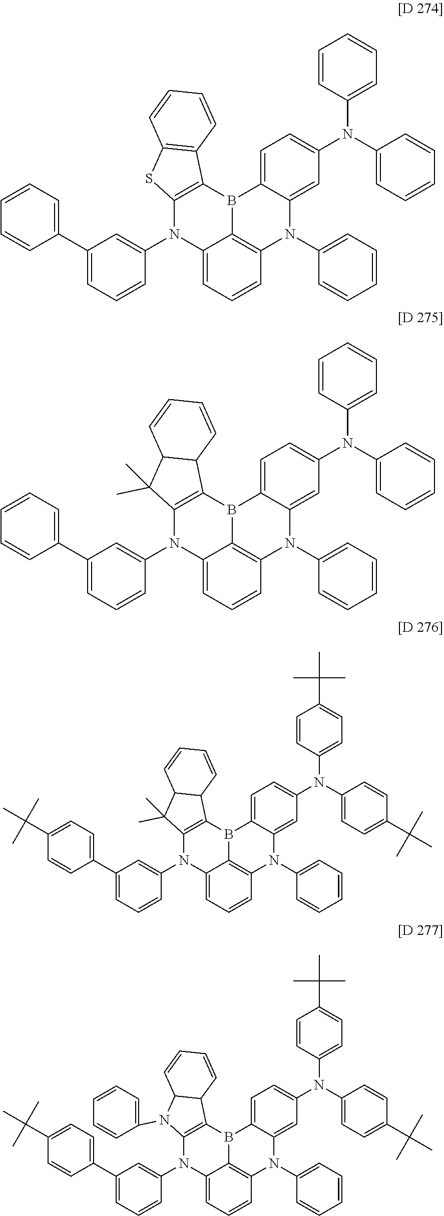

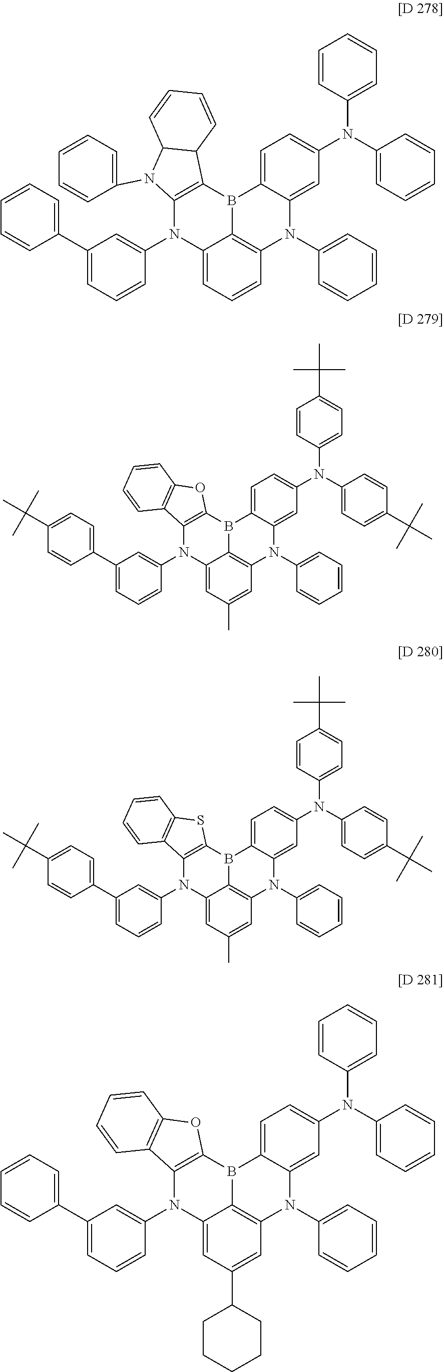

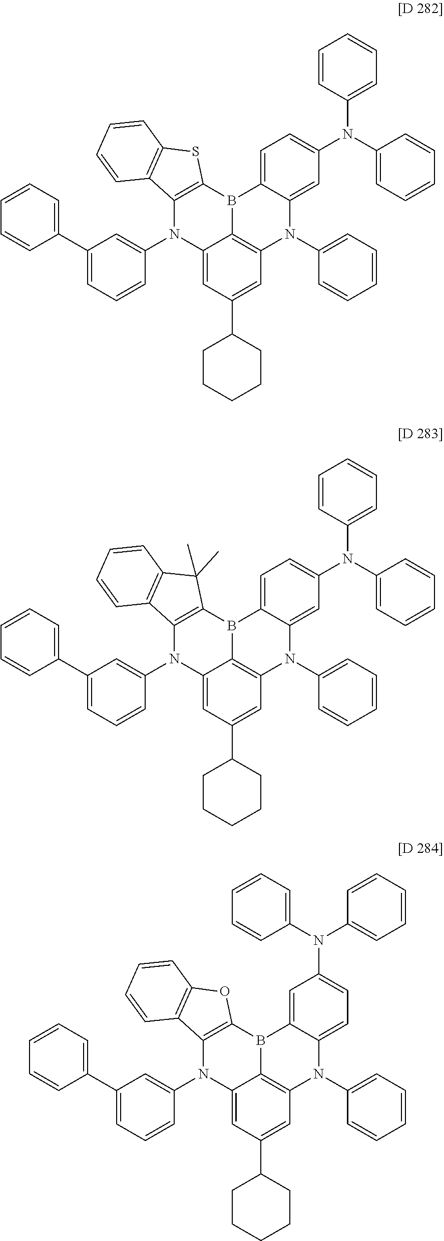 [SMILES: c1ccc(-c2cccc(N3c4cc(C5CCCCC5)cc5c4B(c4ccc(N(c6ccccc6)c6ccccc6)cc4N5c4ccccc4)c4sc5ccccc5c43)c2)cc1 ; CC1(C)C2=C(c3ccccc31)N(c1cccc(-c3ccccc3)c1)c1cc(C3CCCCC3)cc3c1B2c1ccc(N(c2ccccc2)c2ccccc2)cc1N3c1ccccc1 ; c1ccc(-c2cccc(N3c4cc(C5CCCCC5)cc5c4B(c4cc(N(c6ccccc6)c6ccccc6)ccc4N5c4ccccc4)c4oc5ccccc5c43)c2)cc1]

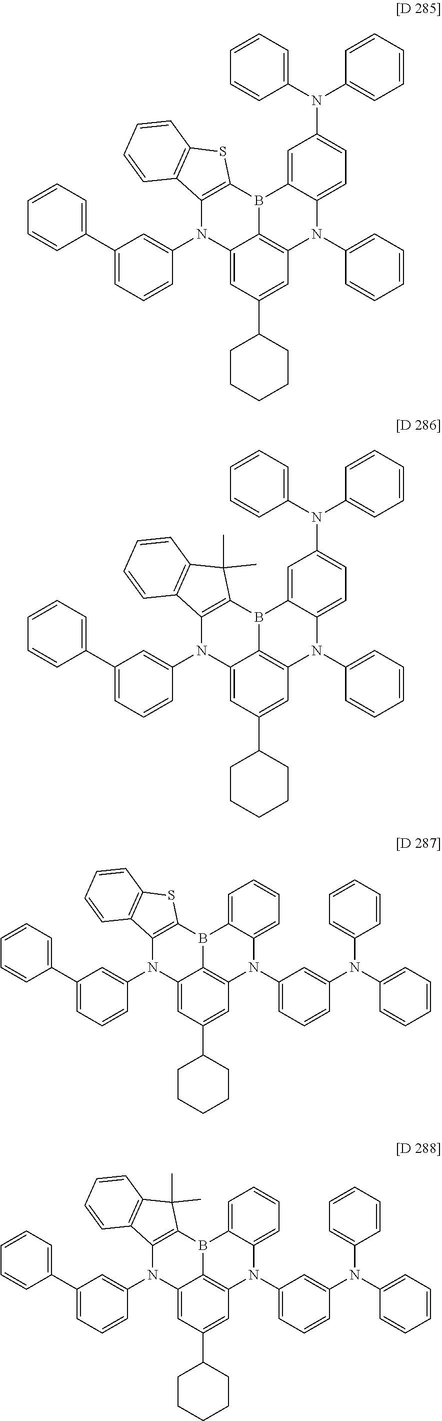 [SMILES: c1ccc(-c2cccc(N3c4cc(C5CCCCC5)cc5c4B(c4cc(N(c6ccccc6)c6ccccc6)ccc4N5c4ccccc4)c4sc5ccccc5c43)c2)cc1 ; CC1(C)C2=C(c3ccccc31)N(c1cccc(-c3ccccc3)c1)c1cc(C3CCCCC3)cc3c1B2c1cc(N(c2ccccc2)c2ccccc2)ccc1N3c1ccccc1 ; c1ccc(-c2cccc(N3c4cc(C5CCCCC5)cc5c4B(c4ccccc4N5c4cccc(N(c5ccccc5)c5ccccc5)c4)c4sc5ccccc5c43)c2)cc1 ; CC1(C)C2=C(c3ccccc31)N(c1cccc(-c3ccccc3)c1)c1cc(C3CCCCC3)cc3c1B2c1ccccc1N3c1cccc(N(c2ccccc2)c2ccccc2)c1]

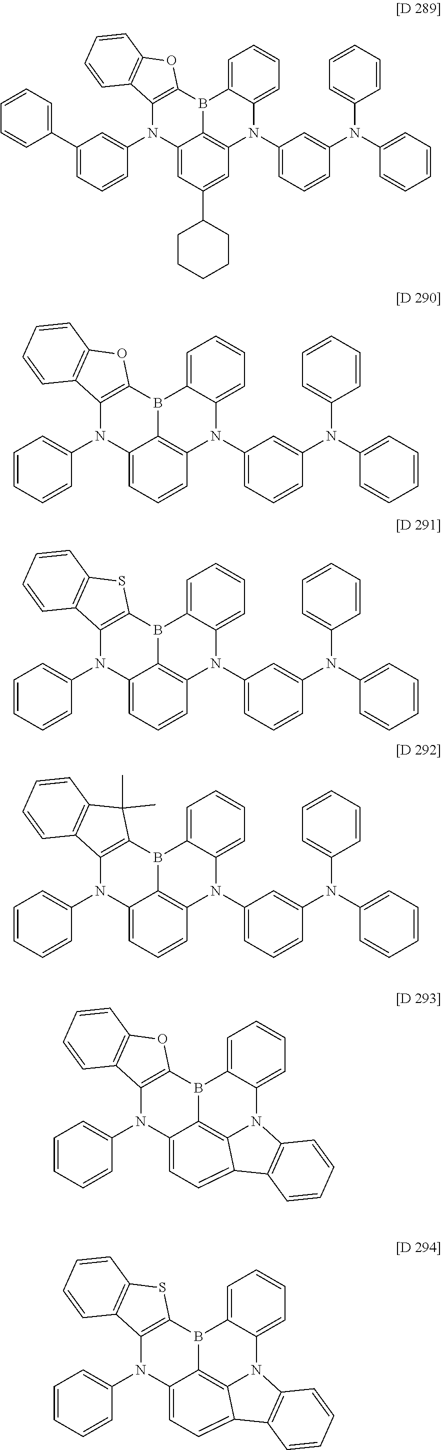 [SMILES: c1ccc(-c2cccc(N3c4cc(C5CCCCC5)cc5c4B(c4ccccc4N5c4cccc(N(c5ccccc5)c5ccccc5)c4)c4oc5ccccc5c43)c2)cc1 ; c1ccc(N(c2ccccc2)c2cccc(N3c4ccccc4B4c5oc6ccccc6c5N(c5ccccc5)c5cccc3c54)c2)cc1 ; c1ccc(N(c2ccccc2)c2cccc(N3c4ccccc4B4c5sc6ccccc6c5N(c5ccccc5)c5cccc3c54)c2)cc1 ; CC1(C)C2=C(c3ccccc31)N(c1ccccc1)c1cccc3c1B2c1ccccc1N3c1cccc(N(c2ccccc2)c2ccccc2)c1 ; c1ccc(N2c3ccc4c5ccccc5n5c4c3B(c3ccccc3-5)c3oc4ccccc4c32)cc1 ; c1ccc(N2c3ccc4c5ccccc5n5c4c3B(c3ccccc3-5)c3sc4ccccc4c32)cc1]

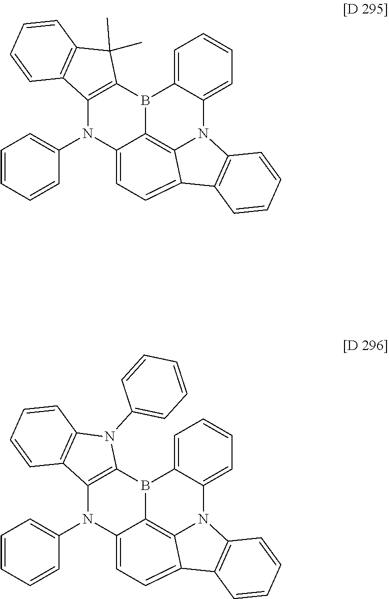 [SMILES: CC1(C)C2=C(c3ccccc31)N(c1ccccc1)c1ccc3c4ccccc4n4c3c1B2c1ccccc1-4 ; c1ccc(N2c3ccc4c5ccccc5n5c4c3B(c3ccccc3-5)c3c2c2ccccc2n3-c2ccccc2)cc1]

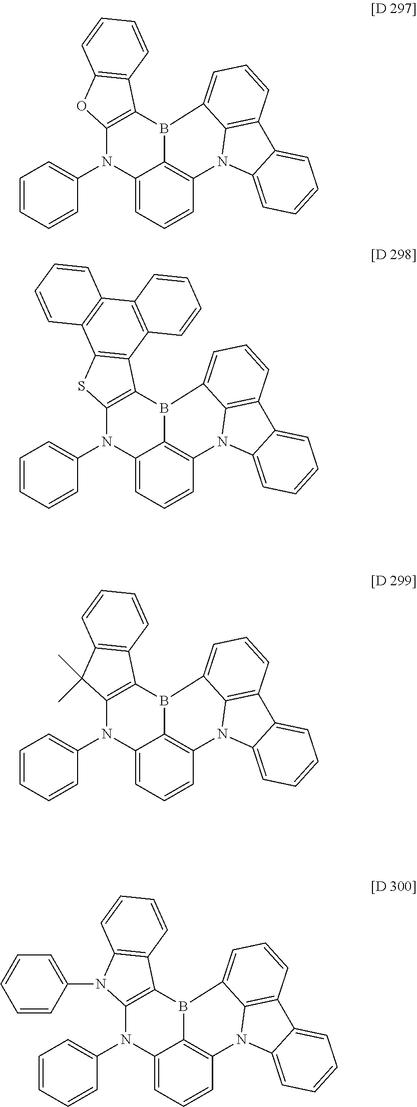

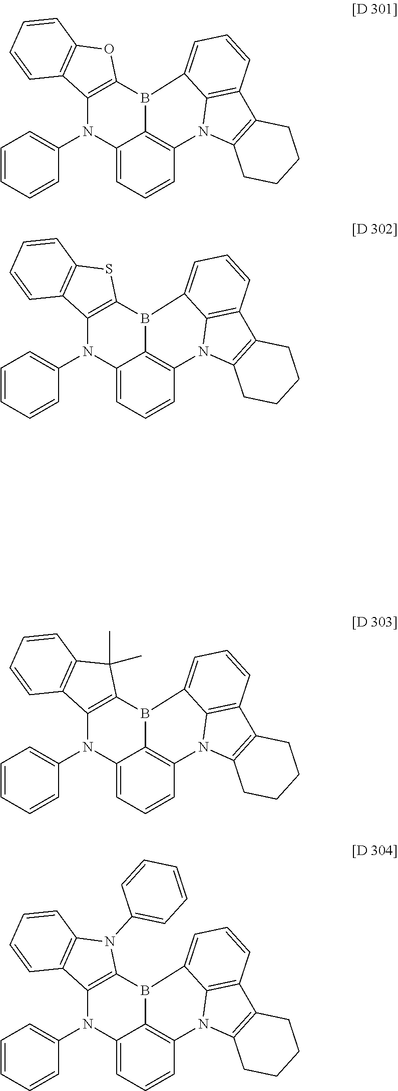

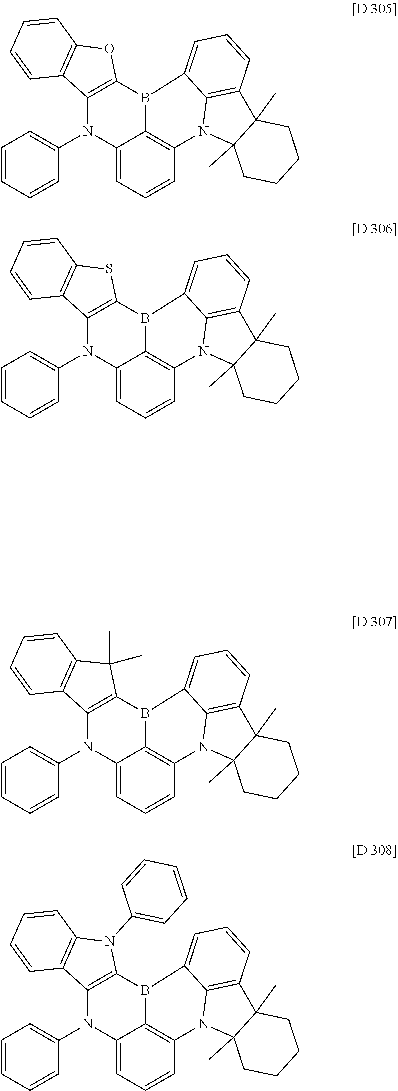

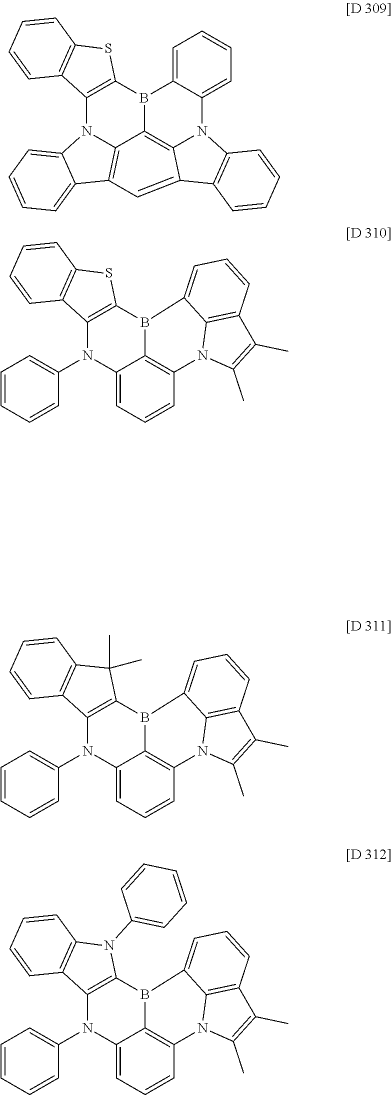

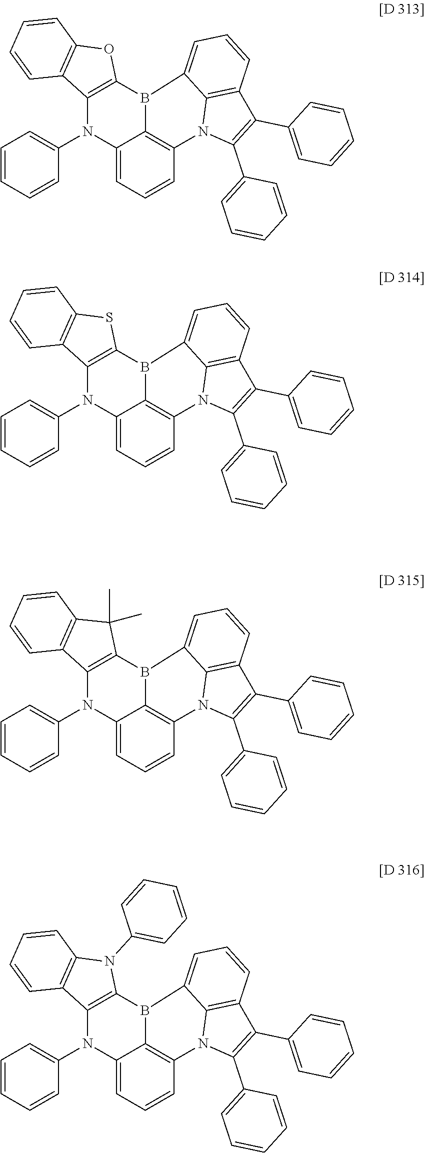 [SMILES: c1ccc(-c2c(-c3ccccc3)n3c4c(cccc24)B2c4oc5ccccc5c4N(c4ccccc4)c4cccc-3c42)cc1 ; c1ccc(-c2c(-c3ccccc3)n3c4c(cccc24)B2c4sc5ccccc5c4N(c4ccccc4)c4cccc-3c42)cc1 ; CC1(C)C2=C(c3ccccc31)N(c1ccccc1)c1cccc3c1B2c1cccc2c(-c4ccccc4)c(-c4ccccc4)n-3c12 ; c1ccc(-c2c(-c3ccccc3)n3c4c(cccc24)B2c4c(cccc4-3)N(c3ccccc3)c3c2n(-c2ccccc2)c2ccccc32)cc1]

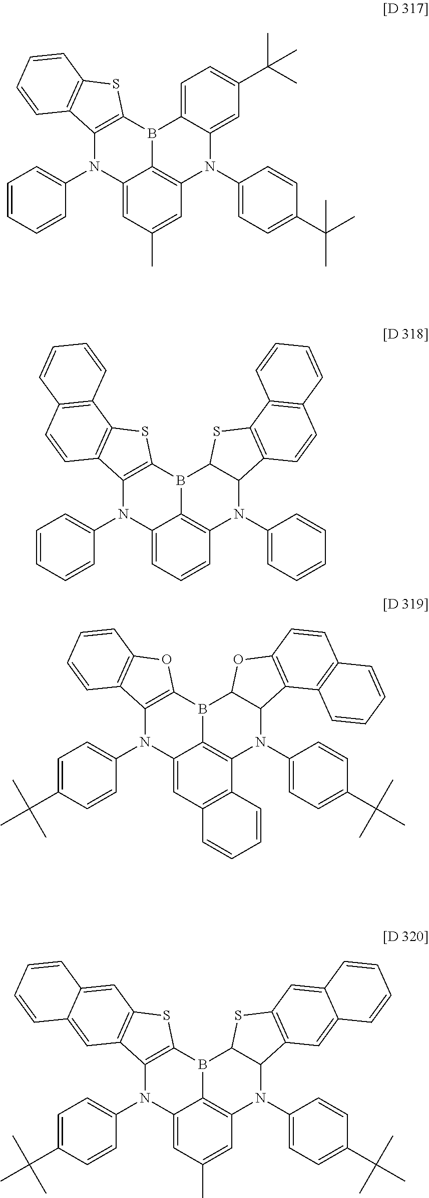

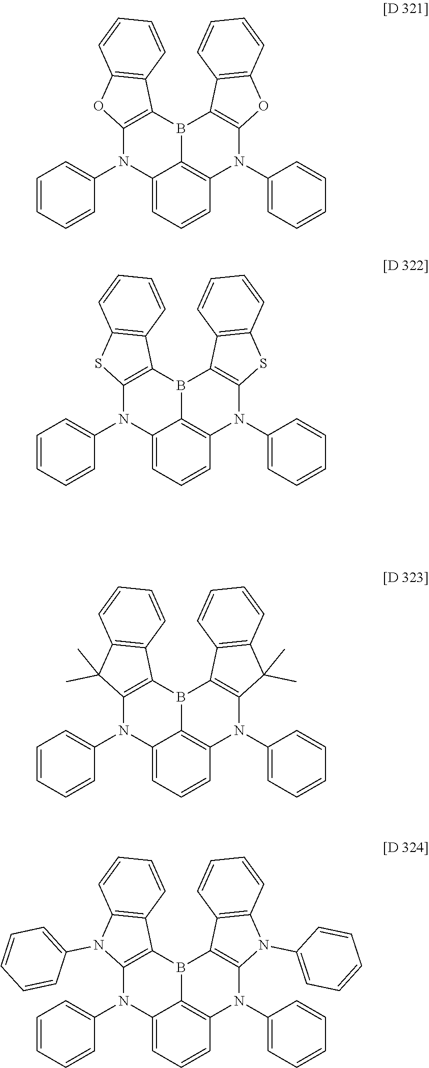 [SMILES: c1ccc(N2c3cccc4c3B(c3c2oc2ccccc32)c2c(oc3ccccc23)N4c2ccccc2)cc1 ; c1ccc(N2c3cccc4c3B(c3c2sc2ccccc32)c2c(sc3ccccc23)N4c2ccccc2)cc1 ; CC1(C)C2=C(B3C4=C(N(c5ccccc5)c5cccc(c53)N2c2ccccc2)C(C)(C)c2ccccc24)c2ccccc21 ; c1ccc(N2c3cccc4c3B(c3c2n(-c2ccccc2)c2ccccc32)c2c(n(-c3ccccc3)c3ccccc23)N4c2ccccc2)cc1]

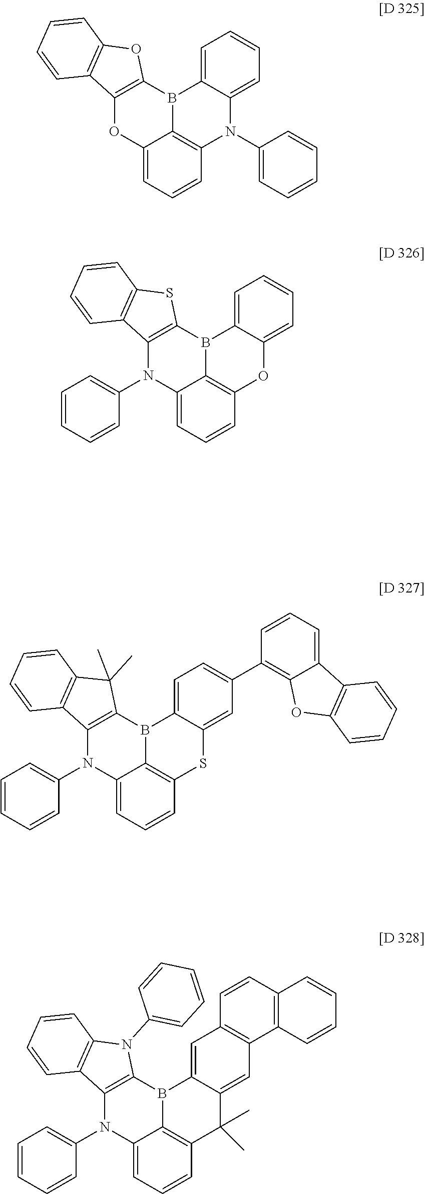 [SMILES: c1ccc(N2c3ccccc3B3c4oc5ccccc5c4Oc4cccc2c43)cc1 ; c1ccc(N2c3cccc4c3B(c3ccccc3O4)c3sc4ccccc4c32)cc1 ; CC1(C)C2=C(c3ccccc31)N(c1ccccc1)c1cccc3c1B2c1ccc(-c2cccc4c2oc2ccccc24)cc1S3 ; CC1(C)c2cc3c(ccc4ccccc43)cc2B2c3c(cccc31)N(c1ccccc1)c1c2n(-c2ccccc2)c2ccccc12]

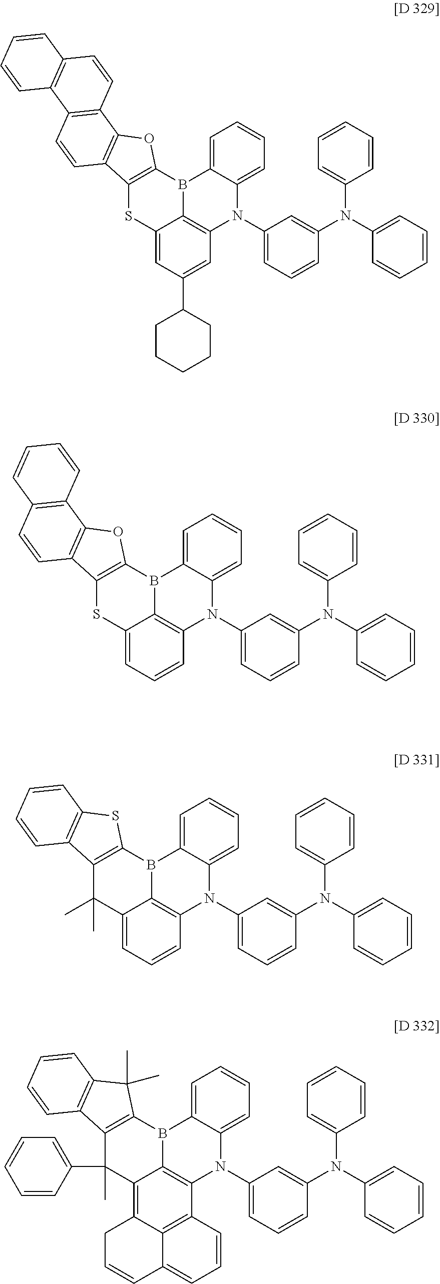 [SMILES: c1ccc(N(c2ccccc2)c2cccc(N3c4ccccc4B4c5oc6c(ccc7c8ccccc8ccc76)c5Sc5cc(C6CCCCC6)cc3c54)c2)cc1 ; c1ccc(N(c2ccccc2)c2cccc(N3c4ccccc4B4c5oc6c(ccc7ccccc76)c5Sc5cccc3c54)c2)cc1 ; CC1(C)c2cccc3c2B(c2ccccc2N3c2cccc(N(c3ccccc3)c3ccccc3)c2)c2sc3ccccc3c21 ; CC1(C)C2=C(c3ccccc31)C(C)(c1ccccc1)c1c3c(c4cccc5c4c1CC=C5)N(c1cccc(N(c4ccccc4)c4ccccc4)c1)c1ccccc1B23]

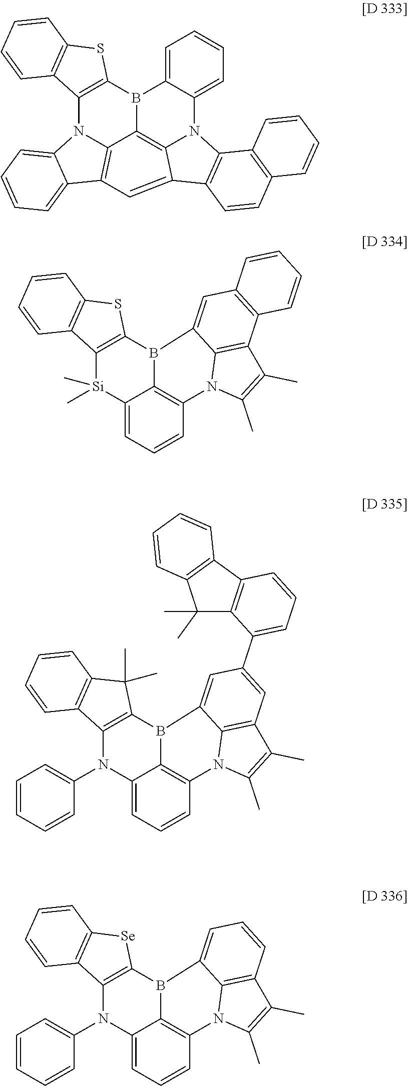 [SMILES: c1ccc2c(c1)B1c3sc4ccccc4c3-n3c4ccccc4c4cc5c6ccc7ccccc7c6n-2c5c1c43 ; Cc1c(C)n2c3c(cc4ccccc4c13)B1c3sc4ccccc4c3[Si](C)(C)c3cccc-2c31 ; Cc1c(C)n2c3c(cc(-c4cccc5c4C(C)(C)c4ccccc4-5)cc13)B1C3=C(c4ccccc4C3(C)C)N(c3ccccc3)c3cccc-2c31 ; Cc1c(C)n2c3c(cccc13)B1c3[se]c4ccccc4c3N(c3ccccc3)c3cccc-2c31]

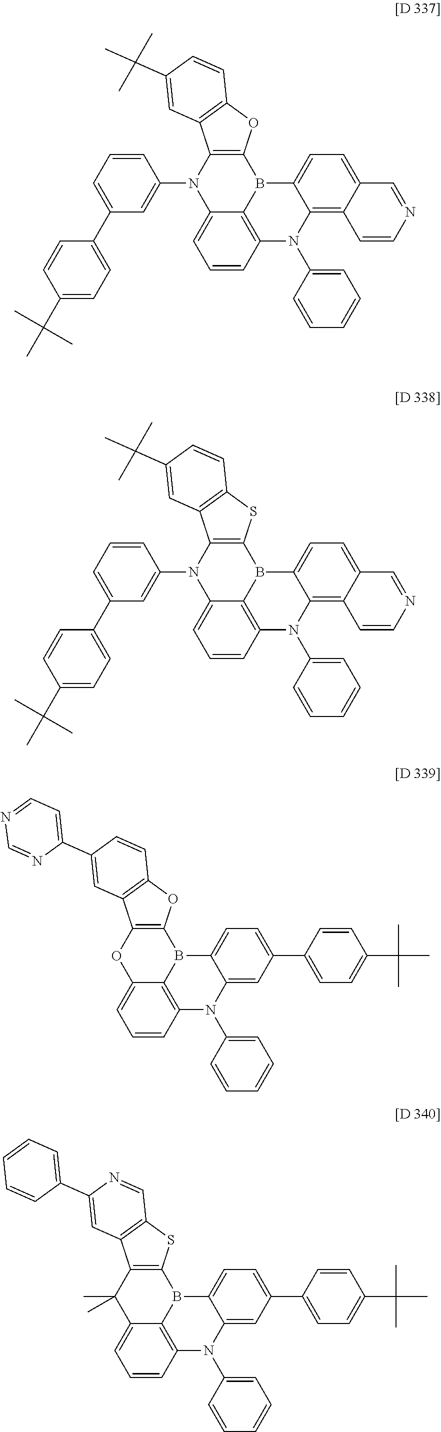 [SMILES: CC(C)(C)c1ccc(-c2cccc(N3c4cccc5c4B(c4ccc6cnccc6c4N5c4ccccc4)c4oc5ccc(C(C)(C)C)cc5c43)c2)cc1 ; CC(C)(C)c1ccc(-c2cccc(N3c4cccc5c4B(c4ccc6cnccc6c4N5c4ccccc4)c4sc5ccc(C(C)(C)C)cc5c43)c2)cc1 ; CC(C)(C)c1ccc(-c2ccc3c(c2)N(c2ccccc2)c2cccc4c2B3c2oc3ccc(-c5ccncn5)cc3c2O4)cc1 ; CC(C)(C)c1ccc(-c2ccc3c(c2)N(c2ccccc2)c2cccc4c2B3c2sc3cnc(-c5ccccc5)cc3c2C4(C)C)cc1]

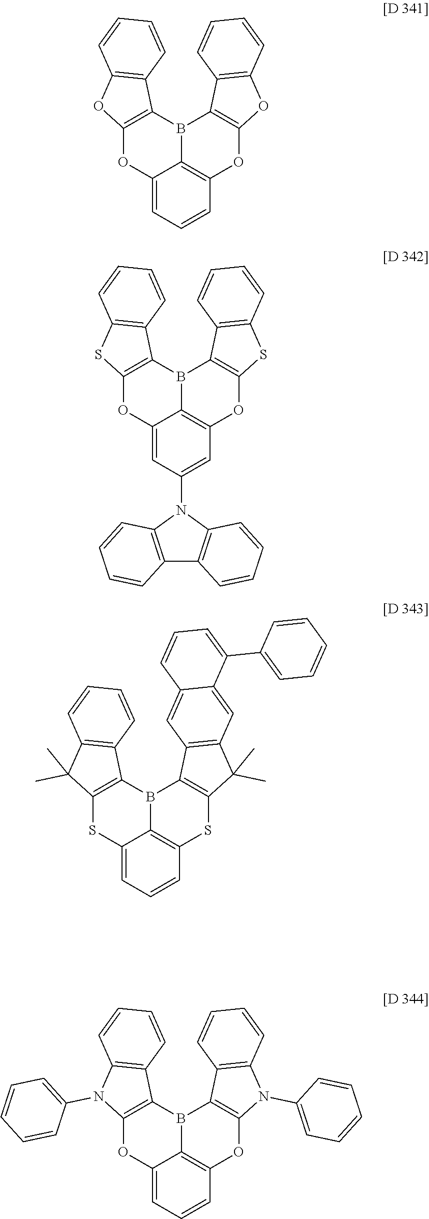

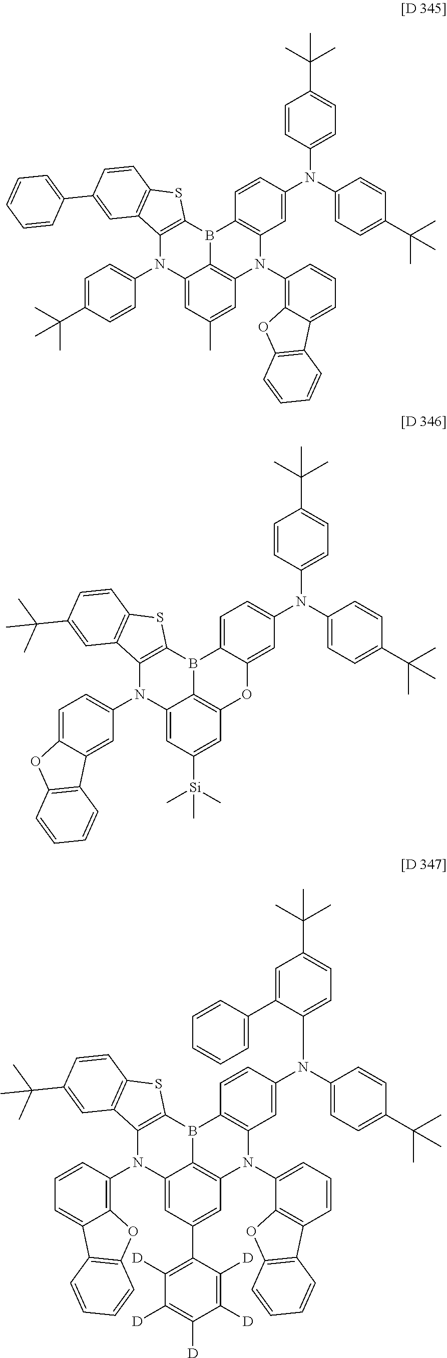

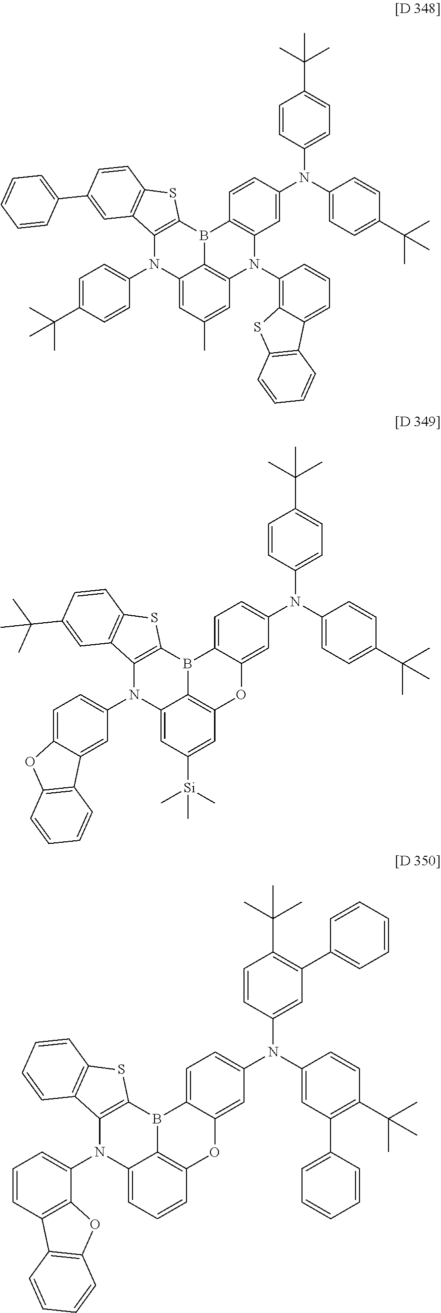

Throughout the description of the present disclosure, the phrase “(organic layer) includes at least one organic compound” may be construed to mean that “(organic layer) may include a single organic compound species or two or more different species of organic compounds falling within the scope of the present disclosure”.

An organic light emitting diode according to the present disclosure includes: an anode as a first electrode; a cathode as a second electrode facing the first electrode; and a light emitting layer interposed between the anode and the cathode, wherein the organic layer contains the compound represented by Chemical Formula A as a host and at least one of the boron compounds represented by Chemical Formulas D-6 or D-7 as a dopant. Having such structural characteristics, the organic light-emitting diode according to the present disclosure can be driven at a low voltage with longevity.

In this regard, the organic light emitting diode according to the present disclosure may include at least one of a hole injection layer, a hole transport layer, a functional layer capable of both hole injection and hole transport, an electron transport layer, and an electron injection layer, in addition to the light-emitting layer.

In a particular embodiment, the content of the dopant in the light-emitting layer may range from about 0.01 to 20 parts by weight, based on 100 parts by weight of the host, but is not limited thereto.

In addition to the above-mentioned dopants and hosts, the light-emitting layer may further include various hosts and dopant materials. For instance, the host within the light emitting layer further comprises one or more host compounds different from the anthracene compound represented by Chemical Formula A, whereby two or more different host compounds are used in mixture in the light emitting layer.

Below, an organic light-emitting diode according to an embodiment of the present disclosure is explained with reference to the drawing.

The FIGURE is a schematic cross-sectional view of the structure of an organic light-emitting diode according to an embodiment of the present disclosure.

As shown in the FIGURE, the organic light-emitting diode according to an embodiment of the present disclosure comprises an anode (20), a hole transport layer (40), an organic light-emitting layer (50) containing a host and a dopant, an electron transport layer (60), and a cathode (80), wherein the anode and the cathode serve as a first electrode and a second electrode, respectively, with the interposition of the hole transport layer between the anode and the light-emitting layer, and the electron transport layer between the light-emitting layer and the cathode.

Furthermore, the organic light-emitting diode according to an embodiment of the present disclosure may include a hole injection layer (30) between the anode (20) and the hole transport layer (40), and an electron injection layer (70) between the electron transport layer (60) and the cathode (80).

Reference is made to the FIGURE with regard to the organic light emitting diode of the present disclosure and the fabrication method therefor.

First, a substrate (10) is coated with an anode electrode material to form an anode (20). So long as it is used in a typical organic electroluminescence device, any substrate may be used as the substrate (10). Preferable is an organic substrate or transparent plastic substrate that exhibits excellent transparency, surface smoothness, ease of handling, and waterproofness. As the anode electrode material, indium tin oxide (ITO), indium zinc oxide (IZO), tin oxide (SnO₂), or zinc oxide (ZnO), which are transparent and superior in terms of conductivity, may be used.

A hole injection layer material is applied on the anode (20) by thermal deposition in a vacuum or by spin coating to form a hole injection layer (30). Subsequently, thermal deposition in a vacuum or by spin coating may also be conducted to form a hole transport layer (40) with a hole transport layer material on the hole injection layer (30).

So long as it is typically used in the art, any material may be selected for the hole injection layer without particular limitations thereto. Examples include, but are not limited to, 2-TNATA [4,4′,4″-tris(2-naphthylphenyl-phenylamino)-triphenylamine], NPD [N,N′-di(1-naphthyl)-N,N′-diphenylbenzidine)], TPD [N,N′-diphenyl-N,N′-bis(3-methylphenyl)-1,1′-biphenyl-4,4′-diamine], and DNTPD [N,N′-diphenyl-N,N′-bis-[4-(phenyl-m-tolyl-amino)-phenyl]-biphenyl-4,4′-diamine].

Any material that is typically used in the art may be selected for the hole transport layer without particular limitations thereto. Examples include, but are not limited to, N,N′-bis(3-methylphenyl)-N,N′-diphenyl-[1,1-biphenyl]-4,4′-diamine (TPD) and N,N′-di(naphthalen-1-yl)-N,N′-diphenylbenzidine (a-NPD).

In an embodiment of the present disclosure, an electron blocking layer may be additionally disposed on the hole transport layer. Functioning to prevent the electrons injected from the electron injection layer from entering the hole transport layer from the light-emitting layer, the electron blocking layer is adapted to increase the life span and luminous efficiency of the diode. The electron blocking layer may be formed at a suitable position between the light emitting layer and the hole injection layer. Particularly, the electron blocking layer may be formed between the light emitting layer and the hole transport layer.

Next, the light-emitting layer (50) may be deposited on the hole transport layer (40) or the electron blocking layer by deposition in a vacuum or by spin coating.

Herein, the light-emitting layer may contain a host and a dopant and the materials are as described above.

In some embodiments of the present disclosure, the light-emitting layer particularly ranges in thickness from 50 to 2,000 Å.

Meanwhile, the electron transport layer (60) is applied on the light-emitting layer by deposition in a vacuum and spin coating.

In this regard, the light emitting layer according to the present disclosure may be a stacked light emitting layer in a bi- or multilayer structure including: a first light emitting layer containing a host represented by Chemical Formula A and a dopant represented by any one of Chemical Formulas D-6 and D-7; and a second light emitting layer.

Here, the second light emitting layer is preferably different from the first light emitting layer in that at least one of the host compound and dopant compound employed in the first light emitting layer is not applied in the second light emitting layer.

That is, the light emitting layer according to the present disclosure has a bi- or multilayer structure in which the first light emitting layer contains: the host represented by Chemical Formula A; and the dopant represented by any one of Chemical Formulas D-6 and D-7 and the second light emitting layer may contain a compound, different from the compound represented by Chemical Formula A, as a host and a compound, different from the compound represented by any one of Chemical Formulas D-6 and D-7, as a dopant.

Also, the second light emitting layer of the present disclosure may employ as a host a compound represented by Chemical Formula A that is different from the compound “used” as a host in the first light emitting layer. Moreover, a compound represented by any one of Chemical Formulas D-6 and D-7, but different from the compound “used” as a dopant in the first light emitting layer, may be used as a dopant in a second light emitting layer. Thus, a second light emitting layer different from the first light emitting layer may be deposited.

For example, the first light emitting layer may contain compound A-3 as a host and compound D 265 as a dopant while the second light emitting layer may contain compound A-6 as a host and compound D 20 as a dopant. The first light emitting layer may contain compound A-13 as a host and compound D 322 as a dopant while the second light emitting layer may contain compound A-6 as a host and a compound, different from that represented by any one of Chemical Formulas D-6 to D-7, as a dopant. In another example, compound A-6 according to the present disclosure is used as a host and D 321 as a dopant in the first light emitting layer while the second light emitting layer employs a compound, different from that represented by Chemical Formula A, as a host and a compound represented by any one of Chemical Formula D-6 and D-7 as a dopant.

A material for use in the electron transport layer functions to stably carry the electrons injected from the electron injection electrode (cathode), and may be an electron transport material known in the art. Examples of the electron transport material known in the art include quinoline derivatives, particularly, tris(8-quinolinolate)aluminum (Alq₃), Liq, TAZ, BAlq, beryllium bis(benzoquinolin-10-olate) (Bebq₂), Compound 201, Compound 202, BCP, and oxadiazole derivatives such as PBD, BMD, and BND, but are not limited thereto:

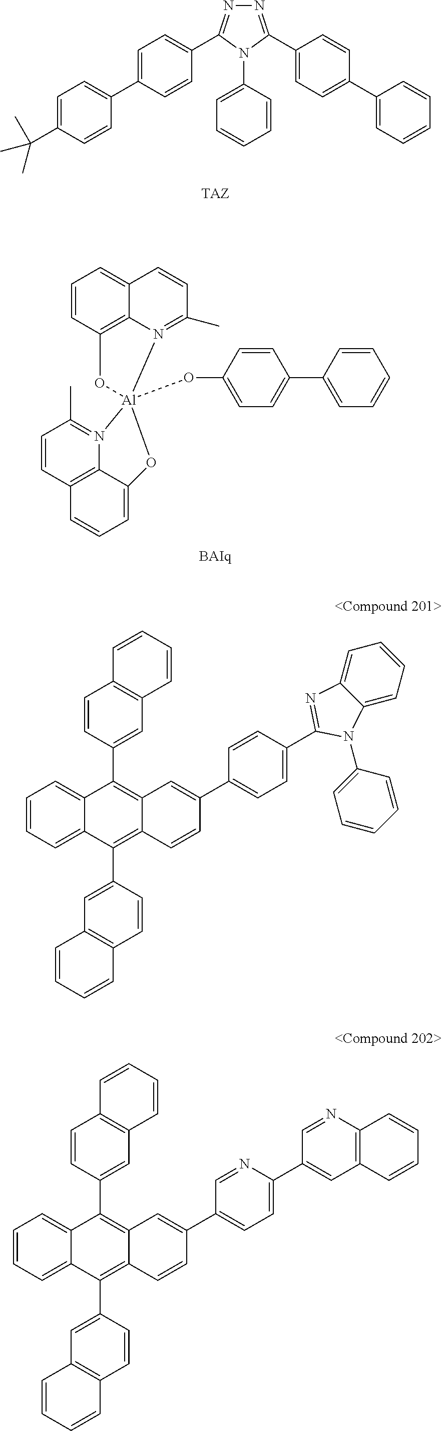

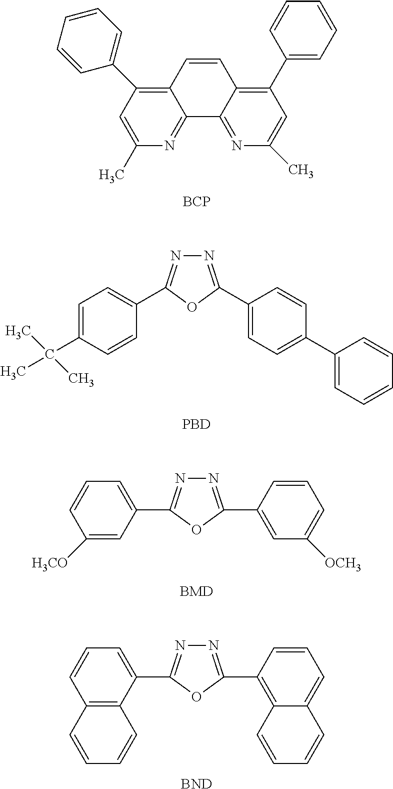

In the organic light emitting diode of the present disclosure, an electron injection layer (EIL) that functions to facilitate electron injection from the cathode may be deposited on the electron transport layer. The material for the EIL is not particularly limited.

Any material that is conventionally used in the art can be available for the electron injection layer without particular limitations. Examples include CsF, NaF, LiF, Li₂O, and BaO. Deposition conditions for the electron injection layer may vary, depending on compounds used, but may be generally selected from condition scopes that are almost same as for the formation of hole injection layers.

The electron injection layer may range in thickness from about 1 Å to about 100 Å, and particularly from about 3 Å to about 90 Å. Given the thickness range for the electron injection layer, the diode can exhibit satisfactory electron injection properties without actually elevating a driving voltage.

In order to facilitate electron injection, the cathode may be made of a material having a small work function, such as metal or metal alloy such as lithium (Li), magnesium (Mg), calcium (Ca), an alloy aluminum (Al) thereof, aluminum-lithium (Al—Li), magnesium-indium (Mg—In), and magnesium-silver (Mg—Ag). Alternatively, ITO or IZO may be employed to form a transparent cathode for an organic light-emitting diode.

Moreover, the organic light-emitting diode of the present disclosure may further comprise a light-emitting layer containing a blue, green, or red luminescent material that emits radiations in the wavelength range of 380 nm to 800 nm. That is, the light-emitting layer in the present disclosure has a multi-layer structure wherein the blue, green, or red luminescent material may be a fluorescent material or a phosphorescent material.

Furthermore, at least one selected from among the layers may be deposited using a single-molecule deposition process or a solution process.

Here, the deposition process is a process by which a material is vaporized in a vacuum or at a low pressure and deposited to form a layer, and the solution process is a method in which a material is dissolved in a solvent and applied for the formation of a thin film by means of inkjet printing, roll-to-roll coating, screen printing, spray coating, dip coating, spin coating, etc.

Also, the organic light-emitting diode of the present disclosure may be applied to a device selected from among flat display devices, flexible display devices, monochrome or grayscale flat illumination devices, and monochrome or grayscale flexible illumination devices.

A better understanding of the present disclosure may be obtained through the following examples which are set forth to illustrate, but are not to be construed as limiting the present disclosure.

### EXAMPLES

### Synthesis Examples: Synthesis of Host Compound (Represented by Chemical Formula A)

### Synthesis Example 1: Synthesis of <A-13>

### Synthesis Example 1-(1): Synthesis of <1-a>

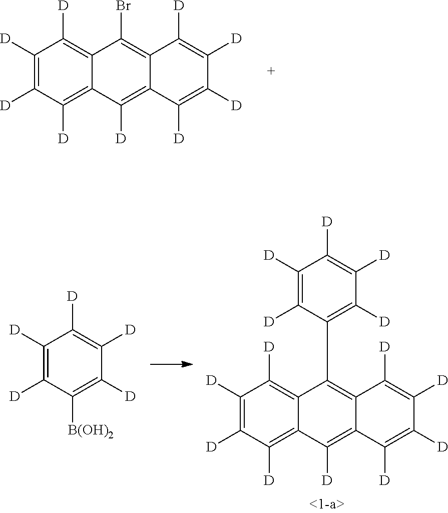 [SMILES: [2H]c1c([2H])c([2H])c2c(Br)c3c([2H])c([2H])c([2H])c([2H])c3c([2H])c2c1[2H] ; [2H]c1c([2H])c([2H])c(B(O)O)c([2H])c1[2H] ; [2H]c1c([2H])c([2H])c(-c2c3c([2H])c([2H])c([2H])c([2H])c3c([2H])c3c([2H])c([2H])c([2H])c([2H])c23)c([2H])c1[2H]]

In a 2 L round-bottom flask reactor, a mixture of 9-bromoanthracene-d9 (100 g, 0.376 mol), phenyl boronic acid-d5 (57.2 g, 0.451 mol), tetrakis(triphenylphosphine)palladium (8.7 g, 8 mmol), and potassium carbonate (103.8 g, 0.751 mol) was added with toluene (600 mL), ethanol (300 mL), and water (300 mL). at an elevated temperature, the resulting mixture was stirred overnight under reflux in the reactor. After completion of the reaction, the reactor was cooled to room temperature. Extraction with ethyl acetate separated an organic layer. The organic layer was concentrated in a vacuum and purified by column chromatography to afford <1-a>. (80.0 g, 79.3%)

### Synthesis Example 1-(2): Synthesis of <1-b>

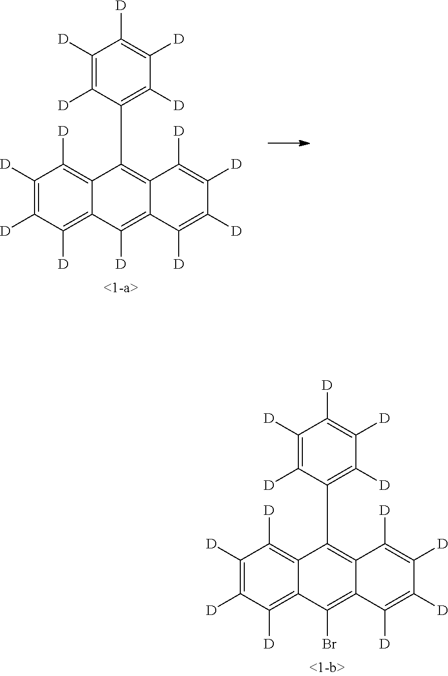 [SMILES: [2H]c1c([2H])c([2H])c(-c2c3c([2H])c([2H])c([2H])c([2H])c3c([2H])c3c([2H])c([2H])c([2H])c([2H])c23)c([2H])c1[2H] ; [2H]c1c([2H])c([2H])c(-c2c3c([2H])c([2H])c([2H])c([2H])c3c(Br)c3c([2H])c([2H])c([2H])c([2H])c23)c([2H])c1[2H]]

In a 2-L round-bottom flask reactor, <1-a> (80.0 g, 0.298 mol) was dissolved in dichloromethane (960 ml). This reaction solution was cooled to 0° C. in a nitrogen atmosphere, and then a solution of N-bromosuccinimide (63.7 g, 0.358 ml) in N,N-dimethylformamide (200 ml) was slowly added in a dropwise manner. Subsequent to completion of dropwise addition, stirring was conducted for 5 hours at room temperature. When the reaction was completed as monitored by thin layer chromatography, the organic layer was washed with an aqueous sodium hydrogen carbonate solution and additionally with water. After being separated, the organic layer was filtered through silica gel pad and concentrated in a vacuum. Recrystallization in dichloromethane and methanol afforded <1-b>. (78.0 g, 75.6%)

### Synthesis Example 1-(3): Synthesis of <1-c>

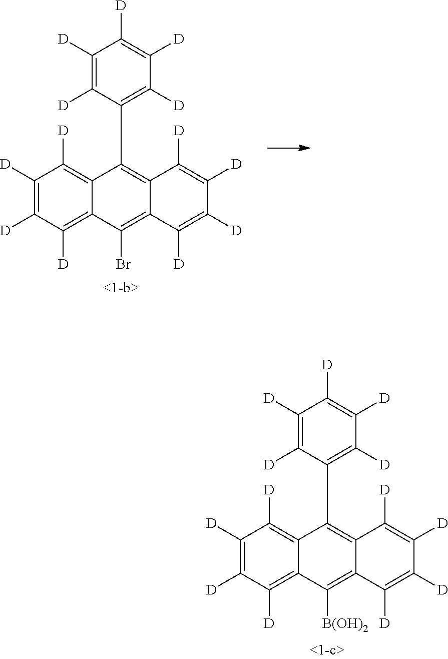 [SMILES: [2H]c1c([2H])c([2H])c(-c2c3c([2H])c([2H])c([2H])c([2H])c3c(Br)c3c([2H])c([2H])c([2H])c([2H])c23)c([2H])c1[2H] ; [2H]c1c([2H])c([2H])c(-c2c3c([2H])c([2H])c([2H])c([2H])c3c(B(O)O)c3c([2H])c([2H])c([2H])c([2H])c23)c([2H])c1[2H]]

In a 2-L round-bottom flask, a solution of <1-b> (78.0 g, 0.225 mol) in tetrahydrofuran (780 ml) was chilled to −78° C. in a nitrogen atmosphere and stirred.

To the chilled reaction solution were slowly added drops of n-butyl lithium (162 ml, 0.259 mol). After stirring at the same temperature for 2 hours, trimethyl borate (29.0 g, 0.282 mol) was dropwise added over 30 minutes and then stirred overnight at room temperature. After completion of the reaction, 2 N HCl was slowly added in a dropwise manner for acidification. Extraction with water and ethyl acetate separated an organic layer which was then dried over magnesium sulfate. The residue was concentrated in a vacuum and crystallized in heptane. The solid thus obtained was filtered and washed with heptane and toluene to afford <intermediate 1-c>. <50.0 g, 71%)

### Synthesis Example 1-(4): Synthesis of <1-d>

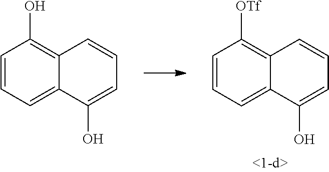 [SMILES: Oc1cccc2c(O)cccc12 ; O=S(=O)(Oc1cccc2c(O)cccc12)C(F)(F)F]

In a 2-L round-bottom flask reactor, 1,5-dihydroxynaphthalene (100.0 g, 0.625 mol) and dichloromethane (1000 ml) were slowly added with pyridine (148 g, 1.875 mol) and then stirred together at room temperature for 30 minutes. The mixture was cooled to 0° C. and then added with drops of trifluoromethane sulfonyl anhydride (176 g, 0.625 mol) at the same temperature. After stirring at room temperature for 5 hours, the reaction mixture was slowly added with water (500 ml) and stirred for 10 minutes. The solid thus formed was filtered and washed with dichloromethane. The filtrate was subjected to extraction and the organic layer was separated and concentrated in a vacuum. The concentrate was purified by column chromatography to afford <1-d>. (78.0 g, 42.7%)

### Synthesis Example 1-(5): Synthesis of <1-e>

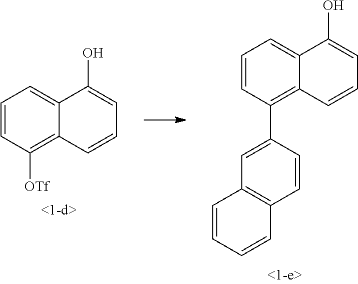 [SMILES: O=S(=O)(Oc1cccc2c(O)cccc12)C(F)(F)F ; Oc1cccc2c(-c3ccc4ccccc4c3)cccc12]

In a 2-L round-bottom flask reactor, a mixture of <1-d> (70 g, 0.240 mol), 2-naphthalene boronic acid (41.2 g, 0.240 mol), tetrakis(triphenylphosphine)palladium (5.5 g, 5 mmol), and potassium carbonate (66.2 g, 0.479 mol) was added with toluene (490 mL), ethanol (210 mL), and water (210 mL). At an elevated temperature, stirring was conducted for 5 hours under reflux. When the reaction was completed as monitored by thin layer chromatography, the reaction mixture was cooled to room temperature. Following extraction with ethyl acetate, an organic layer was separated, concentrated in a vacuum, and purified by column chromatography to afford <1-e>. (50.0 g, 77.3%)

### Synthesis Example 1-(6): Synthesis of <1-f>

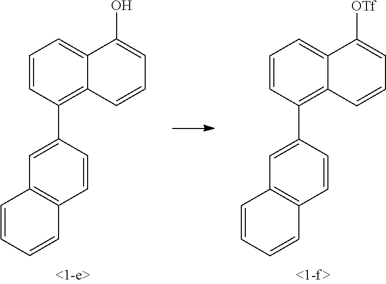 [SMILES: Oc1cccc2c(-c3ccc4ccccc4c3)cccc12 ; O=S(=O)(Oc1cccc2c(-c3ccc4ccccc4c3)cccc12)C(F)(F)F]

The same procedure as in Synthesis Example 1-(4) was carried out, with the exception of using <1-e> instead of 1,5-dihydroxynaphthalene, to afford <1-f> (62.0 g, 83.3%).

### Synthesis Example 1-(7): Synthesis of <A-13>

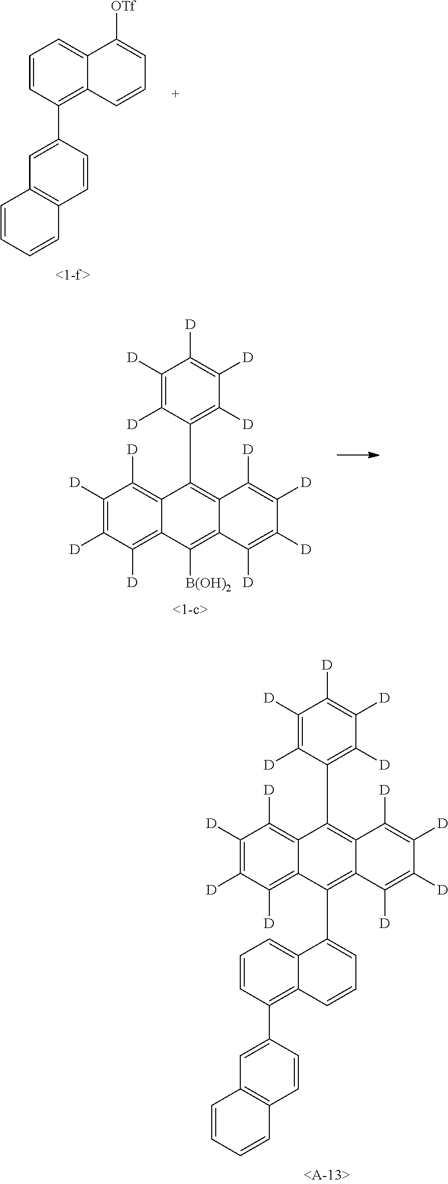 [SMILES: O=S(=O)(Oc1cccc2c(-c3ccc4ccccc4c3)cccc12)C(F)(F)F ; [2H]c1c([2H])c([2H])c(-c2c3c([2H])c([2H])c([2H])c([2H])c3c(B(O)O)c3c([2H])c([2H])c([2H])c([2H])c23)c([2H])c1[2H] ; [2H]c1c([2H])c([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1[2H]]

In a 500-mL round-bottom flask reactor, a mixture of <1-f> (20.0 g, 50 mmol), <1-c> (21.7 g, 70 mmol), tetrakis(triphenylphosphine)palladium (1.20 g, 1 mmol), and potassium carbonate (13.8 g, 99 mmol) was added with toluene (100 mL), ethanol (60 mL), and water (60 mL). At an elevated temperature, the resulting solution was stirred for 4 hours under reflux. After completion of the reaction, the reaction mixture was cooled to room temperature and added with ethanol to precipitate a crystal. The solid was filtered, dissolved in toluene, filtered through silica gel, and concentrated in a vacuum. The concentrate was recrystallized in toluene and acetone to afford <A-13>. (13.5 g, 52.2%)

MS (MALDI-TOF): m/z 519.29 [M⁺]

### Synthesis Example 2: Synthesis of <A-3>

### Synthesis Example 2-(1): Synthesis of <2-a>

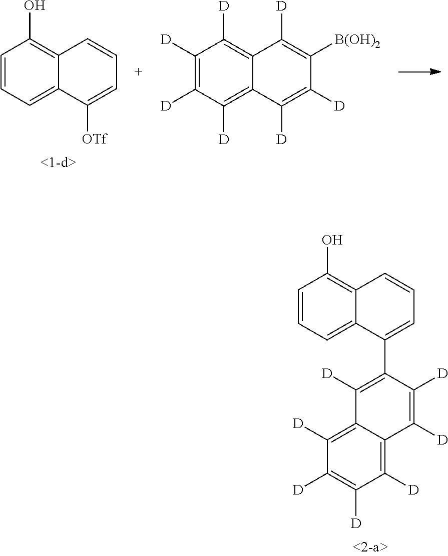 [SMILES: O=S(=O)(Oc1cccc2c(O)cccc12)C(F)(F)F ; [2H]c1c([2H])c([2H])c2c([2H])c(B(O)O)c([2H])c([2H])c2c1[2H] ; [2H]c1c([2H])c([2H])c2c([2H])c(-c3cccc4c(O)cccc34)c([2H])c([2H])c2c1[2H]]

The same procedure as in Synthesis Example 1-(5) was carried out, with the exception of using 2-naphthalene boronic acid (d7) instead of 2-naphthalene boronic acid, to afford <2-a> (52.0 g, 78.0%).

### Synthesis Example 2-(2): Synthesis of <2-b>

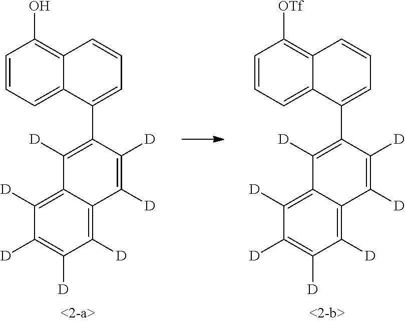 [SMILES: [2H]c1c([2H])c([2H])c2c([2H])c(-c3cccc4c(O)cccc34)c([2H])c([2H])c2c1[2H] ; [2H]c1c([2H])c([2H])c2c([2H])c(-c3cccc4c(OS(=O)(=O)C(F)(F)F)cccc34)c([2H])c([2H])c2c1[2H]]

The same procedure as in Synthesis Example 1-(4) was carried out, with the exception of using <2-a> instead of 1,5-dihydroxynaphthalene, to afford <2-b> (68.5 g, 89.0%).

### Synthesis Example 2-(3): Synthesis of <2-c>

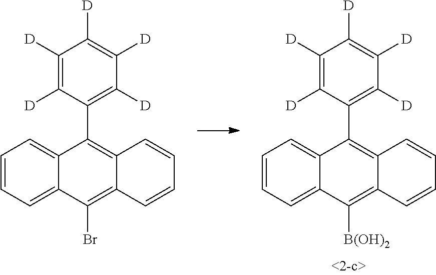 [SMILES: [2H]c1c([2H])c([2H])c(-c2c3ccccc3c(Br)c3ccccc23)c([2H])c1[2H] ; [2H]c1c([2H])c([2H])c(-c2c3ccccc3c(B(O)O)c3ccccc23)c([2H])c1[2H]]

The same procedure as in Synthesis Example 1-(3) was carried out, with the exception of using 9-bromo-10-phenyl(d5) anthracene instead of intermediate <1-b>, to afford intermediate <2-c> (71.0 g, 80.0%).

### Synthesis Example 2-(4): Synthesis of <A-3>

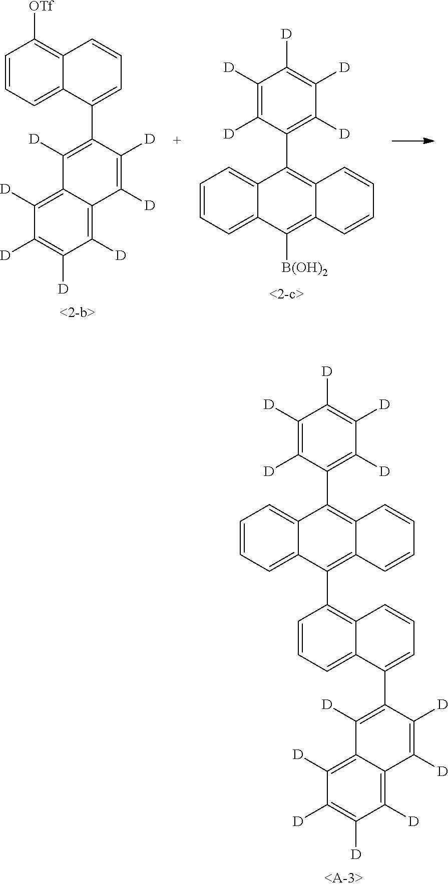 [SMILES: [2H]c1c([2H])c([2H])c2c([2H])c(-c3cccc4c(OS(=O)(=O)C(F)(F)F)cccc34)c([2H])c([2H])c2c1[2H] ; [2H]c1c([2H])c([2H])c(-c2c3ccccc3c(B(O)O)c3ccccc23)c([2H])c1[2H] ; [2H]c1c([2H])c([2H])c(-c2c3ccccc3c(-c3cccc4c(-c5c([2H])c([2H])c6c([2H])c([2H])c([2H])c([2H])c6c5[2H])cccc34)c3ccccc23)c([2H])c1[2H]]

The same procedure as in Synthesis Example 1-(7) was carried out, with the exception of using <2-b> and <2-c> instead of intermediate <1-f> and <1-c>, respectively, to afford <A-3> (6.2 g, 56.0%).

MS (MALDI-TOF): m/z 518.28 [M⁺]

### Synthesis Example 3: Synthesis of <A-10>

### Synthesis Example 3-(1): Synthesis of <3-a>

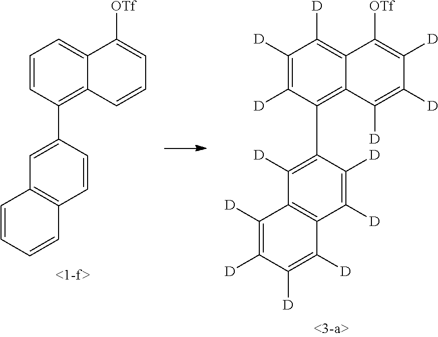 [SMILES: O=S(=O)(Oc1cccc2c(-c3ccc4ccccc4c3)cccc12)C(F)(F)F ; [2H]c1c([2H])c([2H])c2c([2H])c(-c3c([2H])c([2H])c([2H])c4c(OS(=O)(=O)C(F)(F)F)c([2H])c([2H])c([2H])c34)c([2H])c([2H])c2c1[2H]]

In a 1-L round-bottom flask reactor, a solution of <1-f> (50.0 g, 0.124 mol) in dichlorobenzene (250 ml) was added with benzene (d6) and stirred at room temperature under a light-tight condition. Trifluoromethane sulfonic acid (6.34 g, 0.042 mol) was dropwise added to the reaction solution. After completion of the dropwise addition, the mixture was stirred at 70° C. for 2 days. Heavy water (D20) (50 ml) was added, followed by separating the organic layer. A solution of sodium phosphate, tribasic (39.6 g, 0.186 mol) in water (200 ml) was added to the organic layer and stirred. The organic layer was extracted, concentrated in a vacuum, and added with an excess of methanol to precipitate crystals. The solid thus obtained were filtered and washed with methanol to afford <3-a>. (48.2 g, 93%)

### Synthesis Example 3-(2): Synthesis of <A-10>

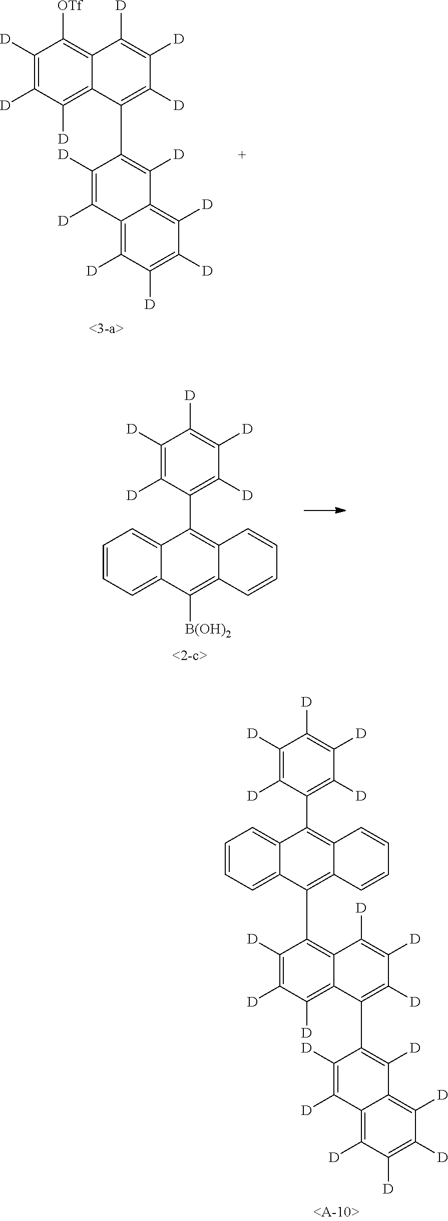 [SMILES: [2H]c1c([2H])c([2H])c2c([2H])c(-c3c([2H])c([2H])c([2H])c4c(OS(=O)(=O)C(F)(F)F)c([2H])c([2H])c([2H])c34)c([2H])c([2H])c2c1[2H] ; [2H]c1c([2H])c([2H])c(-c2c3ccccc3c(B(O)O)c3ccccc23)c([2H])c1[2H] ; [2H]c1c([2H])c([2H])c(-c2c3ccccc3c(-c3c([2H])c([2H])c([2H])c4c(-c5c([2H])c([2H])c6c([2H])c([2H])c([2H])c([2H])c6c5[2H])c([2H])c([2H])c([2H])c34)c3ccccc23)c([2H])c1[2H]]

The same procedure as in Synthesis Example 2-(4) was carried out, with the exception of using <3-a> instead of <2-b>, to afford <A-10> (5.6 g, 50.3%)

MS (MALDI-TOF): m/z 524.32 [M⁺]

### Synthesis Example 4: Synthesis of <A-14>

### Synthesis Example 4-(1): Synthesis of <4-a>

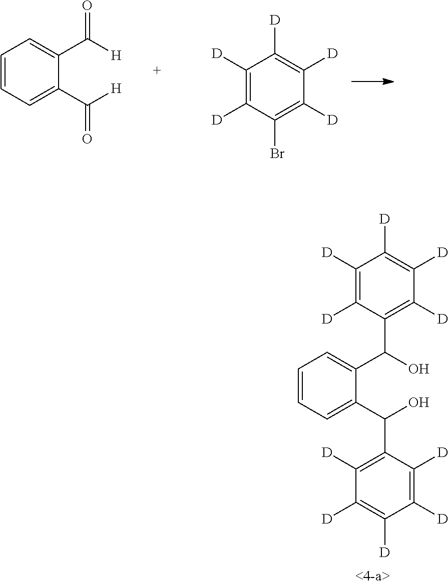 [SMILES: O=Cc1ccccc1C=O ; [2H]c1c([2H])c([2H])c(Br)c([2H])c1[2H] ; [2H]c1c([2H])c([2H])c(C(O)c2ccccc2C(O)c2c([2H])c([2H])c([2H])c([2H])c2[2H])c([2H])c1[2H]]

Under a nitrogen atmosphere, bromobenzene (d5) (217.5 g, 1.342 mol) and tetrahydrofuran (1740 mL) in a 5-L round-bottom flask were stirred at −78° C. To the chilled reaction solution was dropwise added 1.6 M n-butyl lithium (804 mL, 1.286 mol), followed by stirring at the same temperature for 1 hour. A solution of o-phthalaldehyde (75.0 g, 0.559 mol) in tetrahydrofuran (700 mL) was dropwise added and stirred at room temperature. After completion of the reaction, an aqueous ammonium chloride solution (700 mL) was added to terminate the reaction. The reaction mixture was subjected to extraction with ethyl acetate and the organic layer thus formed was separated, concentrated in a vacuum, and purified by column chromatography to afford <4-a> (140 g, 83%).

### Synthesis Example 4-(2): Synthesis of <4-b>

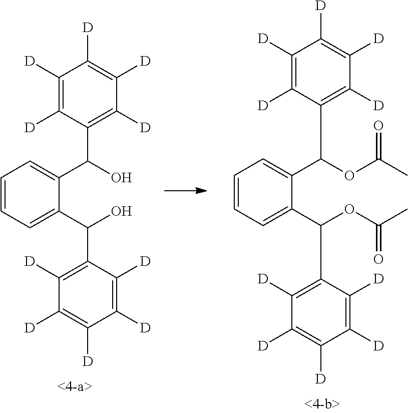 [SMILES: [2H]c1c([2H])c([2H])c(C(O)c2ccccc2C(O)c2c([2H])c([2H])c([2H])c([2H])c2[2H])c([2H])c1[2H] ; [2H]c1c([2H])c([2H])c(C(OC(C)=O)c2ccccc2C(OC(C)=O)c2c([2H])c([2H])c([2H])c([2H])c2[2H])c([2H])c1[2H]]

In a 5-L round-bottom flask, a solution of the intermediate <4-a> (140.0 g, 0.466 mol) in dichloromethane (1400 ml) was added with acetate anhydride (176.2 ml, 1.864 mol) and triethylamine (389.7 ml, 2.796 mol), cooled to 0° C., and stirred. 4-(Dimethylamino)pyridine (11.4 g, 0.093 mol) was added little by little to the mixture and stirred at room temperature for 1 hour. When the reaction was completed as monitored by thin layer chromatography, the reaction mixture was chilled to 0° C. and stirred while slowly adding drops of distilled water (800 ml). After extraction with ethyl acetate and distilled water, the organic layer thus formed was separated and washed once more with an aqueous sodium hydrogen carbonate. The organic layer was dried over magnesium sulfate and then concentrated in a vacuum. The concentrate was purified by column chromatography to afford intermediate <4-b>. (120.0 g, 67%)

### Synthesis Example 4-(3): Synthesis of <4-c>

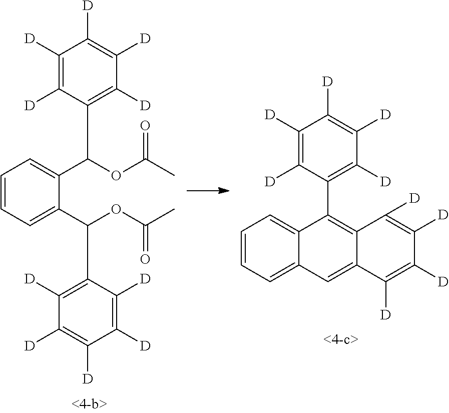 [SMILES: [2H]c1c([2H])c([2H])c(C(OC(C)=O)c2ccccc2C(OC(C)=O)c2c([2H])c([2H])c([2H])c([2H])c2[2H])c([2H])c1[2H] ; [2H]c1c([2H])c([2H])c(-c2c3ccccc3cc3c([2H])c([2H])c([2H])c([2H])c23)c([2H])c1[2H]]

In a 1000-mL round-bottom flask, a solution of intermediate <4-b> (120.0 g, 0.312 mol) in dichloromethane (600 mL) was stirred. To this solution was dropwise added a dilution of boron trifluoride diethyl etherate (7.7 mL, 0.062 mol) in dichloromethane (30 ml), followed by stirring under reflux. When the reaction was completed as monitored by thin layer chromatography, the reaction mixture was slowly poured into distilled water (1,000 ml) in a beaker and then stirred. Extraction was made with dichloromethane and distilled water. The organic layer thus formed was dried over magnesium sulfate and concentrated in a vacuum. The concentrate was purified by column chromatography to afford intermediate <4-c>. (60.0 g, 73%)

### Synthesis Example 4-(4): Synthesis of <4-d>

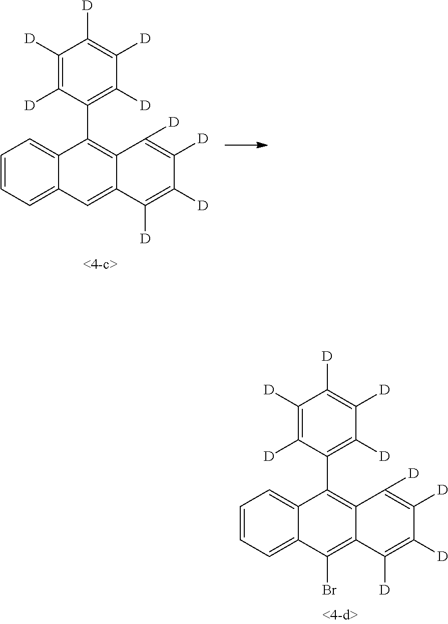 [SMILES: [2H]c1c([2H])c([2H])c(-c2c3ccccc3cc3c([2H])c([2H])c([2H])c([2H])c23)c([2H])c1[2H] ; [2H]c1c([2H])c([2H])c(-c2c3ccccc3c(Br)c3c([2H])c([2H])c([2H])c([2H])c23)c([2H])c1[2H]]

In a 1000-mL round-bottom flask, a solution of intermediate <4-c> (60.0 g, 0.228 mol) in N,N-dimethylamide (500 mL) was stirred at room temperature. A solution of N-bromosuccinimide (44.64 g, 0.251 mol) in N,N-dimethylamide (100 mL) was dropwise added. The reaction was terminated as monitored by thin layer chromatography. The reaction mixture was poured to water (1,000 mL) in a beaker and stirred. The solid thus formed was filtered and washed with water. Purification by column chromatography afforded intermediate <4-d> (72.0 g, 92.6%).

### Synthesis Example 4-(5): Synthesis of <4-e>

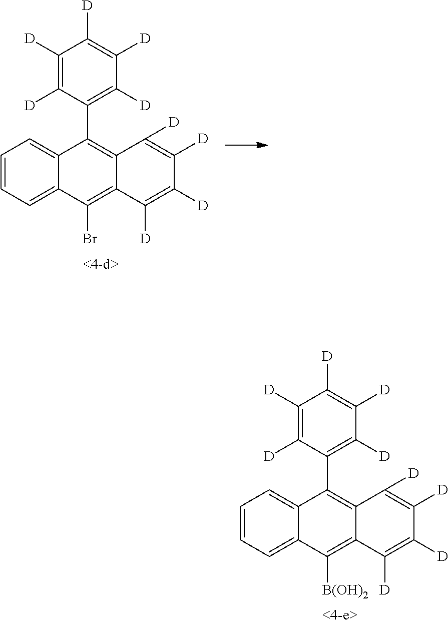 [SMILES: [2H]c1c([2H])c([2H])c(-c2c3ccccc3c(Br)c3c([2H])c([2H])c([2H])c([2H])c23)c([2H])c1[2H] ; [2H]c1c([2H])c([2H])c(-c2c3ccccc3c(B(O)O)c3c([2H])c([2H])c([2H])c([2H])c23)c([2H])c1[2H]]

The same procedure as in Synthesis Example 1-(3) was carried out, with the exception of using intermediate <4-d> instead of intermediate <1-b>, to afford intermediate <4-d> (52.0 g, 88.2%).

### Synthesis Example 4-(6): Synthesis of <A-14>

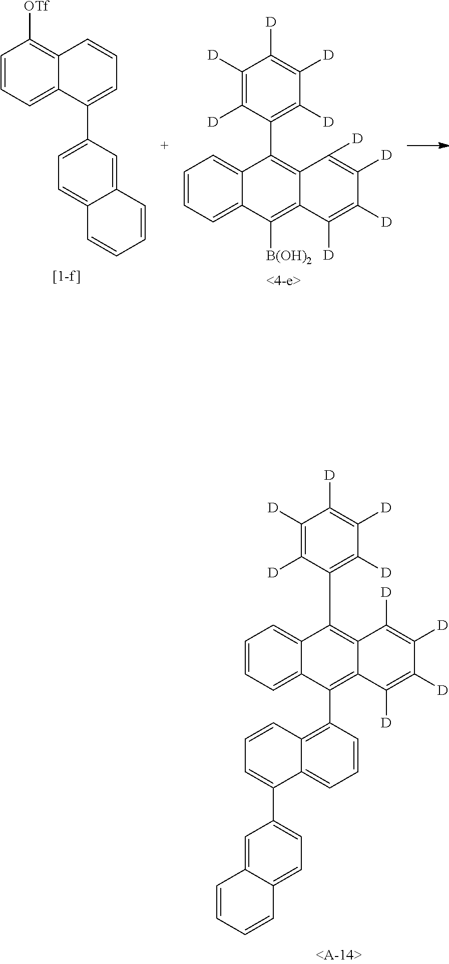

The same procedure as in Synthesis Example 1-(7) was carried out, with the exception of using <4-e> instead of intermediate <1-c>, to afford <A-14> (6.2 g, 49.0%).

MS (MALDI-TOF): m/z 515.26 [M⁺]

### Synthesis Example 5: Synthesis of <A-30>

### Synthesis Example 5-(1): Synthesis of <A-30>

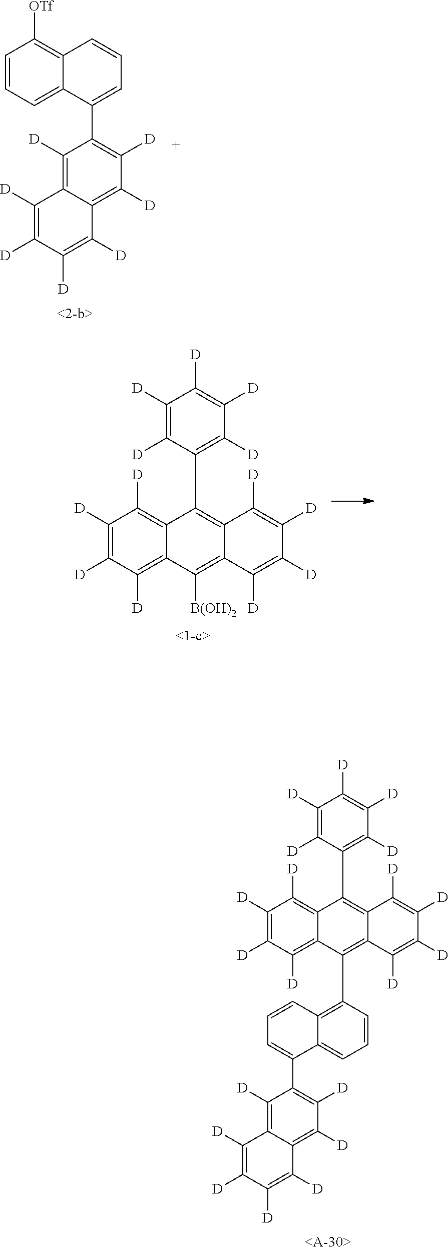 [SMILES: [2H]c1c([2H])c([2H])c2c([2H])c(-c3cccc4c(OS(=O)(=O)C(F)(F)F)cccc34)c([2H])c([2H])c2c1[2H] ; [2H]c1c([2H])c([2H])c(-c2c3c([2H])c([2H])c([2H])c([2H])c3c(B(O)O)c3c([2H])c([2H])c([2H])c([2H])c23)c([2H])c1[2H] ; [2H]c1c([2H])c([2H])c(-c2c3c([2H])c([2H])c([2H])c([2H])c3c(-c3cccc4c(-c5c([2H])c([2H])c6c([2H])c([2H])c([2H])c([2H])c6c5[2H])cccc34)c3c([2H])c([2H])c([2H])c([2H])c23)c([2H])c1[2H]]

The same procedure as in Synthesis Example 2-(4) was carried out, with the exception of using <1-c> instead of <2-c>, to afford <A-30> (5.2 g, 40.4%)

MS (MALDI-TOF): m/z 526.33 [M⁺]

### Synthesis Example 6: Synthesis of <A-37>

### Synthesis Example 6-(1): Synthesis of <A-37>

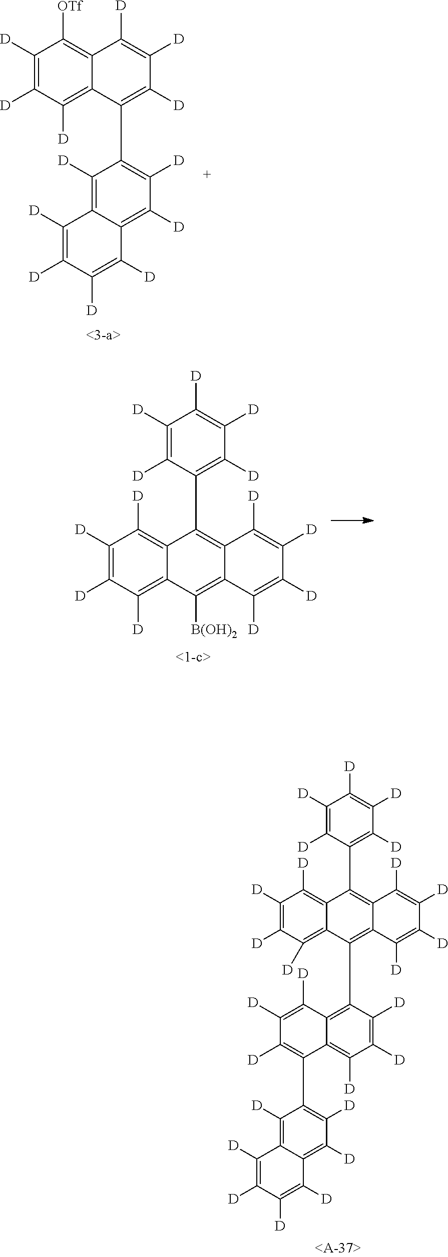 [SMILES: [2H]c1c([2H])c([2H])c2c([2H])c(-c3c([2H])c([2H])c([2H])c4c(OS(=O)(=O)C(F)(F)F)c([2H])c([2H])c([2H])c34)c([2H])c([2H])c2c1[2H] ; [2H]c1c([2H])c([2H])c(-c2c3c([2H])c([2H])c([2H])c([2H])c3c(B(O)O)c3c([2H])c([2H])c([2H])c([2H])c23)c([2H])c1[2H] ; [2H]c1c([2H])c([2H])c(-c2c3c([2H])c([2H])c([2H])c([2H])c3c(-c3c([2H])c([2H])c([2H])c4c(-c5c([2H])c([2H])c6c([2H])c([2H])c([2H])c([2H])c6c5[2H])c([2H])c([2H])c([2H])c34)c3c([2H])c([2H])c([2H])c([2H])c23)c([2H])c1[2H]]

The same procedure as in Synthesis Example 1-(7) was carried out, with the exception of using <3-a> instead of <1-f>, to afford <A-37> (5.4 g, 42.1%).

MS (MALDI-TOF): m/z 532.37 [M⁺]

### Synthesis Example 7<A-41>

### Synthesis Example 7-(1): Synthesis of <A-41>

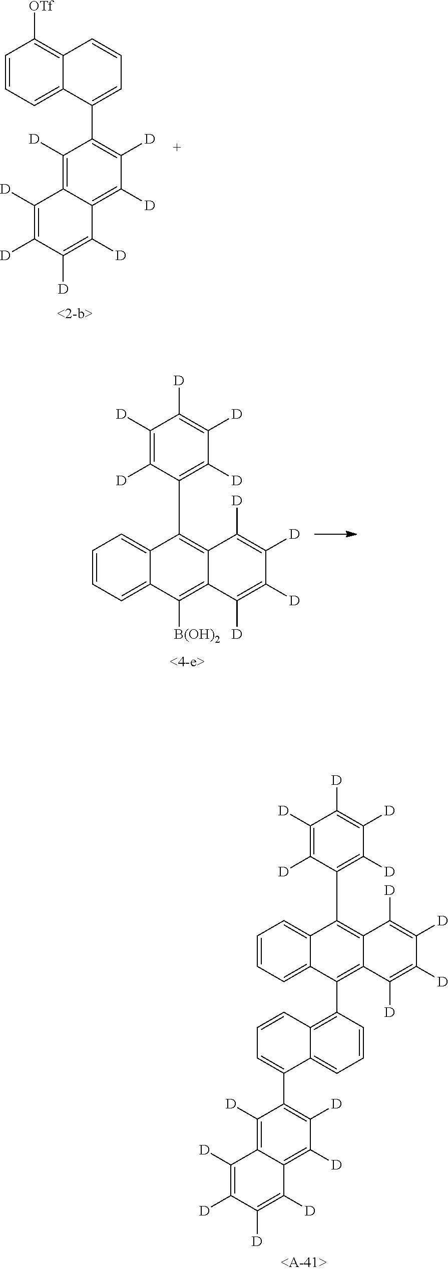 [SMILES: [2H]c1c([2H])c([2H])c2c([2H])c(-c3cccc4c(OS(=O)(=O)C(F)(F)F)cccc34)c([2H])c([2H])c2c1[2H] ; [2H]c1c([2H])c([2H])c(-c2c3ccccc3c(B(O)O)c3c([2H])c([2H])c([2H])c([2H])c23)c([2H])c1[2H] ; [2H]c1c([2H])c([2H])c(-c2c3ccccc3c(-c3cccc4c(-c5c([2H])c([2H])c6c([2H])c([2H])c([2H])c([2H])c6c5[2H])cccc34)c3c([2H])c([2H])c([2H])c([2H])c23)c([2H])c1[2H]]

The same procedure as in Synthesis Example 1-(7) was carried out, with the exception of using <2-b> and <4-e> instead of <1-f> and <1-c>, respectively, to afford <A-41> (6.2 g, 48.1%).

MS (MALDI-TOF): m/z 522.30 [M⁺]

### Synthesis Example 8<A-49>

### Synthesis Example 8-(1): Synthesis of <A-49>

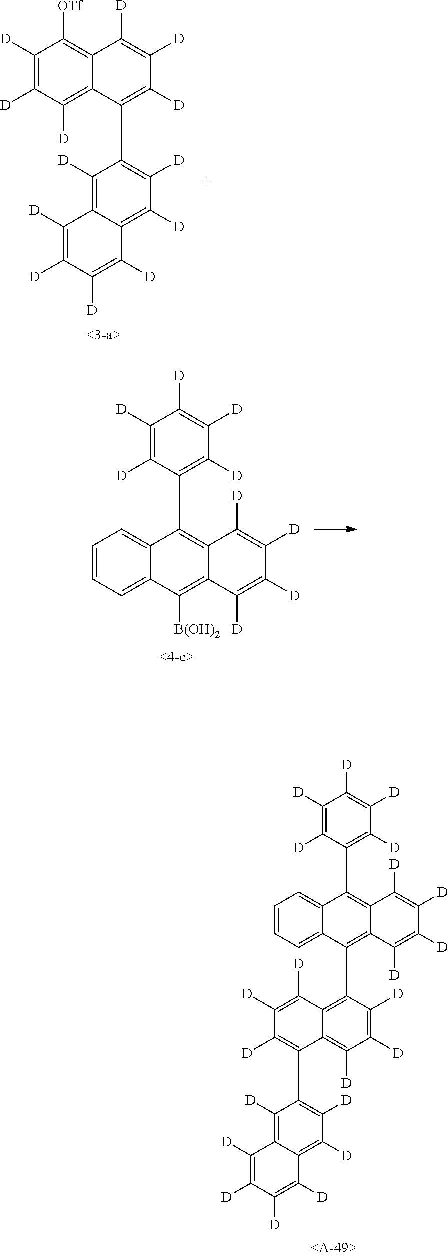 [SMILES: [2H]c1c([2H])c([2H])c2c([2H])c(-c3c([2H])c([2H])c([2H])c4c(OS(=O)(=O)C(F)(F)F)c([2H])c([2H])c([2H])c34)c([2H])c([2H])c2c1[2H] ; [2H]c1c([2H])c([2H])c(-c2c3ccccc3c(B(O)O)c3c([2H])c([2H])c([2H])c([2H])c23)c([2H])c1[2H] ; [2H]c1c([2H])c([2H])c(-c2c3ccccc3c(-c3c([2H])c([2H])c([2H])c4c(-c5c([2H])c([2H])c6c([2H])c([2H])c([2H])c([2H])c6c5[2H])c([2H])c([2H])c([2H])c34)c3c([2H])c([2H])c([2H])c([2H])c23)c([2H])c1[2H]]

The same procedure as in Synthesis Example 1-(7) was carried out, with the exception of using <3-a> and <4-e> instead of <1-f> and <1-c>, respectively, to afford <A-49> (5.8 g, 45.4%).

MS (MALDI-TOF): m/z 528.34 [M⁺]

### Dopant Synthesis Example: Synthesis of Compounds Represented by Chemical Formulas D-6 or D-7

### Synthesis Example 9: Synthesis of [D 345]

### Synthesis Example 9-(1) Synthesis of Intermediate A-1

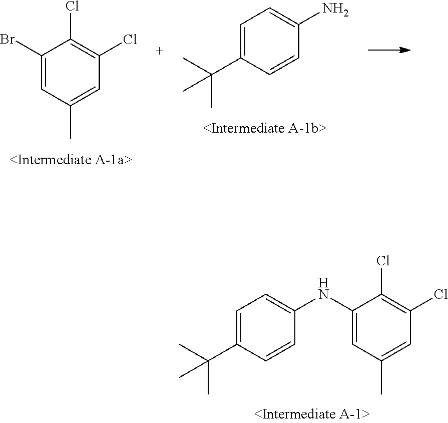

In a reactor, <Intermediate A-1a>35 g, <Intermediate A-1b>23.9 g, tris(dibenzylideneacetone)dipalladium(0) (2.67 g), bis(diphenylphosphino)-1,1′-binaphthyl (1.82 g), sodium tert-butoxide (28 g), and toluene (450 mL) were stirred together for 3 hours under reflux. The reaction mixture was cooled to room temperature, followed by adding ethyl acetate and water thereto. The organic layer thus formed was separated, and purification by silica gel chromatography afforded <Intermediate A-1>. (40.5 g, 90.1%)

### Synthesis Example 9-(2) Synthesis of Intermediate A-2

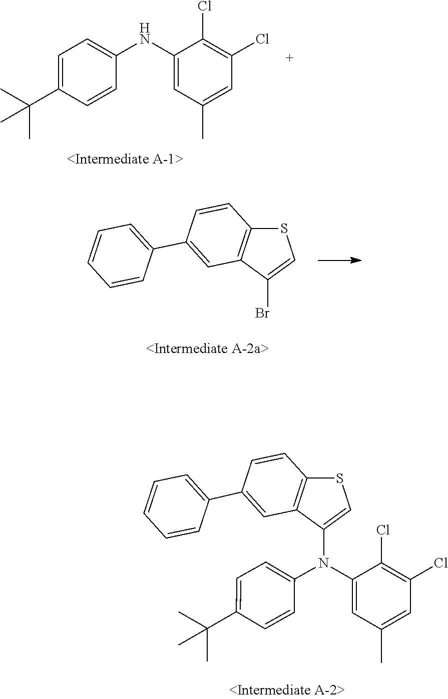 [SMILES: Cc1cc(Cl)c(Cl)c(Nc2ccc(C(C)(C)C)cc2)c1 ; Brc1csc2ccc(-c3ccccc3)cc12 ; Cc1cc(Cl)c(Cl)c(N(c2ccc(C(C)(C)C)cc2)c2csc3ccc(-c4ccccc4)cc23)c1]

In a reactor, <Intermediate A-1> (24 g), <Intermediate A-2a> (24.8 g), bis(tri-tert-butylphosphine)palladium(0) (0.8 g), sodium tert-butoxide (12 g), and toluene (350 mL) were stirred together for 6 hours under reflux. The reaction mixture was cooled to room temperature, followed by adding ethyl acetate and water thereto. The organic layer thus formed was separated, and purification by silica gel chromatography afforded <Intermediate A-2>. (35.2 g, 87.5%)

### Synthesis Example 9-(3): Synthesis of Intermediate A-3

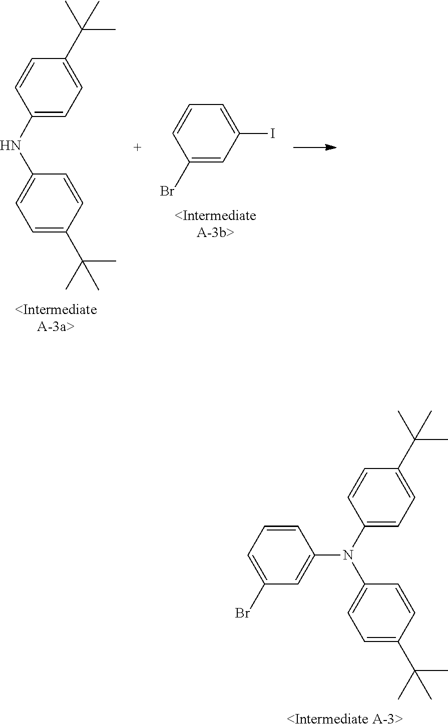 [SMILES: CC(C)(C)c1ccc(Nc2ccc(C(C)(C)C)cc2)cc1 ; Brc1cccc(I)c1 ; CC(C)(C)c1ccc(N(c2ccc(C(C)(C)C)cc2)c2cccc(Br)c2)cc1]

In a reactor, <Intermediate A-3a> (50 g), <Intermediate A-3b> (60.3 g), palladium(II) acetate (0.4 g), sodium tert-butoxide (25.6 g), Xantphos (1 g), and toluene (500 mL) were stirred together for 16 hours under reflux. The reaction mixture was cooled to room temperature, followed by adding ethyl acetate and water thereto. The organic layer thus formed was separated, and purification by silica gel chromatography afforded <Intermediate A-3>. (59.6 g, 76.9%)

### Synthesis Example 9-(4) Synthesis of Intermediate A-4

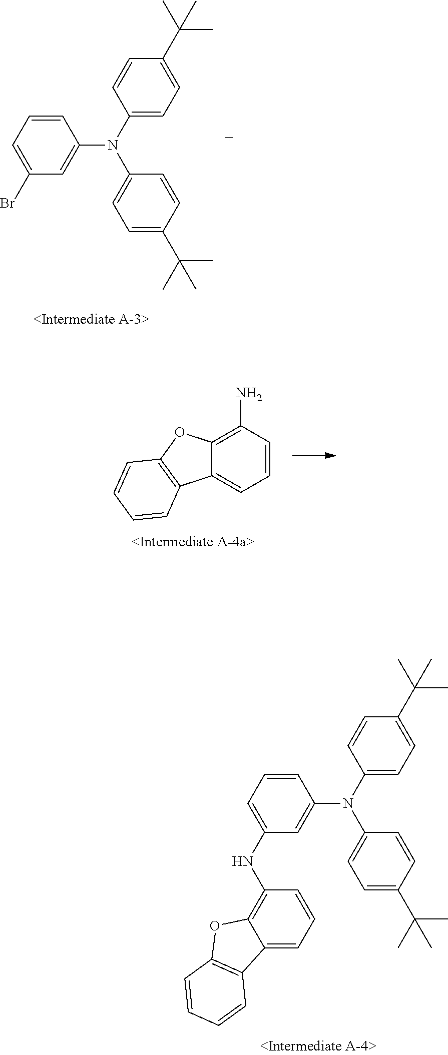 [SMILES: CC(C)(C)c1ccc(N(c2ccc(C(C)(C)C)cc2)c2cccc(Br)c2)cc1 ; Nc1cccc2c1oc1ccccc12 ; CC(C)(C)c1ccc(N(c2ccc(C(C)(C)C)cc2)c2cccc(Nc3cccc4c3oc3ccccc34)c2)cc1]

In a reactor, <Intermediate A-3> (50 g), <Intermediate A-4a> (23.1 g), tris(dibenzylideneacetone)dipalladium(0) (2.1 g), bis(diphenylphosphino)-1,1′-binaphthyl (1.43 g), sodium tert-butoxide (22 g), and toluene (500 mL) were stirred together for 16 hours under reflux. The reaction mixture was cooled to room temperature, followed by adding ethyl acetate and water thereto. The organic layer thus formed was separated, and purification by silica gel chromatography afforded <Intermediate A-4>. (43.4 g, 70.3%)

### Synthesis Example 9-(5): Synthesis of Intermediate A-5

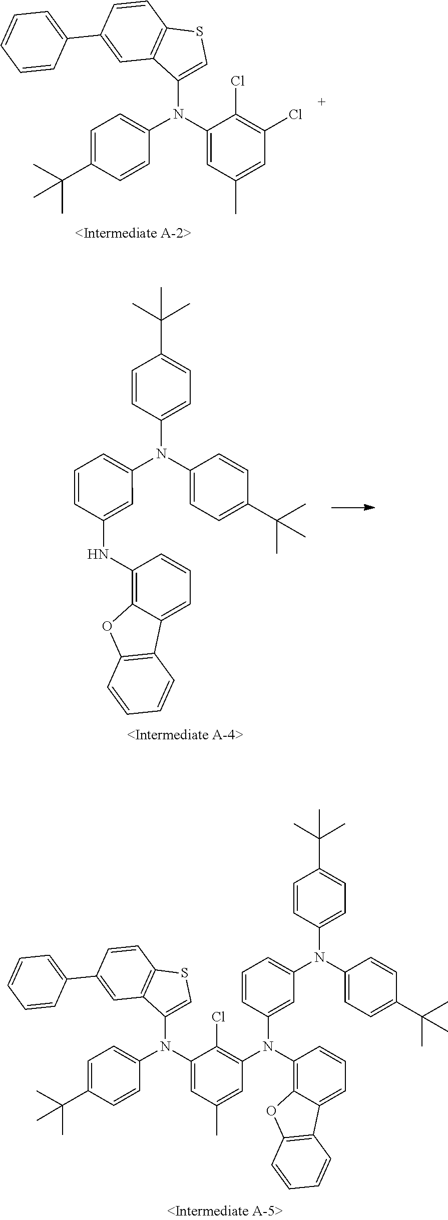 [SMILES: Cc1cc(Cl)c(Cl)c(N(c2ccc(C(C)(C)C)cc2)c2csc3ccc(-c4ccccc4)cc23)c1 ; CC(C)(C)c1ccc(N(c2ccc(C(C)(C)C)cc2)c2cccc(Nc3cccc4c3oc3ccccc34)c2)cc1 ; Cc1cc(N(c2ccc(C(C)(C)C)cc2)c2csc3ccc(-c4ccccc4)cc23)c(Cl)c(N(c2cccc(N(c3ccc(C(C)(C)C)cc3)c3ccc(C(C)(C)C)cc3)c2)c2cccc3c2oc2ccccc23)c1]

In a reactor, <Intermediate A-2> (32 g), <Intermediate A-4> (34.4 g), bis(tri-tert-butylphosphine)palladium(0) (0.63 g), sodium tert-butoxide (11.9 g), and toluene (300 mL) were stirred together for 16 hours under reflux. The reaction mixture was cooled to room temperature, followed by adding ethyl acetate and water thereto. The organic layer thus formed was separated, and purification by silica gel chromatography afforded <Intermediate A-5>. (50.5 g, 80%)

### Synthesis Example 9-(6) Synthesis of [D 345]

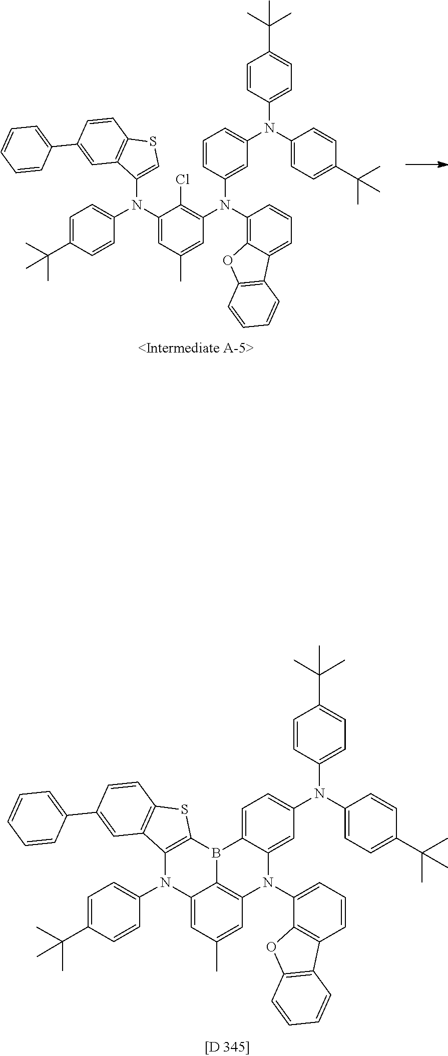 [SMILES: Cc1cc(N(c2ccc(C(C)(C)C)cc2)c2csc3ccc(-c4ccccc4)cc23)c(Cl)c(N(c2cccc(N(c3ccc(C(C)(C)C)cc3)c3ccc(C(C)(C)C)cc3)c2)c2cccc3c2oc2ccccc23)c1 ; Cc1cc2c3c(c1)N(c1cccc4c1oc1ccccc14)c1cc(N(c4ccc(C(C)(C)C)cc4)c4ccc(C(C)(C)C)cc4)ccc1B3c1sc3ccc(-c4ccccc4)cc3c1N2c1ccc(C(C)(C)C)cc1]

In a reactor, <Intermediate A-5> (48 g) and tert-butyl benzene (300 mL) were added with drops of 1.7 M tert-butyl lithium pentane solution (83 mL) at −78° C. The mixture was heated to 60° C., followed by stirring for 2 hours. then, nitrogen was blown into the mixture at 60° C. to completely remove the pentane. After cooling to −78° C., boron tribromide (14.1 mL) was added dropwise. The resulting mixture was allowed to warm to room temperature and stirred for 2 hours. After cooling to 0° C., N, N-diisopropylethylamine (25 mL) was added dropwise. The mixture was heated to 120° C. and stirred for 16 hours. The reaction mixture was cooled to room temperature and a 10% aqueous solution of sodium acetate and ethyl acetate were added thereto. The organic layer was separated, concentrated under reduced pressure, and purified by silica gel chromatography to afford [D 345]. (7.2 g, 15.4%)

MS (MALDI-TOF): m/z 991.47 [M⁺]

### Synthesis Example 10: Synthesis of [D 265]

With reference to the synthesis method for dopant compounds disclosed in Korean Patent No. 10-2148296, the following [D 265] compound was prepared.

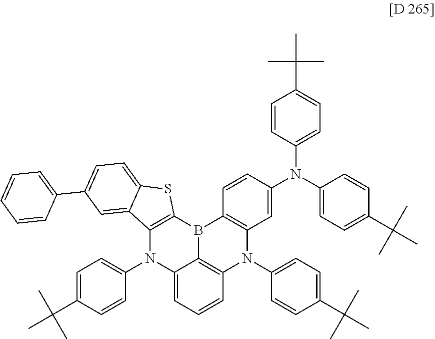 [SMILES: CC(C)(C)c1ccc(N(c2ccc(C(C)(C)C)cc2)c2ccc3c(c2)N(c2ccc(C(C)(C)C)cc2)c2cccc4c2B3c2sc3ccc(-c5ccccc5)cc3c2N4c2ccc(C(C)(C)C)cc2)cc1]

### Examples 1 to 11 Fabrication of Organic Light-Emitting Diodes

An ITO glass substrate was patterned to have a translucent area of 2 mm×2 mm and cleansed. The ITO glass was mounted in a vacuum chamber that was then set to have a base pressure of 1×10⁻⁷ torr. The electron acceptor of the following structural formula [Acceptor-1] and the compound of [Chemical Formula F] were dual-deposited at a deposition rate ratio of [Acceptor-1]:[Chemical Formula F]=2:98 to form a film (100 Å) as a hole injection layer on the ITO glass substrate. As a hole transport layer, a film (550 Å) was formed of [Chemical Formula F], followed by depositing [Chemical Formula G] to form a film (50 Å) as an electron blocking layer. Subsequently, a light-emitting layer (200 Å) was formed of a combination of the compounds of the present disclosure and the dopant compound [D 265] or [D 345](1 wt %) listed below. Then, [Chemical Formula H] was deposited to form an electron blocking layer (50 Å) on which [Chemical Formula E-1] and [Chemical Formula E-2] were deposited at a ratio of 1:1 to form an electron transport layer (250 Å). Subsequently, an electron injection layer of [Chemical Formula E-2](10 Å) was formed and then covered with an Al layer (1000 Å) to fabricate an organic light-emitting diode. The organic light-emitting diodes thus obtained were measured at 0.4 mA/cm² for luminescence properties.

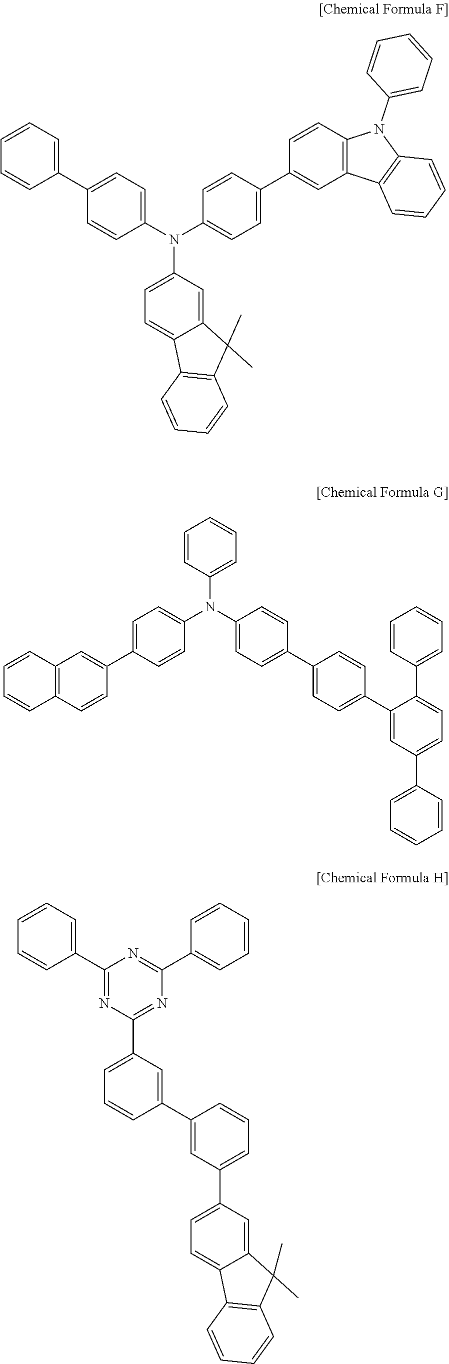 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(-c3ccccc3)c(-c3ccc(-c4ccc(N(c5ccccc5)c5ccc(-c6ccc7ccccc7c6)cc5)cc4)cc3)c2)cc1 ; CC1(C)c2ccccc2-c2ccc(-c3cccc(-c4cccc(-c5nc(-c6ccccc6)nc(-c6ccccc6)n5)c4)c3)cc21]

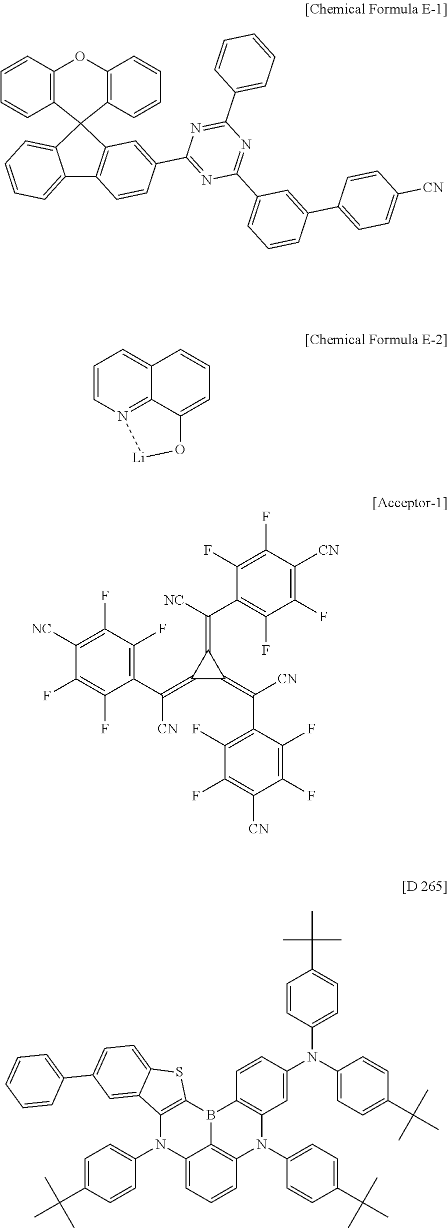

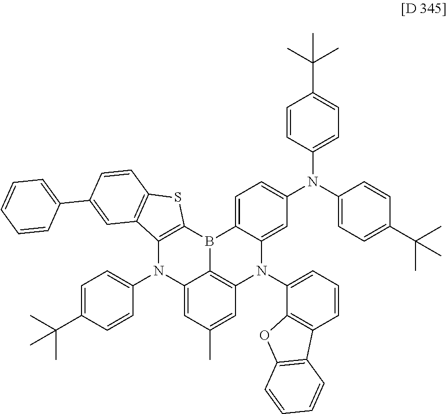 [SMILES: Cc1cc2c3c(c1)N(c1cccc4c1oc1ccccc14)c1cc(N(c4ccc(C(C)(C)C)cc4)c4ccc(C(C)(C)C)cc4)ccc1B3c1sc3ccc(-c4ccccc4)cc3c1N2c1ccc(C(C)(C)C)cc1]

### Comparative Examples 1 to 8: Fabrication of Organic Light-Emitting Diodes

Organic light emitting diodes were fabricated in the same manner as in the Examples 1 to 11, with the exception of using [BH 1], [BH 2], [BH 3], [BD 1], and [BD 2] instead of the compounds used therein. The luminescence of the organic light-emitting diodes thus obtained was measured at 0.4 mA. Structures of compounds [BH 1], [BH 2], [BH 3], [BD 1], and [BD 2] are as follows:

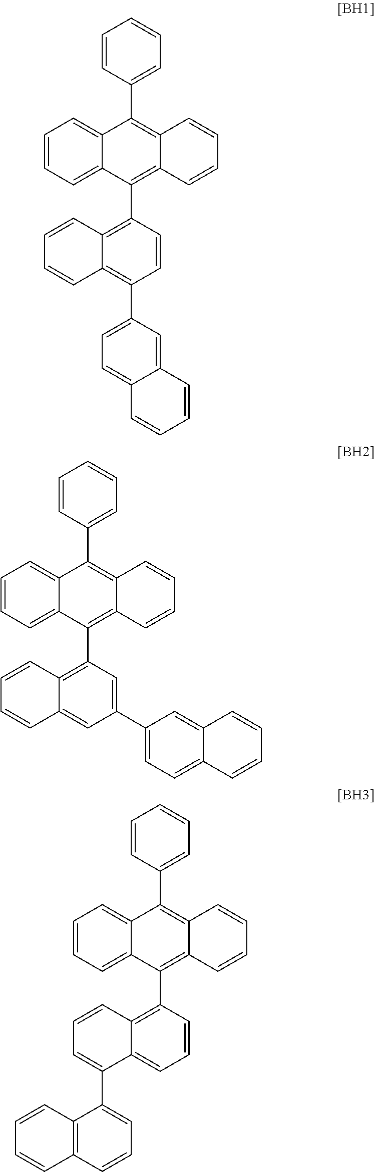

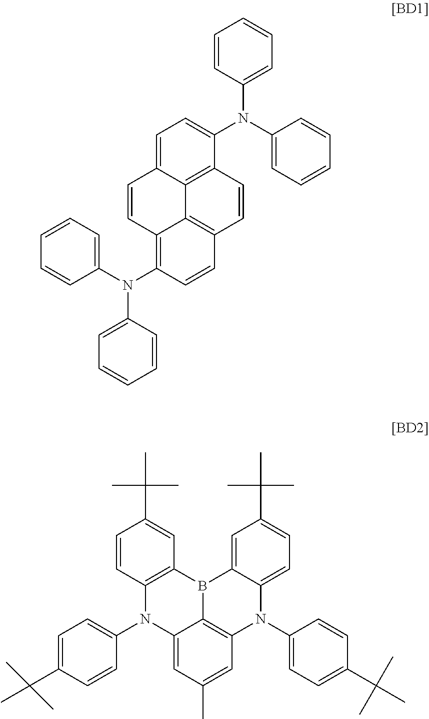 [SMILES: c1ccc(N(c2ccccc2)c2ccc3ccc4c(N(c5ccccc5)c5ccccc5)ccc5ccc2c3c54)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

TABLE 1

| | Host | Dopant | cd/A | LT₉₇ |
|---|---|---|---|---|
| Ex. 1 | A-13 | D 265 | 8.65 | 260 |
| Ex. 2 | A-3 | D 265 | 8.65 | 210 |
| Ex. 3 | A-10 | D 265 | 8.65 | 220 |
| Ex. 4 | A-10 | D 345 | 8.60 | 214 |
| Ex. 5 | A-14 | D 265 | 8.65 | 235 |
| Ex. 6 | A-30 | D 265 | 8.66 | 263 |
| Ex. 7 | A-30 | D 345 | 8.60 | 256 |
| Ex. 8 | A-37 | D 265 | 8.65 | 265 |
| Ex. 9 | A-41 | D 265 | 8.65 | 233 |
| Ex. 10 | A-41 | D 345 | 8.62 | 225 |
| Ex. 11 | A-49 | D 265 | 8.66 | 235 |
| C. Ex. 1 | A-10 | BD 1 | 6.55 | 120 |
| C. Ex. 2 | BH 1 | D 265 | 8.55 | 180 |
| C. Ex. 3 | BH 3 | D 265 | 8.57 | 186 |
| C. Ex. 4 | BH 3 | D 345 | 8.45 | 175 |
| C. Ex. 5 | BH 1 | BD 1 | 6.67 | 115 |
| C. Ex. 6 | BH 2 | BD 1 | 6.66 | 118 |
| C. Ex. 7 | BH 1 | BD 2 | 6.75 | 132 |
| C. Ex. 8 | BH 3 | BD 2 | 6.78 | 130 |

As is understood from data of Table 1, the organic light emitting diodes according to the present disclosure allow for low driving voltages and longevity, compared to Comparative Examples 1 to 8, thus finding high availability for organic light-emitting diodes.

1. An organic light emitting diode, comprising:
a first electrode;
a second electrode facing the first electrode; and
a light-emitting layer disposed between the first electrode and the second electrode,
wherein the light-emitting layer contains a host and a dopant, the host including at least one of anthracene compounds represented by the following Chemical Formula A, the dopant comprising at least one of the compounds represented by the following Chemical Formulas D-6 to D-7: 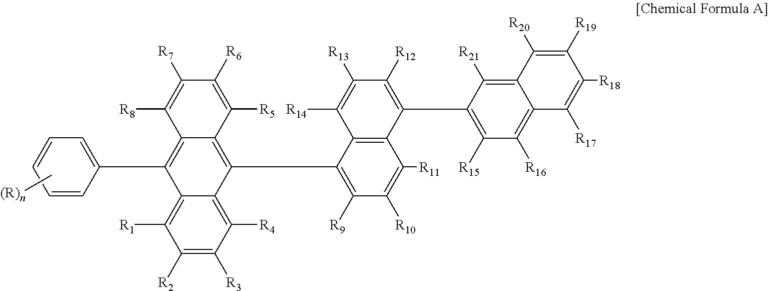
wherein,
R₁ to R₂₁, which are same or different, each are independently any one selected from the group consisting of a hydrogen atom, a deuterium atom, a substituted or unsubstituted alkyl of 1 to 30 carbon atoms, and a substituted or unsubstituted aryl of 6 to 50 carbon atoms;
R is a hydrogen atom or a deuterium atom; and
n is an integer of 1 to 5, wherein when n is 2 to 5, the corresponding R's are same or different and are independently a hydrogen atom or a deuterium atom, and 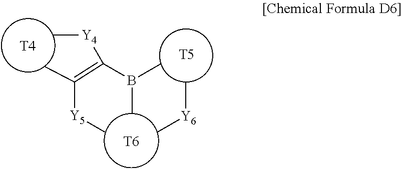 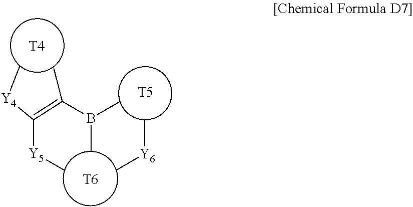
wherein,
T4 to T6 are same or different and are each independently a substituted or unsubstituted aromatic hydrocarbon ring of 6 to 50 carbon atoms, or a substituted or unsubstituted heteroaromatic ring of 2 to 40 carbon atoms;
Y₄ is any one selected from N—R₆₁, CR₆₂R₆₃, O, S, and SiR₆₄R₆₅;
Y₅ is any one selected from N—R₆₆, CR₆₇R₆₈, O, S, and SiR₆₉R₇₀;
Y₆ is any one selected from N—R₇₁, CR₇₂R₇₃, O, S, and SiR₇₄R₇₅;
R₆₁ to R₇₅ are same or different and are each independently any one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a cyano, and a halogen atom,
a linkage can be formed between R₆₂ and R₆₃, between R₆₄ and R₆₅, between R₆₇ and R₆₈, between R₆₉ and R₇₀, between R₇₂ and R₇₃, and/or between R₇₄ and R₇₅ to form respective aliphatic or aromatic mono- or polycyclic rings,
R₆₁ to R₇₅ are each linked to any one ring moiety of T4 to T6 to additionally an aliphatic or aromatic mono- or polycyclic ring,
wherein the term “substituted” in the expression “substituted or unsubstituted” used for compounds of Chemical Formulas A, D6, and D7 means having at least one substituent selected from the group consisting of a deuterium atom, a cyano, a halogen atom, a hydroxy, a nitro, an alkyl of 1 to 24 carbon atoms, a halogenated alkyl of 1 to 24 carbon atoms, an alkenyl of 1 to 24 carbon atoms, an alkynyl of 1 to 24 carbon atoms, a cycloalkyl of 3 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 2 to 24 carbon atoms, an alkoxy of 1 to 24 carbon atoms, an alkylamino of 1 to 24 carbon atoms, a diarylamino of 12 to 24 carbon atoms, an diheteroarylamino of 2 to 24 carbon atoms, an aryl(heteroaryl)amino of 7 to 24 carbon atoms, an alkylsilyl of 1 to 24 carbon atoms, an arylsilyl of 6 to 24 carbon atoms, an aryloxy of 6 to 24 carbon atoms, and an arylthionyl of 6 to 24 carbon atoms.

2. The organic light-emitting diode of claim 1, wherein R and n in Chemical Formula A is a deuterium atom and 5, respectively.

3. The organic light-emitting diode of claim 1, wherein R₁ to R₂₁ in Chemical Formula A are each a hydrogen atom or a deuterium atom.

4. The organic light-emitting diode of claim 3, wherein four or more radicals of R₁ to R₂₁ in Chemical Formula A are each a deuterium atom.

5. The organic light-emitting diode of claim 1, wherein four or more radicals of R₁ to R₈ in Chemical Formula A are each a deuterium atom.

6. The organic light-emitting diode of claim 1, wherein the anthracene derivative represented by Chemical Formula A has a degree of deuteration of 30% or higher.

7. The organic light-emitting diode of claim 1, wherein the compound represented by Chemical Formula A is any one selected from the group consisting of the compounds represented by [A-1] to [A-153]:

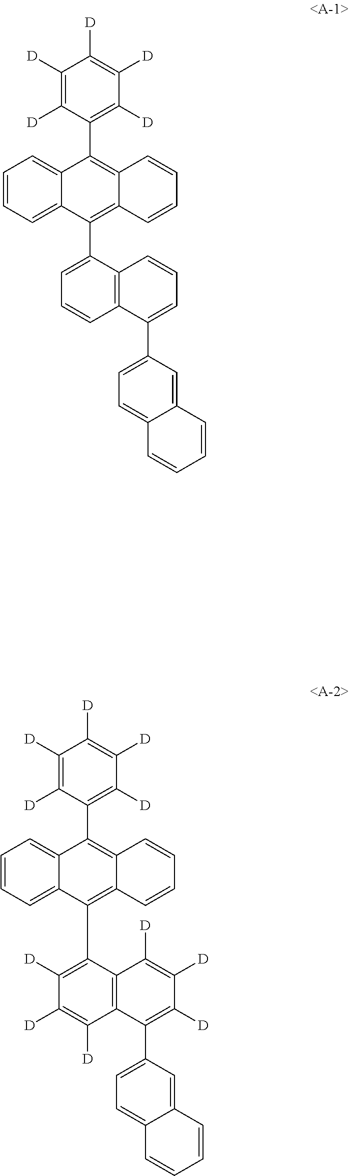 [SMILES: [2H]c1c([2H])c([2H])c(-c2c3ccccc3c(-c3cccc4c(-c5ccc6ccccc6c5)cccc34)c3ccccc23)c([2H])c1[2H] ; [2H]c1c([2H])c([2H])c(-c2c3ccccc3c(-c3c([2H])c([2H])c([2H])c4c(-c5ccc6ccccc6c5)c([2H])c([2H])c([2H])c34)c3ccccc23)c([2H])c1[2H]]

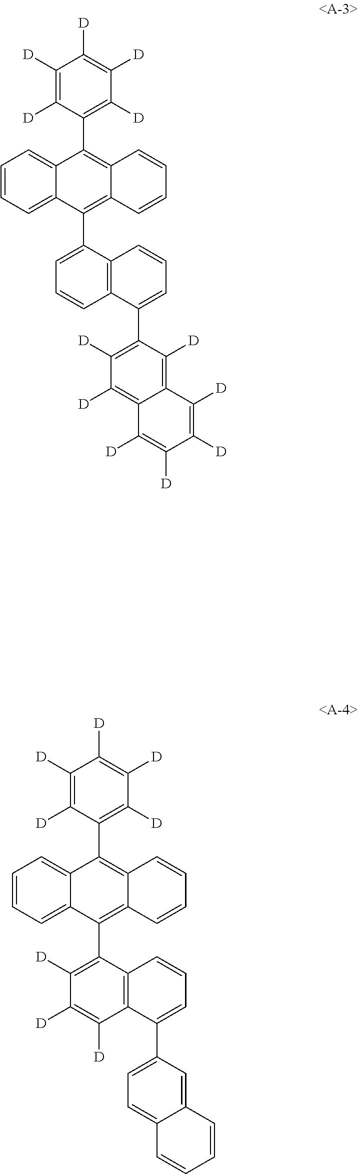 [SMILES: [2H]c1c([2H])c([2H])c(-c2c3ccccc3c(-c3cccc4c(-c5c([2H])c([2H])c6c([2H])c([2H])c([2H])c([2H])c6c5[2H])cccc34)c3ccccc23)c([2H])c1[2H] ; [2H]c1c([2H])c([2H])c(-c2c3ccccc3c(-c3c([2H])c([2H])c([2H])c4c(-c5ccc6ccccc6c5)cccc34)c3ccccc23)c([2H])c1[2H]]

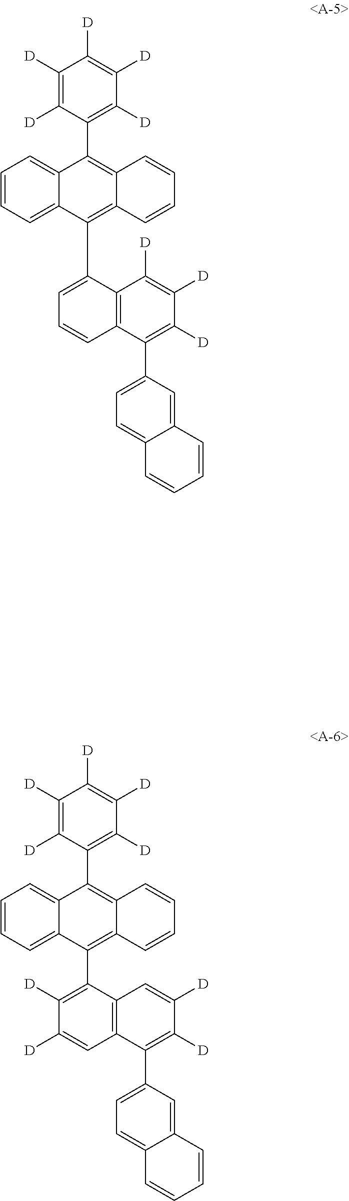 [SMILES: [2H]c1c([2H])c([2H])c(-c2c3ccccc3c(-c3cccc4c(-c5ccc6ccccc6c5)c([2H])c([2H])c([2H])c34)c3ccccc23)c([2H])c1[2H] ; [2H]c1cc2c(-c3c4ccccc4c(-c4c([2H])c([2H])c([2H])c([2H])c4[2H])c4ccccc34)c([2H])c([2H])cc2c(-c2ccc3ccccc3c2)c1[2H]]

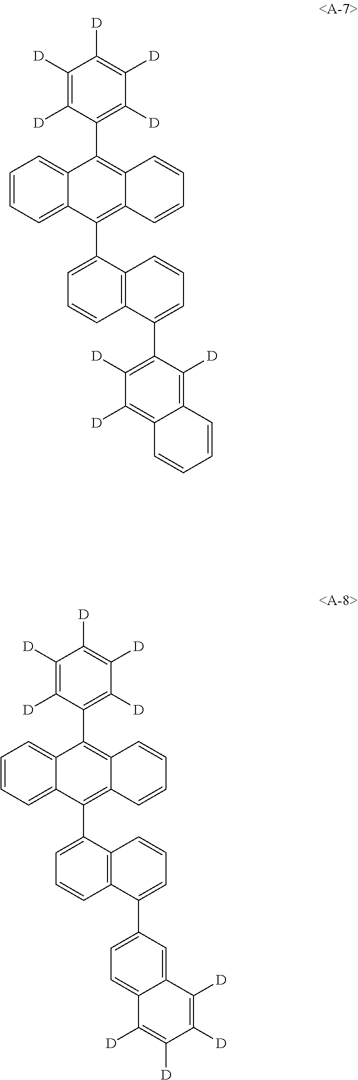 [SMILES: [2H]c1c([2H])c([2H])c(-c2c3ccccc3c(-c3cccc4c(-c5c([2H])c([2H])c6ccccc6c5[2H])cccc34)c3ccccc23)c([2H])c1[2H] ; [2H]c1c([2H])c([2H])c(-c2c3ccccc3c(-c3cccc4c(-c5ccc6c([2H])c([2H])c([2H])c([2H])c6c5)cccc34)c3ccccc23)c([2H])c1[2H]]

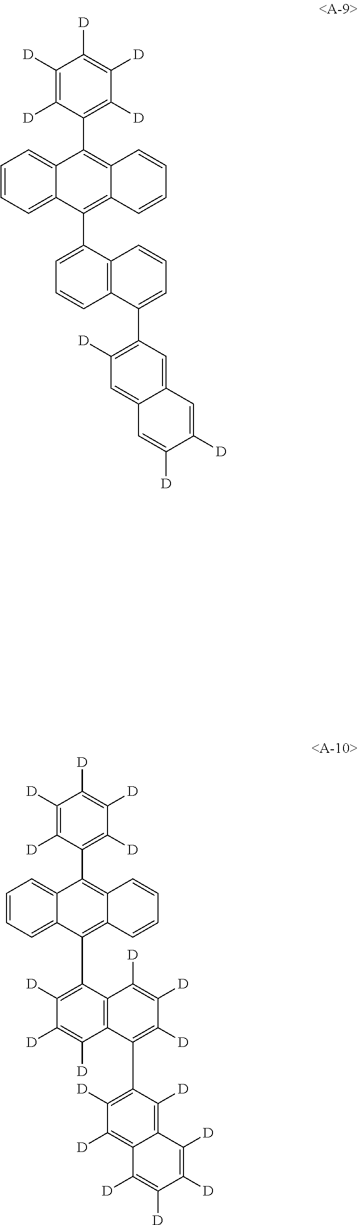 [SMILES: [2H]c1cc2cc([2H])c(-c3cccc4c(-c5c6ccccc6c(-c6c([2H])c([2H])c([2H])c([2H])c6[2H])c6ccccc56)cccc34)cc2cc1[2H] ; [2H]c1c([2H])c([2H])c(-c2c3ccccc3c(-c3c([2H])c([2H])c([2H])c4c(-c5c([2H])c([2H])c6c([2H])c([2H])c([2H])c([2H])c6c5[2H])c([2H])c([2H])c([2H])c34)c3ccccc23)c([2H])c1[2H]]

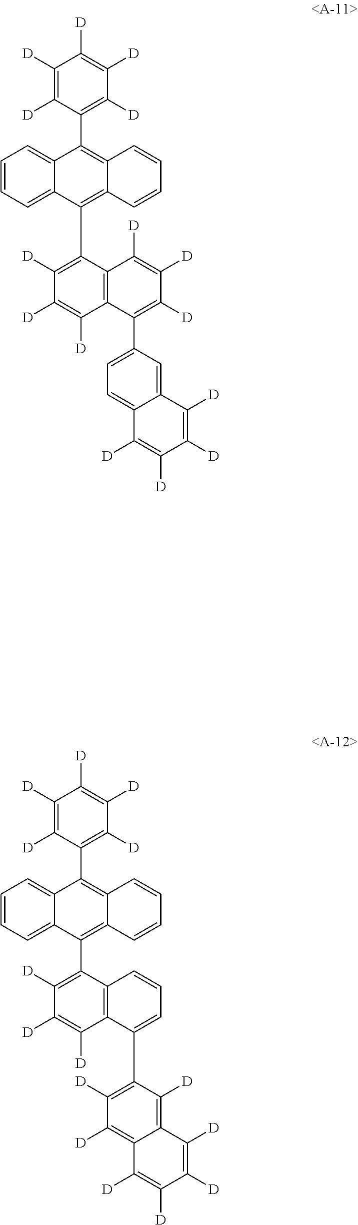 [SMILES: [2H]c1c([2H])c([2H])c(-c2c3ccccc3c(-c3c([2H])c([2H])c([2H])c4c(-c5ccc6c([2H])c([2H])c([2H])c([2H])c6c5)c([2H])c([2H])c([2H])c34)c3ccccc23)c([2H])c1[2H] ; [2H]c1c([2H])c([2H])c(-c2c3ccccc3c(-c3c([2H])c([2H])c([2H])c4c(-c5c([2H])c([2H])c6c([2H])c([2H])c([2H])c([2H])c6c5[2H])cccc34)c3ccccc23)c([2H])c1[2H]]

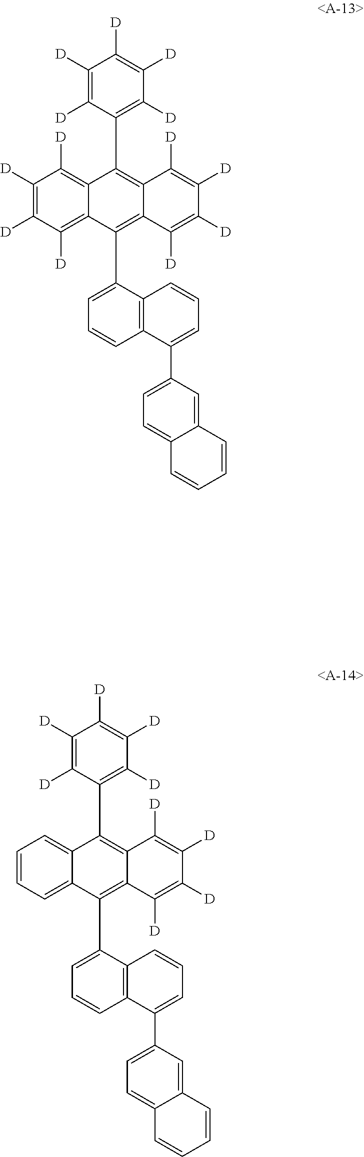 [SMILES: [2H]c1c([2H])c([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1[2H] ; [2H]c1c([2H])c([2H])c(-c2c3ccccc3c(-c3cccc4c(-c5ccc6ccccc6c5)cccc34)c3c([2H])c([2H])c([2H])c([2H])c23)c([2H])c1[2H]]

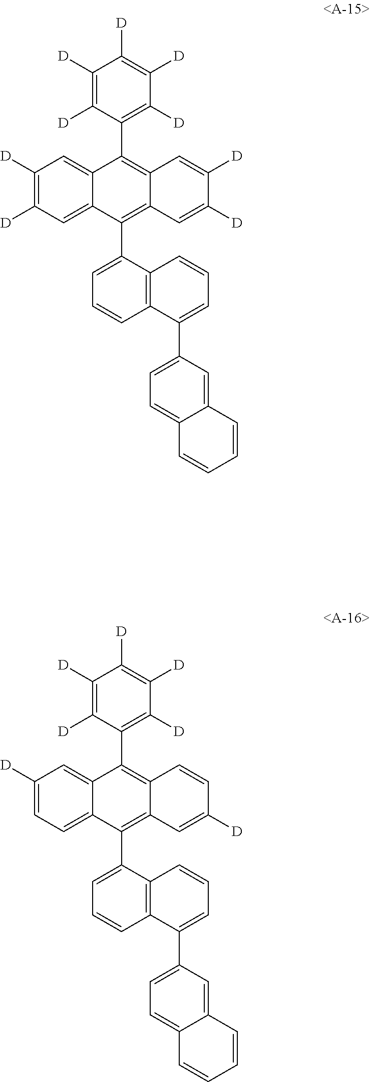 [SMILES: [2H]c1cc2c(-c3c([2H])c([2H])c([2H])c([2H])c3[2H])c3cc([2H])c([2H])cc3c(-c3cccc4c(-c5ccc6ccccc6c5)cccc34)c2cc1[2H] ; [2H]c1ccc2c(-c3cccc4c(-c5ccc6ccccc6c5)cccc34)c3cc([2H])ccc3c(-c3c([2H])c([2H])c([2H])c([2H])c3[2H])c2c1]

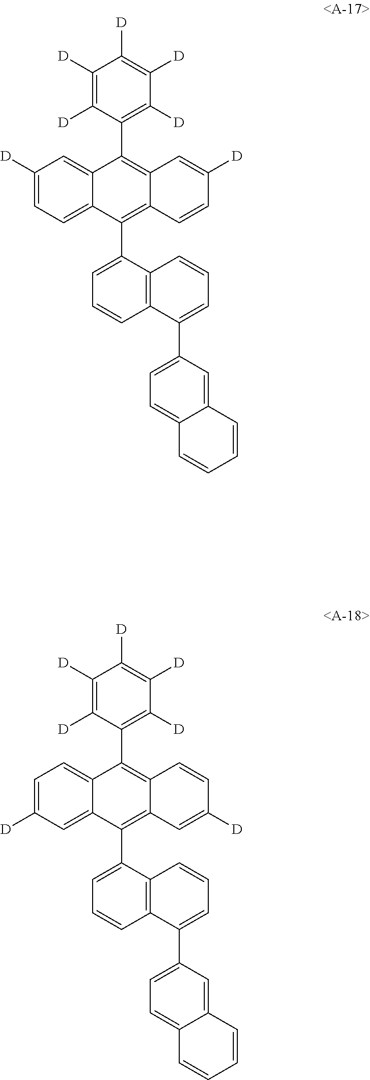 [SMILES: [2H]c1ccc2c(-c3cccc4c(-c5ccc6ccccc6c5)cccc34)c3ccc([2H])cc3c(-c3c([2H])c([2H])c([2H])c([2H])c3[2H])c2c1 ; [2H]c1ccc2c(-c3c([2H])c([2H])c([2H])c([2H])c3[2H])c3ccc([2H])cc3c(-c3cccc4c(-c5ccc6ccccc6c5)cccc34)c2c1]

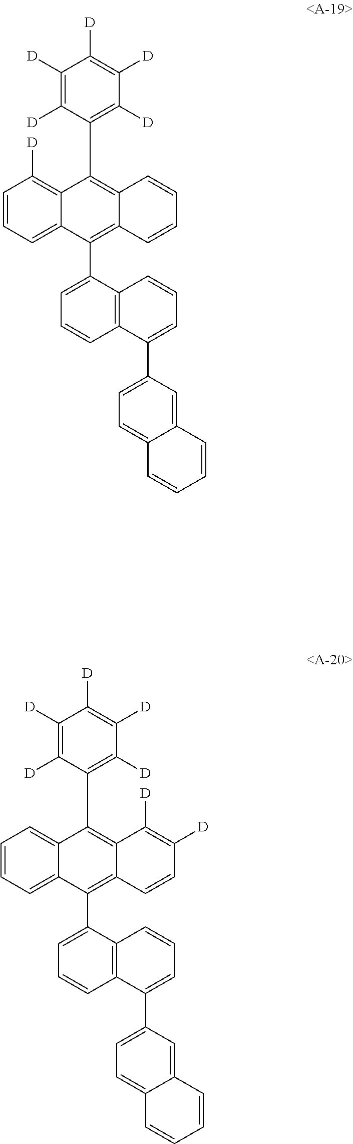 [SMILES: [2H]c1c([2H])c([2H])c(-c2c3ccccc3c(-c3cccc4c(-c5ccc6ccccc6c5)cccc34)c3cccc([2H])c23)c([2H])c1[2H] ; [2H]c1ccc2c(-c3cccc4c(-c5ccc6ccccc6c5)cccc34)c3ccccc3c(-c3c([2H])c([2H])c([2H])c([2H])c3[2H])c2c1[2H]]

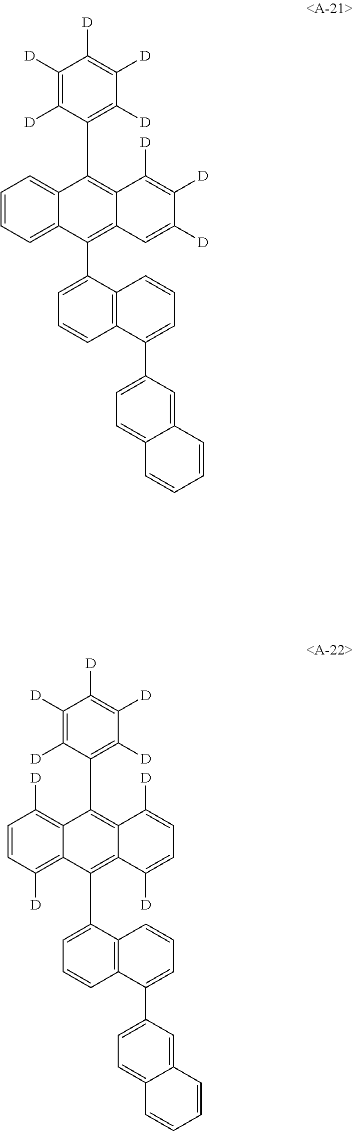 [SMILES: [2H]c1cc2c(-c3cccc4c(-c5ccc6ccccc6c5)cccc34)c3ccccc3c(-c3c([2H])c([2H])c([2H])c([2H])c3[2H])c2c([2H])c1[2H] ; [2H]c1c([2H])c([2H])c(-c2c3c([2H])ccc([2H])c3c(-c3cccc4c(-c5ccc6ccccc6c5)cccc34)c3c([2H])ccc([2H])c23)c([2H])c1[2H]]

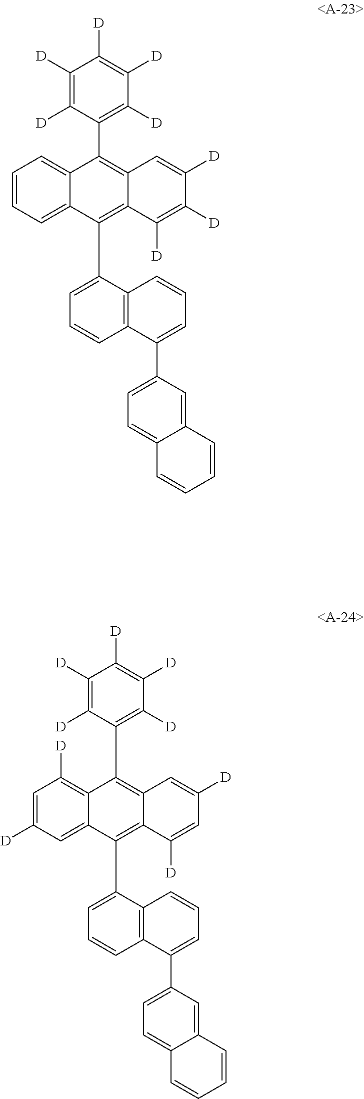 [SMILES: [2H]c1cc2c(-c3c([2H])c([2H])c([2H])c([2H])c3[2H])c3ccccc3c(-c3cccc4c(-c5ccc6ccccc6c5)cccc34)c2c([2H])c1[2H] ; [2H]c1cc([2H])c2c(-c3c([2H])c([2H])c([2H])c([2H])c3[2H])c3cc([2H])cc([2H])c3c(-c3cccc4c(-c5ccc6ccccc6c5)cccc34)c2c1]

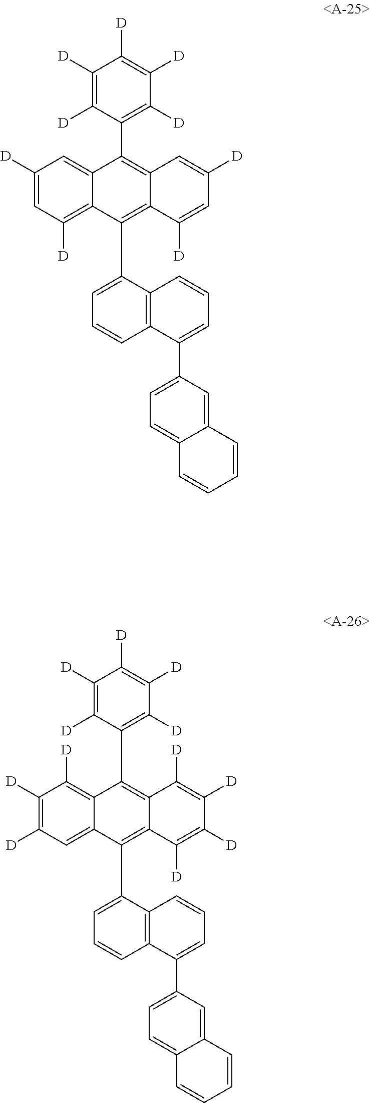 [SMILES: [2H]c1cc([2H])c2c(-c3cccc4c(-c5ccc6ccccc6c5)cccc34)c3c([2H])cc([2H])cc3c(-c3c([2H])c([2H])c([2H])c([2H])c3[2H])c2c1 ; [2H]c1cc2c(-c3cccc4c(-c5ccc6ccccc6c5)cccc34)c3c([2H])c([2H])c([2H])c([2H])c3c(-c3c([2H])c([2H])c([2H])c([2H])c3[2H])c2c([2H])c1[2H]]

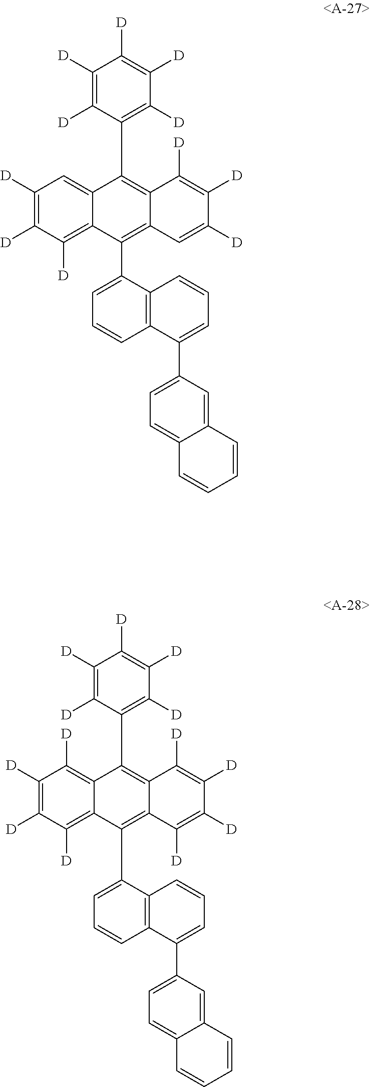 [SMILES: [2H]c1cc2c(-c3cccc4c(-c5ccc6ccccc6c5)cccc34)c3c([2H])c([2H])c([2H])cc3c(-c3c([2H])c([2H])c([2H])c([2H])c3[2H])c2c([2H])c1[2H] ; [2H]c1c([2H])c([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1[2H]]

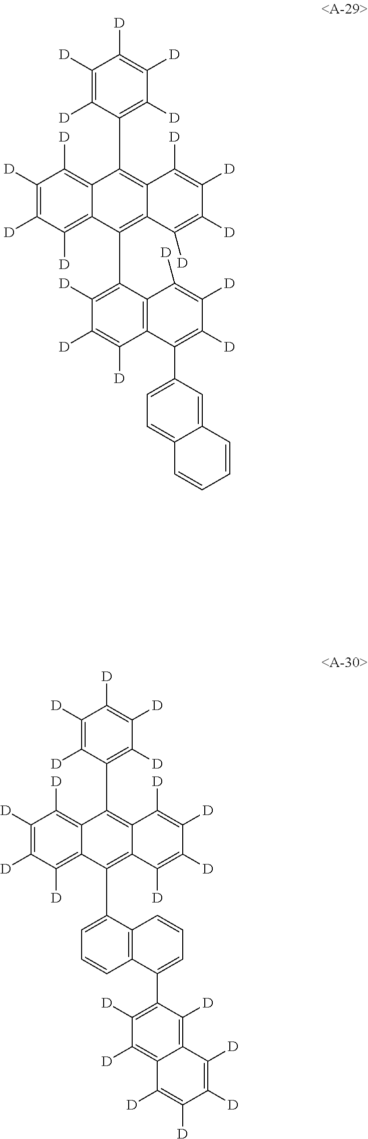 [SMILES: [2H]c1c([2H])c([2H])c(-c2c3c([2H])c([2H])c([2H])c([2H])c3c(-c3c([2H])c([2H])c([2H])c4c(-c5ccc6ccccc6c5)c([2H])c([2H])c([2H])c34)c3c([2H])c([2H])c([2H])c([2H])c23)c([2H])c1[2H] ; [2H]c1c([2H])c([2H])c(-c2c3c([2H])c([2H])c([2H])c([2H])c3c(-c3cccc4c(-c5c([2H])c([2H])c6c([2H])c([2H])c([2H])c([2H])c6c5[2H])cccc34)c3c([2H])c([2H])c([2H])c([2H])c23)c([2H])c1[2H]]

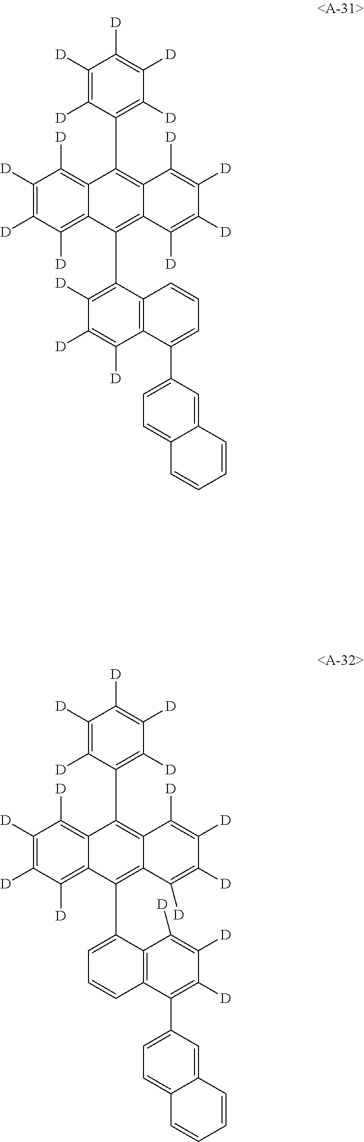 [SMILES: [2H]c1c([2H])c([2H])c(-c2c3c([2H])c([2H])c([2H])c([2H])c3c(-c3c([2H])c([2H])c([2H])c4c(-c5ccc6ccccc6c5)cccc34)c3c([2H])c([2H])c([2H])c([2H])c23)c([2H])c1[2H] ; [2H]c1c([2H])c([2H])c(-c2c3c([2H])c([2H])c([2H])c([2H])c3c(-c3cccc4c(-c5ccc6ccccc6c5)c([2H])c([2H])c([2H])c34)c3c([2H])c([2H])c([2H])c([2H])c23)c([2H])c1[2H]]

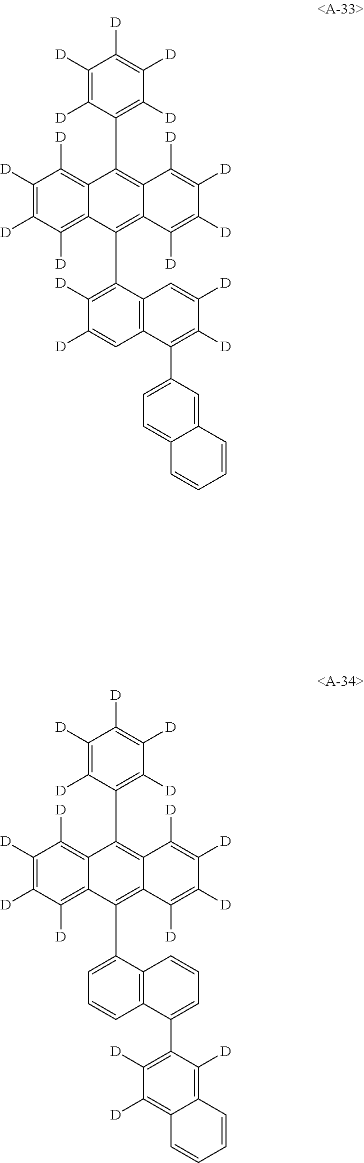 [SMILES: [2H]c1cc2c(-c3c4c([2H])c([2H])c([2H])c([2H])c4c(-c4c([2H])c([2H])c([2H])c([2H])c4[2H])c4c([2H])c([2H])c([2H])c([2H])c34)c([2H])c([2H])cc2c(-c2ccc3ccccc3c2)c1[2H] ; [2H]c1c([2H])c([2H])c(-c2c3c([2H])c([2H])c([2H])c([2H])c3c(-c3cccc4c(-c5c([2H])c([2H])c6ccccc6c5[2H])cccc34)c3c([2H])c([2H])c([2H])c([2H])c23)c([2H])c1[2H]]

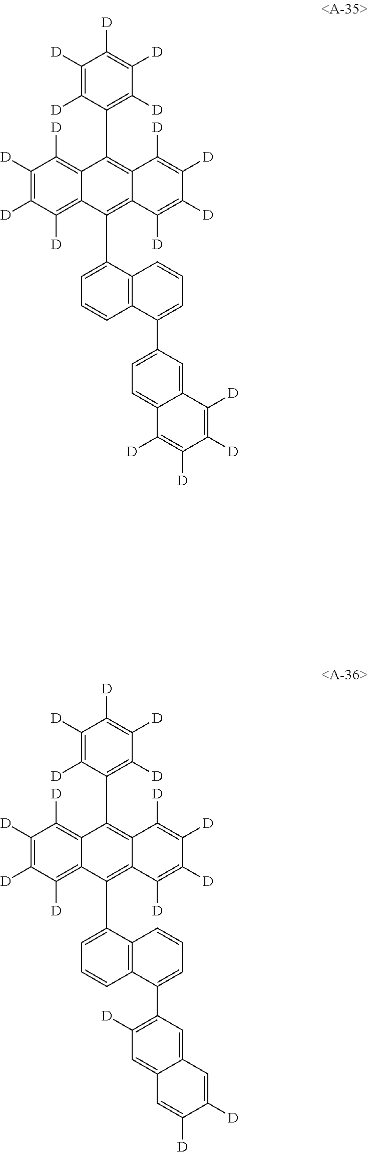 [SMILES: [2H]c1c([2H])c([2H])c(-c2c3c([2H])c([2H])c([2H])c([2H])c3c(-c3cccc4c(-c5ccc6c([2H])c([2H])c([2H])c([2H])c6c5)cccc34)c3c([2H])c([2H])c([2H])c([2H])c23)c([2H])c1[2H] ; [2H]c1cc2cc([2H])c(-c3cccc4c(-c5c6c([2H])c([2H])c([2H])c([2H])c6c(-c6c([2H])c([2H])c([2H])c([2H])c6[2H])c6c([2H])c([2H])c([2H])c([2H])c56)cccc34)cc2cc1[2H]]

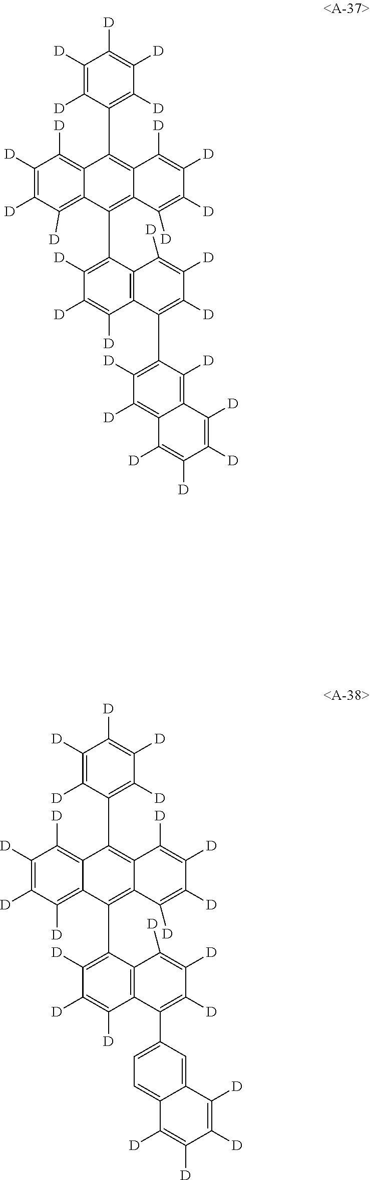 [SMILES: [2H]c1c([2H])c([2H])c(-c2c3c([2H])c([2H])c([2H])c([2H])c3c(-c3c([2H])c([2H])c([2H])c4c(-c5c([2H])c([2H])c6c([2H])c([2H])c([2H])c([2H])c6c5[2H])c([2H])c([2H])c([2H])c34)c3c([2H])c([2H])c([2H])c([2H])c23)c([2H])c1[2H] ; [2H]c1c([2H])c([2H])c(-c2c3c([2H])c([2H])c([2H])c([2H])c3c(-c3c([2H])c([2H])c([2H])c4c(-c5ccc6c([2H])c([2H])c([2H])c([2H])c6c5)c([2H])c([2H])c([2H])c34)c3c([2H])c([2H])c([2H])c([2H])c23)c([2H])c1[2H]]

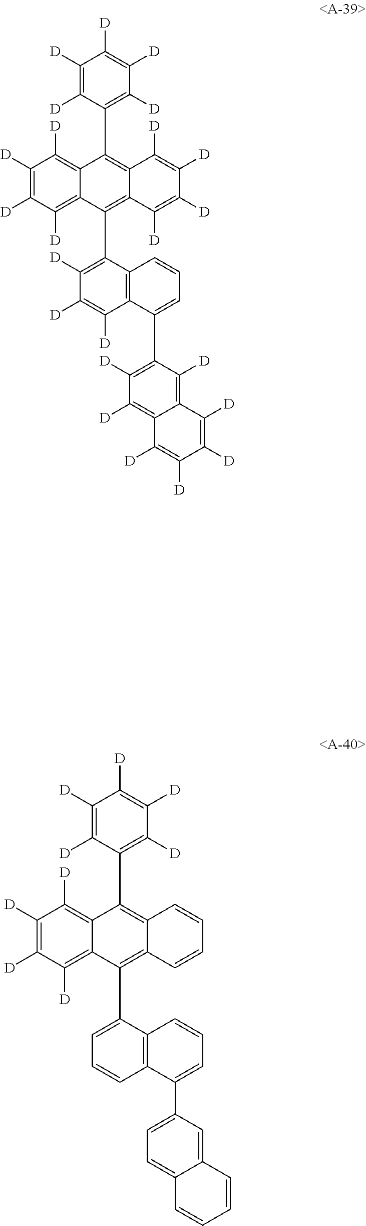 [SMILES: [2H]c1c([2H])c([2H])c(-c2c3c([2H])c([2H])c([2H])c([2H])c3c(-c3c([2H])c([2H])c([2H])c4c(-c5c([2H])c([2H])c6c([2H])c([2H])c([2H])c([2H])c6c5[2H])cccc34)c3c([2H])c([2H])c([2H])c([2H])c23)c([2H])c1[2H] ; [2H]c1c([2H])c([2H])c(-c2c3ccccc3c(-c3cccc4c(-c5ccc6ccccc6c5)cccc34)c3c([2H])c([2H])c([2H])c([2H])c23)c([2H])c1[2H]]

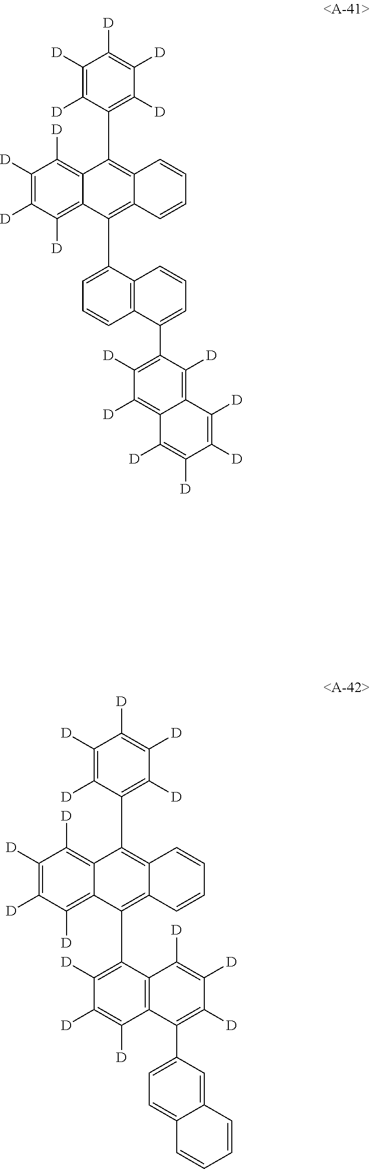 [SMILES: [2H]c1c([2H])c([2H])c(-c2c3ccccc3c(-c3cccc4c(-c5c([2H])c([2H])c6c([2H])c([2H])c([2H])c([2H])c6c5[2H])cccc34)c3c([2H])c([2H])c([2H])c([2H])c23)c([2H])c1[2H] ; [2H]c1c([2H])c([2H])c(-c2c3ccccc3c(-c3c([2H])c([2H])c([2H])c4c(-c5ccc6ccccc6c5)c([2H])c([2H])c([2H])c34)c3c([2H])c([2H])c([2H])c([2H])c23)c([2H])c1[2H]]

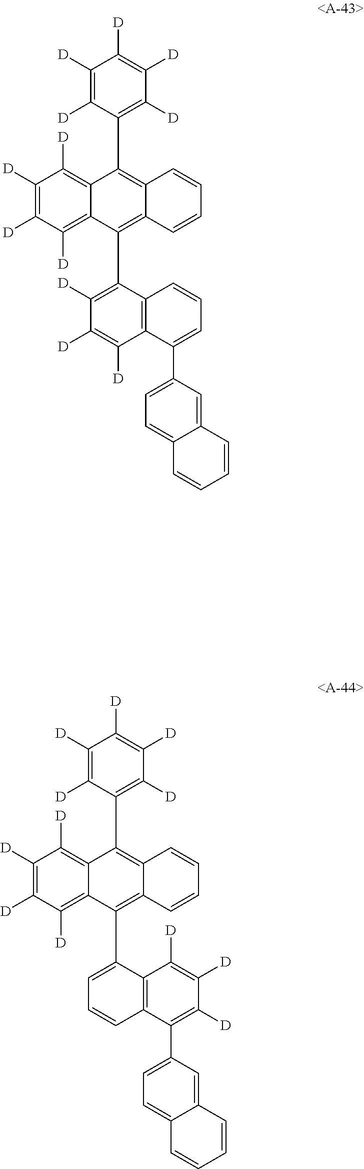 [SMILES: [2H]c1c([2H])c([2H])c(-c2c3ccccc3c(-c3c([2H])c([2H])c([2H])c4c(-c5ccc6ccccc6c5)cccc34)c3c([2H])c([2H])c([2H])c([2H])c23)c([2H])c1[2H] ; [2H]c1c([2H])c([2H])c(-c2c3ccccc3c(-c3cccc4c(-c5ccc6ccccc6c5)c([2H])c([2H])c([2H])c34)c3c([2H])c([2H])c([2H])c([2H])c23)c([2H])c1[2H]]

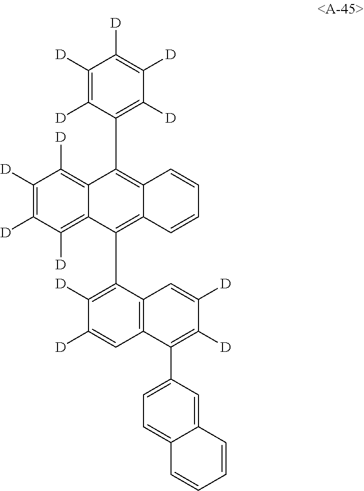 [SMILES: [2H]c1cc2c(-c3c4ccccc4c(-c4c([2H])c([2H])c([2H])c([2H])c4[2H])c4c([2H])c([2H])c([2H])c([2H])c34)c([2H])c([2H])cc2c(-c2ccc3ccccc3c2)c1[2H]]

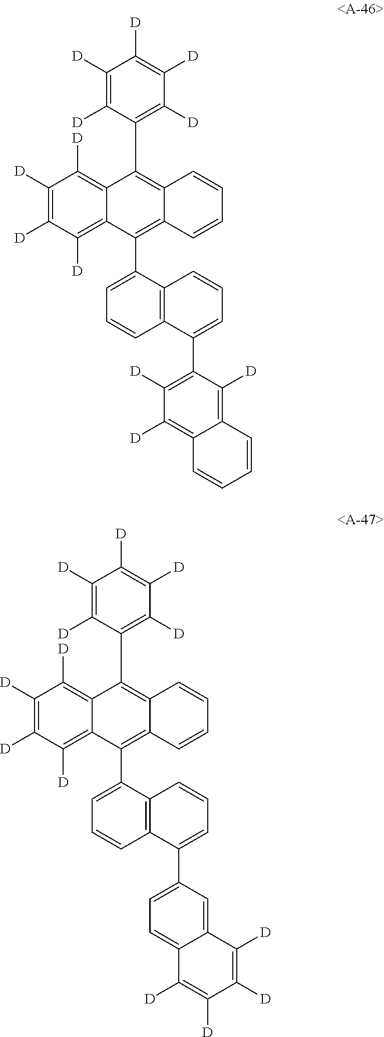 [SMILES: [2H]c1c([2H])c([2H])c(-c2c3ccccc3c(-c3cccc4c(-c5c([2H])c([2H])c6ccccc6c5[2H])cccc34)c3c([2H])c([2H])c([2H])c([2H])c23)c([2H])c1[2H] ; [2H]c1c([2H])c([2H])c(-c2c3ccccc3c(-c3cccc4c(-c5ccc6c([2H])c([2H])c([2H])c([2H])c6c5)cccc34)c3c([2H])c([2H])c([2H])c([2H])c23)c([2H])c1[2H]]

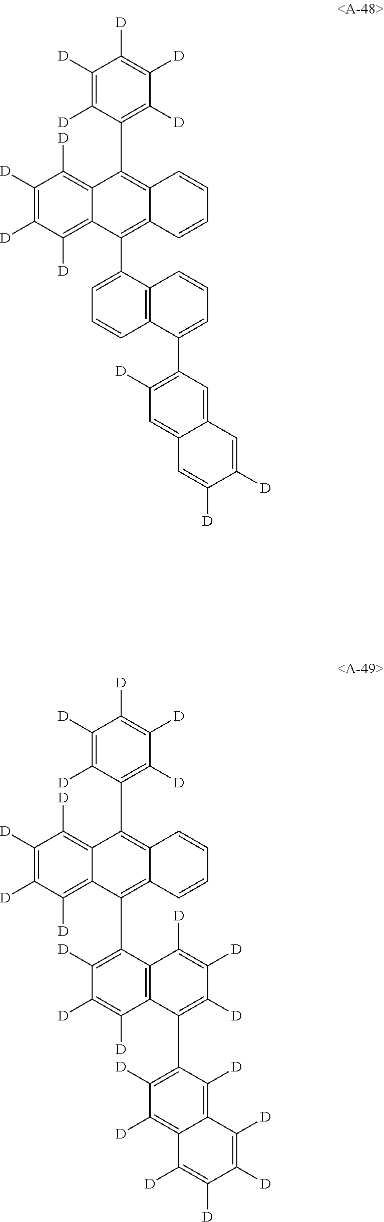 [SMILES: [2H]c1cc2cc([2H])c(-c3cccc4c(-c5c6ccccc6c(-c6c([2H])c([2H])c([2H])c([2H])c6[2H])c6c([2H])c([2H])c([2H])c([2H])c56)cccc34)cc2cc1[2H] ; [2H]c1c([2H])c([2H])c(-c2c3ccccc3c(-c3c([2H])c([2H])c([2H])c4c(-c5c([2H])c([2H])c6c([2H])c([2H])c([2H])c([2H])c6c5[2H])c([2H])c([2H])c([2H])c34)c3c([2H])c([2H])c([2H])c([2H])c23)c([2H])c1[2H]]

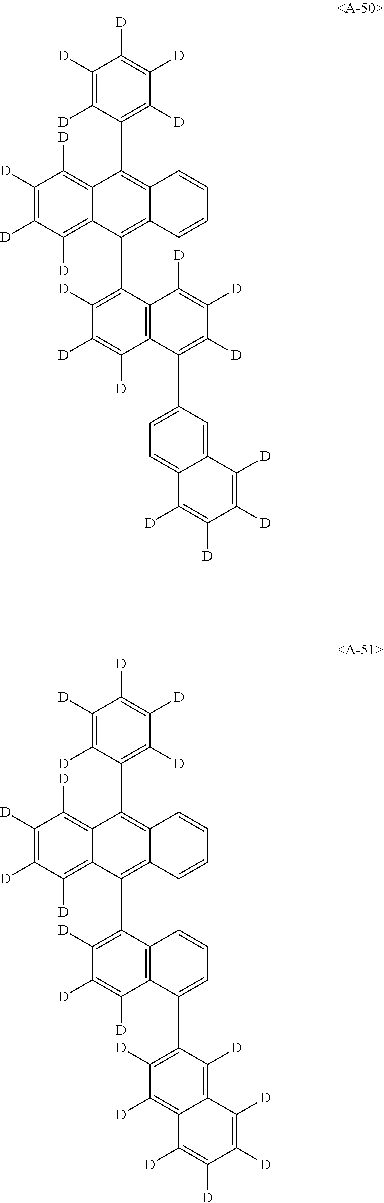 [SMILES: [2H]c1c([2H])c([2H])c(-c2c3ccccc3c(-c3c([2H])c([2H])c([2H])c4c(-c5ccc6c([2H])c([2H])c([2H])c([2H])c6c5)c([2H])c([2H])c([2H])c34)c3c([2H])c([2H])c([2H])c([2H])c23)c([2H])c1[2H] ; [2H]c1c([2H])c([2H])c(-c2c3ccccc3c(-c3c([2H])c([2H])c([2H])c4c(-c5c([2H])c([2H])c6c([2H])c([2H])c([2H])c([2H])c6c5[2H])cccc34)c3c([2H])c([2H])c([2H])c([2H])c23)c([2H])c1[2H]]

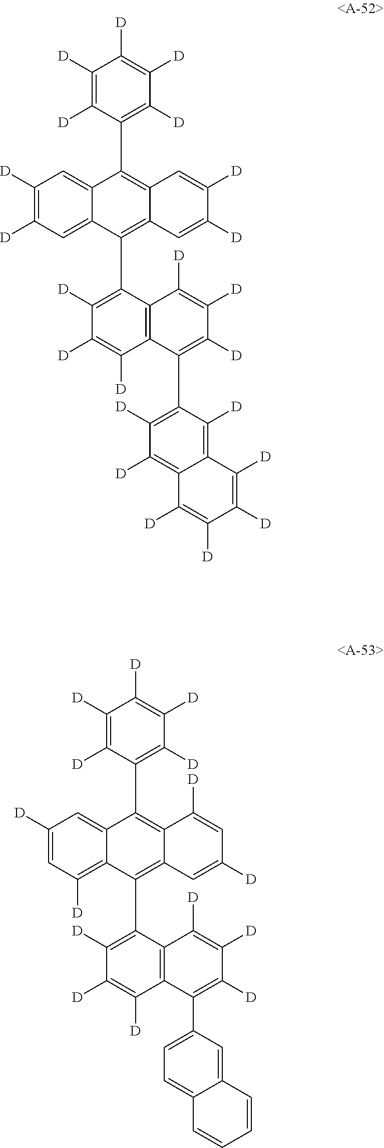 [SMILES: [2H]c1cc2c(-c3c([2H])c([2H])c([2H])c([2H])c3[2H])c3cc([2H])c([2H])cc3c(-c3c([2H])c([2H])c([2H])c4c(-c5c([2H])c([2H])c6c([2H])c([2H])c([2H])c([2H])c6c5[2H])c([2H])c([2H])c([2H])c34)c2cc1[2H] ; [2H]c1cc([2H])c2c(-c3c([2H])c([2H])c([2H])c([2H])c3[2H])c3cc([2H])cc([2H])c3c(-c3c([2H])c([2H])c([2H])c4c(-c5ccc6ccccc6c5)c([2H])c([2H])c([2H])c34)c2c1]

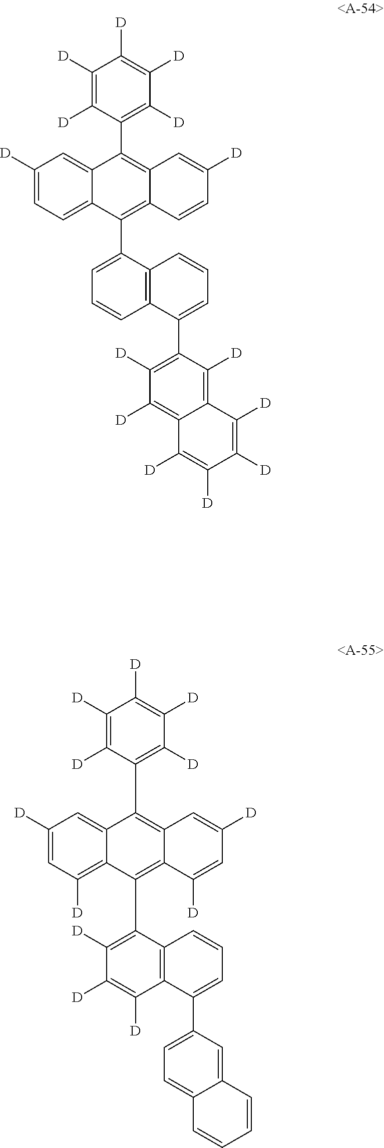 [SMILES: [2H]c1ccc2c(-c3cccc4c(-c5c([2H])c([2H])c6c([2H])c([2H])c([2H])c([2H])c6c5[2H])cccc34)c3ccc([2H])cc3c(-c3c([2H])c([2H])c([2H])c([2H])c3[2H])c2c1 ; [2H]c1cc([2H])c2c(-c3c([2H])c([2H])c([2H])c4c(-c5ccc6ccccc6c5)cccc34)c3c([2H])cc([2H])cc3c(-c3c([2H])c([2H])c([2H])c([2H])c3[2H])c2c1]

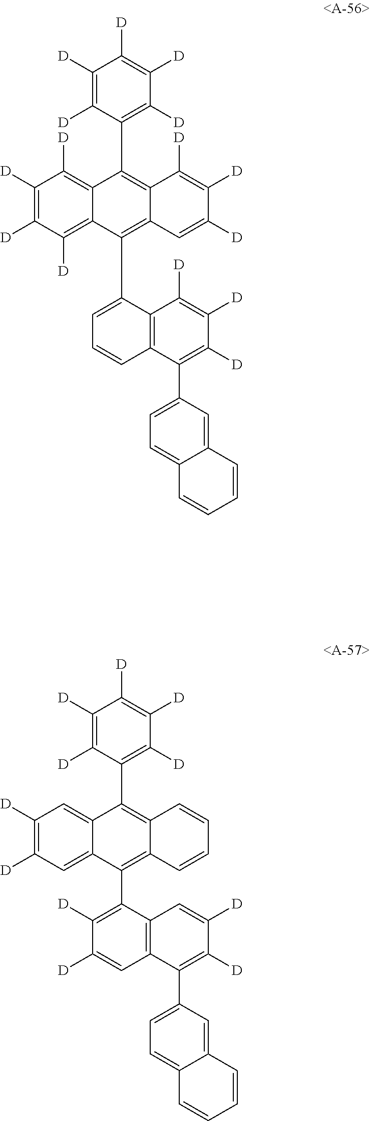 [SMILES: [2H]c1cc2c(-c3cccc4c(-c5ccc6ccccc6c5)c([2H])c([2H])c([2H])c34)c3c([2H])c([2H])c([2H])c([2H])c3c(-c3c([2H])c([2H])c([2H])c([2H])c3[2H])c2c([2H])c1[2H] ; [2H]c1cc2c(-c3c([2H])c([2H])c([2H])c([2H])c3[2H])c3ccccc3c(-c3c([2H])c([2H])cc4c(-c5ccc6ccccc6c5)c([2H])c([2H])cc34)c2cc1[2H]]

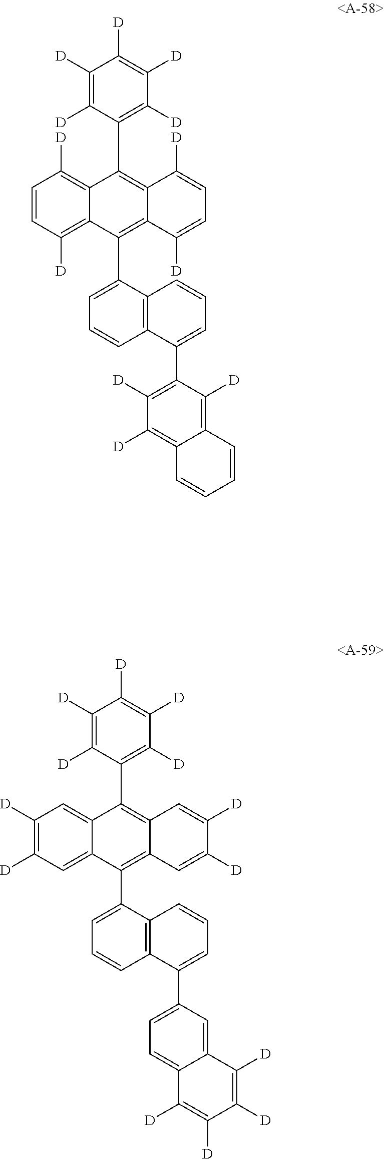 [SMILES: [2H]c1c([2H])c([2H])c(-c2c3c([2H])ccc([2H])c3c(-c3cccc4c(-c5c([2H])c([2H])c6ccccc6c5[2H])cccc34)c3c([2H])ccc([2H])c23)c([2H])c1[2H] ; [2H]c1cc2c(-c3c([2H])c([2H])c([2H])c([2H])c3[2H])c3cc([2H])c([2H])cc3c(-c3cccc4c(-c5ccc6c([2H])c([2H])c([2H])c([2H])c6c5)cccc34)c2cc1[2H]]

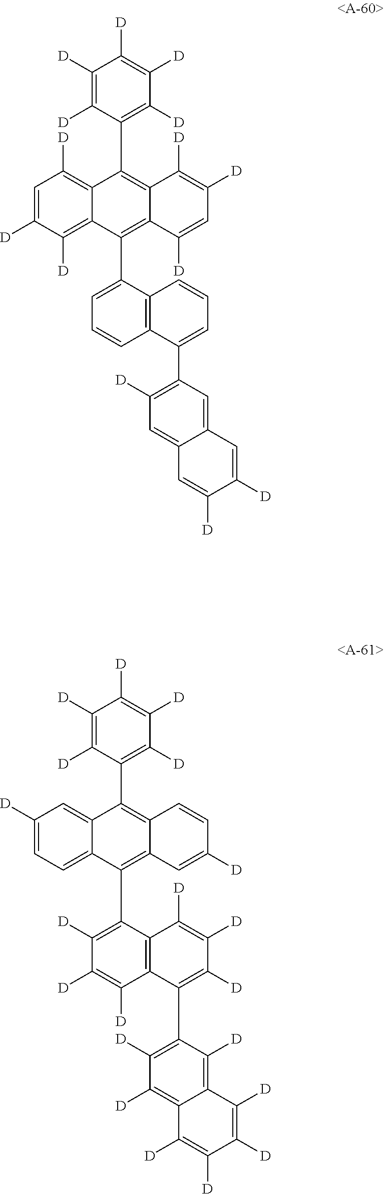 [SMILES: [2H]c1cc2cc([2H])c(-c3cccc4c(-c5c6c([2H])cc([2H])c([2H])c6c(-c6c([2H])c([2H])c([2H])c([2H])c6[2H])c6c([2H])cc([2H])c([2H])c56)cccc34)cc2cc1[2H] ; [2H]c1ccc2c(-c3c([2H])c([2H])c([2H])c4c(-c5c([2H])c([2H])c6c([2H])c([2H])c([2H])c([2H])c6c5[2H])c([2H])c([2H])c([2H])c34)c3cc([2H])ccc3c(-c3c([2H])c([2H])c([2H])c([2H])c3[2H])c2c1]

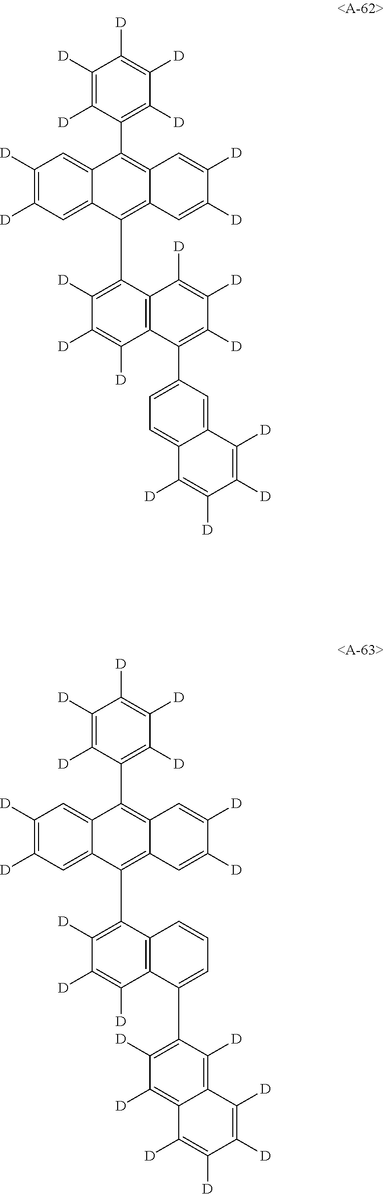 [SMILES: [2H]c1cc2c(-c3c([2H])c([2H])c([2H])c([2H])c3[2H])c3cc([2H])c([2H])cc3c(-c3c([2H])c([2H])c([2H])c4c(-c5ccc6c([2H])c([2H])c([2H])c([2H])c6c5)c([2H])c([2H])c([2H])c34)c2cc1[2H] ; [2H]c1cc2c(-c3c([2H])c([2H])c([2H])c([2H])c3[2H])c3cc([2H])c([2H])cc3c(-c3c([2H])c([2H])c([2H])c4c(-c5c([2H])c([2H])c6c([2H])c([2H])c([2H])c([2H])c6c5[2H])cccc34)c2cc1[2H]]

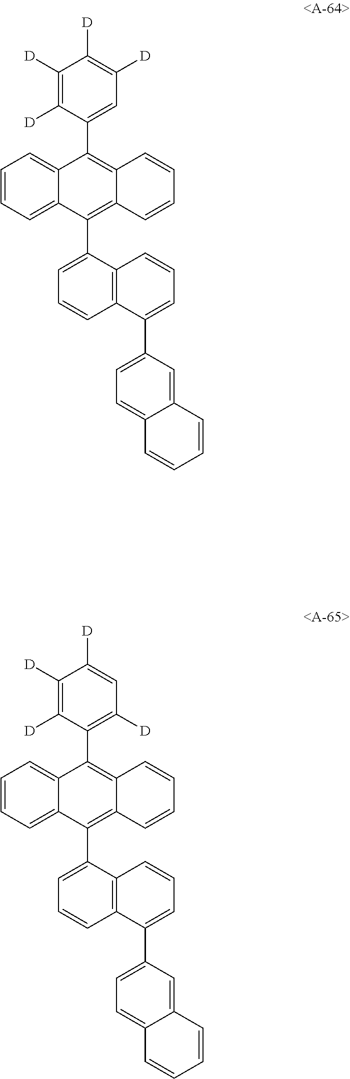 [SMILES: [2H]c1cc(-c2c3ccccc3c(-c3cccc4c(-c5ccc6ccccc6c5)cccc34)c3ccccc23)c([2H])c([2H])c1[2H] ; [2H]c1cc([2H])c(-c2c3ccccc3c(-c3cccc4c(-c5ccc6ccccc6c5)cccc34)c3ccccc23)c([2H])c1[2H]]

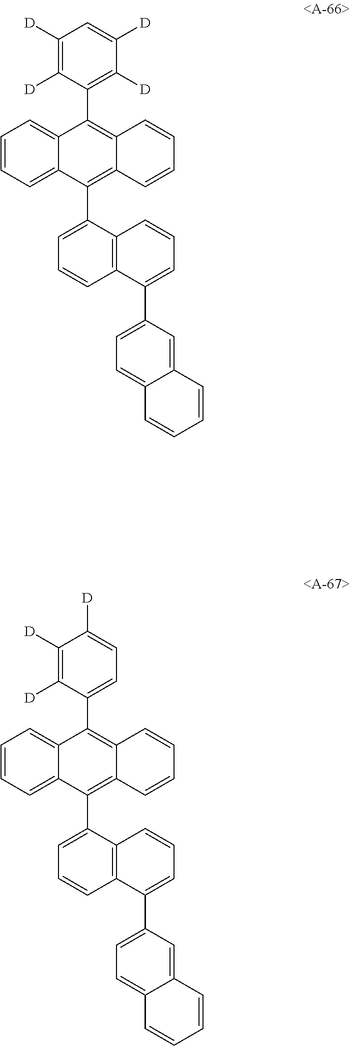 [SMILES: [2H]c1cc([2H])c([2H])c(-c2c3ccccc3c(-c3cccc4c(-c5ccc6ccccc6c5)cccc34)c3ccccc23)c1[2H] ; [2H]c1ccc(-c2c3ccccc3c(-c3cccc4c(-c5ccc6ccccc6c5)cccc34)c3ccccc23)c([2H])c1[2H]]

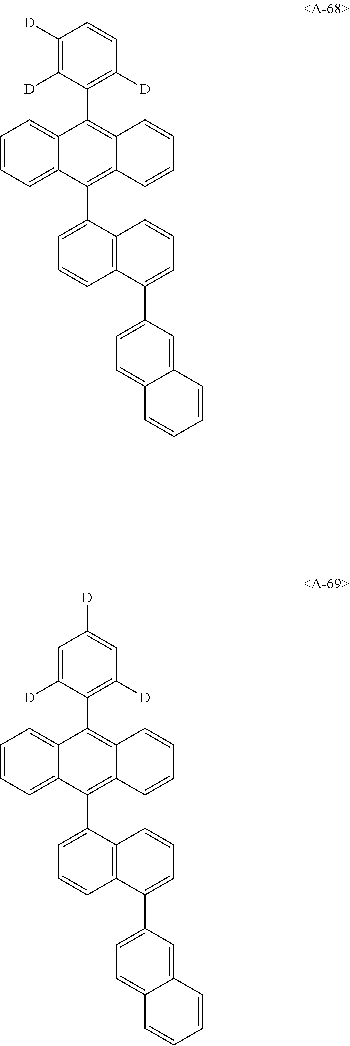 [SMILES: [2H]c1ccc([2H])c(-c2c3ccccc3c(-c3cccc4c(-c5ccc6ccccc6c5)cccc34)c3ccccc23)c1[2H] ; [2H]c1cc([2H])c(-c2c3ccccc3c(-c3cccc4c(-c5ccc6ccccc6c5)cccc34)c3ccccc23)c([2H])c1]

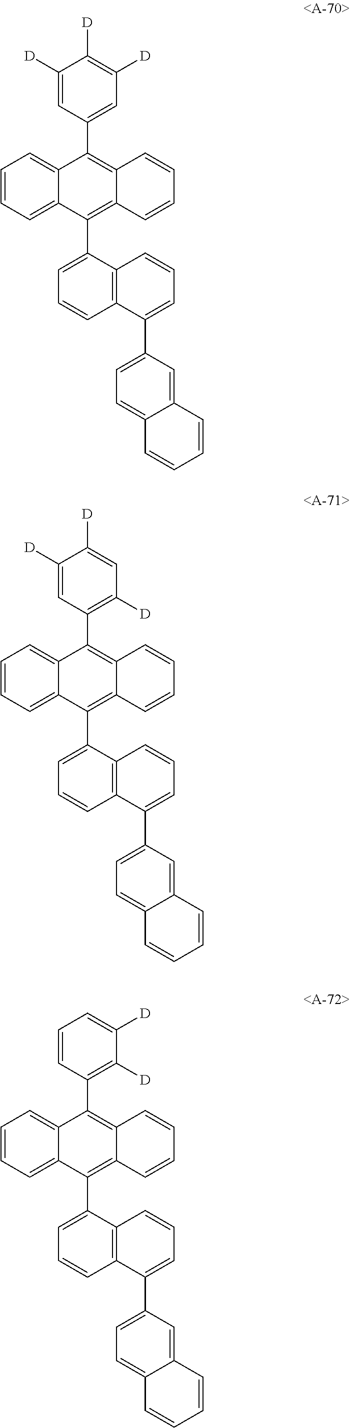 [SMILES: [2H]c1cc(-c2c3ccccc3c(-c3cccc4c(-c5ccc6ccccc6c5)cccc34)c3ccccc23)cc([2H])c1[2H] ; [2H]c1cc([2H])c(-c2c3ccccc3c(-c3cccc4c(-c5ccc6ccccc6c5)cccc34)c3ccccc23)cc1[2H] ; [2H]c1cccc(-c2c3ccccc3c(-c3cccc4c(-c5ccc6ccccc6c5)cccc34)c3ccccc23)c1[2H]]

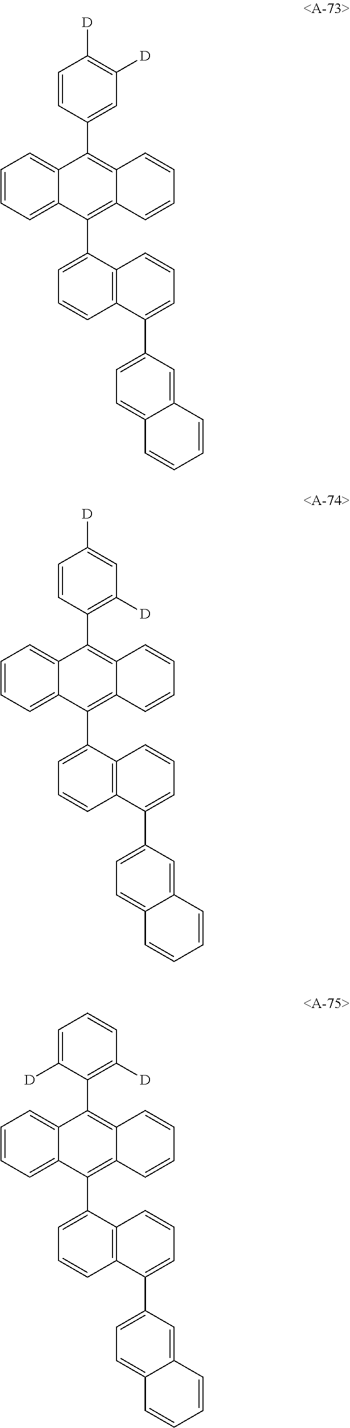 [SMILES: [2H]c1ccc(-c2c3ccccc3c(-c3cccc4c(-c5ccc6ccccc6c5)cccc34)c3ccccc23)cc1[2H] ; [2H]c1ccc(-c2c3ccccc3c(-c3cccc4c(-c5ccc6ccccc6c5)cccc34)c3ccccc23)c([2H])c1 ; [2H]c1cccc([2H])c1-c1c2ccccc2c(-c2cccc3c(-c4ccc5ccccc5c4)cccc23)c2ccccc12]

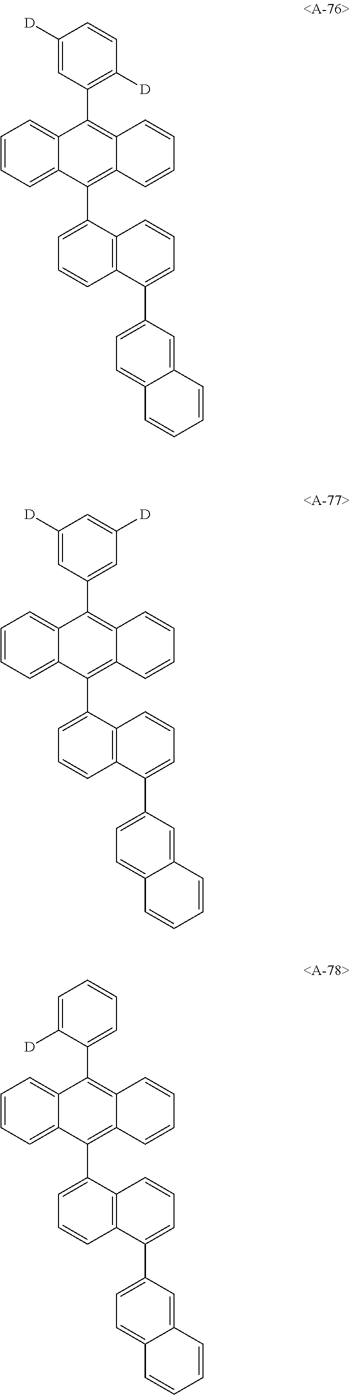 [SMILES: [2H]c1ccc([2H])c(-c2c3ccccc3c(-c3cccc4c(-c5ccc6ccccc6c5)cccc34)c3ccccc23)c1 ; [2H]c1cc([2H])cc(-c2c3ccccc3c(-c3cccc4c(-c5ccc6ccccc6c5)cccc34)c3ccccc23)c1 ; [2H]c1ccccc1-c1c2ccccc2c(-c2cccc3c(-c4ccc5ccccc5c4)cccc23)c2ccccc12]

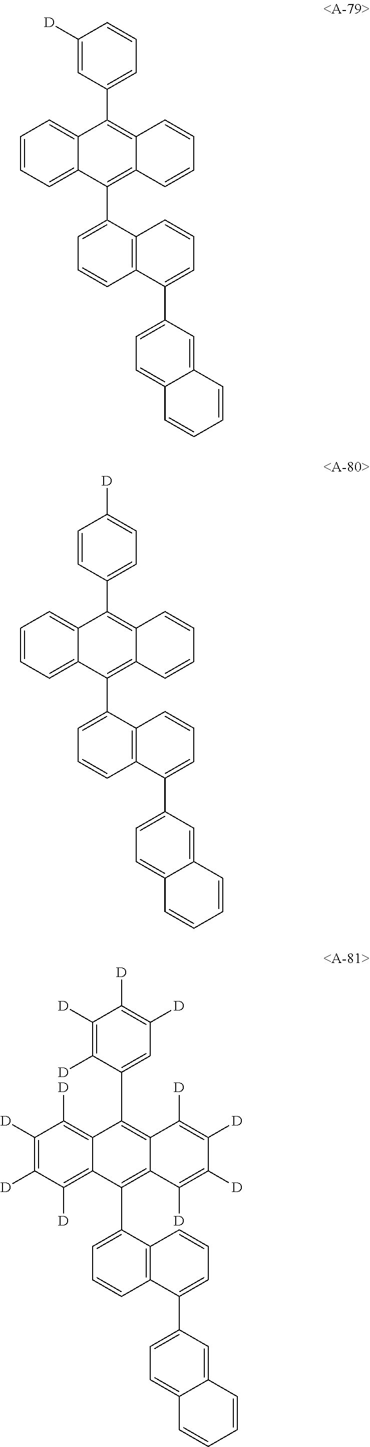 [SMILES: [2H]c1cccc(-c2c3ccccc3c(-c3cccc4c(-c5ccc6ccccc6c5)cccc34)c3ccccc23)c1 ; [2H]c1ccc(-c2c3ccccc3c(-c3cccc4c(-c5ccc6ccccc6c5)cccc34)c3ccccc23)cc1 ; [2H]c1cc(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c([2H])c1[2H]]

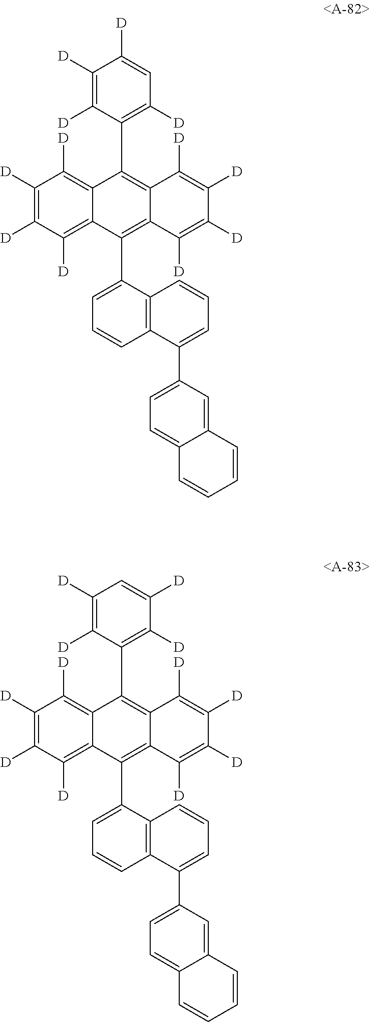 [SMILES: [2H]c1cc([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1[2H] ; [2H]c1cc([2H])c([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c1[2H]]

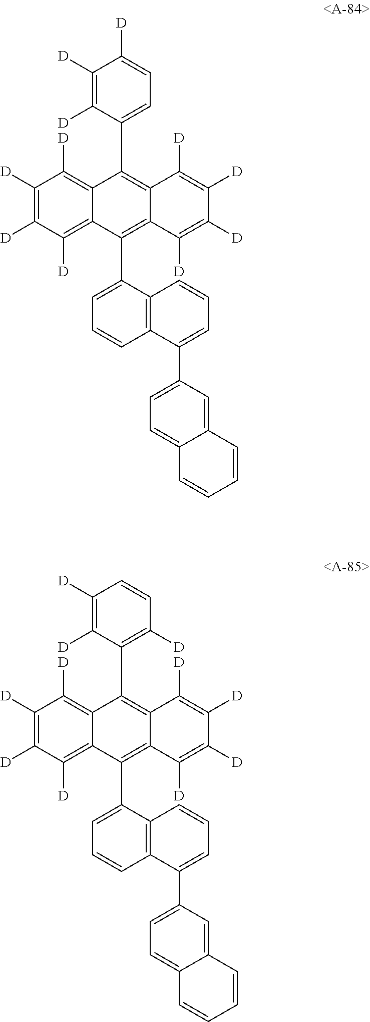 [SMILES: [2H]c1ccc(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1[2H] ; [2H]c1ccc([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c1[2H]]

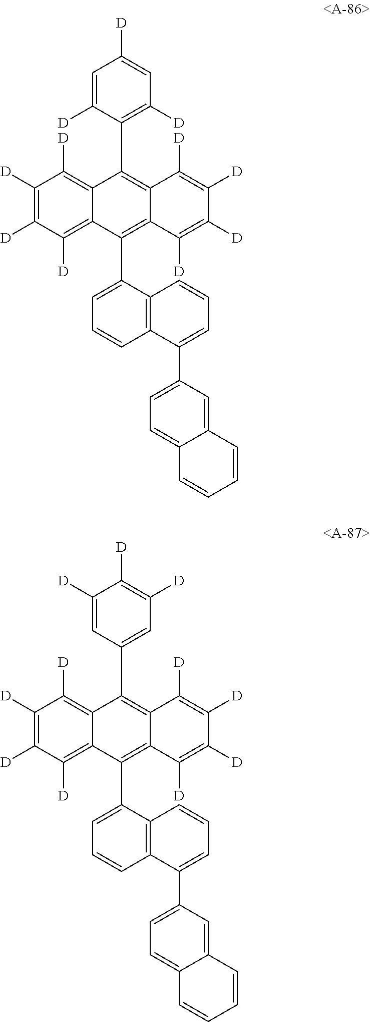 [SMILES: [2H]c1cc([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1 ; [2H]c1cc(-c2c3c([2H])c([2H])c([2H])c([2H])c3c(-c3cccc4c(-c5ccc6ccccc6c5)cccc34)c3c([2H])c([2H])c([2H])c([2H])c23)cc([2H])c1[2H]]

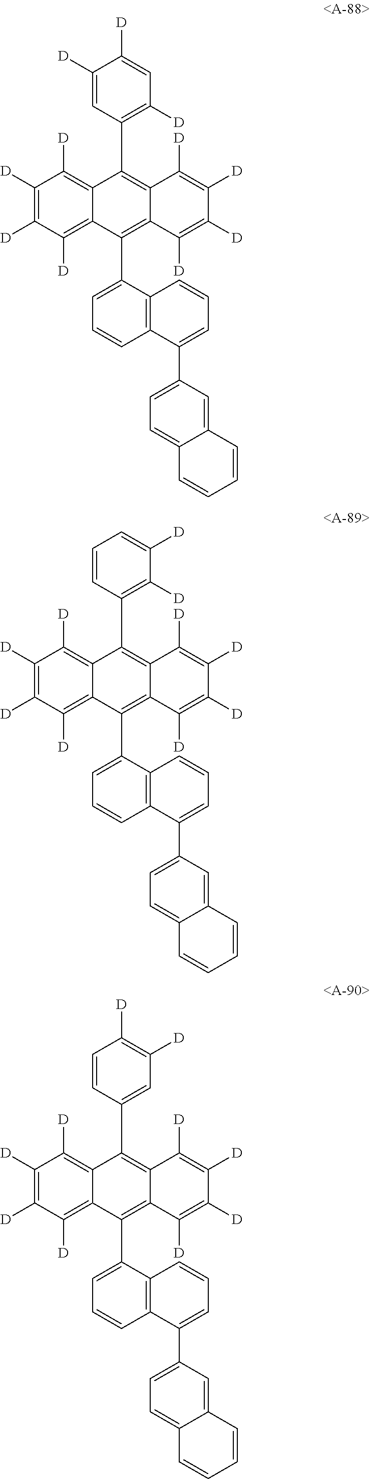

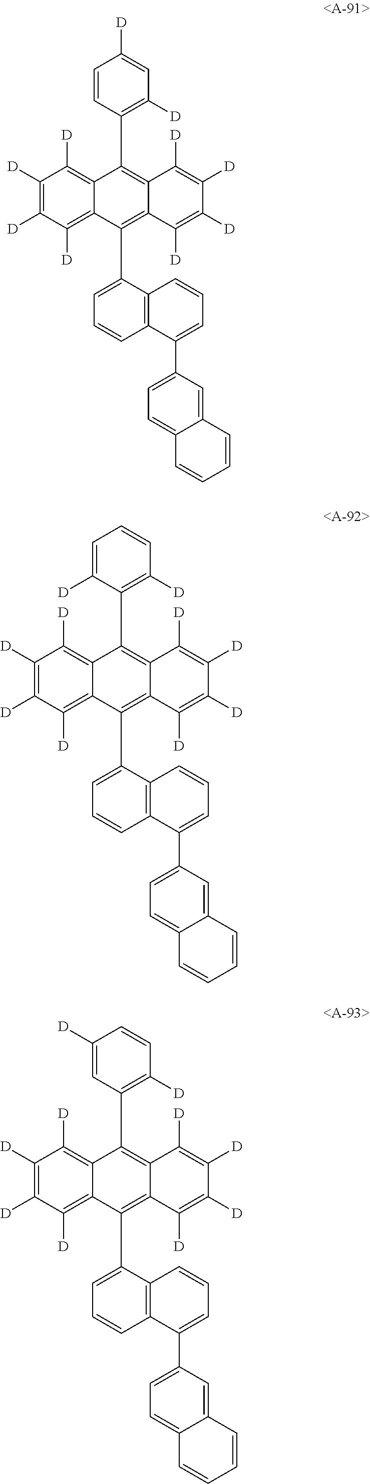 [SMILES: [2H]c1ccc(-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1 ; [2H]c1cccc([2H])c1-c1c2c([2H])c([2H])c([2H])c([2H])c2c(-c2cccc3c(-c4ccc5ccccc5c4)cccc23)c2c([2H])c([2H])c([2H])c([2H])c12 ; [2H]c1ccc([2H])c(-c2c3c([2H])c([2H])c([2H])c([2H])c3c(-c3cccc4c(-c5ccc6ccccc6c5)cccc34)c3c([2H])c([2H])c([2H])c([2H])c23)c1]

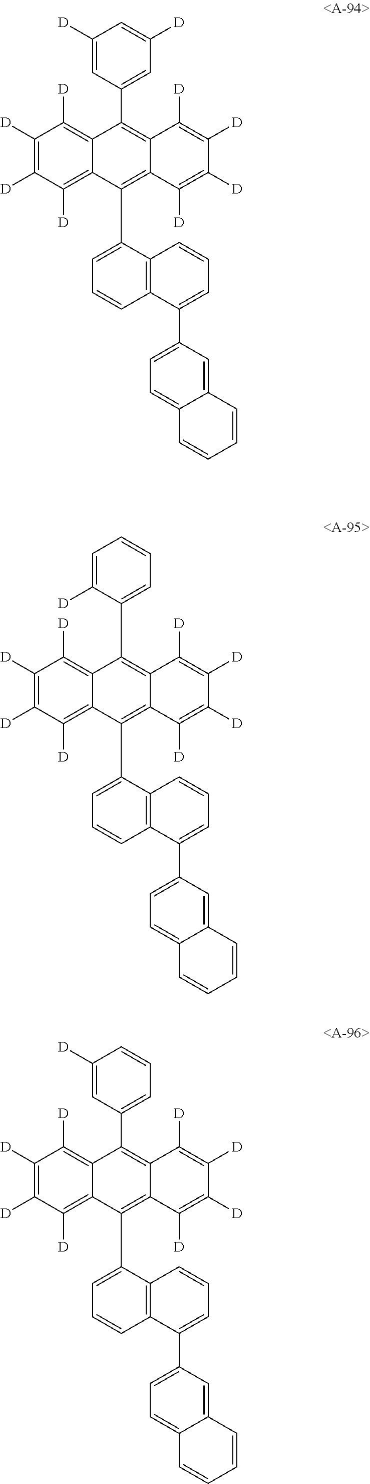 [SMILES: [2H]c1cc([2H])cc(-c2c3c([2H])c([2H])c([2H])c([2H])c3c(-c3cccc4c(-c5ccc6ccccc6c5)cccc34)c3c([2H])c([2H])c([2H])c([2H])c23)c1 ; [2H]c1ccccc1-c1c2c([2H])c([2H])c([2H])c([2H])c2c(-c2cccc3c(-c4ccc5ccccc5c4)cccc23)c2c([2H])c([2H])c([2H])c([2H])c12 ; [2H]c1cccc(-c2c3c([2H])c([2H])c([2H])c([2H])c3c(-c3cccc4c(-c5ccc6ccccc6c5)cccc34)c3c([2H])c([2H])c([2H])c([2H])c23)c1]

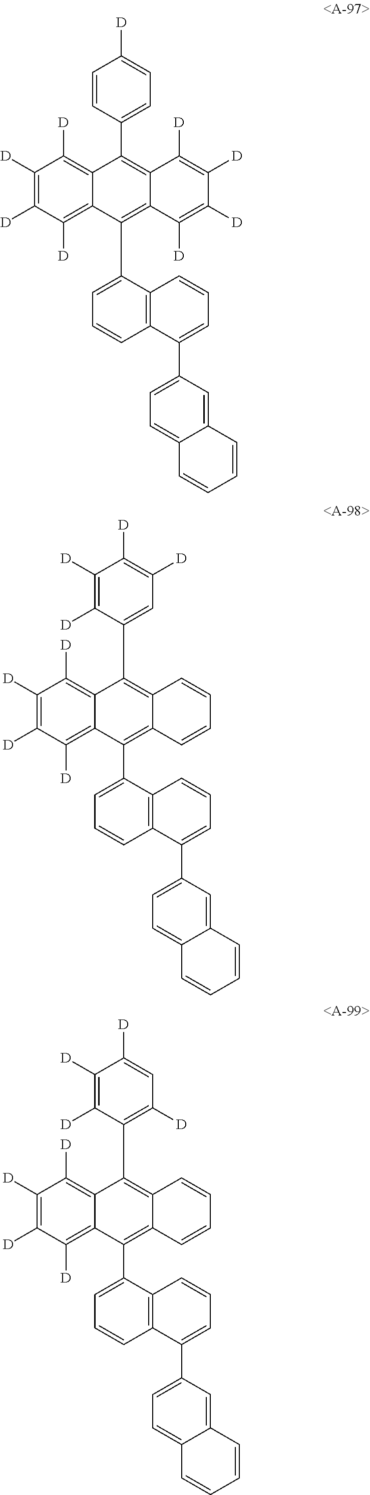 [SMILES: [2H]c1ccc(-c2c3c([2H])c([2H])c([2H])c([2H])c3c(-c3cccc4c(-c5ccc6ccccc6c5)cccc34)c3c([2H])c([2H])c([2H])c([2H])c23)cc1 ; [2H]c1cc(-c2c3ccccc3c(-c3cccc4c(-c5ccc6ccccc6c5)cccc34)c3c([2H])c([2H])c([2H])c([2H])c23)c([2H])c([2H])c1[2H] ; [2H]c1cc([2H])c(-c2c3ccccc3c(-c3cccc4c(-c5ccc6ccccc6c5)cccc34)c3c([2H])c([2H])c([2H])c([2H])c23)c([2H])c1[2H]]

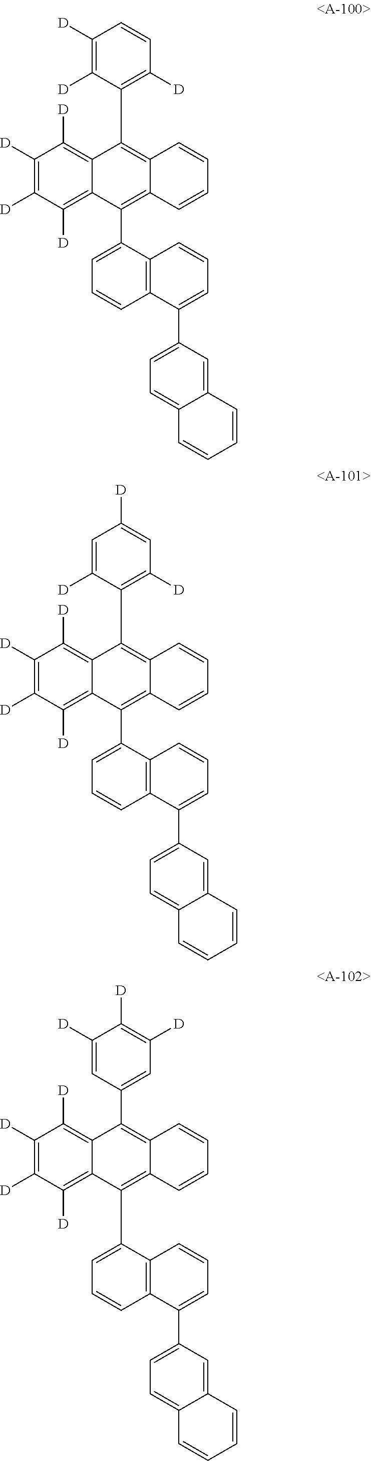 [SMILES: [2H]c1ccc([2H])c(-c2c3ccccc3c(-c3cccc4c(-c5ccc6ccccc6c5)cccc34)c3c([2H])c([2H])c([2H])c([2H])c23)c1[2H] ; [2H]c1cc([2H])c(-c2c3ccccc3c(-c3cccc4c(-c5ccc6ccccc6c5)cccc34)c3c([2H])c([2H])c([2H])c([2H])c23)c([2H])c1 ; [2H]c1cc(-c2c3ccccc3c(-c3cccc4c(-c5ccc6ccccc6c5)cccc34)c3c([2H])c([2H])c([2H])c([2H])c23)cc([2H])c1[2H]]

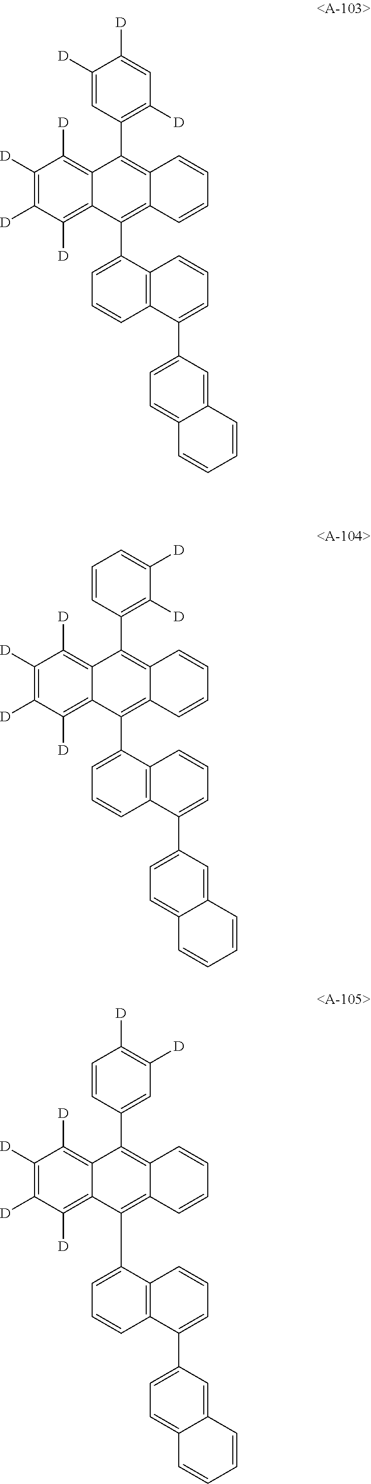 [SMILES: [2H]c1cc([2H])c(-c2c3ccccc3c(-c3cccc4c(-c5ccc6ccccc6c5)cccc34)c3c([2H])c([2H])c([2H])c([2H])c23)cc1[2H] ; [2H]c1cccc(-c2c3ccccc3c(-c3cccc4c(-c5ccc6ccccc6c5)cccc34)c3c([2H])c([2H])c([2H])c([2H])c23)c1[2H] ; [2H]c1ccc(-c2c3ccccc3c(-c3cccc4c(-c5ccc6ccccc6c5)cccc34)c3c([2H])c([2H])c([2H])c([2H])c23)cc1[2H]]

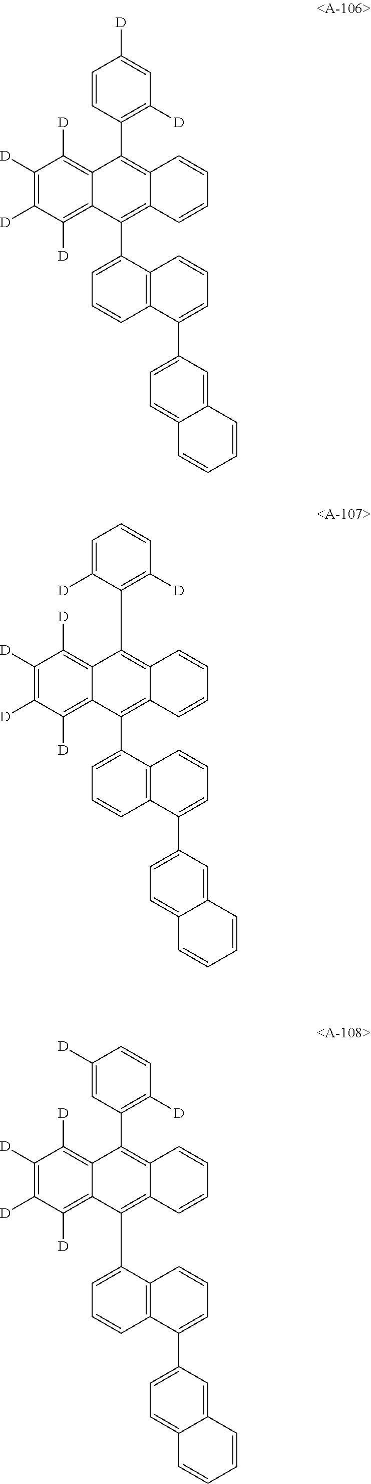 [SMILES: [2H]c1ccc(-c2c3ccccc3c(-c3cccc4c(-c5ccc6ccccc6c5)cccc34)c3c([2H])c([2H])c([2H])c([2H])c23)c([2H])c1 ; [2H]c1cccc([2H])c1-c1c2ccccc2c(-c2cccc3c(-c4ccc5ccccc5c4)cccc23)c2c([2H])c([2H])c([2H])c([2H])c12 ; [2H]c1ccc([2H])c(-c2c3ccccc3c(-c3cccc4c(-c5ccc6ccccc6c5)cccc34)c3c([2H])c([2H])c([2H])c([2H])c23)c1]

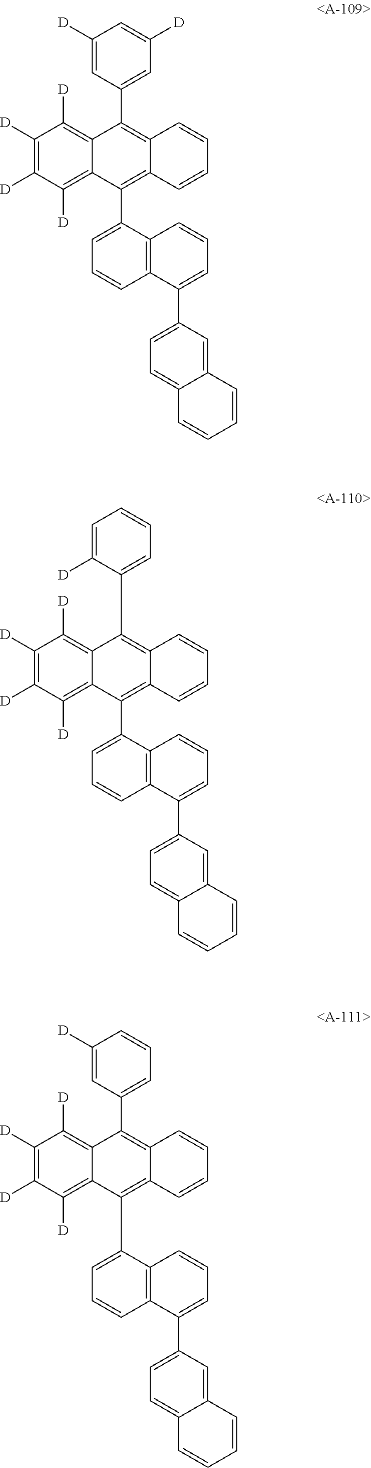 [SMILES: [2H]c1cc([2H])cc(-c2c3ccccc3c(-c3cccc4c(-c5ccc6ccccc6c5)cccc34)c3c([2H])c([2H])c([2H])c([2H])c23)c1 ; [2H]c1ccccc1-c1c2ccccc2c(-c2cccc3c(-c4ccc5ccccc5c4)cccc23)c2c([2H])c([2H])c([2H])c([2H])c12 ; [2H]c1cccc(-c2c3ccccc3c(-c3cccc4c(-c5ccc6ccccc6c5)cccc34)c3c([2H])c([2H])c([2H])c([2H])c23)c1]

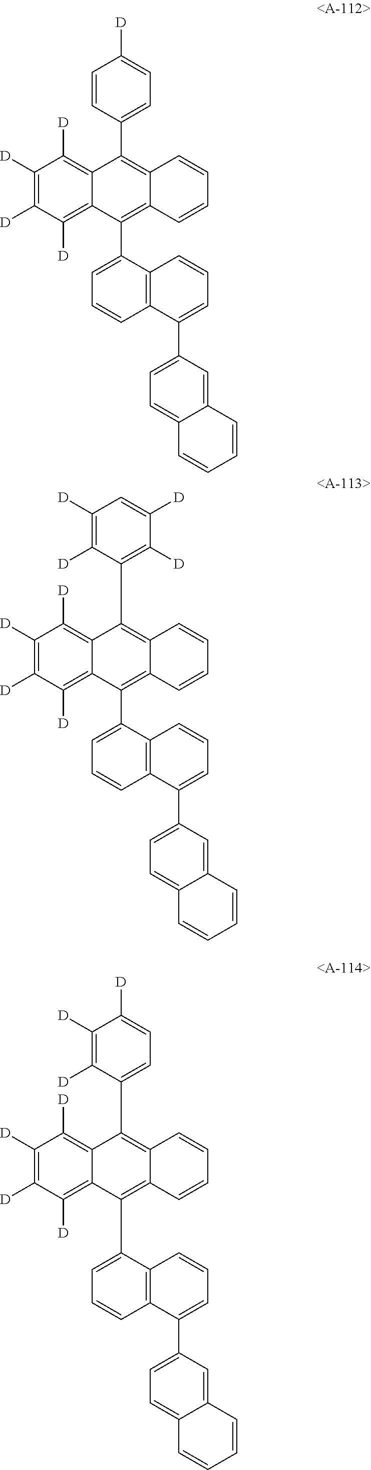

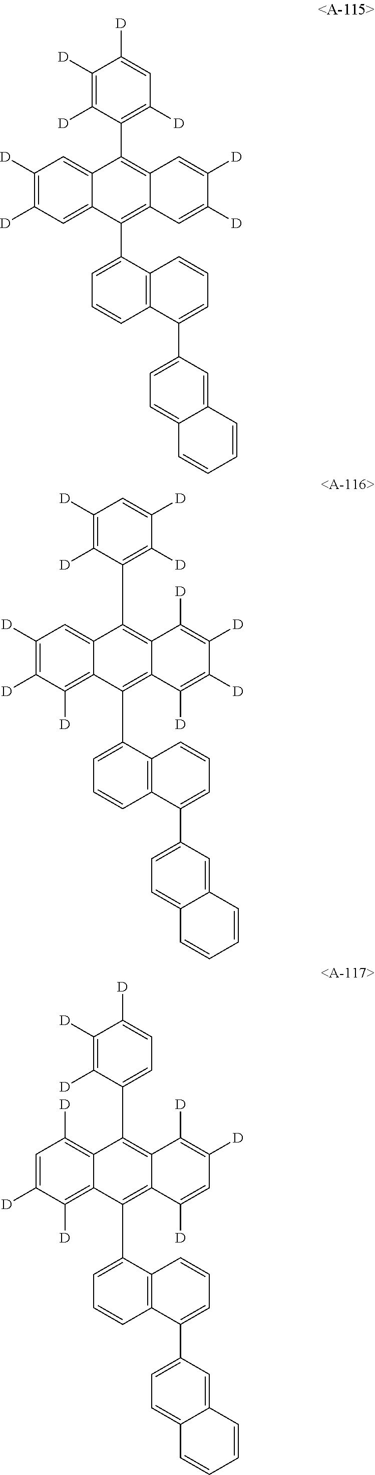

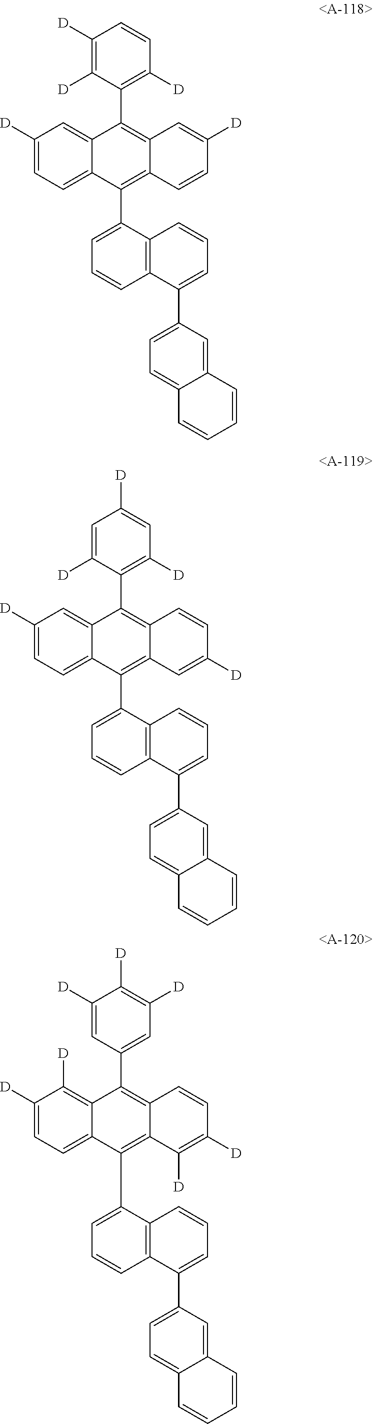 [SMILES: [2H]c1ccc2c(-c3cccc4c(-c5ccc6ccccc6c5)cccc34)c3ccc([2H])cc3c(-c3c([2H])ccc([2H])c3[2H])c2c1 ; [2H]c1cc([2H])c(-c2c3cc([2H])ccc3c(-c3cccc4c(-c5ccc6ccccc6c5)cccc34)c3cc([2H])ccc23)c([2H])c1 ; [2H]c1cc(-c2c3ccc([2H])c([2H])c3c(-c3cccc4c(-c5ccc6ccccc6c5)cccc34)c3ccc([2H])c([2H])c23)cc([2H])c1[2H]]

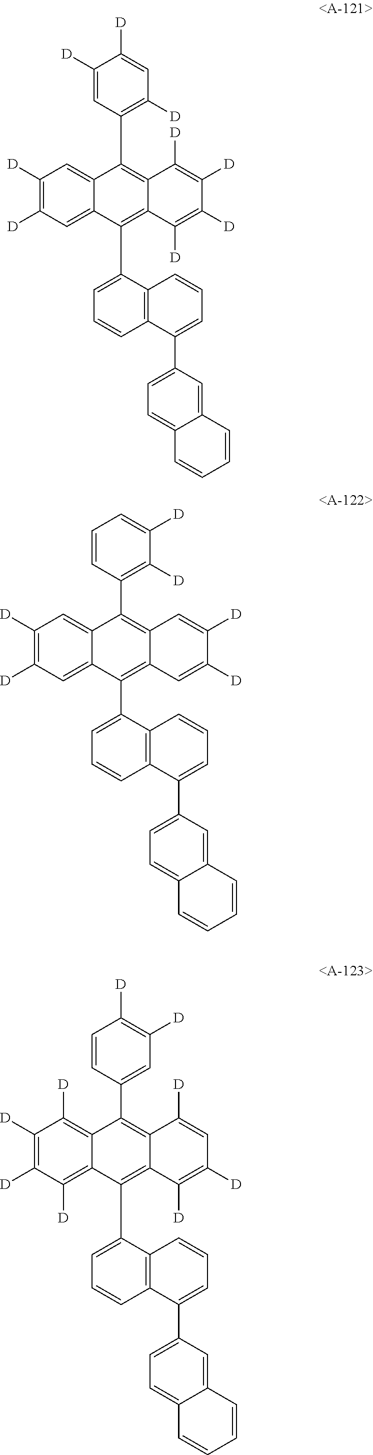

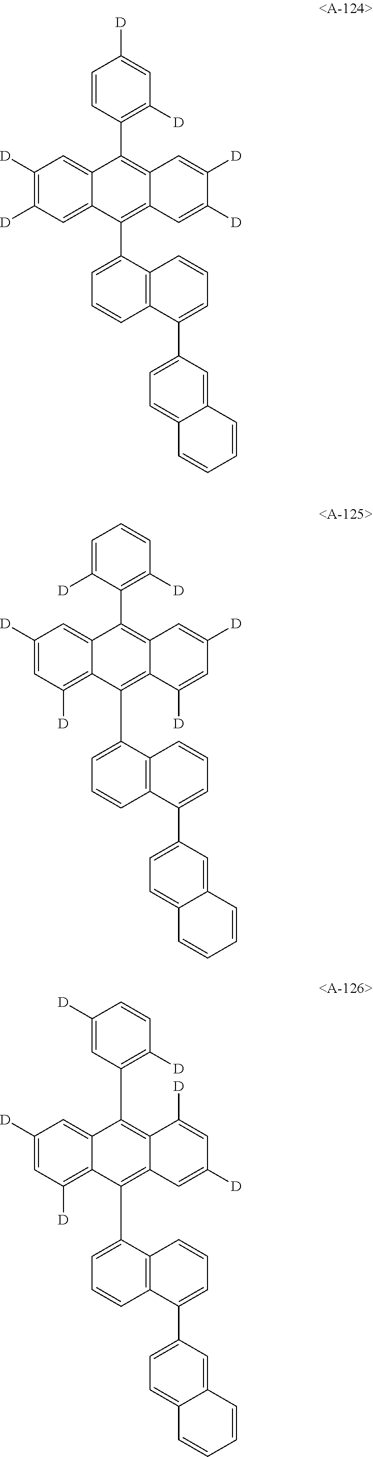 [SMILES: [2H]c1ccc(-c2c3cc([2H])c([2H])cc3c(-c3cccc4c(-c5ccc6ccccc6c5)cccc34)c3cc([2H])c([2H])cc23)c([2H])c1 ; [2H]c1cc([2H])c2c(-c3cccc4c(-c5ccc6ccccc6c5)cccc34)c3c([2H])cc([2H])cc3c(-c3c([2H])cccc3[2H])c2c1 ; [2H]c1ccc([2H])c(-c2c3cc([2H])cc([2H])c3c(-c3cccc4c(-c5ccc6ccccc6c5)cccc34)c3cc([2H])cc([2H])c23)c1]

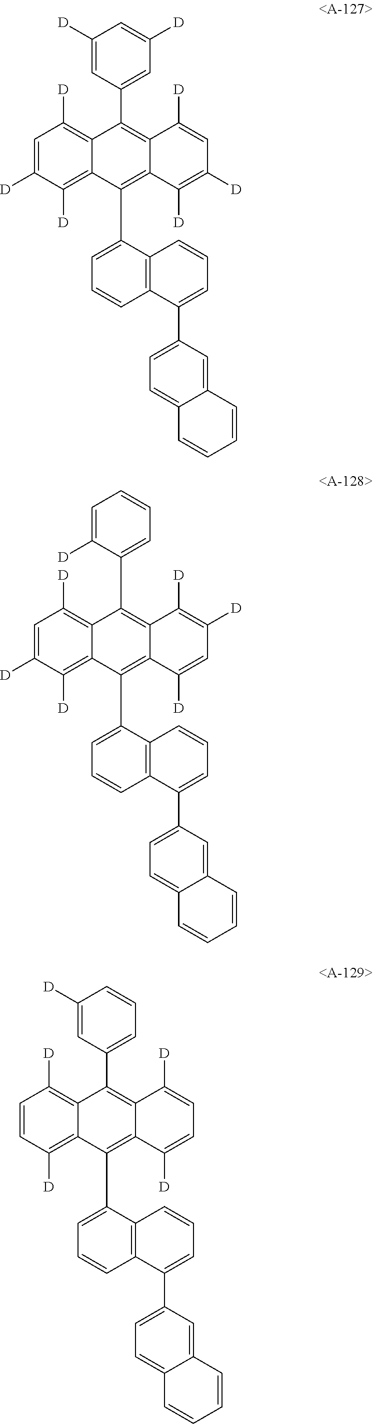 [SMILES: [2H]c1cc([2H])cc(-c2c3c([2H])cc([2H])c([2H])c3c(-c3cccc4c(-c5ccc6ccccc6c5)cccc34)c3c([2H])c([2H])cc([2H])c23)c1 ; [2H]c1ccccc1-c1c2c([2H])cc([2H])c([2H])c2c(-c2cccc3c(-c4ccc5ccccc5c4)cccc23)c2c([2H])cc([2H])c([2H])c12 ; [2H]c1cccc(-c2c3c([2H])ccc([2H])c3c(-c3cccc4c(-c5ccc6ccccc6c5)cccc34)c3c([2H])ccc([2H])c23)c1]

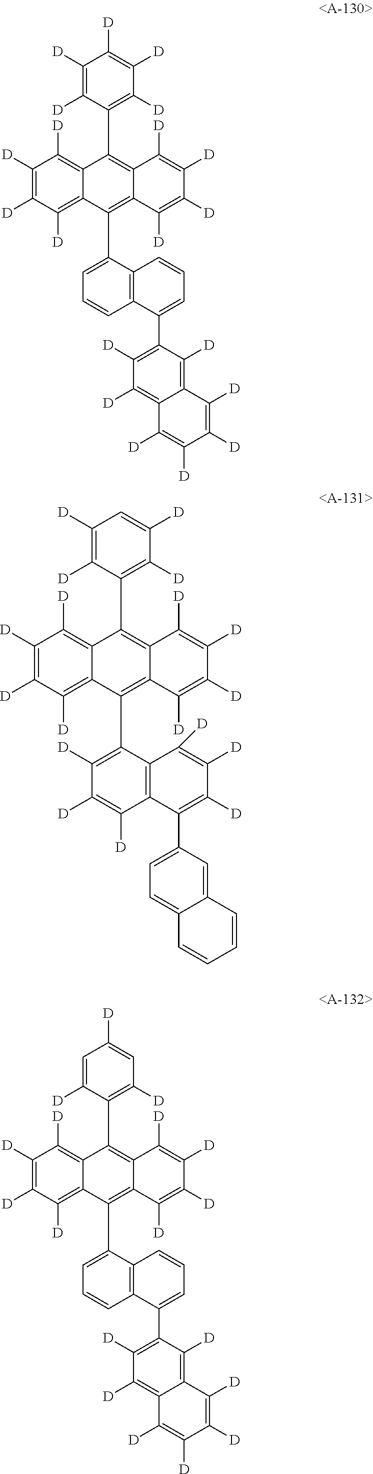

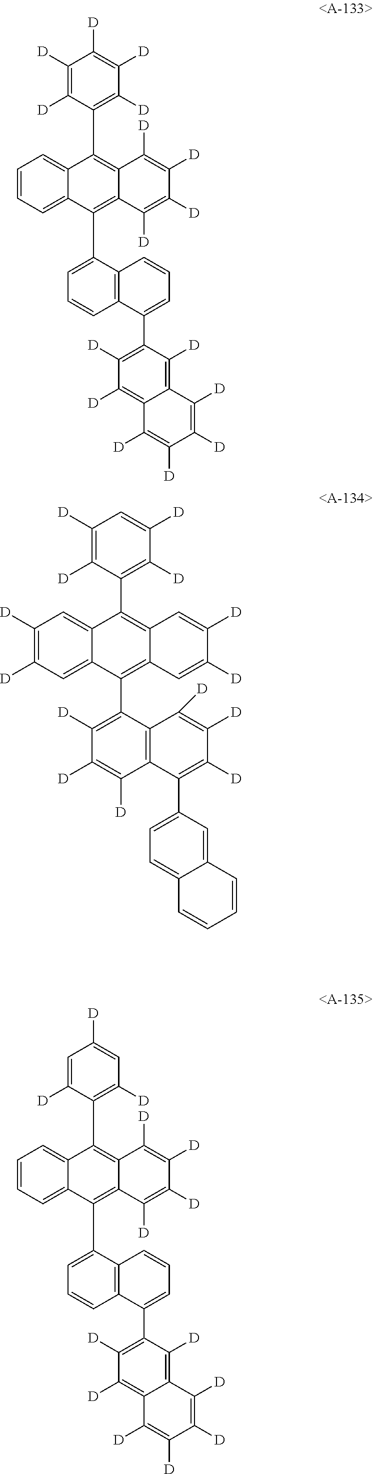

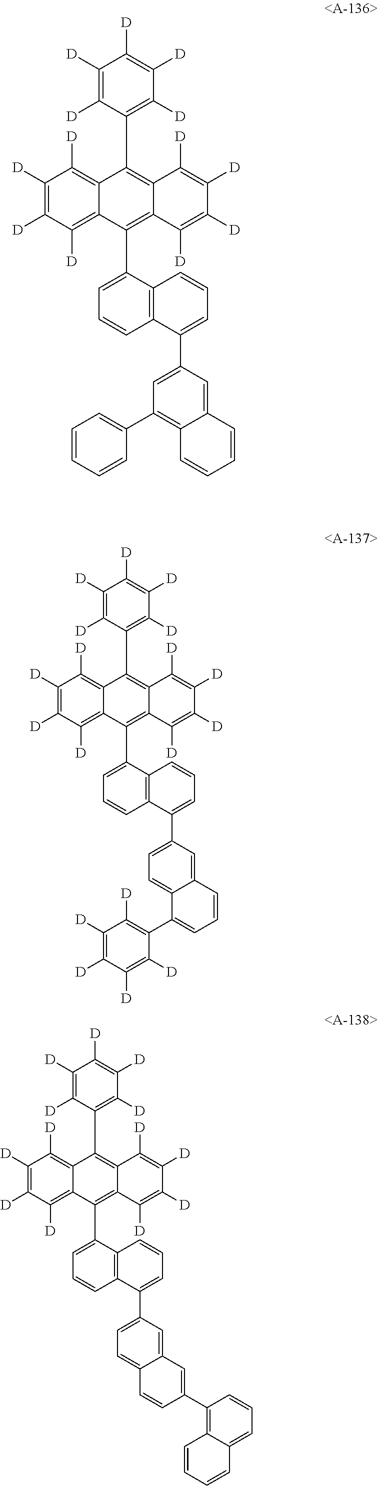 [SMILES: [2H]c1c([2H])c([2H])c(-c2c3c([2H])c([2H])c([2H])c([2H])c3c(-c3cccc4c(-c5cc(-c6ccccc6)c6ccccc6c5)cccc34)c3c([2H])c([2H])c([2H])c([2H])c23)c([2H])c1[2H] ; [2H]c1c([2H])c([2H])c(-c2cccc3cc(-c4cccc5c(-c6c7c([2H])c([2H])c([2H])c([2H])c7c(-c7c([2H])c([2H])c([2H])c([2H])c7[2H])c7c([2H])c([2H])c([2H])c([2H])c67)cccc45)ccc23)c([2H])c1[2H] ; [2H]c1c([2H])c([2H])c(-c2c3c([2H])c([2H])c([2H])c([2H])c3c(-c3cccc4c(-c5ccc6ccc(-c7cccc8ccccc78)cc6c5)cccc34)c3c([2H])c([2H])c([2H])c([2H])c23)c([2H])c1[2H]]

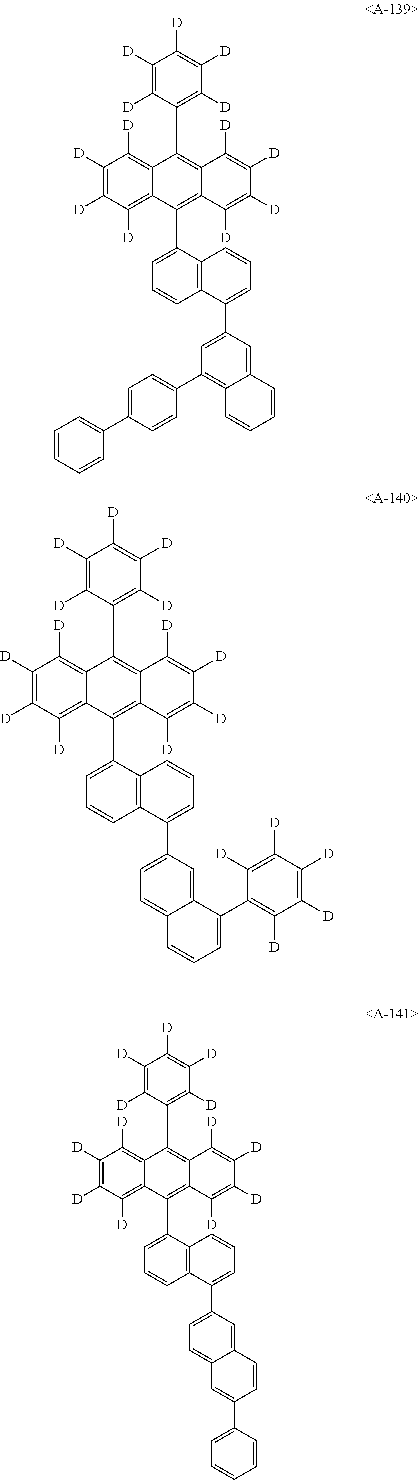 [SMILES: [2H]c1c([2H])c([2H])c(-c2c3c([2H])c([2H])c([2H])c([2H])c3c(-c3cccc4c(-c5cc(-c6ccc(-c7ccccc7)cc6)c6ccccc6c5)cccc34)c3c([2H])c([2H])c([2H])c([2H])c23)c([2H])c1[2H] ; [2H]c1c([2H])c([2H])c(-c2cccc3ccc(-c4cccc5c(-c6c7c([2H])c([2H])c([2H])c([2H])c7c(-c7c([2H])c([2H])c([2H])c([2H])c7[2H])c7c([2H])c([2H])c([2H])c([2H])c67)cccc45)cc23)c([2H])c1[2H] ; [2H]c1c([2H])c([2H])c(-c2c3c([2H])c([2H])c([2H])c([2H])c3c(-c3cccc4c(-c5ccc6cc(-c7ccccc7)ccc6c5)cccc34)c3c([2H])c([2H])c([2H])c([2H])c23)c([2H])c1[2H]]

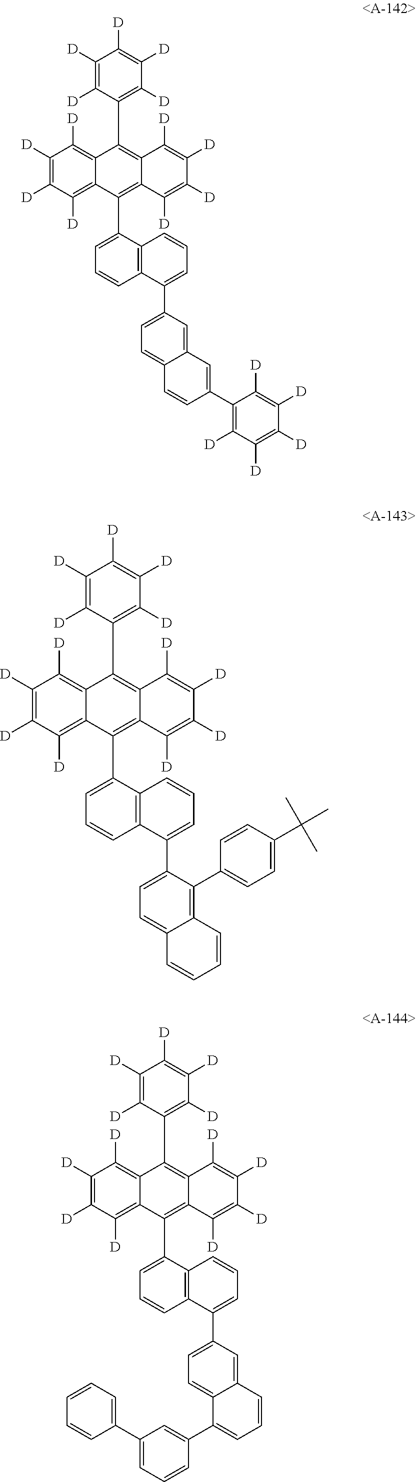

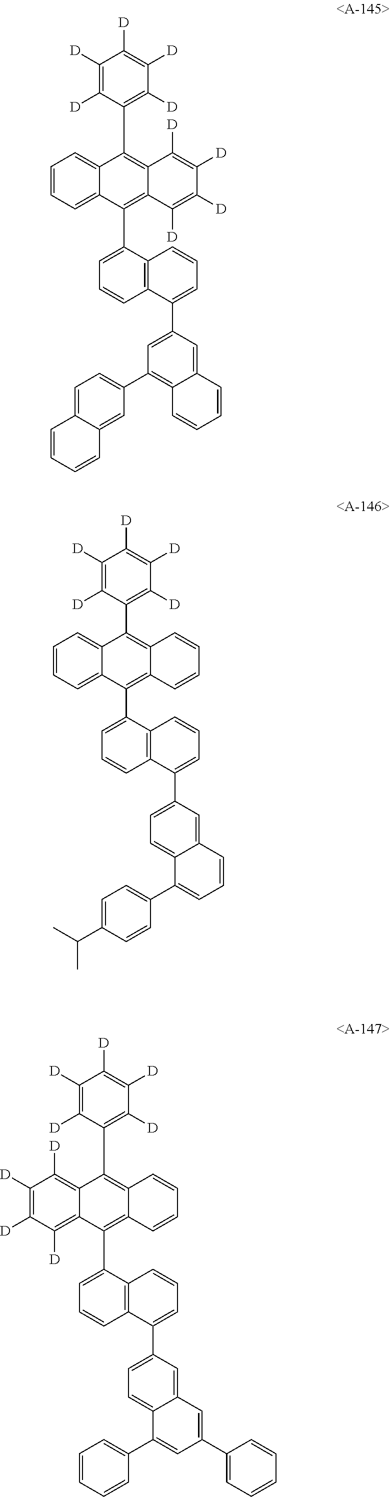 [SMILES: [2H]c1c([2H])c([2H])c(-c2c3ccccc3c(-c3cccc4c(-c5cc(-c6ccc7ccccc7c6)c6ccccc6c5)cccc34)c3c([2H])c([2H])c([2H])c([2H])c23)c([2H])c1[2H] ; [2H]c1c([2H])c([2H])c(-c2c3ccccc3c(-c3cccc4c(-c5ccc6c(-c7ccc(C(C)C)cc7)cccc6c5)cccc34)c3ccccc23)c([2H])c1[2H] ; [2H]c1c([2H])c([2H])c(-c2c3ccccc3c(-c3cccc4c(-c5ccc6c(-c7ccccc7)cc(-c7ccccc7)cc6c5)cccc34)c3c([2H])c([2H])c([2H])c([2H])c23)c([2H])c1[2H]]

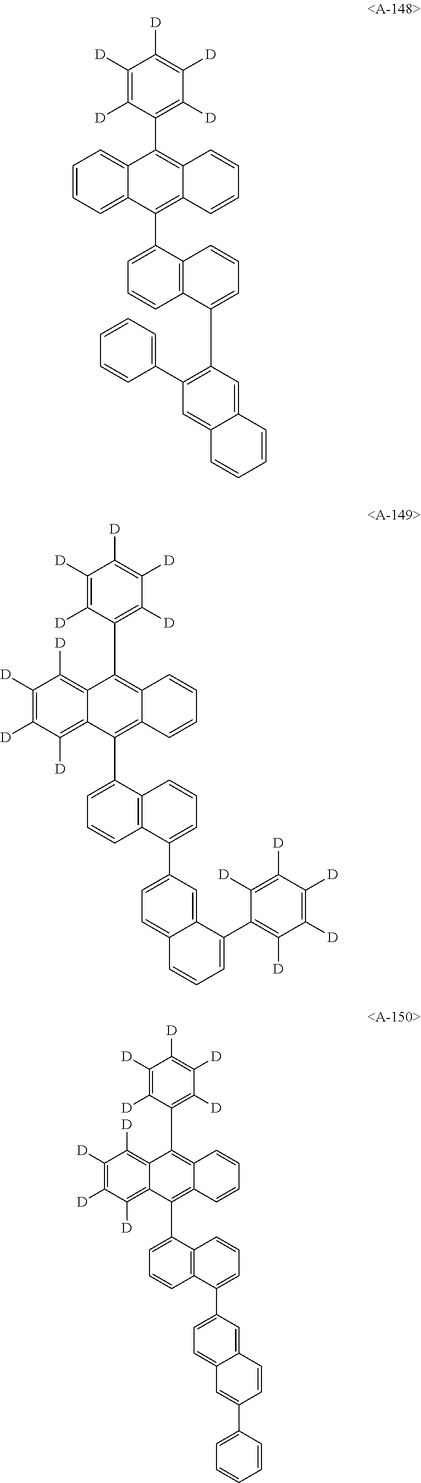 [SMILES: [2H]c1c([2H])c([2H])c(-c2c3ccccc3c(-c3cccc4c(-c5cc6ccccc6cc5-c5ccccc5)cccc34)c3ccccc23)c([2H])c1[2H] ; [2H]c1c([2H])c([2H])c(-c2cccc3ccc(-c4cccc5c(-c6c7ccccc7c(-c7c([2H])c([2H])c([2H])c([2H])c7[2H])c7c([2H])c([2H])c([2H])c([2H])c67)cccc45)cc23)c([2H])c1[2H] ; [2H]c1c([2H])c([2H])c(-c2c3ccccc3c(-c3cccc4c(-c5ccc6cc(-c7ccccc7)ccc6c5)cccc34)c3c([2H])c([2H])c([2H])c([2H])c23)c([2H])c1[2H]]

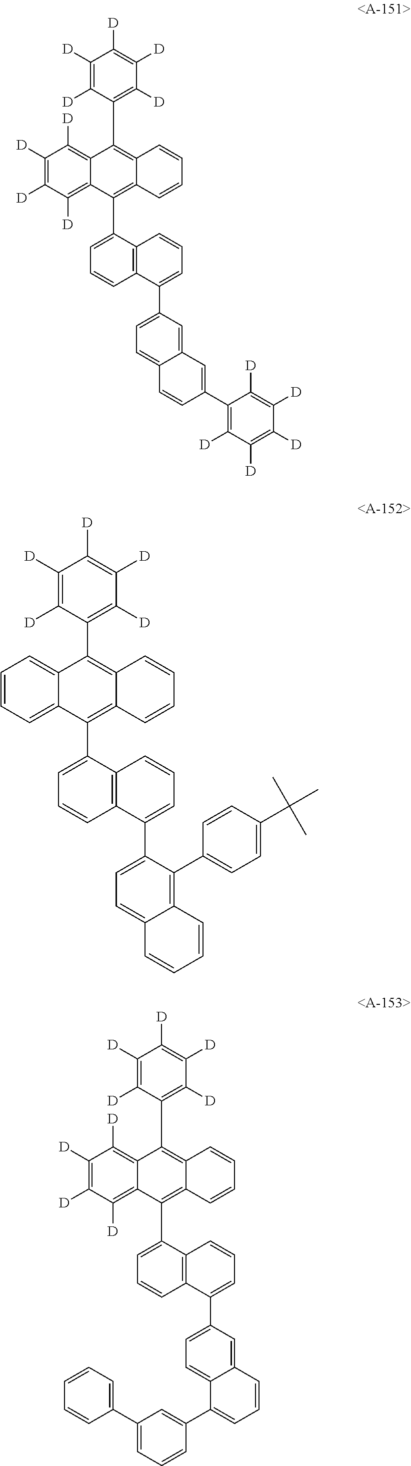 [SMILES: [2H]c1c([2H])c([2H])c(-c2ccc3ccc(-c4cccc5c(-c6c7ccccc7c(-c7c([2H])c([2H])c([2H])c([2H])c7[2H])c7c([2H])c([2H])c([2H])c([2H])c67)cccc45)cc3c2)c([2H])c1[2H] ; [2H]c1c([2H])c([2H])c(-c2c3ccccc3c(-c3cccc4c(-c5ccc6ccccc6c5-c5ccc(C(C)(C)C)cc5)cccc34)c3ccccc23)c([2H])c1[2H] ; [2H]c1c([2H])c([2H])c(-c2c3ccccc3c(-c3cccc4c(-c5ccc6c(-c7cccc(-c8ccccc8)c7)cccc6c5)cccc34)c3c([2H])c([2H])c([2H])c([2H])c23)c([2H])c1[2H]]

8. The organic light-emitting diode of claim 1, wherein T4 and T5 in Chemical Formulas D6 and D7 is same or different and are each independently a substituted or unsubstituted aromatic hydrocarbon ring of 6 to 30 carbon atoms.

9. The organic light-emitting diode of claim 1, wherein at least one of Y₅ and Y₆ in Chemical Formulas D6 and D7 is NR₆₆ or NR₇₁, wherein R₆₆ and R₇₁ are as defined in claim 1.

10. The organic light-emitting diode of claim 9, wherein R₆₆ and R₇₁ in Chemical Formulas D6 and D7 are same or different and are each independently a substituted or unsubstituted aryl of 6 to 30 carbon atoms.

11. The organic light-emitting diode of claim 1, wherein, in Chemical Formulas D6 and D7, Y₅ is NR₆₆ and Y₆ is NR₇₁, wherein NR₆₆ and NR₇₁ are as defined in claim 1.

12. The organic light-emitting diode of claim 11, wherein R₆₆ and R₇₁ in Chemical Formulas D6 and D7 are same or different and are each independently a substituted or unsubstituted aryl of 6 to 30 carbon atoms.

13. The organic light-emitting diode of claim 1, wherein the aromatic hydrocarbon ring of 6 to 50 carbon atoms or the heteroaromatic ring of 2 to 40 carbon atoms of at least one of the T4 to T6 ring moieties may be bonded to an aryl amino radical represented by the following Structural Formula F: 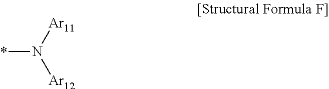
wherein,
“-*” denotes a bonding site participating in forming a bond to a carbon aromatic ring member of any one of T4 to T6, and
Ar₁₁ and Ar₁₂, which are same or different, are each independently a substituted or unsubstituted aryl of 6 to 18 carbon atoms, and are linked to each other to form a ring.

14. The organic light-emitting diode of claim 1, wherein Y₄ in Chemical Formulas D6 and D7 is S.

15. The organic light-emitting diode of claim 1, wherein the compounds represented by Chemical Formula D6 or D7 are each any one selected from <D201> to <D350>:

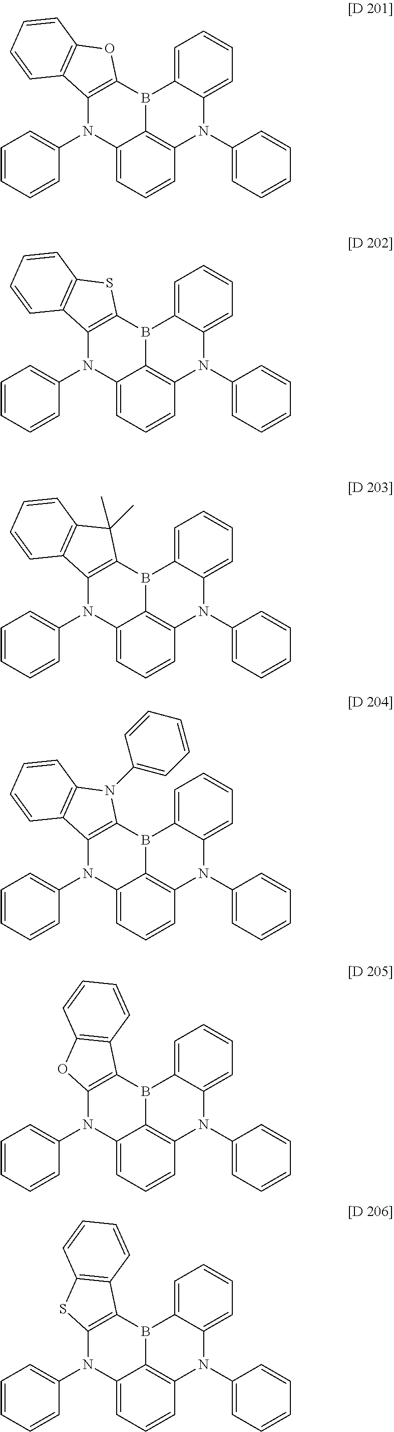

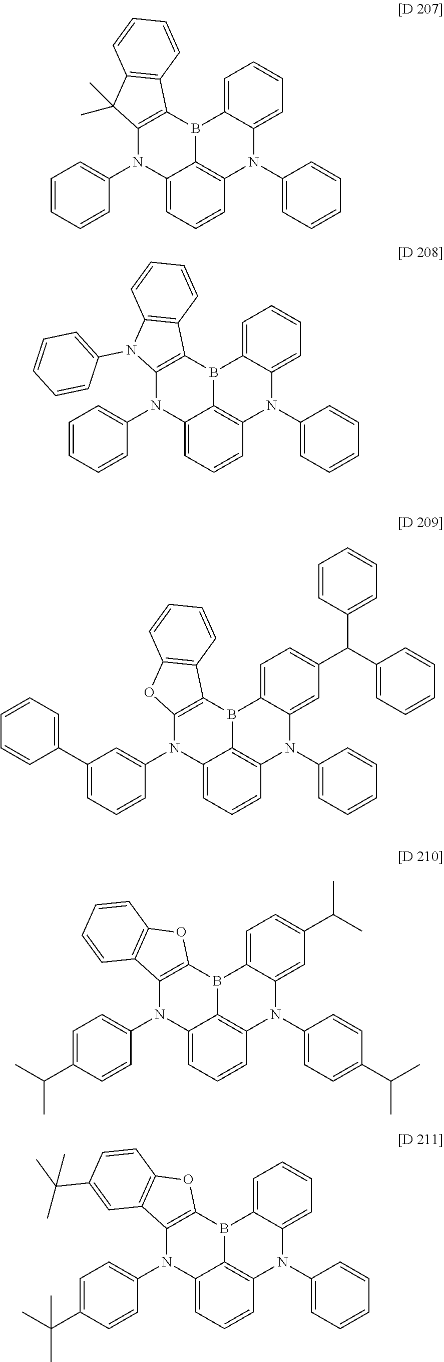

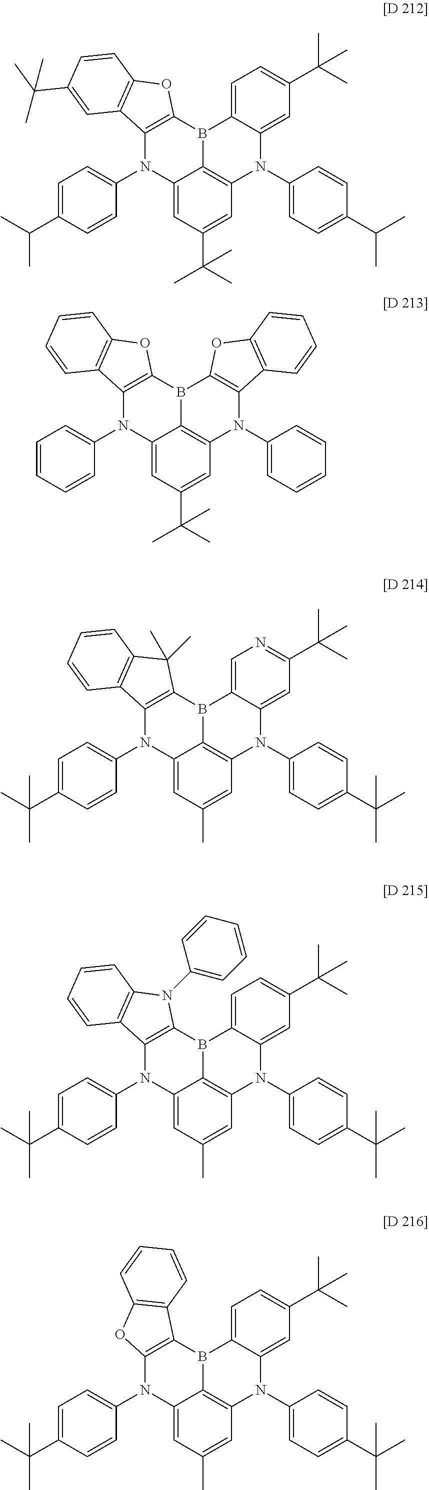

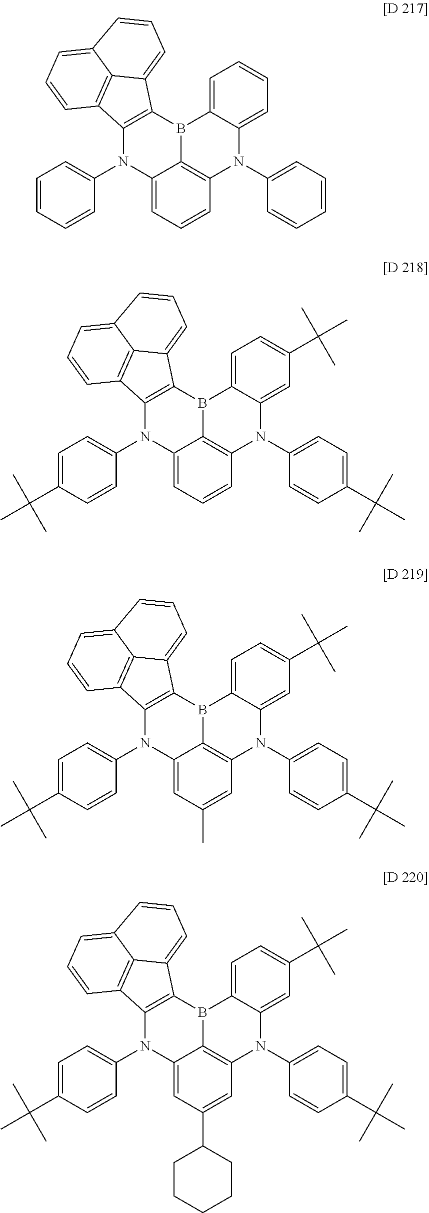

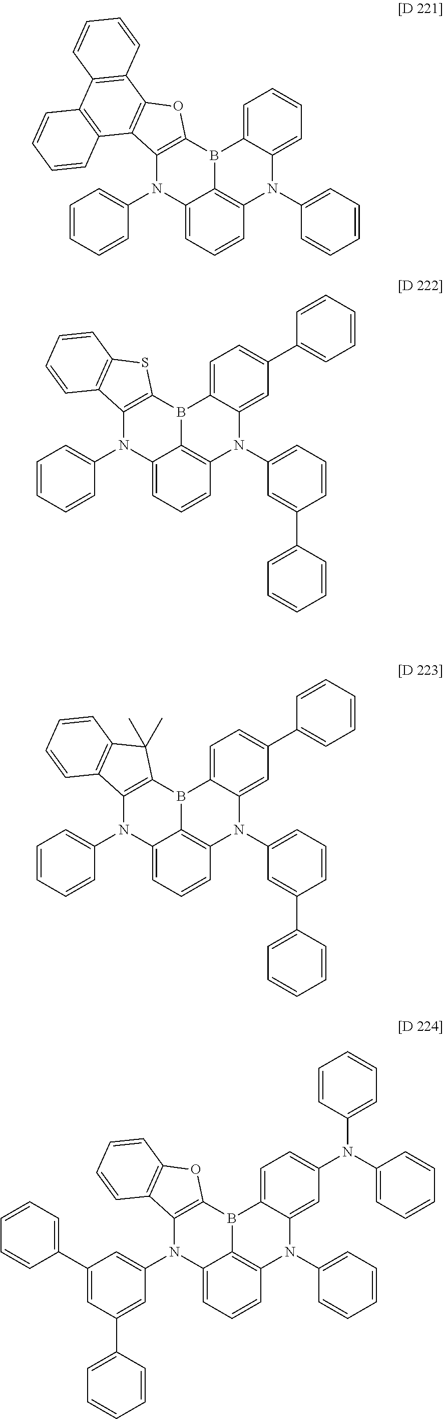

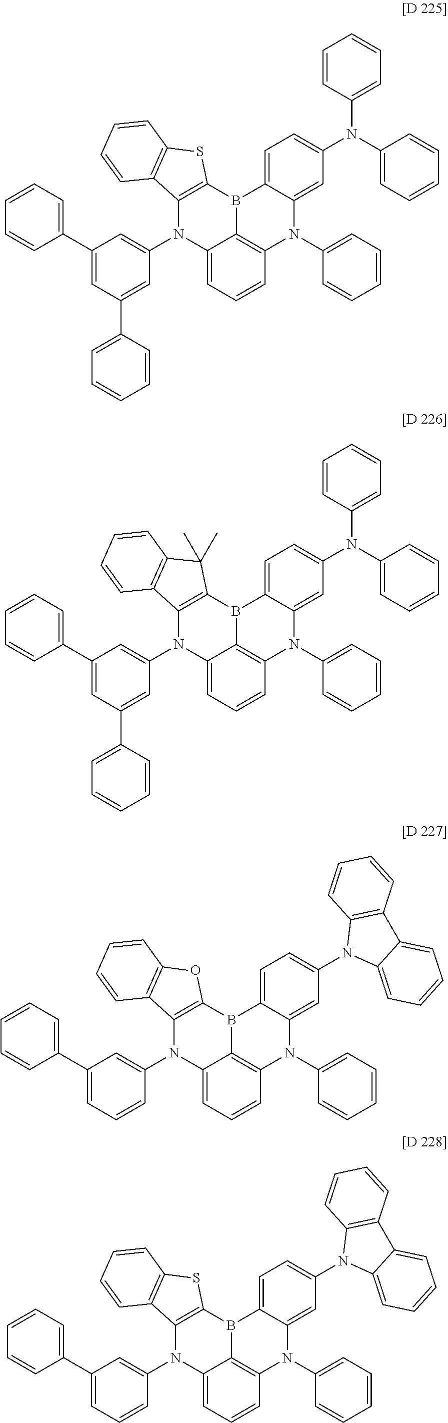 [SMILES: c1ccc(-c2cc(-c3ccccc3)cc(N3c4cccc5c4B(c4ccc(N(c6ccccc6)c6ccccc6)cc4N5c4ccccc4)c4sc5ccccc5c43)c2)cc1 ; CC1(C)C2=C(c3ccccc31)N(c1cc(-c3ccccc3)cc(-c3ccccc3)c1)c1cccc3c1B2c1ccc(N(c2ccccc2)c2ccccc2)cc1N3c1ccccc1 ; c1ccc(-c2cccc(N3c4cccc5c4B(c4ccc(-n6c7ccccc7c7ccccc76)cc4N5c4ccccc4)c4oc5ccccc5c43)c2)cc1 ; c1ccc(-c2cccc(N3c4cccc5c4B(c4ccc(-n6c7ccccc7c7ccccc76)cc4N5c4ccccc4)c4sc5ccccc5c43)c2)cc1]

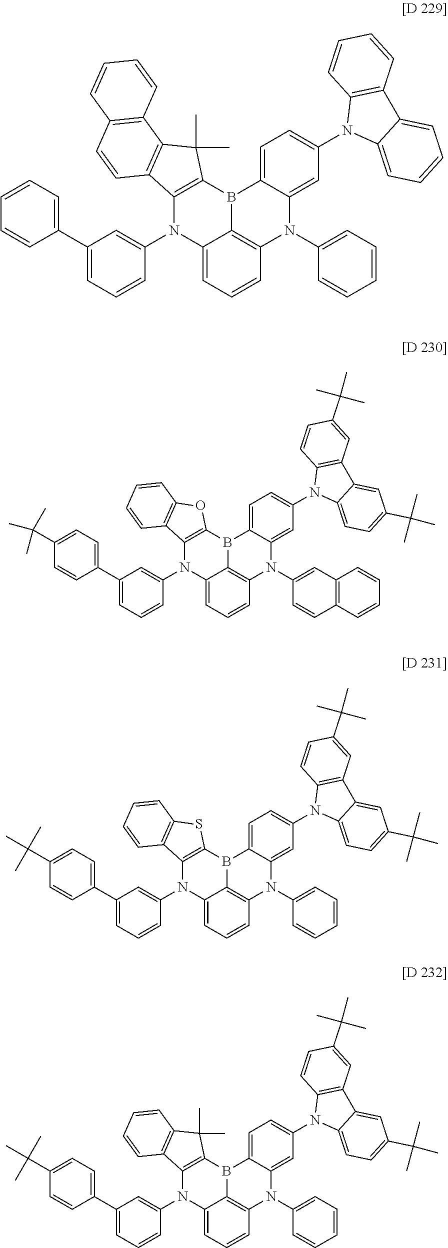

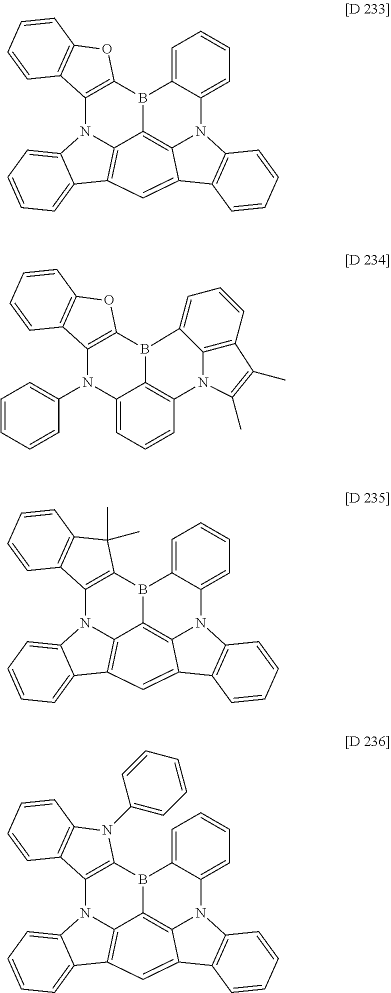 [SMILES: c1ccc2c(c1)B1c3oc4ccccc4c3-n3c4ccccc4c4cc5c6ccccc6n-2c5c1c43 ; Cc1c(C)n2c3c(cccc13)B1c3oc4ccccc4c3N(c3ccccc3)c3cccc-2c31 ; CC1(C)C2=C(c3ccccc31)n1c3ccccc3c3cc4c5ccccc5n5c4c(c31)B2c1ccccc1-5 ; c1ccc(-n2c3c(c4ccccc42)-n2c4ccccc4c4cc5c6ccccc6n6c5c(c42)B3c2ccccc2-6)cc1]

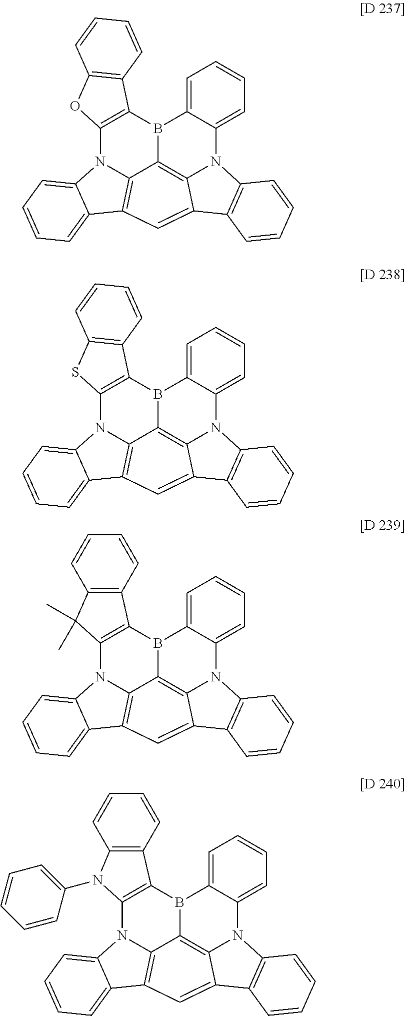

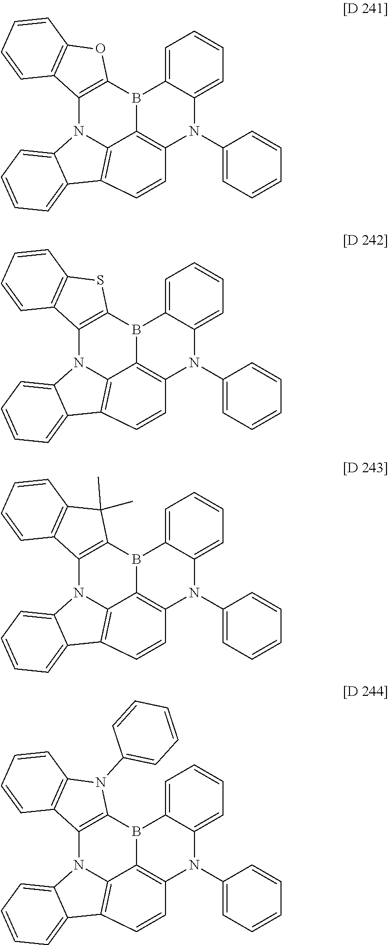

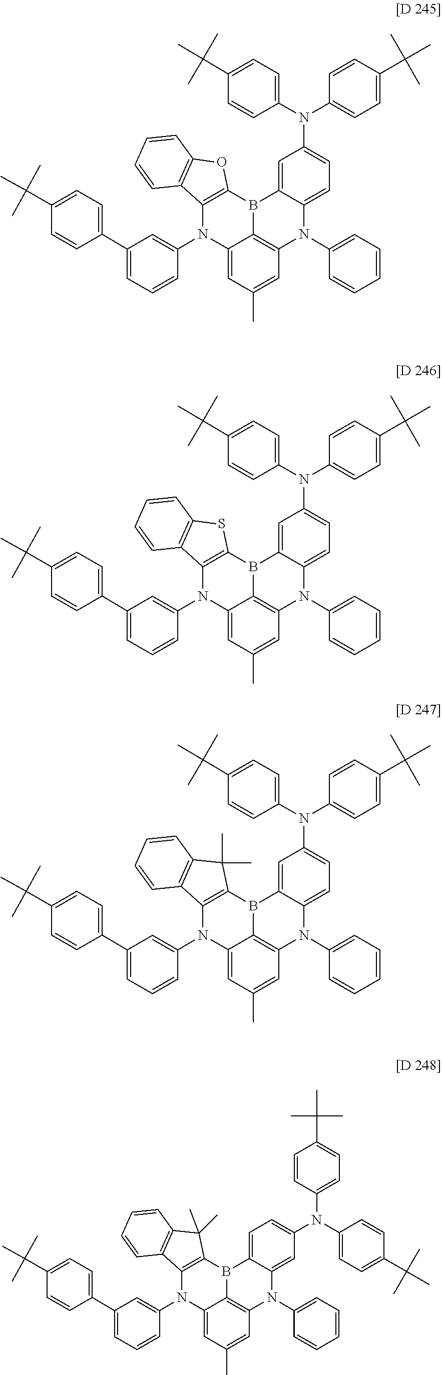

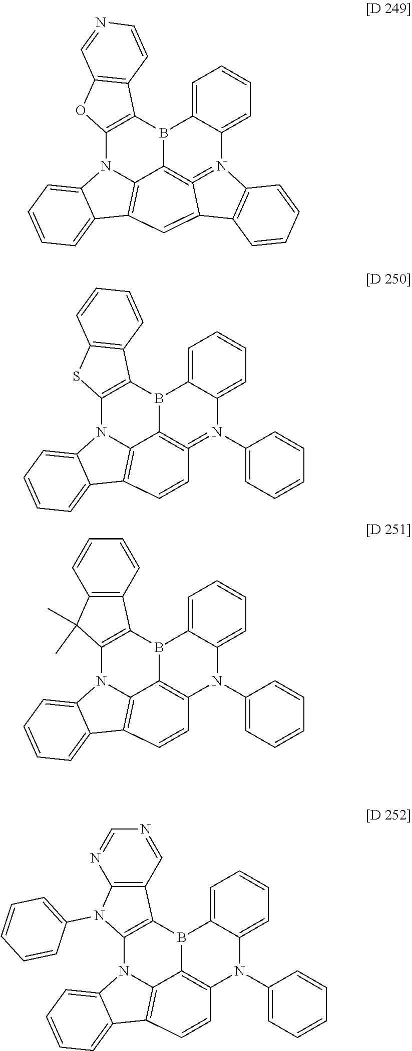

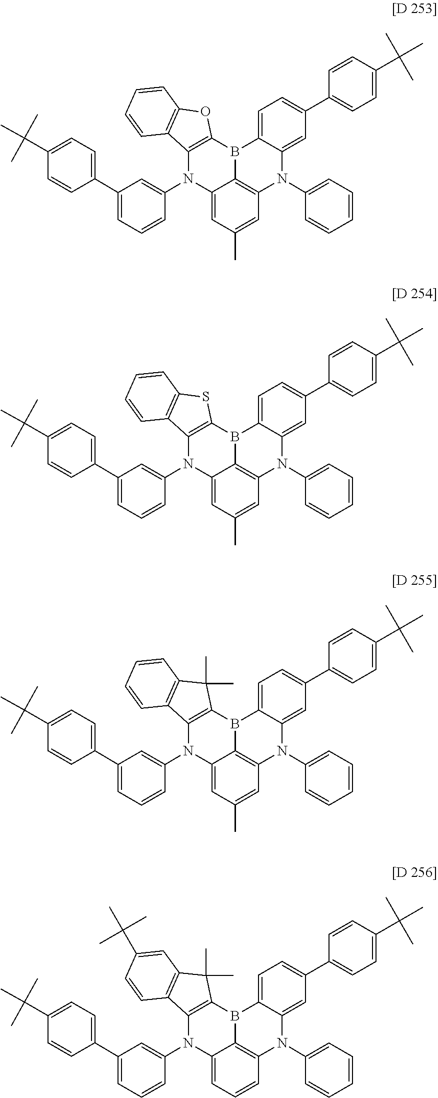

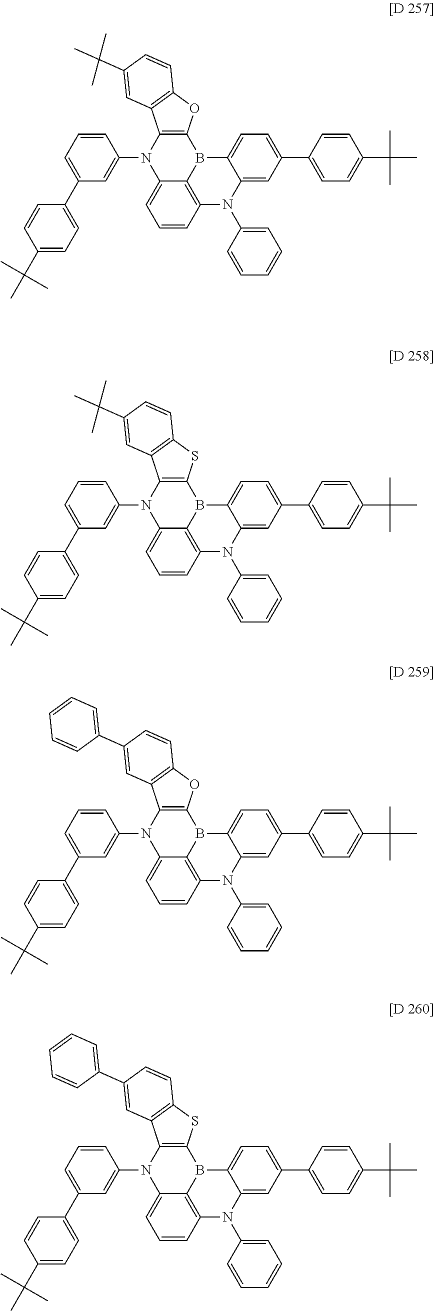

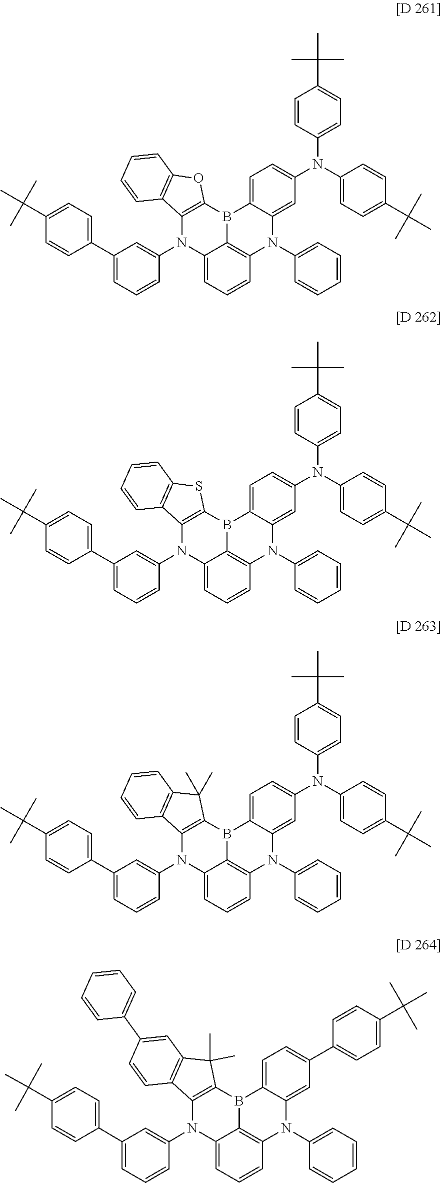

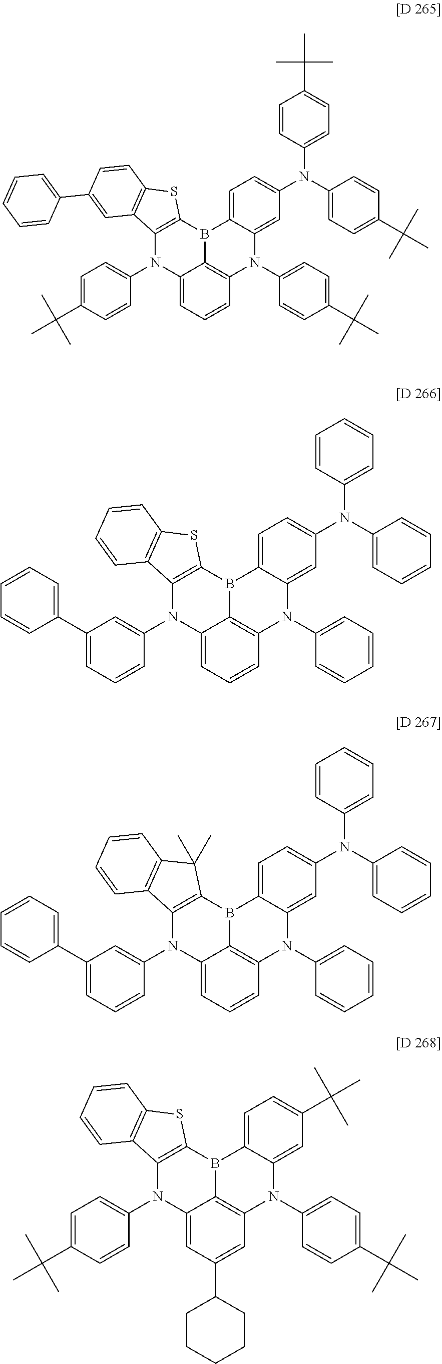

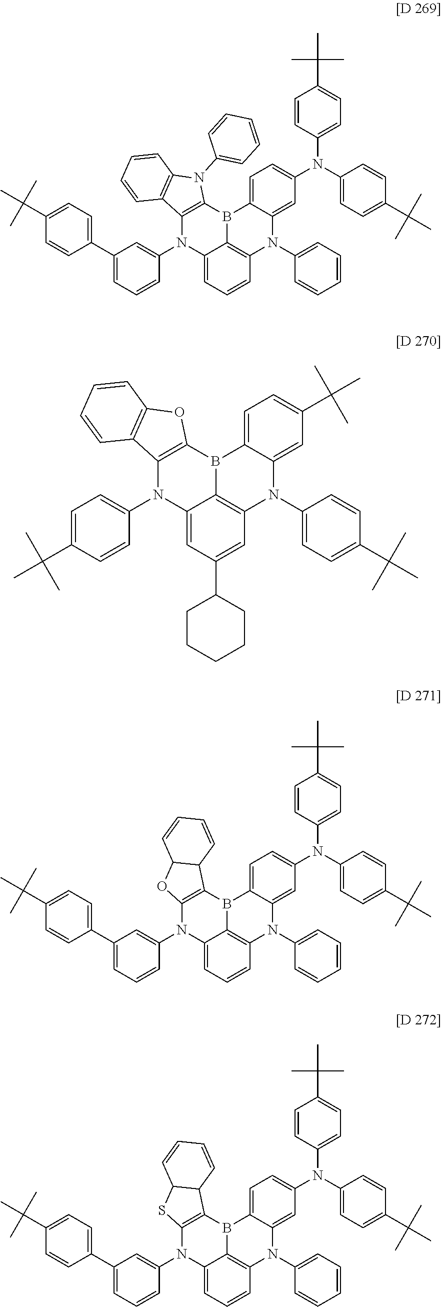

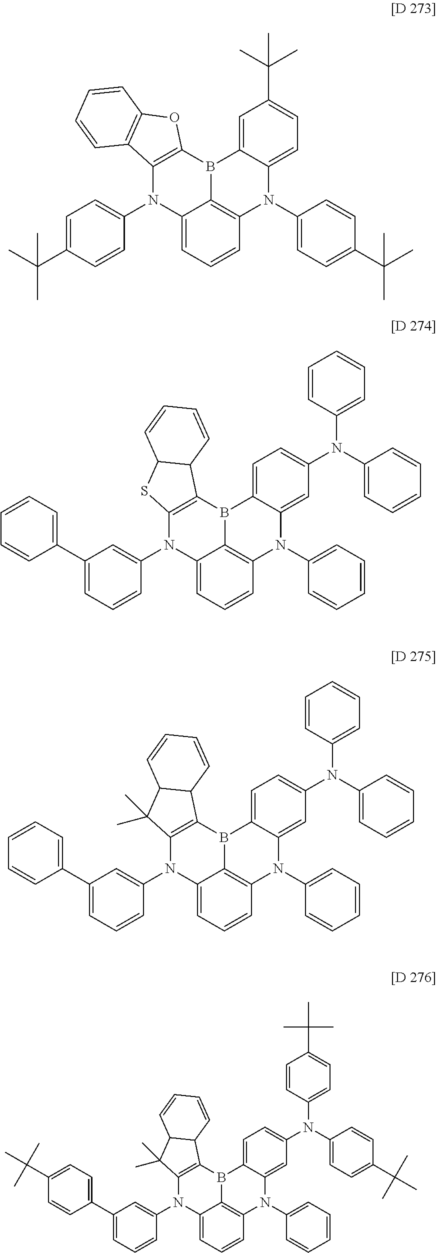

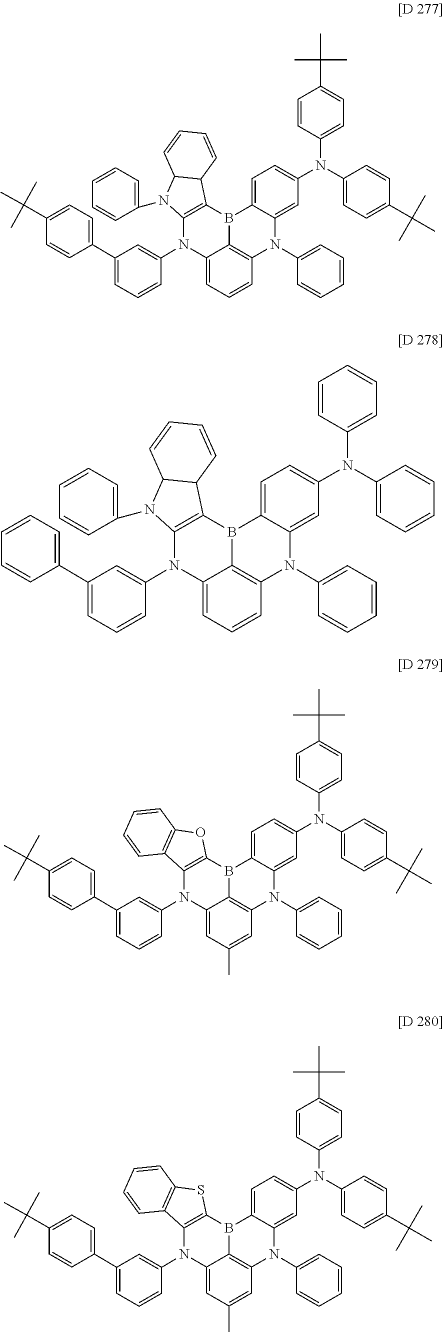

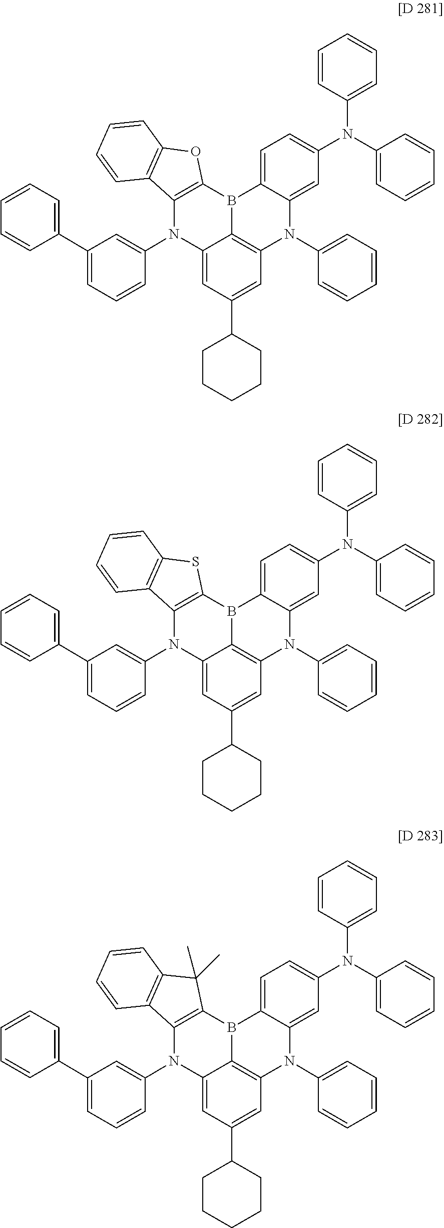 [SMILES: c1ccc(-c2cccc(N3c4cc(C5CCCCC5)cc5c4B(c4ccc(N(c6ccccc6)c6ccccc6)cc4N5c4ccccc4)c4oc5ccccc5c43)c2)cc1 ; c1ccc(-c2cccc(N3c4cc(C5CCCCC5)cc5c4B(c4ccc(N(c6ccccc6)c6ccccc6)cc4N5c4ccccc4)c4sc5ccccc5c43)c2)cc1 ; CC1(C)C2=C(c3ccccc31)N(c1cccc(-c3ccccc3)c1)c1cc(C3CCCCC3)cc3c1B2c1ccc(N(c2ccccc2)c2ccccc2)cc1N3c1ccccc1]

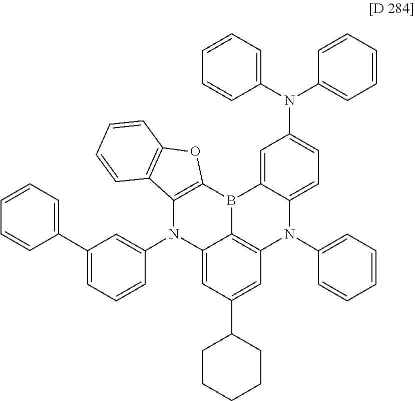 [SMILES: c1ccc(-c2cccc(N3c4cc(C5CCCCC5)cc5c4B(c4cc(N(c6ccccc6)c6ccccc6)ccc4N5c4ccccc4)c4oc5ccccc5c43)c2)cc1]

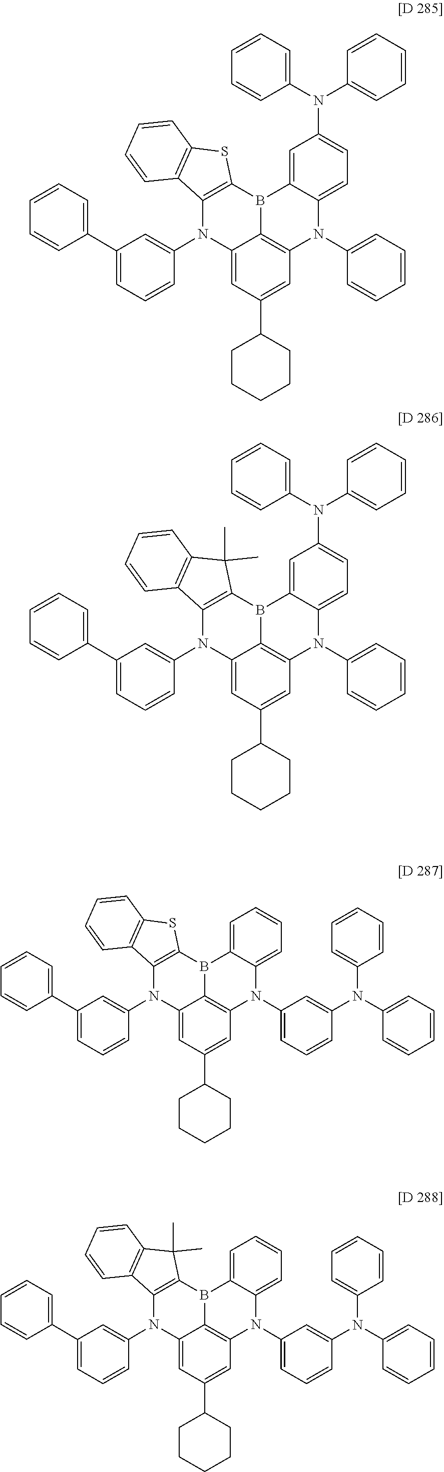

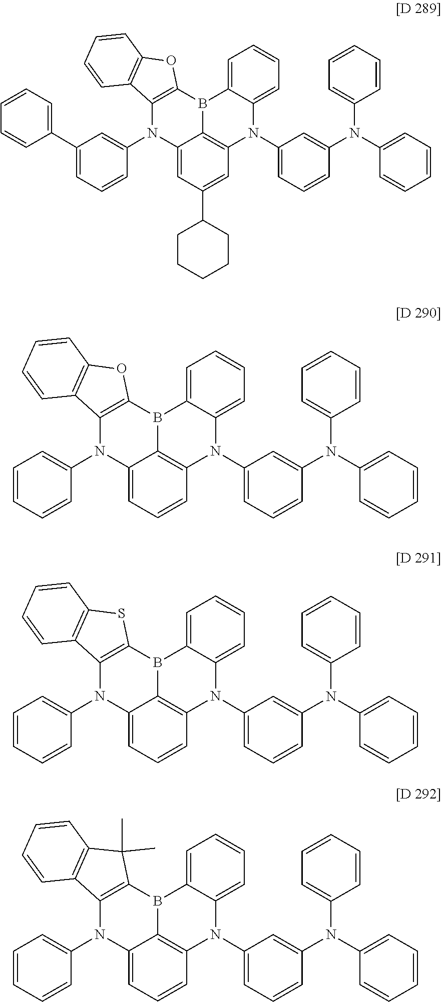 [SMILES: c1ccc(-c2cccc(N3c4cc(C5CCCCC5)cc5c4B(c4ccccc4N5c4cccc(N(c5ccccc5)c5ccccc5)c4)c4oc5ccccc5c43)c2)cc1 ; c1ccc(N(c2ccccc2)c2cccc(N3c4ccccc4B4c5oc6ccccc6c5N(c5ccccc5)c5cccc3c54)c2)cc1 ; c1ccc(N(c2ccccc2)c2cccc(N3c4ccccc4B4c5sc6ccccc6c5N(c5ccccc5)c5cccc3c54)c2)cc1 ; CC1(C)C2=C(c3ccccc31)N(c1ccccc1)c1cccc3c1B2c1ccccc1N3c1cccc(N(c2ccccc2)c2ccccc2)c1]

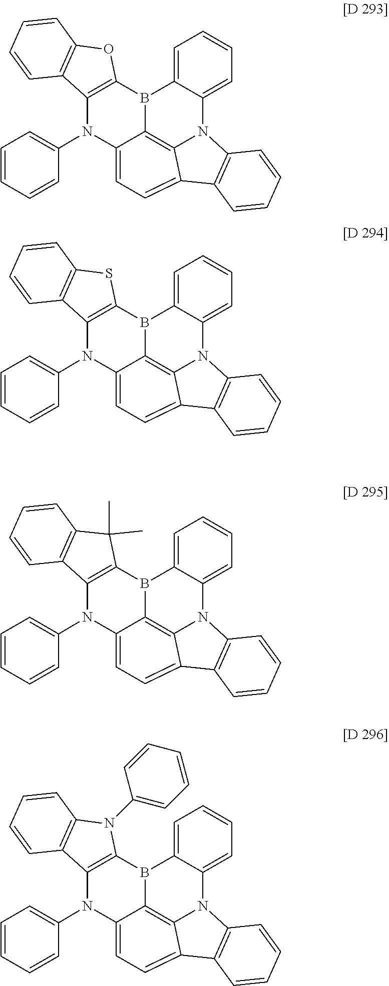 [SMILES: c1ccc(N2c3ccc4c5ccccc5n5c4c3B(c3ccccc3-5)c3oc4ccccc4c32)cc1 ; c1ccc(N2c3ccc4c5ccccc5n5c4c3B(c3ccccc3-5)c3sc4ccccc4c32)cc1 ; CC1(C)C2=C(c3ccccc31)N(c1ccccc1)c1ccc3c4ccccc4n4c3c1B2c1ccccc1-4 ; c1ccc(N2c3ccc4c5ccccc5n5c4c3B(c3ccccc3-5)c3c2c2ccccc2n3-c2ccccc2)cc1]

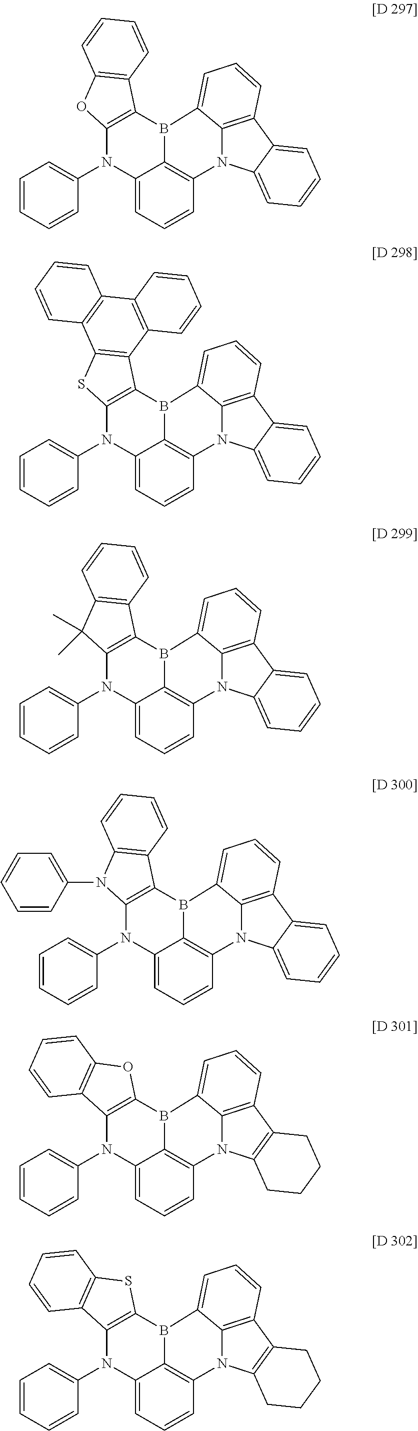

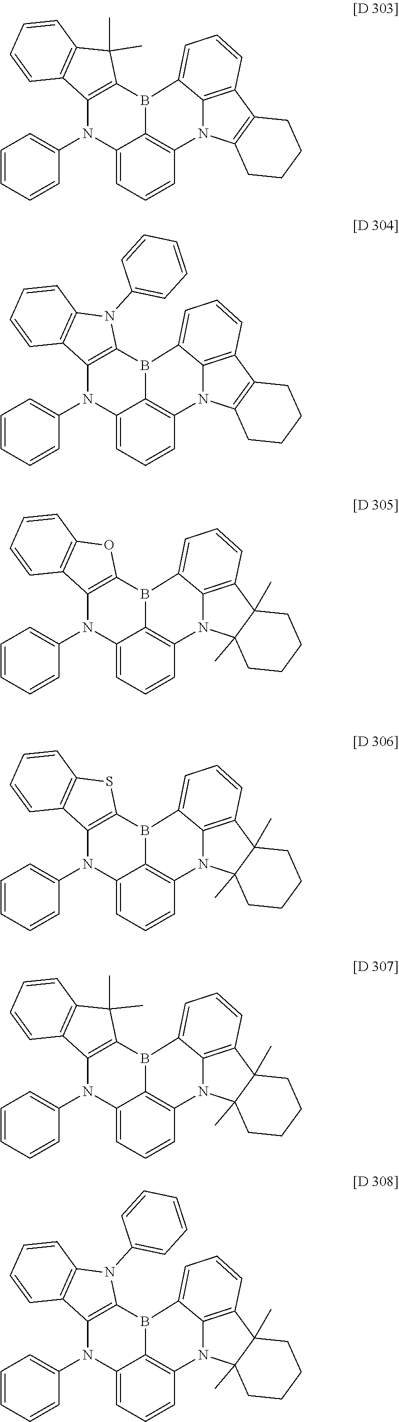

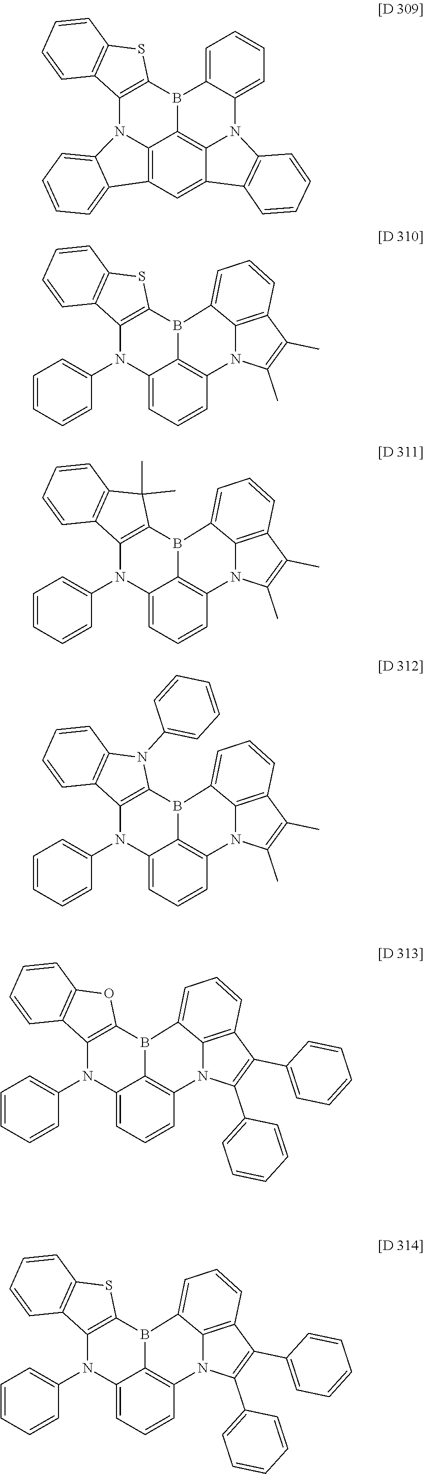

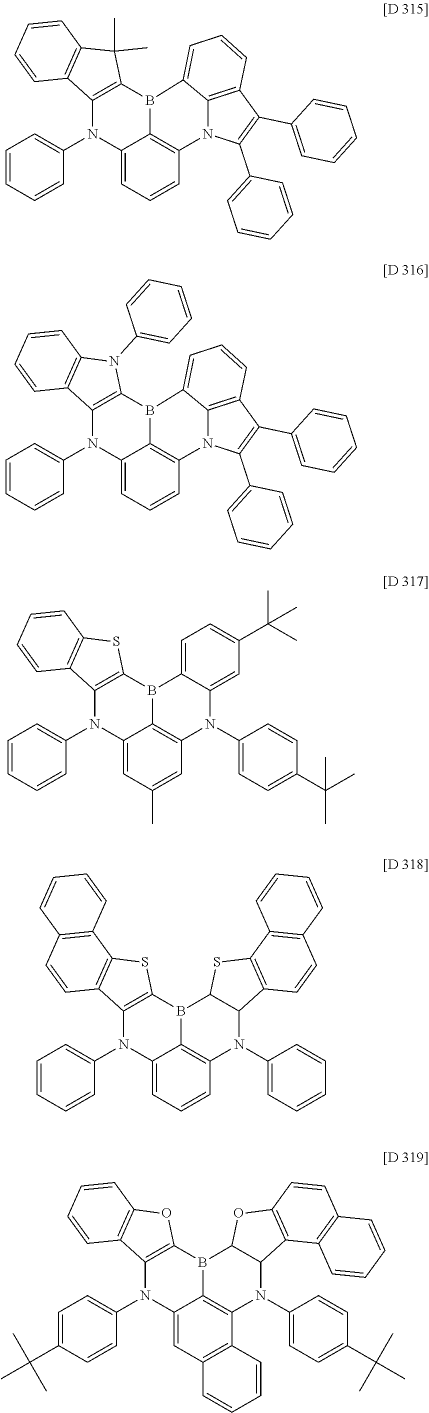

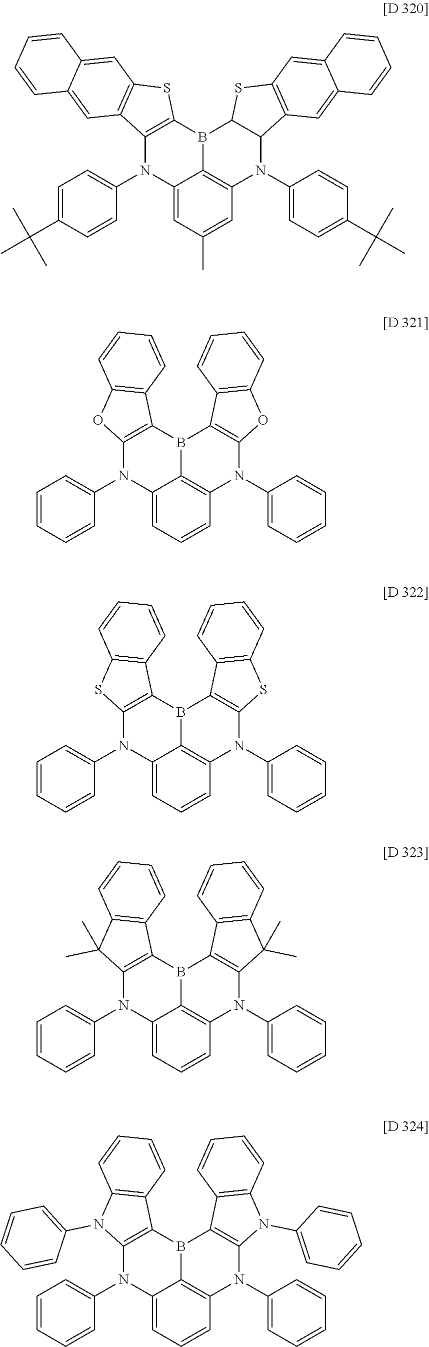 [SMILES: Cc1cc2c3c(c1)N(c1ccc(C(C)(C)C)cc1)C1c4cc5ccccc5cc4SC1B3c1sc3cc4ccccc4cc3c1N2c1ccc(C(C)(C)C)cc1 ; c1ccc(N2c3cccc4c3B(c3c2oc2ccccc32)c2c(oc3ccccc23)N4c2ccccc2)cc1 ; c1ccc(N2c3cccc4c3B(c3c2sc2ccccc32)c2c(sc3ccccc23)N4c2ccccc2)cc1 ; CC1(C)C2=C(B3C4=C(N(c5ccccc5)c5cccc(c53)N2c2ccccc2)C(C)(C)c2ccccc24)c2ccccc21 ; c1ccc(N2c3cccc4c3B(c3c2n(-c2ccccc2)c2ccccc32)c2c(n(-c3ccccc3)c3ccccc23)N4c2ccccc2)cc1]

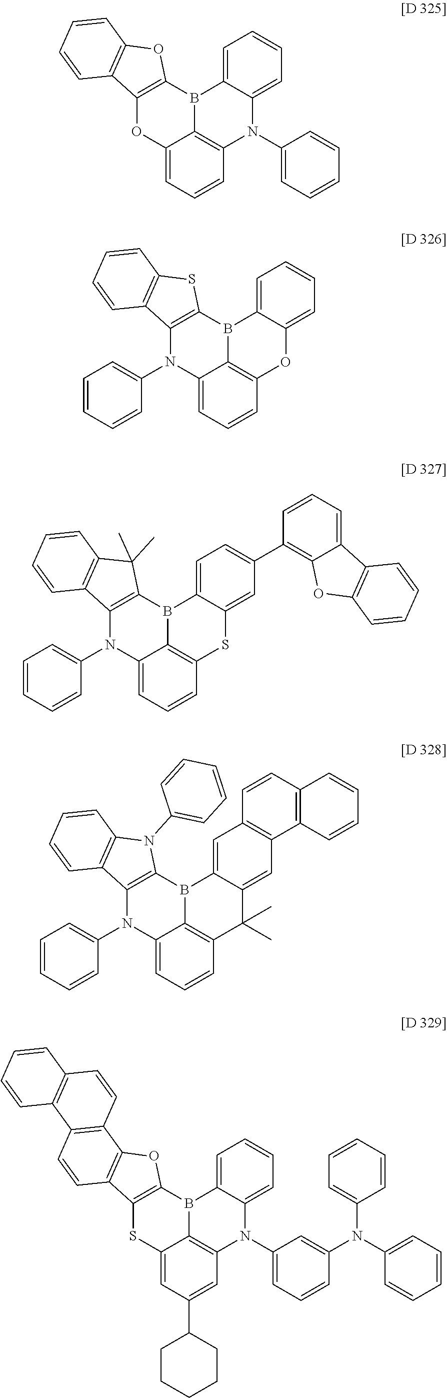 [SMILES: c1ccc(N2c3ccccc3B3c4oc5ccccc5c4Oc4cccc2c43)cc1 ; c1ccc(N2c3cccc4c3B(c3ccccc3O4)c3sc4ccccc4c32)cc1 ; CC1(C)C2=C(c3ccccc31)N(c1ccccc1)c1cccc3c1B2c1ccc(-c2cccc4c2oc2ccccc24)cc1S3 ; CC1(C)c2cc3c(ccc4ccccc43)cc2B2c3c(cccc31)N(c1ccccc1)c1c2n(-c2ccccc2)c2ccccc12 ; c1ccc(N(c2ccccc2)c2cccc(N3c4ccccc4B4c5oc6c(ccc7c8ccccc8ccc76)c5Sc5cc(C6CCCCC6)cc3c54)c2)cc1]

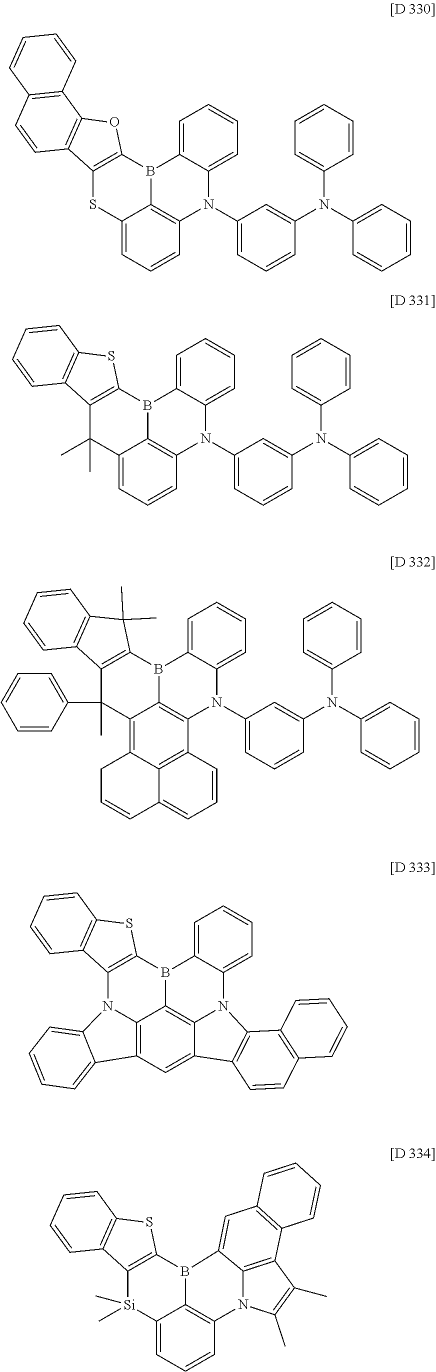 [SMILES: c1ccc(N(c2ccccc2)c2cccc(N3c4ccccc4B4c5oc6c(ccc7ccccc76)c5Sc5cccc3c54)c2)cc1 ; CC1(C)c2cccc3c2B(c2ccccc2N3c2cccc(N(c3ccccc3)c3ccccc3)c2)c2sc3ccccc3c21 ; CC1(C)C2=C(c3ccccc31)C(C)(c1ccccc1)c1c3c(c4cccc5c4c1CC=C5)N(c1cccc(N(c4ccccc4)c4ccccc4)c1)c1ccccc1B23 ; c1ccc2c(c1)B1c3sc4ccccc4c3-n3c4ccccc4c4cc5c6ccc7ccccc7c6n-2c5c1c43 ; Cc1c(C)n2c3c(cc4ccccc4c13)B1c3sc4ccccc4c3[Si](C)(C)c3cccc-2c31]

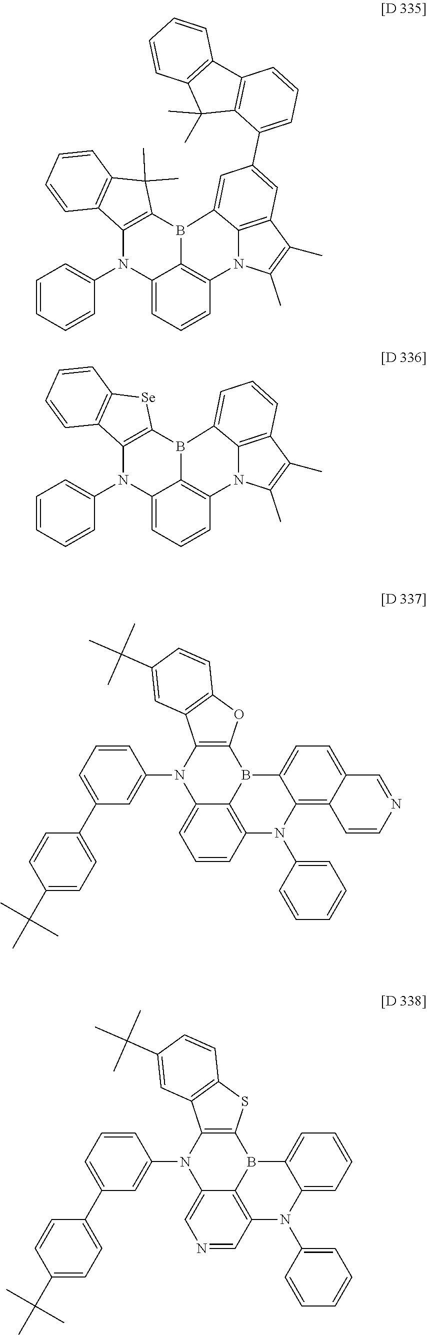

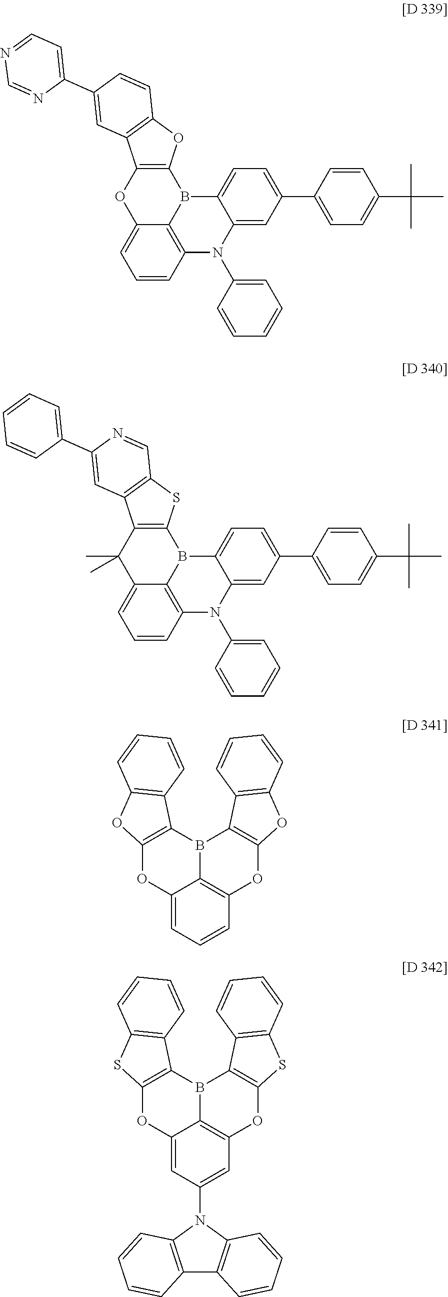

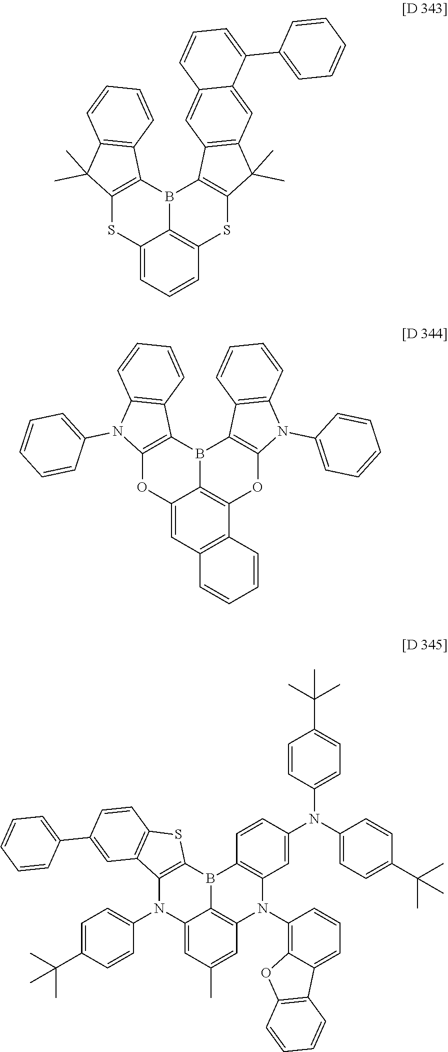 [SMILES: CC1(C)C2=C(B3C4=C(Sc5cccc(c53)S2)C(C)(C)c2cc3c(-c5ccccc5)cccc3cc24)c2ccccc21 ; c1ccc(-n2c3c(c4ccccc42)B2c4c(cc5ccccc5c4Oc4c2c2ccccc2n4-c2ccccc2)O3)cc1 ; Cc1cc2c3c(c1)N(c1cccc4c1oc1ccccc14)c1cc(N(c4ccc(C(C)(C)C)cc4)c4ccc(C(C)(C)C)cc4)ccc1B3c1sc3ccc(-c4ccccc4)cc3c1N2c1ccc(C(C)(C)C)cc1]

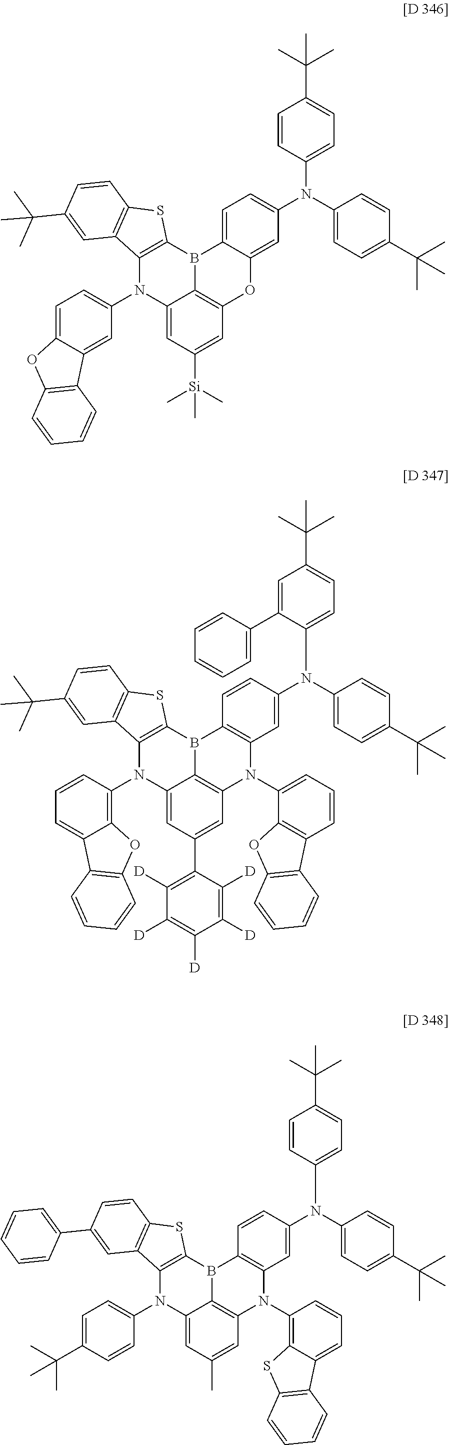

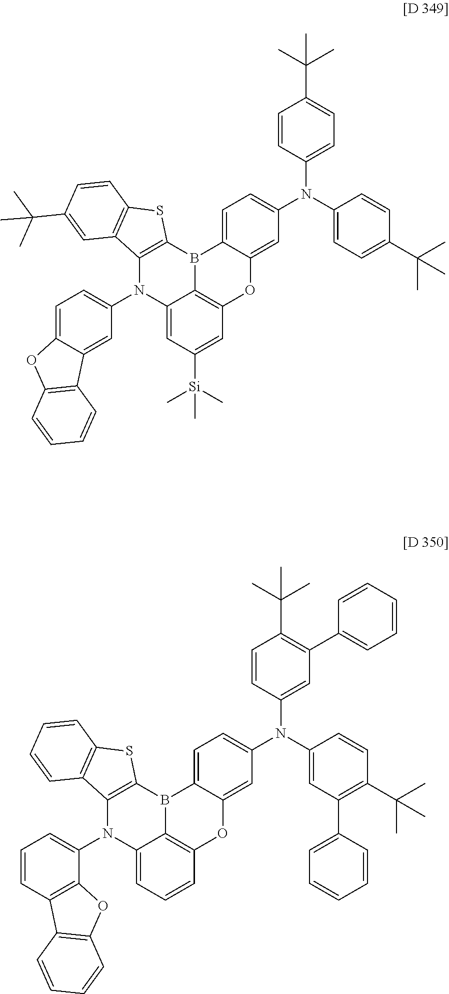 [SMILES: CC(C)(C)c1ccc(N(c2ccc(C(C)(C)C)cc2)c2ccc3c(c2)Oc2cc([Si](C)(C)C)cc4c2B3c2sc3ccc(C(C)(C)C)cc3c2N4c2ccc3oc4ccccc4c3c2)cc1 ; CC(C)(C)c1ccc(N(c2ccc3c(c2)Oc2cccc4c2B3c2sc3ccccc3c2N4c2cccc3c2oc2ccccc23)c2ccc(C(C)(C)C)c(-c3ccccc3)c2)cc1-c1ccccc1]

16. The organic light-emitting diode of claim 1, wherein the organic light emitting diode comprises at least one of a hole injection layer, a hole transport layer, a functional layer capable of both hole injection and hole transport, an electron transport layer, and an electron injection layer, in addition to the light-emitting layer.

17. The organic light emitting diode of claim 1, wherein the host within the light emitting layer further comprises one or more compounds different from the anthracene compound represented by Chemical Formula A, whereby two or more different host compounds are used in mixture in the light emitting layer.

18. The organic light emitting diode of claim 1, wherein the light emitting layer is a stacked light emitting layer in a bi- or multilayer structure comprising: a first light emitting layer containing a host represented by Chemical Formula A and a dopant represented by any one of Chemical Formulas D-6 and D-7; and a second light emitting layer.

19. The organic light emitting diode of claim 1, wherein the organic light-emitting diode is used for a device selected from among a flat display device; a flexible display device; a monochrome or grayscale flat illumination; and a monochrome or grayscale flexible illumination.
